(19) United States
(12) Patent Application Publication
(10) Pub. No.: US 2026/0356839 A1
(43) Pub. Date: Nov. 12, 2026

(54) DIRECT SYNTHESIS OF N-(3-SUBSTITUTED-CHROMAN-4-YL)-7H- PYRROLO[2,3-D]PYRIMIDIN-4-AMINES AND DERIVATIVES THEREOF

(71) Applicant: MITOKININ, INC., San Francisco, CA (US)

(72) Inventors: Nicholas Thomas HERTZ, San Francisco, CA (US); Robert DEVITA, Westerfield, NY (US); Paul O'SHEA, Princeton, NJ (US); Lorna DUFFY, Nottingham (GB); Abid SULEMAN, Nottingham (GB); Johan BARTHOLOMEUS, Montreal, Québec (CA); Philippe MCGEE, Québec (CA); Julien DANSEREAU, Québec (CA); Jeffrey SANTANDREA, Québec (CA)

(73) Assignee: MITOKININ, INC., San Francisco, CA (US)

(21) Appl. No.: 19/104,963

(22) PCT Filed: Aug. 21, 2023
(86) PCT No.: PCT/US2023/072592

§ 371 Date: Feb. 19, 2025

Related U.S. Application Data
(60) Provisional application No. 63/399,504, filed on Aug. 19, 2022.

(51) Int. Cl. C07D 487/04; C07D 311/22; C07D 413/04

(52) U.S. Cl. CPC C07D 487/04; C07D 311/22; C07D 413/04

(57) ABSTRACT

The present disclosure is directed to N-(3-substituted-chroman-4-yl)-7H-pyrrolo[2,3-d]pyrimidin-4-amine compounds useful as PINK1 modulators, compounds useful in preparing N-(3-substituted-chroman-4-yl)-7H-pyrrolo[2,3-d]pyrimidin-4-amine compounds, and methods of making and using same. This abstract is intended as a scanning tool for purposes of searching in the particular art and is not intended to be limiting of the present invention.

### CROSS-REFERENCE TO RELATED PATENT APPLICATIONS

This application claims priority to U.S. Appl. No. 63/399,504, entitled DIRECT SYNTHESIS OF N-(3-SUBSTITUTED-CHROMAN-4-YL)-7H-PYRROLO [2,3-D]PYRIMIDIN-4-AMINES AND DERIVATIVES THEREOF and filed Aug. 19, 2022, the content of which is incorporated herein by reference in its entirety for all purposes.

### BACKGROUND

Maintenance of mitochondrial function is essential for the health and survival of numerous cell types, including cardiomyoctes, hepatocytes, renal cells and neurons. Aberrant mitochondrial quality control has been demonstrated to be an important factor in the development of neurodegenerative diseases, kidney disease, and cardiomyopathy (Schapira, A. H. Mitochondrial disease. Lancet 379, 1825-1834, (2012) and Chen, Y. and Dorn, G. PINK1-Phosphorylated Mitofusin-2 Is a Parkin Receptor for Culling Damaged Mitochondria. Science 340, 471-475, (2013)). The mitochondrial kinase PTEN Induced Kinase 1 (PINK1) plays an important role in the mitochondrial quality control processes by responding to damage at the level of individual mitochondria. The PINK1 pathway has also been linked to the induction of mitochondrial biogenesis and, critically, to the reduction of mitochondrially-induced apoptosis. See e.g., Narendra, D. P. et al. PINK1 is selectively stabilized on impaired mitochondria to activate Parkin. PLoS Biol 8, e1000298 (2010), Wang, X., (2011). et al. PINK1 and Parkin target Miro for phosphorylation and degradation to arrest mitochondrial motility. Cell 147, 893-906, (2011), and Shin, J. H. et al. PARIS (ZNF746) repression of PGC-1alpha contributes to neurodegeneration in Parkinson's disease. Cell 144, 689-702, (2011).

Parkinson's Disease (PD) is one of the most common neurodegenerative disorders; however, no disease modifying therapies are currently approved to treat PD. Both environmental and genetic factors lead to progressive apoptosis of dopaminergic neurons, lowered dopamine levels, and, ultimately, PD. PINK1 kinase activity appears essential to mediate its neuroprotective activity. The regulation of mitochondrial movement, distribution, and clearance is a key part of neuronal oxidative stress response. Disruptions to these regulatory pathways have been shown to contribute to chronic neurodegenerative disease. See Schapira and Chen cited above.

Cardiomyopathy refers to a disease of cardiac muscle tissue, and it is estimated that cardiomyopathy accounts for 5-10% of the 5-6 million patients already diagnosed with heart failure in the United States. Based on etiology and pathophysiology, the World Health Organization created a classification of cardiomyopathy types which includes dilated cardiomyopathy, hypertrophic cardiomyopathy, restrictive cardiomyopathy, arrhythmogenic right ventricular cardiomyopathy, and unclassified cardiomyopathy. See e.g., Richardson P, et al. Report of the 1995 World Health Organization/International Society and Federation of Cardiology Task Force on the Definition and Classification of cardiomyopathies. Circulation 1996; 93:841. PINK1 kinase activity appears to mediate its cardio-protective activity. The regulation of mitochondrial movement, distribution, and clearance is a part of cardiac cell oxidative stress response. Disruptions to these regulatory pathways have been shown to contribute to cardiomyopathy. See Schapira and Chen cited above. Wang, X., (2011) et al. PINK1 and Parkin target Miro for phosphorylation and degradation to arrest mitochondrial motility Cell 147, 893-906, (2011) and Richardson P, et al. Report of the 1995 World Health Organization/International Society and Federation of Cardiology Task Force on the Definition and Classification of cardiomyopathies. Circulation 1996; 93:841. Several cases of adult-onset LS have also been reported recently. See e.g., Longo, D, et al. Harrison's Internal Medicine. 18th ed. (online), Ch. 238 (2011), Koh, H. & Chung, J. PINK1 as a molecular checkpoint in the maintenance of mitochondrial function and integrity, Mol Cells 34, 7-13, (2012), Martins-Branco, D. et al. Ubiquitin proteasome system in Parkinson's disease: a keeper or a witness? Exp Neurol, 238, 89-99, (2012), and Geisler, S. et al. The PINK1/Parkin-mediated mitophagy is compromised by PD-associated mutations. Autophagy 6, 871-878, (2010).

In vivo imaging techniques such as MRI reveal bilateral hyperintense lesions in the basal ganglia, thalamus, substantia nigra, brainstem, cerebellar white matter and cortex, cerebral white matter, or spinal cord of LS patients. See e.g., Longo cited above and Shin, J. H. et al. PARIS (ZNF746) repression of PGC-1alpha contributes to neurodegeneration in Parkinson's disease. Cell 144, 689-702, (2011), Henchcliffe, C. & Beal, M. F. Mitochondrial biology and oxidative stress in Parkinson disease pathogenesis. Nat Clin Pract Neurol 4, 600-609 (2008), Pridgeon, J. W., Olzmann, J. A., Chin, L. S. & Li, L. PINK1 Protects against Oxidative Stress by Phosphorylating Mitochondrial Chaperone TRAP1. PLoS Biol 5, e172 (2007), and Haque, M. E. et al. Cytoplasmic Pink1 activity protects neurons from dopaminergic neurotoxin MPTP. Proc Natl Acad Sci USA 105, 1716-1721 (2008). The lesions usually correlate with gliosis, demyelination, capillary proliferation, and/or necrosis See Geisler, S. et al. The PINK1/Parkin-mediated mitophagy is compromised by PD-associated mutations. Autophagy 6, 871-878, (2010) and Gautier, C. A., Kitada, T. & Shen, J. Loss of PINK1 causes mitochondrial functional defects and increased sensitivity to oxidative stress. Proc Natl Acad Sci USA 105, 11364-11369 (2008). Behavioral symptoms of LS patients can include (with a wide variety of clinical presentation) developmental retardation, hypotonia, ataxia, spasticity, dystonia, weakness, optic atrophy, defects in eye or eyelid movement, hearing impairment, breathing abnormalities, dysarthria, swallowing difficulties, failure to thrive, and gastrointestinal problems. See e.g., Wang and Richardson cited above, and Samaranch, L. et al. PINK1-linked parkinsonism is associated with Lewy body pathology. Brain 133, 1128-1142, (2010) and Merrick, K. A. et al. Switching Cdk2 on or off with small molecules to reveal requirements in human cell proliferation. Mol Cell 42, 624-636, (2011). The cause of death in most LS cases is unclear, and the lack of a genetic model to study the disease progression and cause of death has impeded the development of adequate treatment. Prognosis for LS (and most diseases resulting from mitochondrial dysfunction) is very poor; there is no cure and treatment is often ineffective.

Parkinson's Disease (PD) is one of the most common neurodegenerative disorders; however, no disease modifying therapies are currently approved to treat PD. Both environmental and genetic factors lead to progressive apoptosis of dopaminergic neurons, lowered dopamine levels, and, ultimately, PD. PINK1 kinase activity appears to mediate its neuroprotective activity. The regulation of mitochondrial movement, distribution, and clearance is a key part of neuronal oxidative stress response. Disruptions to these regulatory pathways have been shown to contribute to chronic neurodegenerative disease. See Schapira and Chen cited above. Despite the widespread therapeutic utility of modulators of PINK1 kinase activity, methods of making these compounds that are amenable to scale-up for large-scale manufacturing processes have remained elusive.

### SUMMARY

In accordance with the purpose(s) of the invention, as embodied and broadly described herein, the invention, in some embodiments, relates to N-(3-substituted-chroman-4-yl)-7H-pyrrolo[2,3-d]pyrimidin-4-amine compounds useful as PINK1 kinase modulators, compounds useful in preparing N-(3-substituted-chroman-4-yl)-7H-pyrrolo[2,3-d]pyrimidin-4-amine compounds, and methods of making and using same.

Thus, provided herein are compounds having a structure represented by a formula:

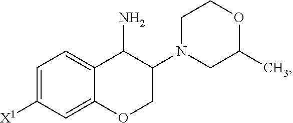

wherein X¹ is halogen, or a salt thereof.

Also provided herein are compounds having a structure represented by a formula:

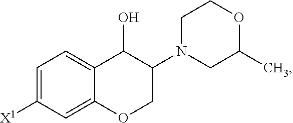

wherein X¹ is halogen, or a salt thereof.

Also provided herein are compounds having a structure represented by a formula:

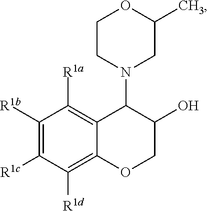

wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, provided that at least one of R^{1a}, R^{1b}, R^{1c}, and R^{1d} is not hydrogen, or a salt thereof.

Also provided herein are compounds having a structure represented by a formula:

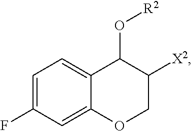

wherein X² is a halogen; and wherein R² is —C(O)(C1-C4 alkyl), or a salt thereof. Also provided herein are compounds having a structure:

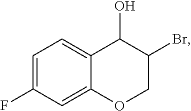 [SMILES: OC1c2ccc(F)cc2OCC1Br]

or a salt thereof.

Also provided herein are compounds having a structure represented by a formula:

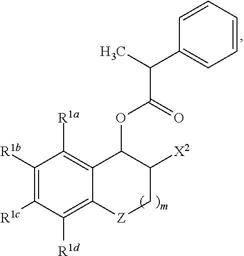

wherein m is 0 or 1; wherein X² is a halogen; wherein Z is selected from —CH₂—, —O—, and —NH—; and wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a salt thereof.

Also provided herein are methods comprising: (a) activating an alcohol having a structure represented by a formula:

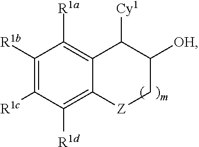

wherein m is 0 or 1; wherein Z is selected from —CH₂—, —O—, and —NH—; wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein Cy¹ is a 3- to 10-membered nitrogen-linked heterocycle substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a salt thereof, thereby forming an activated alcohol; and (b) displacing the activated alcohol via addition of an amine, thereby providing a chromanamine derivative having a structure represented by a formula:

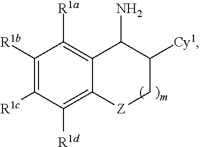

or a salt thereof.

Also provided herein are methods comprising: (a) epoxidizing a chromane derivative having a structure represented by a formula:

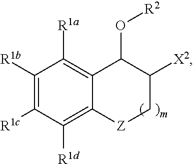

wherein m is 0 or 1; wherein X² is a halogen; and wherein Z is selected from —CH₂—, —O—, and —NH—; wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein R² is —C(O)(C1-C4 alkyl) or a residue of a chiral auxiliary group, or a salt thereof; and (b) reacting the epoxide with a cyclic amine having a structure represented by a formula:

Cy¹-H,
wherein Cy¹ is a 3- to 10-membered nitrogen-linked heterocycle substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, thereby making an alcohol having a structure represented by a formula:

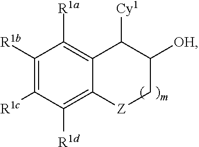

or a salt thereof.

Also provided herein are methods comprising activating a chromanol derivative having a structure represented by a formula:

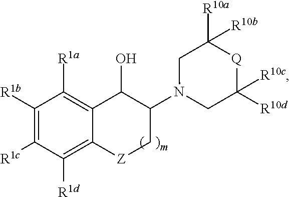

wherein m is 0 or 1; wherein Q is selected form —CH₂—, —O—, and —NR¹¹; wherein R¹¹, when present, is selected from hydrogen and C1-C4 alkyl; wherein Z is selected from —CH₂—, —O—, and —NH—; wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein each of R^{10a}, R^{10b}, R^{10c}, and R^{10d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a salt thereof, thereby forming an activated alcohol, and displacing the activated alcohol via addition of an amine, thereby providing a chromanamine derivative having a structure represented by a formula:

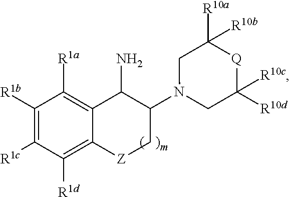

or a salt thereof.

Still other objects and advantages of the present disclosure will become readily apparent by those skilled in the art from the following detailed description, wherein it is shown and described only the preferred embodiments, simply by way of illustration of the best mode. As will be realized, the disclosure is capable of other and different embodiments, and its several details are capable of modifications in various obvious respects, without departing from the disclosure. Accordingly, the description is to be regarded as illustrative in nature and not as restrictive.

### BRIEF DESCRIPTION OF THE DRAWINGS

The accompanying figures, which are incorporated in and constitute a part of this specification, illustrate several embodiments and together with the description serve to explain the principles of the invention.

FIG. 1 shows a representative ¹H NMR spectra of 7-fluorochroman-4-ol.

FIG. 2 shows a representative ¹H NMR spectra of 7-fluoro-2H-chromene.

FIG. 3 shows a representative overlay of ¹H NMR spectra 7-fluoro-2H-chromene before and after storage at room temperature for 24 hours.

FIG. 4 shows a representative ¹H NMR spectra of 3-bromo-7-fluorochroman-4-ol.

FIG. 5 shows a representative chromatography trace of a 1:1 mixture of 3-bromo-7-fluorochroman-4-yl (R)-phenylpropanoate diastereomers.

FIG. 6A-D show representative spectral data of 3-bromo-7-fluorochroman-4-yl (R)-phenylpropanoate diastereomers. Specifically, FIG. 6A and FIG. 6B show representative ¹H NMR spectra of the first eluting isomer, (3R,4R)-3-bromo-7-fluorochroman-4-yl (R)-2-phenylpropanoate (FIG. 6A) and the second eluting isomer, (3S,4S)-3-bromo-7-fluorochroman-4-yl (R)-2-phenylpropanoate (FIG. 6B). FIG. 6C and FIG. 6D show representative overlays of the ¹H NMR spectra for each isomer. Both full (FIG. 6C) and expanded (FIG. 6D) versions of the overlay are shown.

FIG. 7 shows a representative ¹H NMR spectra of the intermediate epoxide mixture, as shown in Step 5 of the exemplary Route 1.

FIG. 8 shows a representative ¹H NMR spectra of (3S,4R)-7-fluoro-4-((R)-2-methylmorpholino)chroman-3-ol.

FIG. 9 shows a representative ¹H NMR spectra of (3S,4R)-7-fluoro-((R)-2-methylmorpholino)chroman-4-amine.

FIG. 10 shows a representative ¹H NMR spectra of N-((3R,4S)-7-fluoro-3-((R)-2-methylmorpholino)chroman-4-yl)-6-(trifluoromethyl)-7H-pyrrolo[2,3-d]pyrimidin-4-amine.

FIG. 11 shows representative data illustrating LCMS analysis of N-((3R,4S)-7-fluoro-3-((R)-2-methylmorpholino)chroman-4-yl)-6-(trifluoromethyl)-7H-pyrrolo[2,3-d]pyrimidin-4-amine.

FIG. 12A-C show representative spectral data of racemic-trans-3-bromo-7-fluorochroman-4-yl acetate. Specifically, ¹H NMR (FIG. 12A), mass spectrometry (FIG. 12B), and UPLC (FIG. 12C) spectra are shown.

FIG. 13 shows representative data illustrating separation of the bromohydrin isomers via supercritical fluid chromatography (SFC).

FIG. 14 shows representative data illustrating separation of the racemic bromohydrin and the acylated product via SFC.

FIG. 15A and FIG. 15B show representative ¹H NMR spectra of racemic-trans-7-fluoro-4-((R)-2-methylmorpholino)chroman-3-ol. Both full (FIG. 15A) and expanded (FIG. 15B) versions of the spectra are shown.

FIG. 16A-C show representative SFC traces of the enzymatic hydrolysis product mixture (FIG. 16A), the unreacted ester after chromatographic purification (FIG. 16B), and the hydrolysed product after chromatographic purification (FIG. 16C).

FIG. 17 shows a representative ¹H NMR spectra of ¹H NMR spectra of a mixture of 7-fluoro-4-((R)-2-methylmorpholino)chroman-3-ol isomers. As shown, the major product is the (3R,4S) isomer and the minor product is the (3S,4R) isomer.

FIG. 18A and FIG. 18B show representative SFC traces of the products obtained via enzymatic transesterification. As shown in FIG. 18B, the esterified product was obtained with >94% e.e. after chromatographic purification.

FIG. 19 shows a representative ¹H NMR spectra of 7-fluoro-4-((R)-2-methylmorpholino)chroman-3-ol isomers. As shown, the major product is the (3S,4R) isomer and the minor product is the (3R,4S) isomer.

FIG. 20 shows a representative SFC trace assigning the stereochemistry of bromohydrin and acylated products.

FIG. 21 shows a representative HPLC spectra for Compound 2 of Scheme 9.

FIG. 22 shows a representative HPLC spectra for Compound 3 of Scheme 9.

FIG. 23 shows a representative ¹H NMR of crude Compound 3 of Scheme 9.

FIG. 24 shows a representative HPLC spectra for Compound 4 of Scheme 9.

FIG. 25 shows a representative HPLC spectra for Compound 6 of Scheme 9.

FIG. 26 shows a representative HPLC spectra for the chiral Compound 6 of Scheme 9.

FIG. 27 shows a representative HPLC spectra for Compound 7 of Scheme 9.

FIGS. 28 and 29 shows representative HPLC spectra for Compound 8 of Scheme 9.

FIG. 30 shows a representative IR spectra for the amorphous end product of Scheme 9.

FIG. 31 shows a representative HPLC spectra for the amorphous end product of Scheme 9.

FIG. 32 shows a representative HPLC spectra for the desired end product of Scheme 9.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular embodiments only and is not intended to be limiting. Although any methods and materials similar or equivalent to those described herein can be used in the practice or testing of the present invention, example methods and materials are now described.

While embodiments of the present invention can be described and claimed in a particular statutory class, such as the system statutory class, this is for convenience only and one of skill in the art will understand that each embodiment of the present invention can be described and claimed in any statutory class. Unless otherwise expressly stated, it is in no way intended that any method or embodiment set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of embodiments described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

Listed below are definitions of various terms used to describe this invention. These definitions apply to the terms as they are used throughout this specification, unless otherwise limited in specific instances, either individually or as part of a larger group.

As used herein, the terms “a” or “an” means that “at least one” or “one or more” unless the context clearly indicates otherwise. The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in various embodiments, to A without B (optionally including elements other than B); in another embodiment, to B without A (optionally including elements other than A); in yet another embodiment, to both A and B (optionally including other elements); etc.

The term “or” as used herein shall only be interpreted as indicating exclusive alternatives (i.e., “one or the other but not both”) when preceded by terms of exclusivity, “either,” “one of,” “only one of,” or “exactly one of.”

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “about” means that the numerical value is approximate and small variations would not significantly affect the practice of the disclosed embodiments. Where a numerical limitation is used, unless indicated otherwise by the context, “about” means the numerical value can vary by ±10%, +/−5%, +/−4%, +/−3%, +/−2%, +/−1%, and remain within the scope of the disclosed embodiments.

The abbreviations used herein have their conventional meaning within the chemical and biological arts. The chemical structures and formulae set forth herein are constructed according to the standard rules of chemical valency known in the chemical arts.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” mean that the subsequently described event or circumstance can or cannot occur, and that the description includes instances where said event or circumstance occurs and instances where it does not.

As used herein, the term “diagnosed” means having been subjected to a physical examination by a person of skill, for example, a physician, and found to have a condition that can be diagnosed or treated by the compounds, compositions, or methods disclosed herein. In some embodiments of the disclosed methods, the subject has been diagnosed with a need for treatment of a disorder associated with PINK1 kinase activity such as, for example, a neurodegenerative disease, a mitochondrial disease, fibrosis, and/or cardiomyopathy, prior to the administering step. As used herein, the phrase “identified to be in need of treatment for a disorder,” or the like, refers to selection of a subject based upon need for treatment of the disorder. It is contemplated that the identification can, in some embodiments, be performed by a person different from the person making the diagnosis. It is also contemplated, in further embodiments, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various embodiments, a preparation can be administered therapeutically; that is, administered to treat an existing disease or condition. In further various embodiments, a preparation can be administered prophylactically; that is, administered for prevention of a disease or condition.

The term “contacting” as used herein refers to bringing a disclosed compound and a cell, target receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, “IC₅₀,” is intended to refer to the concentration of a substance (e.g., a compound or a drug) that is required for about 50% inhibition of a biological process, or component of a process, including a protein, subunit, organelle, ribonucleoprotein, etc. In some embodiments, an IC₅₀ can refer to the concentration of a substance that is required for about 50% inhibition in vivo, as further defined elsewhere herein. In some embodiments, an IC50 can refer to the concentration of a substance that is required for about 40% inhibition in vivo, as further defined elsewhere herein. In some embodiments, an IC50 can refer to the concentration of a substance that is required for about 30% inhibition in vivo, as further defined elsewhere herein. In some embodiments, an IC50 can refer to the concentration of a substance that is required for about 20% inhibition in vivo, as further defined elsewhere herein. In some embodiments, an IC50 can refer to the concentration of a substance that is required for about 10% inhibition in vivo, as further defined elsewhere herein. In some embodiments, an IC50 can refer to the concentration of a substance that is required for about 60% inhibition in vivo, as further defined elsewhere herein.

As used herein, “EC₅₀,” is intended to refer to the concentration of a substance (e.g., a compound or a drug) that is results in a half-maximal response (i.e., 50% of the maximum response) of a biological process, or component of a process, including a protein, subunit, organelle, ribonucleoprotein, etc. In some embodiments, an EC₅₀ can refer to the concentration of a substance that is required to achieve about 50% of the maximum response in vivo, as further defined elsewhere herein. In some embodiments, an EC50 can refer to the concentration of a substance that is required to achieve about 10% of the maximum response in vivo, as further defined elsewhere herein. In some embodiments, an EC50 can refer to the concentration of a substance that is required to achieve about 20% of the maximum response in vivo, as further defined elsewhere herein. In some embodiments, an EC50 can refer to the concentration of a substance that is required to achieve about 30% of the maximum response in vivo, as further defined elsewhere herein. In some embodiments, an EC50 can refer to the concentration of a substance that is required to achieve about 40% of the maximum response in vivo, as further defined elsewhere herein. In some embodiments, an EC50 can refer to the concentration of a substance that is required to achieve about 60% of the maximum response in vivo, as further defined elsewhere herein. In some embodiments, an EC50 can refer to the concentration of a substance that is required to achieve about 70% of the maximum response in vivo, as further defined elsewhere herein.

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radio-actively labeled forms, isomers, solvates and combinations thereof. The “combinations” mentioned in this context are refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

The term “leaving group” refers to an atom (or a group of atoms) with electron withdrawing ability that can be displaced as a stable species, taking with it the bonding electrons. Examples of suitable leaving groups include sulfonate esters, including triflate, mesylate, tosylate, brosylate, and halides.

As used herein, the term “substituted” is contemplated to include all permissible substituents of organic compounds. In a broad embodiment, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain embodiments, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The terms “halo” and “halogen” as used herein refer to an atom selected from fluorine (fluoro, —F), chlorine (chloro, —Cl), bromine (bromo, —Br), and iodine (iodo, —I).

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl,” as used herein, refers to a monovalent saturated, straight- or branched-chain hydrocarbon radical, having unless otherwise specified, 1-6 carbon atoms. Examples of alkyl radicals include, but are not limited to, methyl, ethyl, n-propyl, isopropyl, n-butyl, iso-butyl, sec-butyl, n-pentyl, tert-pentyl, neopentyl, sec-pentyl, 3-pentyl, sec-isopentyl, hexyl, 2-methylpentane, 3-methylpentane, 2,2-dimethylbutane, 2,3-dimentybutane and the like. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “heteroalkyl,” as used herein refers to an alkyl group containing at least one heteroatom. Suitable heteroatoms include, but are not limited to, O, N, Si, P, and S, wherein the nitrogen, phosphorous and sulfur atoms are optionally oxidized, and the nitrogen heteroatom is optionally quaternized. Heteroalkyls can be substituted as defined above for alkyl groups.

The term “haloalkyl” includes mono, poly, and perhaloalkyl groups where the halogens are independently selected from fluorine, chlorine, bromine, and iodine.

“Alkoxy” is an alkyl group which is attached to another moiety via an oxygen linker (—O(alkyl)). Non-limiting examples include methoxy, ethoxy, propoxy, and butoxy.

“Haloalkoxy” is a haloalkyl group which is attached to another moiety via an oxygen atom such as, e.g., but are not limited to —OCHCF₂ or —OCF₃.

The term “9- to 10-membered carbocyclyl” means a 9- or 10-membered monocyclic, bicyclic (e.g., a bridged or spiro bicyclic ring), polycyclic (e.g., tricyclic), or fused hydrocarbon ring system that is saturated or partially unsaturated. The term “9- to 10-membered carbocyclyl” also includes saturated or partially unsaturated hydrocarbon rings that are fused to one or more aromatic or partically saturated hydrocarbon rings (e.g., dihydroindenyl and tetrahydronaphthalenyl). Bridged bicyclic cycloalkyl groups include, without limitation, bicyclo[4.3.1]decanyl and the like. Spiro bicyclic cycloalkyl groups include, e.g., spiro[3.6]decanyl, spiro[4.5]decanyl, spiro[4.4]nonyl and the like. Fused cycloalkyl rings include, e.g., decahydronaphthalenyl, dihydroindenyl, decahydroazulenyl, octahydroazulenyl, tetrahydronaphthalenyl, and the like. It will be understood that when specified, optional substituents on a carbocyclyl (e.g., in the case of an optionally substituted cycloalkyl) may be present on any substitutable position and, include, e.g., the position at which the carbocyclyl group is attached.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, cycloheptyl, cyclooctyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein. In various aspects, the cycloalkyl group and heterocycloalkyl group can be monocyclic, bicyclic (e.g., bridged such as, for example, bicyclo[4.3.1]decanyl or spiro such as, for example, spiro[3.6]decanyl, spiro[4.5]decanyl, spiro[4.4]nonyl), polycyclic (e.g., tricyclic), or a fused hydrocarbon ring system that is saturated or partially unsaturated (e.g., decahydronaphthalenyl, dihydroindenyl, decahydroazulenyl, octahydroazulenyl, tetrahydronaphthalenyl).

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” The heterocycle can be monocyclic, bicyclic (e.g., spiro or bridged), polycyclic, or a fused system that is saturated or partially saturated. Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, ¹H-pyrazolo[4,3-c]pyridin-3-yl; ¹H-pyrrolo[3,2-b]pyridin-3-yl; and ¹H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “9-membered fused heterocyclyl” means a 9-membered saturated or partially unsaturated fused monocyclic heterocyclic ring comprising at least one oxygen heteroatom and optionally two to four additional heteroatoms independently selected from N, O, and S. The terms “heterocycle,” “heterocyclyl,” “heterocyclyl ring,” “heterocyclic group,” “heterocyclic moiety,” and “heterocyclic radical,” are used interchangeably herein. A heterocyclyl ring can be attached to its pendant group at any heteroatom or carbon atom that results in a stable structure. Examples of fused saturated or partially unsaturated heterocyclic radicals compristing at least one oxygen atom include, without limitation, dihydrobenzofuranyl, dihydrofuropyridinyl, octahydrobenzofuranyl, and the like. Where specified as being optionally substituted, substituents on a heterocyclyl (e.g., in the case of an optionally substituted heterocyclyl) may be present on any substitutable position and include, e.g., the position at which the heterocyclyl group is attached.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The term “5- or 6-membered heteroaryl” refers to a 5- or 6-membered aromatic radical containing 1-4 heteroatoms selected from N, O, and S. Nonlimiting examples include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, etc. When specified, optional substituents on a heteroaryl group may be present on any substitutable position and, include, e.g., the position at which the heteroaryl is attached.

The term “aldehyde” as used herein is represented by the formula —C(O) H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain embodiments, unless expressly indicated to the contrary, individual substituents can be further optionally substituted (i.e., further substituted or unsubstituted).

In some embodiments, a structure of a compound can be represented by a formula:

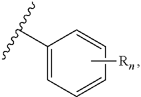

which is understood to be equivalent to a formula:

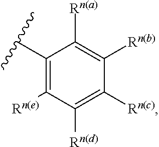

wherein n is typically an integer. That is, Rⁿ is understood to represent five independent substituents, R^{n(a)}, R^{n(b)}, R^{n(c)}, R^{n(d)}, R^{n(e)}. In each such case, each of the five Rⁿ can be hydrogen or a recited substituent. By “independent substituents,” it is meant that each R substituent can be independently defined. For example, if in one instance R^{n(a)} is halogen, then R^{n(b)} is not necessarily halogen in that instance.

In some yet further embodiments, a structure of a compound can be represented by a formula:

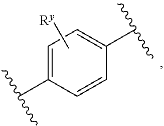

wherein R^{y} represents, for example, 0-2 independent substituents selected from A¹, A², and A³, which is understood to be equivalent to the groups of formulae:
wherein R^{y} represents 0 independent substituents

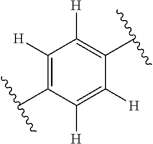

wherein R^{y} represents 1 independent substituent

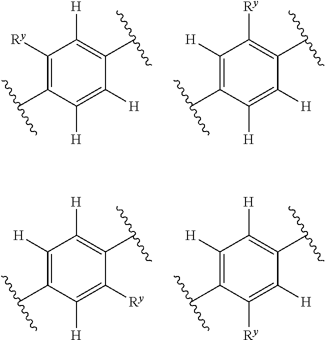

wherein R^{y} represents 2 independent substituents

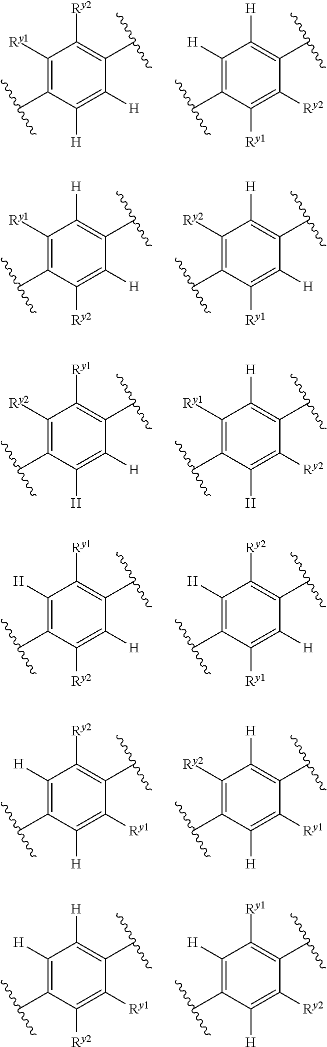

Again, by “independent substituents,” it is meant that each R substituent can be independently defined. For example, if in one instance R^{y1} is A¹, then R^{y2} is not necessarily A¹ in that instance.

In some further embodiments, a structure of a compound can be represented by a formula,

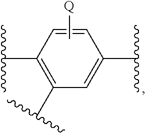

wherein, for example, Q comprises three substituents independently selected from hydrogen and A, which is understood to be equivalent to a formula:

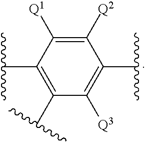

Again, by “independent substituents,” it is meant that each Q substituent is independently defined as hydrogen or A, which is understood to be equivalent to the groups of formulae:
wherein Q comprises three substituents independently selected from H and A

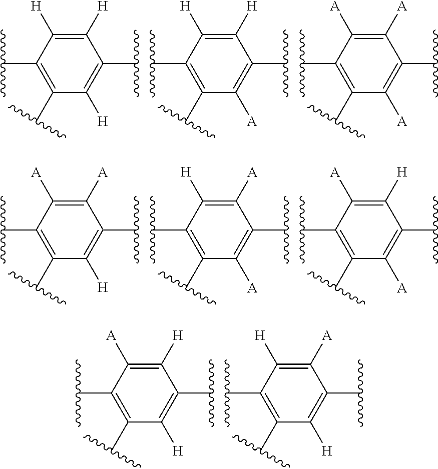

In some embodiment, the disclosed compounds exists as geometric isomers. “Geometric isomer” refers to isomers that differ in the orientation of substituent atoms in relationship to a cycloalkyl ring, i.e., cis or trans isomers. When a disclosed compound is named or depicted by structure without indicating a particular cis or trans geometric isomer form, it is to be understood that the name or structure encompasses one geometric isomer free of other geometric isomers, mixtures of geometric isomers, or mixtures enriched in one geometric isomer relative to its corresponding geometric isomer. When a particular geometric isomer is depicted, i.e., cis or trans, the depicted isomer is at least about 60%, 70%, 80%, 90%, 99%, or 99.9% by weight pure relative to the other geometric isomer.

The compounds described herein may be present in the form of pharmaceutically acceptable salts. For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Examples of pharmaceutically acceptable base addition salts include e.g., sodium, potassium, calcium, ammonium, organic amino, or magnesium salt. Specific pharmaceutical salts include, but are not limited to, salts of acids such as hydrochloric, phosphoric, hydrobromic, malic, glycolic, fumaric, sulfuric, sulfamic, suifanilic, formic, toluenesulfonie, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenyiacetic, alkanoic such as acetic, HOOC—(CH₂)n-COOH where n is 0-4, and the like.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

Disease, disorder, and condition are used interchangeably herein.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed, i.e., therapeutic treatment. In other embodiments, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. The term “preventing” refers to preventing a disease, disorder, or condition from occurring in a human or an animal that may be predisposed to the disease, disorder and/or condition, but has not yet been diagnosed as having it; and/or inhibiting the disease, disorder, or condition, i.e., arresting its development.

The term “effective amount” or “therapeutically effective amount” refers to an amount that is sufficient to achieve the desired result (e.g., that will elicit a biological or medical response of a subject. As a non-limiting example, a dosage from about 0.01 to about 100 mg/kg body weight/day) or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various embodiments, a preparation can be administered in a “prophylactically effective amount”; that is, an amount effective for prevention of a disease or condition.

As used herein, the term “salt” refers to acid or base salts of the compounds used in the methods of the present disclosure. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). In some embodiments, the subject is a human in need of treatment.

The term “associated” or “associated with” in the context of a substance or substance activity or function associated with a disease (e.g., a protein associated disease, a symptom associated with a cardiomyopathy, neurodegenerative disease, or symptom associated with Parkinson's disease) means that the disease (e.g., cardiomyopathy, neurodegenerative disease or Parkinson's disease) is caused by (in whole or in part), or a symptom of the disease is caused by (in whole or in part) the substance or substance activity or function. For example, a symptom of a disease or condition associated with a reduction in the level of PINK1 activity may be a symptom that results (entirely or partially) from a reduction in the level of PINK1 activity (e.g., loss of function mutation or gene deletion or modulation of PINK1 signal transduction pathway). As used herein, what is described as being associated with a disease, if a causative agent, could be a target for treatment of the disease. For example, a disease associated with PINK1, may be treated with an agent (e.g., compound as described herein) effective for increasing the level of activity of PINK1.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme (e.g., PINK1). In embodiments contacting includes allowing a compound described herein to interact with a protein or enzyme that is involved in a signaling pathway.

As defined herein, the term “inhibition,” “inhibit,” “inhibiting,” and the like in reference to a protein-modulator (e.g., antagonist) interaction means negatively affecting (e.g., decreasing) the activity or function of the protein relative to the activity or function of the protein in the absence of the modulator. In embodiments inhibition refers to reduction of a disease or symptoms of disease. In embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

The symbol “ [symbol] ” denotes the point of attachment of a chemical moiety to the remainder of a molecule or chemical formula.

As defined herein, the term “activation,” “activate,” “activating” and the like in reference to a protein-activator (e.g., agonist) interaction means positively affecting (e.g., increasing) the activity or function of the protein (e.g., PINK1) relative to the activity or function of the protein in the absence of the activator (e.g., compound described herein). In embodiments, activation refers to an increase in the activity of a signal transduction pathway or signaling pathway (e.g., PINK1 pathway). Thus, activation may include, at least in part, partially or totally increasing stimulation, increasing or enabling activation, or activating, sensitizing, or up-regulating signal transduction or enzymatic activity or the amount of a protein decreased in a disease (e.g., reduction of the level of PINK1 activity or protein associated with a cardiomyopathy or a neurodegenerative disease such as Parkinson's disease). Activation may include, at least in part, partially or totally increasing stimulation, increasing or enabling activation, or activating, sensitizing, or up-regulating signal transduction or enzymatic activity or the amount of a protein (e.g., PINK1) that may modulate the level of another protein or increase cell survival (e.g., increase in PINK1 activity may increase cell survival in cells that may or may not have a reduction in PINK1 activity relative to a non-disease control).

The term “modulator” refers to a composition that increases or decreases the level of a target molecule or the function of a target molecule. In embodiments, the modulator is a modulator of PINK1. In embodiments, the modulator is a modulator of PINK1 and is a compound that reduces the severity of one or more symptoms of a disease associated with PINK1 (e.g., reduction of the level of PINK1 activity or protein associated with a cardiomyopathy, neurodegenerative disease such as Parkinson's disease). In embodiments, a modulator is a compound that reduces the severity of one or more symptoms of a cardiomyopathy or neurodegenerative disease that is not caused or characterized by PINK1 (e.g., loss of PINK1 function) but may benefit from modulation of PINK1 activity (e.g., increase in level of PINK1 or PINK1 activity).

“Patient” or “subject in need thereof” refers to a living organism suffering from or prone to a disease or condition that can be treated by administration of a compound or pharmaceutical composition, as provided herein. Non-limiting examples include humans, other mammals, bovines, rats, mice, dogs, monkeys, goat, sheep, cows, deer, and other non-mammalian animals. In embodiments, a patient is human.

“Disease” or “condition” refer to a state of being or health status of a patient or subject capable of being treated with a compound, pharmaceutical composition, or method provided herein. In embodiments, the disease is a disease related to (e.g., characterized by) a reduction in the level of PINK1. In embodiments, the disease is a disease characterized by loss of dopamine-producing cells (e.g., Parkinson's disease). In embodiments, the disease is a disease characterized by neurodegeneration. In embodiments, the disease is a disease characterized by neural cell death. In embodiments, the disease is a disease characterized by a reduction in the level of PINK1 activity. In embodiments, the disease is Parkinson's disease. In embodiments, the disease is a neurodegenerative disease. In embodiments, the disease is a cardiomyopathy.

As used herein, the term “cardiomyopathy” refers to a disease condition that adversely affects cardiac cell tissue leading to a measurable deterioration in myocardial function (e.g., systolic function, diastolic function). Dilated cardiomyopathy is characterized by ventricular chamber enlargement with systolic dysfunction and no hypertrophy. Hypertrophic cardiomyopathy, is a genetic disease transmitted as an autosomal dominant trait. Hypertrophic cardiomyopathy is morphologically characterized by a hypertrophied and non-dialated left ventricle. Restrictive cardiomyopathy is characterized by nondialated nonhypertrophied morphology with diminished ventricular volume leading to poor ventricular filling. Arrhythmogenic right ventricular cardiomyopathy is an inheritable heart disease characterized by myocardial electric instability. Unclassified cardiomyopathy is a category for cardiomyopathies that do not match the features of any one of the other types. Unclassified cardiomyopathies may have features of multiple types or, for example, have the features of fibroelastosis, noncompacted myocardium, or systolic dysfunction with minimal dilatation.

As used herein, the term “neurodegenerative disease” refers to a disease or condition in which the function of a subject's nervous system becomes impaired. Examples of neurodegenerative diseases that may be treated with a compound or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, epilepsy, Friedreich ataxia, frontotemporal dementia, Gerstmann-Sträussler-Scheinker syndrome, Huntington's disease, HIV-associated dementia, Kennedy's disease, Krabbe's disease, kuru, Leigh's disease (Leigh syndrome), Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Shy-Drager syndrome, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, Tabes dorsalis, drug-induced Parkinsonism, progressive supranuclear palsy, corticobasal degeneration, multiple system atrophy, Idiopathic Parkinson's disease, Autosomal dominant Parkinson disease, Parkinson disease, familial, type 1 (PARK1), Parkinson disease 3, autosomal dominant Lewy body (PARK3), Parkinson disease 4, autosomal dominant Lewy body (PARK4), Parkinson disease 5 (PARK5), Parkinson disease 6, autosomal recessive early-onset (PARK6), Parkinson disease 2, autosomal recessive juvenile (PARK2), Parkinson disease 7, autosomal recessive early-onset (PARK7), Parkinson disease 8 (PARK8), Parkinson disease 9 (PARK9), Parkinson disease 10 (PARK10), Parkinson disease 11 (PARK11), Parkinson disease 12 (PARK12), Parkinson disease 13 (PARK13), or Mitochondrial Parkinson's disease. In embodiments, dysautonomia is not a neurodegenerative disease.

The term “signaling pathway” as used herein refers to a series of interactions between cellular and optionally extra-cellular components (e.g., proteins, nucleic acids, small molecules, ions, lipids) that conveys a change in one component to one or more other components, which in turn may convey a change to additional components, which is optionally propagated to other signaling pathway components.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., cardiomyopathy therapies including, for example, Angiotensin Converting Enzyme Modulators (e.g., Enalipril, Lisinopril), Angiotensin Receptor Blockers (e.g., Losartan, Valsartan), Beta Blockers (e.g., Lopressor, Toprol-XL), Digoxin, or Diuretics (e.g., Lasix; or Parkinson's disease therapies including, for example, levodopa, dopamine agonists (e.g., bromocriptine, pergolide, pramipexole, ropinirole, piribedil, cabergoline, apomorphine, lisuride), MAO-B modulators (e.g., selegiline or rasagiline), amantadine, anticholinergics, antipsychotics (e.g., clozapine), cholinesterase modulators, modafinil, or non-steroidal anti-inflammatory drugs.

The compound of the disclosure can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compositions of the present disclosure can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present disclosure can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present disclosure can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present disclosure can also be delivered as nanoparticles.

Pharmaceutical compositions provided by the present disclosure include compositions wherein the active ingredient (e.g., compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule (e.g., PINK1), and/or reducing, eliminating, or slowing the progression of disease symptoms (e.g., symptoms of cardiomyopathy or a neurodegeneration such as symptoms of Parkinson's disease). Determination of a therapeutically effective amount of a compound of the disclosure is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g., symptoms of cardiomyopathy or neurodegeneration such as Parkinson's disease and severity of such symptoms), kind of concurrent treatment, complications from the disease being treated or other health-related problems. Other therapeutic regimens or agents can be used in conjunction with the methods and compounds of Applicants' disclosure. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present disclosure should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds described herein can be used in combination with one another, with other active agents known to be useful in treating a disease associated neurodegeneration (e.g., Parkinson's disease, such as levodopa or amantadine, dopamine agonists (e.g., bromocriptine, pergolide, pramipexole, ropinirole, piribedil, cabergoline, apomorphine, lisuride, tetrabenazine), MAO-B modulators (e.g., selegiline or rasagiline), amantadine, anticholinergics, antipsychotics (e.g., clozapine, haloperidol), cholinesterase modulators, modafinil, or non-steroidal anti-inflammatory drugs), or with adjunctive agents that may not be effective alone, but may contribute to the efficacy of the active agent. The disclosure relates to a method of treating a neurodegenerative disorder in a subject in need thereof comprising administering to the subject a therapeutically effective amount of any of the compositions or pharmaceutical compositions disclosed herein. The disclosure relates to a method of treating a mitochondrial disorder in a subject in need thereof comprising administering to the subject a therapeutically effective amount of any of the compositions or pharmaceutical compositions disclosed herein. The disclosure relates to a method of treating cardiomyopathy in a subject in need thereof comprising administering to the subject a therapeutically effective amount of any of the compositions or pharmaceutical compositions disclosed herein.

The compounds described herein can be used in combination with one another, with other active agents known to be useful in treating a cardiomyopathy such as Angiotensin Converting Enzyme Modulators (e.g., Enalipril, Lisinopril), Angiotensin Receptor Blockers (e.g., Losartan, Valsartan), Beta Blockers (e.g., Lopressor, Toprol-XL), Digoxin, or Diuretics (e.g., Lasixdisease associated neurodegeneration (e.g., Parkinson's disease such as levodopa, dopamine agonists (e.g., bromocriptine, pergolide, pramipexole, ropinirole, piribedil, cabergoline, apomorphine, lisuride), MAO-B modulators (e.g., selegiline or rasagiline), amantadine, anticholinergics, antipsychotics (e.g., clozapine), cholinesterase modulators, modafinil, or non-steroidal anti-inflammatory drugs), or with adjunctive agents that may not be effective alone, but may contribute to the efficacy of the active agent.

In embodiments, co-administration includes administering one active agent within about 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In embodiments, co-administration can be accomplished by co-formulation, i.e., preparing a single pharmaceutical composition including both active agents. In other embodiments, the active agents can be formulated separately. In embodiments, the active and/or adjunctive agents may be linked or conjugated to one another. In embodiments, the compounds described herein may be combined with treatments for neurodegeneration such as surgery. In embodiments, the compounds described herein may be combined with treatments for cardiomyopathy such as surgery. In certain embodiments, provided is a method by administering any of the pharmaceutical compositions disclosed herein, wherein the pharmaceutical composition may include two or more compounds disclosed herein. In some cases, one of the compounds in the pharmaceutical compositions is one of the above disclosed active agents known to be useful in treating a disease associated neurodegeneration. In further embodiments, provided is a method of treatment comprising manufacturing one or more of compound disclosed herein (e.g., an intermediate disclosed herein); and administering any of the pharmaceutical compositions disclosed herein, wherein the pharmaceutical composition may include one or more compounds disclosed herein.

“PINK1” is used according to its common, ordinary meaning and refers to proteins of the same or similar names and functional fragments and homologs thereof. The term includes and recombinant or naturally occurring form of PINK1 (e.g., “PTEN induced putative kinase 1”; Entrez Gene 65018, OMIM 608309, UniProtKB Q9BXM7, and/or RefSeq (protein) NP_115785.1). The term includes PINK1 and variants thereof that maintain PINK1 activity (e.g., within at least about 30%, 40%, 50%, 60%, 70%, 80%, 90%, 95%, or 100% activity as compared to PINK1).

The term “neo-substrate” refers to a composition that is structurally similar to a composition that is a substrate for a protein or enzyme during the normal functioning of the protein or enzyme, but that is structurally distinct from the normal substrate of the protein or enzyme. In some embodiments, the composition comprises a neo-substrate. In embodiments, the neo-substrate is a better substrate for the protein or enzyme than the normal substrate (e.g., the reaction kinetics are better (e.g., faster), binding is stronger, turnover rate is higher, reaction is more productive, equilibrium favors product formation). In embodiments, the neo-substrate is a derivative of adenine, adenosine, AMP, ADP, or ATP. In embodiments, the neo-substrate is a substrate for PINK1. In embodiments, the neo-substrate is an N6 substituted adenine, adenosine, AMP, ADP, or ATP.

The term “derivative” as applied to a phosphate containing, monophosphate, diphosphate, or triphosphate group or moiety refers to a chemical modification of such group wherein the modification may include the addition, removal, or substitution of one or more atoms of the phosphate containing, monophosphate, diphosphate, or triphosphate group or moiety. In embodiments, such a derivative is a prodrug of the phosphate containing, monophosphate, diphosphate, or triphosphate group or moiety, which is converted to the phosphate containing, monophosphate, diphosphate, or triphosphate group or moiety from the derivative following administration to a subject, patient, cell, biological sample, or following contact with a subject, patient, cell, biological sample, or protein (e.g., enzyme). In an embodiment, a triphosphate derivative is a gamma-thio triphosphate. In an embodiment, a derivative is a phosphoramidate. In embodiments, the derivative of a phosphate containing, monophosphate, diphosphate, or triphosphate group or moiety is as described in Murakami et al. J. Med Chem., 2011, 54, 5902; Sofia et al., J. Med Chem. 2010, 53, 7202; Lam et al. ACC, 2010, 54, 3187; Chang et al., ACS Med Chem Lett., 2011, 2, 130; Furman et al., Antiviral Res., 2011, 91, 120; Vernachio et al., ACC, 2011, 55, 1843; Zhou et al, AAC, 2011, 44, 76; Reddy et al., BMCL, 2010, 20, 7376; Lam et al., J. Virol., 2011, 85, 12334; Sofia et al., J. Med. Chem., 2012, 55, 2481, Hecker et al., J. Med. Chem., 2008, 51, 2328; or Rautio et al., Nature Rev. Drug. Discov., 2008, 7, 255, all of which are incorporated herein by reference in their entirety for all purposes.

The term “mitochondrial dysfunction” is used in accordance with its ordinary meaning and refers to aberrant activity of function of the mitochondria, including for example aberrant respiratory chain activity, reactive oxygen species levels, calcium homeostasis, programmed cell death mediated by the mitochondria, mitochondrial fusion, mitochondrial fission, mitophagy, lipid concentrations in the mitochondrial membrane, and/or mitochondrial permeability transition.

As used herein, the term “mitochondrial disease” refers to a disease, disorder, or condition in which the function of a subject's mitochondria becomes impaired or dysfunctional. Examples of mitochondrial diseases that may be treated with a compound or method described herein include Alzheimer's disease, amyotrophic lateral sclerosis, Asperger's Disorder, Autistic Disorder, bipolar disorder, cancer, cardiomyopathy, Charcot Marie Tooth disease (CMT, including various subtypes such as CMT type 2b and 2b), Childhood Disintegrative Disorder (CDD), diabetes, diabetic nephropathy, epilepsy, Friedreich's Ataxia (FA), Hereditary motor and sensory neuropathy (HMSN), Huntington's Disease, Keams-Sayre Syndrome (KSS), Leber's Hereditary Optic Neuropathy (LHON, also referred to as Leber's Disease, Leber's Optic Atrophy (LOA), or Leber's Optic Neuropathy (LON)), Leigh Disease or Leigh Syndrome, macular degeneration, Mitochondrial Myopathy, Lactacidosis, and Stroke (MELAS), mitochondrial neurogastrointestinal encephalomyophathy (MNGIE), motor neuron diseases, Myoclonic Epilepsy With Ragged Red Fibers (MERRF), Neuropathy, ataxia, retinitis pigmentosa, and ptosis (NARP), Parkinson's disease, Peroneal muscular atrophy (PMA), Pervasive Developmental Disorder Not Otherwise Specified (PDD-NOS), renal tubular acidosis, Rett's Disorder, Schizophrenia, and types of stroke.

The term “oxidative stress” is used in accordance with its ordinary meaning and refers to aberrant levels of reactive oxygen species.

As used herein, the term “animal” includes, but is not limited to, humans and non-human vertebrates such as wild, domestic, and farm animals.

As used herein, the term “antagonize” or “antagonizing” means reducing or completely eliminating an effect, such as an activity of GPR109a.

As used herein, the phrase “anti-receptor effective amount” of a compound can be measured by the anti-receptor effectiveness of the compound. In some embodiments, an anti-receptor effective amount inhibits an activity of the receptor by at least 10%, by at least 20%, by at least 30%, by at least 40%, by at least 50%, by at least 60%, by at least 70%, by at least 80%, by at least 90%, or by at least 95%. In some embodiments, an “anti-receptor effective amount” is also a “therapeutically effective amount” whereby the compound reduces or eliminates at least one effect of GPR109a. In some embodiments, the effect is the B-arrestin effect. In some embodiments, the effect is the G-protein mediated effect.

As used herein, the term “carrier” means a diluent, adjuvant, or excipient with which a compound is administered. Pharmaceutical carriers can be liquids, such as water and oils, including those of petroleum, animal, vegetable or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “contacting” means bringing together of two elements in an in vitro system or an in vivo system. For example, “contacting” a compound disclosed herein with an individual or patient or cell includes the administration of the compound to an individual or patient, such as a human, as well as, for example, introducing a compound into a sample containing a cellular or purified preparation containing the compounds or pharmaceutical compositions disclosed herein.

As used herein, the terms “individual,” “subject” or “patient,” used interchangeably, means any animal, including mammals, such as mice, rats, other rodents, rabbits, dogs, cats, swine, cattle, sheep, horses, or primates, such as humans.

As used herein, the phrase “inhibiting activity,” such as enzymatic or receptor activity means reducing by any measurable amount the activity of PINK1.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

As used herein, the phrase “integer from X to Y” means any integer that includes the endpoints. For example, the phrase “integer from 1 to 5” means 1, 2, 3, 4, or 5.

As used herein, the term “isolated” means that the compounds described herein are separated from other components of either (a) a natural source, such as a plant or cell, or (b) a synthetic organic chemical reaction mixture, such as by conventional techniques.

As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the term “prodrug” means a derivative of a known direct acting drug, which derivative has enhanced delivery characteristics and therapeutic value as compared to the drug, and is transformed into the active drug by an enzymatic or chemical process. The compounds described herein also include derivatives referred to as prodrugs, which can be prepared by modifying functional groups present in the compounds in such a way that the modifications are cleaved, either in routine manipulation or in vivo, to the parent compounds. Examples of prodrugs include compounds of the disclosure as described herein that contain one or more molecular moieties appended to a hydroxyl, amino, sulfhydryl, or carboxyl group of the compound, and that when administered to a patient, cleaves in vivo to form the free hydroxyl, amino, sulfhydryl, or carboxyl group, respectively. Examples of prodrugs include, but are not limited to, acetate, formate and benzoate derivatives of alcohol and amine functional groups in the compounds of the disclosure. Preparation and use of prodrugs is discussed in T. Higuchi et al., “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference in their entireties.

As used herein, the term “purified” means that when isolated, the isolate contains at least 90%, at least 95%, at least 98%, or at least 99% of a compound described herein by weight of the isolate.

As used herein, the phrase “solubilizing agent” means agents that result in formation of a micellar solution or a true solution of the drug.

As used herein, the term “solution/suspension” means a liquid composition wherein a first portion of the active agent is present in solution and a second portion of the active agent is present in particulate form, in suspension in a liquid matrix.

As used herein, the phrase “substantially isolated” means a compound that is at least partially or substantially separated from the environment in which it is formed or detected.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size and sex of the subject. Optimal amounts can also be determined based on monitoring of the subject's response to treatment.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable subcombination.

It should be noted that any embodiment of the invention can optionally exclude one or more embodiment for purposes of claiming the subject matter.

In some embodiments, the compounds, or salts thereof, are substantially isolated. Partial separation can include, for example, a composition enriched in the compound of the disclosure. Substantial separation can include compositions containing at least about 50%, at least about 60%, at least about 70%, at least about 80%, at least about 90%, at least about 95%, at least about 97%, or at least about 99% by weight of the compound of the disclosure, or salt thereof. Methods for isolating compounds and their salts are routine in the art.

### B. N-(3-SUBSTITUTED-CHROMAN-4-YL)-7H-PYRROLO [2,3-D]PYRIMIDIN-4-AMINES

In various embodiments, disclosed are N-(3-substituted-chroman-4-yl)-7H-pyrrolo[2,3-d]pyrimidin-4-amine compounds that can be prepared by the disclosed methods. It is understood that a disclosed compound can be provided by the disclosed methods.

In various embodiments, the disclosed N-(3-substituted-chroman-4-yl)-7H-pyrrolo[2,3-d]pyrimidin-4-amine compounds are useful as PINK1 modulators.

In various embodiments, the disclosed N-(3-substituted-chroman-4-yl)-7H-pyrrolo[2,3-d]pyrimidin-4-amine compounds are useful in treating a disorder associated with PINK1 dysfunction in a mammal. In a further embodiment, the disclosed N-(3-substituted-chroman-4-yl)-7H-pyrrolo[2,3-d]pyrimidin-4-amine compounds are useful in treating a disorder associated with PINK1 dysfunction in a human. Examples of disorders associated with PINK1 dysfunction include, but are not limited to, neurodegenerative diseases, mitochondrial diseases, fibrosis, and cardiomyopathy.

### 1. Structure

In some embodiments, the present disclosure provides pyrrolopyridines having a structure represented by a formula:

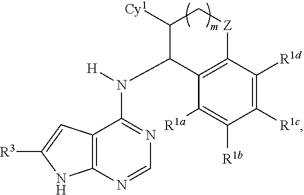

wherein m is 0 or 1; wherein Z is selected from —CH₂—, —O—, and —NH—; wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; wherein R³ is selected from C1-C6 haloalkyl, C1-C6 haloalkoxy, C1-C6 halohydroxy alkyl, and an unsubstituted 3- to 6-membered cycloalkyl; and wherein Cy¹ is a 3- to 10-membered nitrogen-linked heterocycle substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a pharmaceutically acceptable salt thereof.

In some further embodiments, the pyrrolopyridine has a structure represented by a formula:

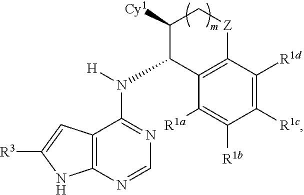

or a pharmaceutically acceptable salt thereof.

In some further embodiments, the pyrrolopyridine has a structure represented by a formula:

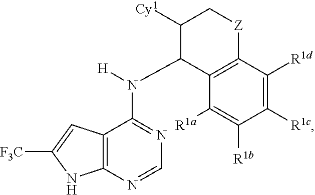

or a pharmaceutically acceptable salt thereof.

In some further embodiments, the pyrrolopyridine has a structure represented by a

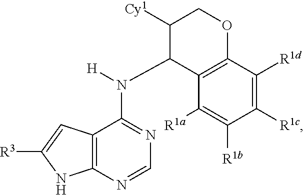

or a pharmaceutically acceptable salt thereof.

In some further embodiments, the pyrrolopyridine has a structure represented by a formula:

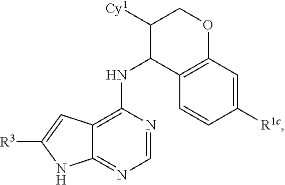

or a pharmaceutically acceptable salt thereof.

In some further embodiments, the pyrrolopyridine has a structure represented by a formula:

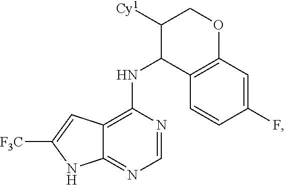

or a pharmaceutically acceptable salt thereof.

In some embodiments, the present disclosure provides pyrrolopyridines having a structure represented by a formula:

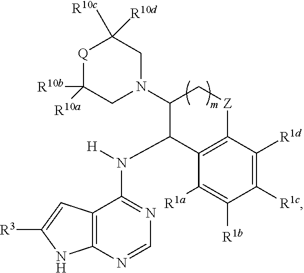

wherein m is 0 or 1; wherein Q is selected form —CH₂—, —O—, and —NR¹¹—; wherein R¹¹, when present, is selected from hydrogen and C1-C4 alkyl; wherein Z is selected from —CH₂—, —O—, and —NH—; wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; wherein R³ is selected from C1-C6 haloalkyl, C1-C6 haloalkoxy, C1-C6 halohydroxy alkyl, and an unsubstituted 3- to 6-membered cycloalkyl; and wherein each of R^{10a}, R^{10b}, R^{10c}, and R^{10d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a pharmaceutically acceptable salt thereof.

In some further embodiments, the pyrrolopyridine has a structure represented by a formula:

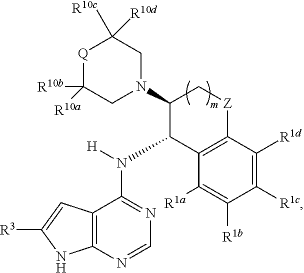

or a pharmaceutically acceptable salt thereof.

In some further embodiments, the pyrrolopyridine has a structure represented by a formula:

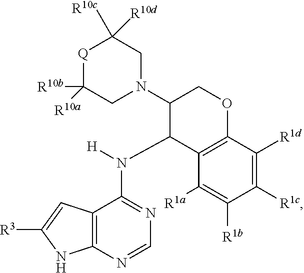

or a pharmaceutically acceptable salt thereof.

In some further embodiments, the pyrrolopyridine has a structure represented by a

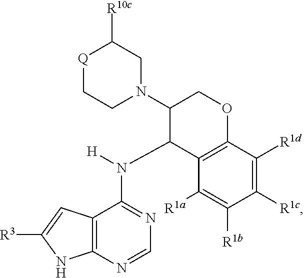

or a pharmaceutically acceptable salt thereof.

In some further embodiments, the pyrrolopyridine has a structure represented by a formula:

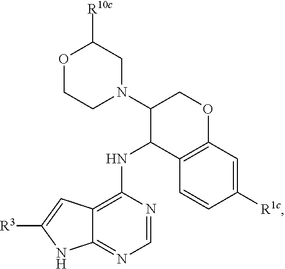

or a pharmaceutically acceptable salt thereof.

In some further embodiments, the pyrrolopyridine has a structure represented by a formula:

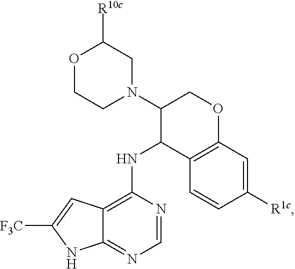

or a pharmaceutically acceptable salt thereof.

In some further embodiments, the pyrrolopyridine has a structure represented by a

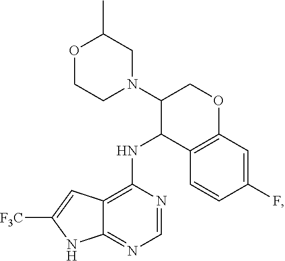 [SMILES: CC1CN(C2COc3cc(F)ccc3C2Nc2ncnc3[nH]c(C(F)(F)F)cc23)CCO1]

or a pharmaceutically acceptable salt thereof.

In some further embodiments, the pyrrolopyridine has a structure represented by a formula:

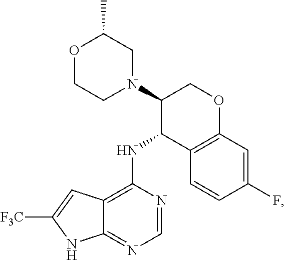 [SMILES: C[C@@H]1CN([C@H]2COc3cc(F)ccc3[C@@H]2Nc2ncnc3[nH]c(C(F)(F)F)cc23)CCO1]

or a pharmaceutically acceptable salt thereof.

In some further embodiments, the pyrrolopyridine has a structure represented by a formula:

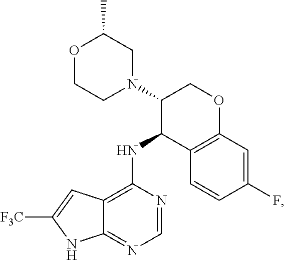 [SMILES: C[C@@H]1CN([C@@H]2COc3cc(F)ccc3[C@H]2Nc2ncnc3[nH]c(C(F)(F)F)cc23)CCO1]

or a pharmaceutically acceptable salt thereof.

In some embodiments, m is 0 or 1. In a further embodiment, m is 0. In a still further embodiment, m is 1. In some embodiments, the disclosure relates to a pharmaceutical composition comprising any of the above compounds with formulae above by synthesizing the compound by one or a combination of the steps provided herein.

Specific examples of compounds are provided in the EXAMPLES section and are included herein. Pharmaceutically acceptable salts as well as the neutral forms of these compounds are also included.

a. Q Groups

In some embodiments, Q is selected form —CH₂—, —O—, and —NR¹¹—. In a further embodiment, Q is selected from —CH₂— and —O—. In a still further embodiment, Q is selected from —CH₂— and —NR¹¹—. In yet a further embodiment, Q is selected from —O— and —NR¹¹—. In an even further embodiment, Q is —CH₂—. In a still further embodiment, Q is —O—. In yet a further embodiment, Q is —NR¹¹—.

b. Z Groups

In some embodiments, Z is selected from —CH₂—, —O—, and —NH—. In a further embodiment, Z is selected from —CH₂— and —O—. In a still further embodiment, Z is selected from —CH₂— and —NH—. In yet a further embodiment, Z is selected from —O— and —NH—. In an even further embodiment, Z is —CH₂—. In a still further embodiment, Z is —O—. In yet a further embodiment, Z is —NH—.

c. R^{1A}, R^{1B}, R^{1C}, and R^{1D} Groups

In some embodiments, each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In some embodiments, each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, provided that at least one of R^{1a}, R^{1b}, R^{1c}, and R^{1d} is not hydrogen, or a salt thereof.

In further embodiments, each of R^{1a}, R^{1b}, R^{1c}, and R^{1d} is independently selected from hydrogen, F, —Cl, —CN, —NH₂, —OH, —NO₂, methyl, ethyl, n-propyl, isopropyl, ethenyl, propenyl, —CH₂F, —CH₂CH₂F, —CH(CH₃) CH₂F, —CH₂CH₂CH₂F, —CH₂Cl, —CH₂CH₂Cl, —CH(CH₃) CH₂Cl, —CH₂CH₂CH₂Cl, —CH₂CN, —CH₂CH₂CN, —CH(CH₃) CH₂CN, —CH₂CH₂CH₂CN, —CH₂OH, —CH₂CH₂OH, —CH(CH₃) CH₂OH, —CH₂CH₂CH₂OH, methoxy, ethoxy, n-propoxy, isopropoxy, —OCF₃, —OCHF₂, —OCH₂F, —OCH₂CH₂F, —OCH(CH₃) CH₂F, —OCH₂CH₂CH₂F, —OCCl₃, —OCHCl₂, —OCH₂Cl, —OCH₂CH₂Cl, —OCH(CH₃) CH₂Cl, —OCH₂CH₂CH₂Cl, —NHCH₃, —NHCH₂CH₃, —NHCH(CH₃) CH₃, —NHCH₂CH₂CH₃, —N(CH₃)₂, —N(CH₃) CH₂CH₃, —N(CH₃) CH(CH₃) CH₃, and —N(CH₃) CH₂CH₂CH₃. In still further embodiments, each of R^{1a}, R^{1b}, R^{1c}, and R^{1d} is independently selected from hydrogen, F, —Cl, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, methoxy, ethoxy, —OCF₃, —OCHF₂, —OCH₂F, —OCH₂CH₂F, —OCCl₃, —OCHCl₂, —OCH₂Cl, —OCH₂CH₂Cl, —NHCH₃, —NHCH₂CH₃, —N(CH₃)₂, and —N(CH₃) CH₂CH₃. In yet further embodiments, each of R^{1a}, R^{1b}, R^{1c}, and R^{1d} is independently selected from hydrogen, F, —Cl, —CN, —NH₂, —OH, —NO₂, methyl, —CH₂F, —CH₂Cl, —CH₂CN, —CH₂OH, methoxy, —OCF₃, —OCHF₂, —OCH₂F, —OCCl₃, —OCHCl₂, —OCH₂Cl, —NHCH₃, and —N(CH₃)₂.

In further embodiments, each of R^{1a}, R^{1b}, R^{1c}, and R^{1d} is hydrogen.

In various embodiments, each of R^{1a}, R^{1b}, R^{1c}, and R^{1d} is independently selected from hydrogen, halogen, —CN, —NH₂, —OH, —NO₂, C1-C4 hydroxyalkyl, C1-C4 haloalkoxy, C1-C4 alkoxy. In further embodiments, each of R^{1a}, R^{1b}, R^{1c}, and R^{1d} is independently selected from hydrogen, F, —Cl, —CN, —NH₂, —OH, —NO₂, —CH₂OH, —CH₂CH₂OH, —CH(CH₃) CH₂OH, —CH₂CH₂CH₂OH, methoxy, ethoxy, n-propoxy, isopropoxy, —OCF₃, —OCHF₂, —OCH₂F, —OCH₂CH₂F, —OCH(CH₃) CH₂F, —OCH₂CH₂CH₂F, —OCCl₃, —OCHCl₂, —OCH₂Cl, —OCH₂CH₂Cl, —OCH(CH₃) CH₂Cl, and —OCH₂CH₂CH₂Cl. In still further embodiments, each of R^{1a}, R^{1b}, R^{1c}, and R^{1d} is independently selected from hydrogen, F, —Cl, —CN, —NH₂, —OH, —NO₂, —CH₂OH, —CH₂CH₂OH, methoxy, ethoxy, —OCF₃, —OCHF₂, —OCH₂F, —OCH₂CH₂F, —OCCl₃, —OCHCl₂, —OCH₂Cl, and —OCH₂CH₂Cl. In yet further embodiments, each of R^{1a}, R^{1b}, R^{1c}, and R^{1d} is independently selected from hydrogen, F, —Cl, —CN, —NH₂, —OH, —NO₂, —CH₂OH, methoxy, —OCF₃, —OCHF₂, —OCH₂F, —OCCl₃, —OCHCl₂, and —OCH₂Cl.

In various embodiments, each of R^{1a}, R^{1b}, R^{1c}, and R^{1d} is independently selected from hydrogen, halogen, —CN, —NH₂, —OH, —NO₂, C1-C4 alkylamino, and (C1-C4)(C1-C4) dialkylamino. In further embodiments, each of R^{1a}, R^{1b}, R^{1c}, and R^{1d} is independently selected from hydrogen, F, —Cl, —CN, —NH₂, —OH, —NO₂, —NHCH₃, —NHCH₂CH₃, —NHCH(CH₃) CH₃, —NHCH₂CH₂CH₃, —N(CH₃)₂, —N(CH₃) CH₂CH₃, —N(CH₃) CH(CH₃) CH₃, and —N(CH₃) CH₂CH₂CH₃. In still further embodiments, each of R^{1a}, R^{1b}, R^{1c}, and R^{1d} is independently selected from hydrogen, F, —Cl, —CN, —NH₂, —OH, —NO₂, —NHCH₃, —NHCH₂CH₃, —N(CH₃)₂, and —N(CH₃) CH₂CH₃. In yet further embodiments, each of R^{1a}, R^{1b}, R^{1c}, and R^{1d} is independently selected from hydrogen, F, —Cl, —CN, —NH₂, —OH, —NO₂, —NHCH₃, and —N(CH₃)₂.

In various embodiments, each of R^{1a}, R^{1b}, R^{1c}, and R^{1d} is independently selected from hydrogen, halogen, —CN, —NH₂, —OH, —NO₂, C1-C4 haloalkyl, and C1-C4 cyanoalkyl. In further embodiments, each of R^{1a}, R^{1b}, R^{1c}, and R^{1d} is independently selected from hydrogen, F, —Cl, —CN, —NH₂, —OH, —NO₂, —CH₂F, —CH₂CH₂F, —CH(CH₃) CH₂F, —CH₂CH₂CH₂F, —CH₂Cl, —CH₂CH₂Cl, —CH(CH₃) CH₂Cl, —CH₂CH₂CH₂Cl, —CH₂CN, —CH₂CH₂CN, —CH(CH₃) CH₂CN, and —CH₂CH₂CH₂CN. In still further embodiments, each of R^{1a}, R^{1b}, R^{1c}, and R^{1d} is independently selected from hydrogen, F, —Cl, —CN, —NH₂, —OH, —NO₂, —CH₂F, —CH₂CH₂F, —CH₂Cl, —CH₂CH₂Cl, —CH₂CN, and —CH₂CH₂CN. In yet further embodiments, each of R^{1a}, R^{1b}, R^{1c}, and R^{1d} is independently selected from hydrogen, F, —Cl, —CN, —NH₂, —OH, —NO₂, —CH₂F, —CH₂Cl, and —CH₂CN.

In various embodiments, each of R^{1a}, R^{1b}, R^{1c}, and R^{1d} is independently selected from hydrogen, halogen, —CN, —NH₂, —OH, —NO₂, C1-C4 alkyl, and C2-C4 alkenyl. In further embodiments, each of R^{1a}, R^{1b}, R^{1c}, and R^{1d} is independently selected from hydrogen, F, —Cl, —CN, —NH₂, —OH, —NO₂, methyl, ethyl, n-propyl, isopropyl, ethenyl, and propenyl. In still further embodiments, each of R^{1a}, R^{1b}, R^{1c}, and R^{1d} is independently selected from hydrogen, F, —Cl, —CN, —NH₂, —OH, —NO₂, methyl, ethyl, and ethenyl. In yet further embodiments, each of R^{1a}, R^{1b}, R^{1c}, and R^{1d} is independently selected from hydrogen, F, —Cl, —CN, —NH₂, —OH, —NO₂, and methyl.

In various embodiments, each of R^{1a}, R^{1b}, R^{1c}, and R^{1d} is independently selected from hydrogen and C1-C4 alkyl. In further embodiments, each of R^{1a}, R^{1b}, R^{1c}, and R^{1d} is independently selected from hydrogen, methyl, ethyl, n-propyl, and isopropyl. In still further embodiments, each of R^{1a}, R^{1b}, R^{1c}, and R^{1d} is independently selected from hydrogen, methyl, and ethyl. In yet further embodiments, each of R^{1a}, R^{1b}, R^{1c}, and R^{1d} is independently selected from hydrogen and methyl.

In various embodiments, each of R^{1a}, R^{1b}, R^{1c}, and R^{1d} is independently selected from hydrogen and halogen. In further embodiments, each of R^{1a}, R^{1b}, R^{1c}, and R^{1d} is independently selected from hydrogen, —F, —Cl, and —Br. In still further embodiments, each of R^{1a}, R^{1b}, R^{1c}, and R^{1d} is independently selected from hydrogen, —F, and —Cl. In yet further embodiments, each of R^{1a}, R^{1b}, R^{1c}, and R^{1d} is independently selected from hydrogen and —Cl. In still further embodiments, each of R^{1a}, R^{1b}, R^{1c}, and R^{1d} is independently selected from hydrogen and —F.

In some embodiments, each of R^{1a}, R^{1b}, R^{1c}, and R^{1d} is independently hydrogen, halogen, or C1-C4 alkyl. In further embodiments, each of R^{1a}, R^{1b}, R^{1c}, and R^{1d} is independently hydrogen, —F, —Cl, —Br, methyl, ethyl, n-propyl, or isopropyl. In still further embodiments, each of R^{1a}, R^{1b}, R^{1c}, and R^{d} is independently hydrogen, —F, —Cl, methyl, and ethyl. In yet further embodiments, each of R^{1a}, R^{1b}, R^{1c}, and R^{1d} is independently hydrogen, —F, and methyl.

In some embodiments, at least one of R^{1a}, R^{1b}, R^{1c}, and R^{1d} is halogen. In a further embodiment, at least one of R^{1a}, R^{1b}, R^{1c}, and R^{1d} is fluoro.

In some embodiments, R^{1c} is halogen. In a further embodiment, R^{1c} is fluoro.

d. R³ Groups

In some embodiments, R³ is selected from C1-C6 haloalkyl, C1-C6 haloalkoxy, C1-C6 halohydroxy alkyl, and an unsubstituted 3- to 6-membered cycloalkyl. In further embodiments, R³ is a 3- to 6-membered cycloalkyl, C1-C4 haloalkyl, C1-C4 haloalkoxy, or C1-C4 halohydroxyalkyl. In still further embodiments, R³ is a 3- to 6-membered cycloalkyl, —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, —CH₂CH₂Cl, —OCF₃, —OCHF₂, —OCH₂F, —OCH₂CF₃, —OCH₂CHF₂, —OCH₂CH₂F, —OCCl₃, —OCHCl₂, —OCH₂Cl, —OCH₂CCl₃, —OCH₂CHCl₂, —OCH₂CH₂Cl, —CH(OH) CF₃, —CH(OH)CHF₂, —CH(OH) CH₂F, —CH(OH) CCl₃, —CH(OH) CHCl₂, or —CH(OH) CH₂Cl. In yet further embodiments, R³ is a 3- to 6-membered cycloalkyl, —CF₃, —CHF₂, —CH₂F, —CCl₃, —CHCl₂, —CH₂Cl, —OCF₃, —OCHF₂, —OCH₂F, —OCCl₃, —OCHCl₂, or —OCH₂Cl.

In some embodiments, R³ is C1-C6 haloalkyl, C1-C6 haloalkoxy, or C1-C6 halohydroxyalkyl. In further embodiments, R³ is C1-C4 haloalkyl, C1-C4 haloalkoxy, or C1-C4 halohydroxyalkyl. In still further embodiments, R³ is —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, —CH₂CH₂Cl, —OCF₃, —OCHF₂, —OCH₂F, —OCH₂CF₃, —OCH₂CHF₂, —OCH₂CH₂F, —OCCl₃, —OCHCl₂, —OCH₂Cl, —OCH₂CCl₃, —OCH₂CHCl₂, —OCH₂CH₂Cl, —CH(OH) CF₃, —CH(OH)CHF₂, —CH(OH) CH₂F, —CH(OH) CCl₃, —CH(OH) CHCl₂, or —CH(OH) CH₂Cl. In yet further embodiments, R³ is —CF₃, —CHF₂, —CH₂F, —CCl₃, —CHCl₂, —CH₂Cl, —OCF₃, —OCHF₂, —OCH₂F, —OCCl₃, —OCHCl₂, or —OCH₂Cl.

In some embodiments, R³ is C1-C6 haloalkyl. In further embodiments, R³ is C1-C4 haloalkyl. In still further embodiments, R³ is —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, or —CH₂CH₂Cl. In yet further embodiments, R³ is —CF₃, —CHF₂, —CH₂F, —CCl₃, —CHCl₂, or —CH₂Cl.

In some embodiments, R³ is a 3- to 6-membered cycloalkyl. In further embodiments, R³ is a 3- to 5-membered cycloalkyl. In still further embodiments, R³ is a 3- to 4-membered cycloalkyl. In yet further embodiments, R³ is a 3-membered cycloalkyl. In an even further embodiment, R³ is a 4-membered cycloalkyl.

In some embodiments, R³ is hydrogen.

In some embodiments, R³ is hydrogen, halogen, (C1-C4)alkyl, or 3- to 6-membered cycloalkyl. In further embodiments, R³ is hydrogen.

In further embodiments, R³ is hydrogen, —F, —Cl, methyl, ethyl, n-propyl, isopropyl, or 3- to 6-membered cycloalkyl. In still further embodiments, R³ is hydrogen, —F, —Cl, methyl, ethyl, or 3- to 6-membered cycloalkyl. In yet further embodiments, R³ is hydrogen, —F, —Cl, methyl, or 3- to 6-membered cycloalkyl.

In further embodiments, R³ is hydrogen or (C1-C4)alkyl. In still further embodiments, R³ is hydrogen, methyl, ethyl, n-propyl, or isopropyl. In yet further embodiments, R³ is hydrogen, methyl, or ethyl. In an even further embodiment, R³ is hydrogen or ethyl. In still further embodiments, R³ is hydrogen or methyl.

In further embodiments, R³ is (C1-C4)alkyl. In still further embodiments, R³ is methyl, ethyl, n-propyl, or isopropyl. In yet further embodiments, R³ is methyl or ethyl. In an even further embodiment, R³ is ethyl. In still further embodiments, R³ is methyl.

In further embodiments, R³ is (C1-C4)alkyl. In still further embodiments, R³ is methyl, ethyl, n-propyl, isopropyl, halogenated methyl, halogenated ethyl, halogenated propyl, CF₃, CCl₃, or CBr₃. In yet further embodiments, R³ is methyl or ethyl. In an even further embodiment, R³ is ethyl. In still further embodiments, R³ is methyl. In still further embodiments, R³ is CF₃, CCl₃, or CBr₃.

In further embodiments, R³ is hydrogen or halogen. In still further embodiments, R³ is hydrogen, —F, —Cl, or —Br. In yet further embodiments, R³ is hydrogen, —F, or —Cl. In an even further embodiment, R³ is hydrogen or —F. In still further embodiments, R³ is hydrogen or —Cl.

In further embodiments, R³ is halogen. In still further embodiments, R³ is —F, —Cl, or —Br. In yet further embodiments, R³ is —F or —Cl. In an even further embodiment, R³ is —F. In still further embodiments, R³ is —Cl.

In further embodiments, R³ is hydrogen or 3- to 6-membered cycloalkyl. In still further embodiments, R³ is hydrogen, cyclopropyl, cyclobutyl, or cyclopentyl. In yet further embodiments, R³ is hydrogen, cyclopropyl, or cyclobutyl. In an even further embodiment, R³ is hydrogen or cyclopropyl. In some embodiments, R³ is not a methyl, ethyl or butyl. In some embodiments, R³ is not an acyclic alkyl chain comprising from about 1 to about 5 substituted or unsubstituted carbons.

In further embodiments, R³ is 3- to 6-membered cycloalkyl. In still further embodiments, R³ is 3- to 5-membered cycloalkyl. In yet further embodiments, R³ is 3- to 4-membered cycloalkyl. In an even further embodiment, R³ is cyclohexyl. In still further embodiments, R³ is cyclopentyl. In yet further embodiments, R³ is cyclobutyl. In an even further embodiment, R³ is cyclopropyl.

In further embodiments, R³ is a 3- to 6-membered cycloalkyl or a C1-C6 haloalkyl. In still further embodiments, R³ is cyclopropyl, cyclobutyl, cyclopentyl, CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, or —CH₂CH₂Cl. In yet further embodiments, R³ is cyclopropyl, cyclobutyl, CF₃, —CHF₂, —CH₂F, —CH₂CF₃, —CH₂CHF₂, —CH₂CH₂F, —CCl₃, —CHCl₂, —CH₂Cl, or —CH₂CCl₃. In even further embodiments, R³ is cyclopropyl, CF₃, —CHF₂, —CH₂F, —CCl₃, or —CHCl₂.

In further embodiments, R³ is a 3-membered cycloalkyl or —CF₃. In still further embodiments, R³ is a 3-membered cycloalkyl. In yet further embodiments, R³ is —CF₃.

e. R^{10A}, R^{10B}, R^{10C}, and R^{10D} Groups

In further embodiments, R³ is a 3-membered cycloalkyl or —CF₃. In still further embodiments, R³ is a 3-membered cycloalkyl. In yet further embodiments, R³ is —CF₃.

In some embodiments, each of R^{10a}, R^{10b}, R^{10c}, and R^{10d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, each of R^{10a}, R^{10b}, R^{10c}, and R^{10d} is independently selected from hydrogen, F, —Cl, —CN, —NH₂, —OH, —NO₂, methyl, ethyl, n-propyl, isopropyl, ethenyl, propenyl, —CH₂F, —CH₂CH₂F, —CH(CH₃) CH₂F, —CH₂CH₂CH₂F, —CH₂Cl, —CH₂CH₂Cl, —CH(CH₃) CH₂Cl, —CH₂CH₂CH₂Cl, —CH₂CN, —CH₂CH₂CN, —CH(CH₃) CH₂CN, —CH₂CH₂CH₂CN, —CH₂OH, —CH₂CH₂OH, —CH(CH₃) CH₂OH, —CH₂CH₂CH₂OH, methoxy, ethoxy, n-propoxy, isopropoxy, —OCF₃, —OCHF₂, —OCH₂F, —OCH₂CH₂F, —OCH(CH₃) CH₂F, —OCH₂CH₂CH₂F, —OCCl₃, —OCHCl₂, —OCH₂Cl, —OCH₂CH₂Cl, —OCH(CH₃) CH₂Cl, —OCH₂CH₂CH₂Cl, —NHCH₃, —NHCH₂CH₃, —NHCH(CH₃) CH₃, —NHCH₂CH₂CH₃, —N(CH₃)₂, —N(CH₃) CH₂CH₃, —N(CH₃) CH(CH₃) CH₃, and —N(CH₃) CH₂CH₂CH₃. In still further embodiments, each of R^{10a}, R^{10b}, R^{10c}, and R^{10d} is independently selected from hydrogen, F, —Cl, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, methoxy, ethoxy, —OCF₃, —OCHF₂, —OCH₂F, —OCH₂CH₂F, —OCCl₃, —OCHCl₂, —OCH₂Cl, —OCH₂CH₂Cl, —NHCH₃, —NHCH₂CH₃, —N(CH₃)₂, and —N(CH₃) CH₂CH₃. In yet further embodiments, each of R^{10a}, R^{10b}, R^{10c}, and R^{10d} is independently selected from hydrogen, F, —Cl, —CN, —NH₂, —OH, —NO₂, methyl, —CH₂F, —CH₂Cl, —CH₂CN, —CH₂OH, methoxy, —OCF₃, —OCHF₂, —OCH₂F, —OCCl₃, —OCHCl₂, —OCH₂Cl, —NHCH₃, and —N(CH₃)₂.

In further embodiments, each of R^{10a}, R^{10b}, R^{10c}, and R^{10d} is hydrogen.

In various embodiments, each of R^{10a}, R^{10b}, R^{10c}, and R^{10d} is independently selected from hydrogen, halogen, —CN, —NH₂, —OH, —NO₂, C1-C4 hydroxyalkyl, C1-C4 haloalkoxy, C1-C4 alkoxy. In further embodiments, each of R^{10a}, R^{10b}, R^{10c}, and R^{10d} is independently selected from hydrogen, F, —Cl, —CN, —NH₂, —OH, —NO₂, —CH₂OH, —CH₂CH₂OH, —CH(CH₃) CH₂OH, —CH₂CH₂CH₂OH, methoxy, ethoxy, n-propoxy, isopropoxy, —OCF₃, —OCHF₂, —OCH₂F, —OCH₂CH₂F, —OCH(CH₃) CH₂F, —OCH₂CH₂CH₂F, —OCCl₃, —OCHCl₂, —OCH₂Cl, —OCH₂CH₂Cl, —OCH(CH₃) CH₂Cl, and —OCH₂CH₂CH₂Cl. In still further embodiments, each of R^{10a}, R^{10b}, R^{10c}, and R^{10d} is independently selected from hydrogen, F, —Cl, —CN, —NH₂, —OH, —NO₂, —CH₂OH, —CH₂CH₂OH, methoxy, ethoxy, —OCF₃, —OCHF₂, —OCH₂F, —OCH₂CH₂F, —OCCl₃, —OCHCl₂, —OCH₂Cl, and —OCH₂CH₂Cl. In yet further embodiments, each of R^{10a}, R^{10b}, R^{10c}, and R^{10d} is independently selected from hydrogen, F, —Cl, —CN, —NH₂, —OH, —NO₂, —CH₂OH, methoxy, —OCF₃, —OCHF₂, —OCH₂F, —OCCl₃, —OCHCl₂, and —OCH₂Cl.

In various embodiments, each of R^{10a}, R^{10b}, R^{10c}, and R^{10d} is independently selected from hydrogen, halogen, —CN, —NH₂, —OH, —NO₂, C1-C4 alkylamino, and (C1-C4)(C1-C4) dialkylamino. In further embodiments, each of R^{10a}, R^{10b}, R^{10c}, and R^{10d} is independently selected from hydrogen, F, —Cl, —CN, —NH₂, —OH, —NO₂, —NHCH₃, —NHCH₂CH₃, —NHCH(CH₃) CH₃, —NHCH₂CH₂CH₃, —N(CH₃)₂, —N(CH₃) CH₂CH₃, —N(CH₃) CH(CH₃) CH₃, and —N(CH₃) CH₂CH₂CH₃. In still further embodiments, each of R^{10a}, R^{10b}, R^{10c}, and R^{10d} is independently selected from hydrogen, F, —Cl, —CN, —NH₂, —OH, —NO₂, —NHCH₃, —NHCH₂CH₃, —N(CH₃)₂, and —N(CH₃) CH₂CH₃. In yet further embodiments, each of R^{10a}, R^{10b}, R^{10c}, and R^{10d} is independently selected from hydrogen, F, —Cl, —CN, —NH₂, —OH, —NO₂, —NHCH₃, and —N(CH₃)₂.

In various embodiments, each of R^{10a}, R^{10b}, R^{10c}, and R^{10d} is independently selected from hydrogen, halogen, —CN, —NH₂, —OH, —NO₂, C1-C4 haloalkyl, and C1-C4 cyanoalkyl. In further embodiments, each of R^{10a}, R^{10b}, R^{10c}, and R^{10d} is independently selected from hydrogen, F, —Cl, —CN, —NH₂, —OH, —NO₂, —CH₂F, —CH₂CH₂F, —CH(CH₃) CH₂F, —CH₂CH₂CH₂F, —CH₂Cl, —CH₂CH₂Cl, —CH(CH₃) CH₂Cl, —CH₂CH₂CH₂Cl, —CH₂CN, —CH₂CH₂CN, —CH(CH₃) CH₂CN, and —CH₂CH₂CH₂CN. In still further embodiments, each of R^{10a}, R^{10b}, R^{10c}, and R^{10d} is independently selected from hydrogen, F, —Cl, —CN, —NH₂, —OH, —NO₂, —CH₂F, —CH₂CH₂F, —CH₂Cl, —CH₂CH₂Cl, —CH₂CN, and —CH₂CH₂CN. In yet further embodiments, each of R^{10a}, R^{10b}, R^{10c}, and R^{10d} is independently selected from hydrogen, F, —Cl, —CN, —NH₂, —OH, —NO₂, —CH₂F, —CH₂Cl, and —CH₂CN.

In various embodiments, each of R^{10a}, R^{10b}, R^{10c}, and R^{10d} is independently selected from hydrogen, halogen, —CN, —NH₂, —OH, —NO₂, C1-C4 alkyl, and C2-C4 alkenyl. In further embodiments, each of R^{10a}, R^{10b}, R^{10c}, and R^{10d} is independently selected from hydrogen, F, —Cl, —CN, —NH₂, —OH, —NO₂, methyl, ethyl, n-propyl, isopropyl, ethenyl, and propenyl. In still further embodiments, each of R^{10a}, R^{10b}, R^{10c}, and R^{10d} is independently selected from hydrogen, F, —Cl, —CN, —NH₂, —OH, —NO₂, methyl, ethyl, and ethenyl. In yet further embodiments, each of R^{10a}, R^{10b}, R^{10c}, and R^{10d} is independently selected from hydrogen, F, —Cl, —CN, —NH₂, —OH, —NO₂, and methyl.

In various embodiments, each of R^{10a}, R^{10b}, R^{10c}, and R^{10d} is independently selected from hydrogen and C1-C4 alkyl. In further embodiments, each of R^{10a}, R^{10b}, R^{10c}, and R^{10d} is independently selected from hydrogen, methyl, ethyl, n-propyl, and isopropyl. In still further embodiments, each of R^{10a}, R^{10b}, R^{10c}, and R^{10d} is independently selected from hydrogen, methyl, and ethyl. In yet further embodiments, each of R^{10a}, R^{10b}, R^{10c}, and R^{10d} is independently selected from hydrogen and methyl.

In various embodiments, each of R^{10a}, R^{10b}, R^{10c}, and R^{10d} is independently selected from hydrogen and halogen. In further embodiments, each of R^{10a}, R^{10b}, R^{10c}, and R^{10d} is independently selected from hydrogen, —F, —Cl, and —Br. In still further embodiments, each of R^{10a}, R^{10b}, R^{10c}, and R^{10d} is independently selected from hydrogen, —F, and —Cl. In yet further embodiments, each of R^{10a}, R^{10b}, R^{10c}, and R^{10d} is independently selected from hydrogen and —Cl. In still further embodiments, each of R^{10a}, R^{10b}, R^{10c}, and R^{10d} is independently selected from hydrogen and —F.

In some embodiments, each of R^{10a}, R^{10b}, R^{10c}, and R^{10d} is independently hydrogen, halogen, or C1-C4 alkyl. In further embodiments, each of R^{10a}, R^{10b}, R^{10c}, and R^{10d} is independently hydrogen, —F, —Cl, —Br, methyl, ethyl, n-propyl, or isopropyl. In still further embodiments, each of R^{10a}, R^{10b}, R^{10c}, and R^{10d} is independently hydrogen, —F, —Cl, methyl, and ethyl. In yet further embodiments, each of R^{10a}, R^{10b}, R^{10c}, and R^{10d} is independently hydrogen, —F, and methyl.

f. R¹¹ Groups

In some embodiments, R¹¹, when present, is selected from hydrogen and C1-C4 alkyl. In further embodiments, R¹¹, when present, is selected from hydrogen, methyl, ethyl, n-propyl, and isopropyl. In still further embodiments, R¹¹, when present, is selected from hydrogen, methyl, and ethyl. In yet further embodiments, R¹¹, when present, is selected from hydrogen and ethyl. In even further embodiments, R¹¹, when present, is selected from hydrogen and methyl.

In some embodiments, R¹¹, when present, is C1-C4 alkyl. In further embodiments, R¹¹, when present, is selected from methyl, ethyl, n-propyl, and isopropyl. In still further embodiments, R¹¹, when present, is selected from methyl and ethyl. In yet further embodiments, R¹¹, when present, is ethyl. In even further embodiments, R¹¹, when present, is methyl.

In some embodiments, R¹¹, when present, is hydrogen.

g. Cy¹ Groups

In some embodiments, Cy¹ is a 3- to 10-membered nitrogen-linked heterocycle substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In a further embodiment, Cy¹ is a 3- to 10-membered nitrogen-linked heterocycle substituted with 0, 1, 2, or 3 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In a still further embodiment, Cy¹ is a 3- to 10-membered nitrogen-linked heterocycle substituted with 0, 1, or 2 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet a further embodiment, Cy¹ is a 3- to 10-membered nitrogen-linked heterocycle substituted with 0 or 1 group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In an even further embodiment, Cy¹ is a 3- to 10-membered nitrogen-linked heterocycle monosubstituted with a group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In a still further aspect, Cy¹ is a 3- to 10-membered nitrogen-linked heterocycle monosubstituted with a C1-C4 alkyl group. In yet a further aspect, Cy¹ is a 3- to 10-membered nitrogen-linked heterocycle monosubstituted with a methyl group. In an even further aspect, Cy¹ is an unsubstituted 3- to 10-membered nitrogen-linked heterocycle.

In some embodiments, Cy¹ is selected from pyrrolidinyl, imidazolidinyl, piperidinyl, piperazinyl, and morpholinyl, and is substituted with 0, 1, 2, 3, 4, 5, 6, 7, or 8 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In a further embodiment, Cy¹ is selected from pyrrolidinyl, imidazolidinyl, piperidinyl, piperazinyl, and morpholinyl, and is substituted with 0, 1, 2, or 3 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In a still further embodiment, Cy¹ is selected from pyrrolidinyl, imidazolidinyl, piperidinyl, piperazinyl, and morpholinyl, and is substituted with 0, 1, or 2 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet a further embodiment, Cy¹ is a 3- to 10-membered nitrogen-linked heterocycle substituted with 0 or 1 group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In an even further embodiment, Cy¹ is selected from pyrrolidinyl, imidazolidinyl, piperidinyl, piperazinyl, and morpholinyl, and is monosubstituted with a group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In a still further aspect, Cy¹ is selected from pyrrolidinyl, imidazolidinyl, piperidinyl, piperazinyl, and morpholinyl, and is monosubstituted with a C1-C4 alkyl group. In yet a further aspect, Cy¹ is selected from pyrrolidinyl, imidazolidinyl, piperidinyl, piperazinyl, and morpholinyl, and is monosubstituted with a methyl group. In an even further aspect, Cy¹ is selected from pyrrolidinyl, imidazolidinyl, piperidinyl, piperazinyl, and morpholinyl, and is unsubstituted.

In some embodiments, Cy¹ is a structure represented by a formula:

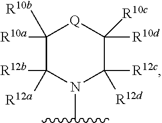

wherein Q is selected form —CH₂—, —O—, and —NR¹¹; wherein R¹¹, when present, is selected from hydrogen and C1-C4 alkyl; wherein each of R^{10a}, R^{10b}, R^{10c}, and R^{10d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein each of R^{12a}, R^{12b}, R^{12c}, and R^{12d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino.

In some embodiments, Cy¹ is a structure represented by a formula:

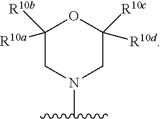

In some embodiments, Cy¹ is a structure represented by a formula:

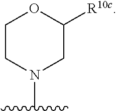

In some embodiments, Cy¹ is a structure represented by a formula:

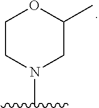 [SMILES: *N1CCOC(C)C1]

In some embodiments, Cy¹ is a structure represented by a formula:

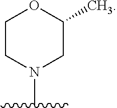 [SMILES: *N1CCO[C@H](C)C1]

### 2. EXAMPLE N-(3-substituted-chroman-4-yl)-7H-pyrrolo[2,3-d]pyrimidin-4-amine Compounds

In some embodiments, a compound can be present as the following structure:

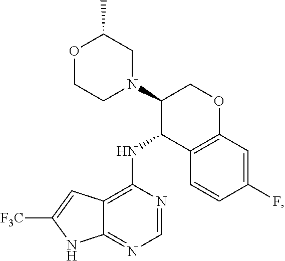 [SMILES: C[C@@H]1CN([C@H]2COc3cc(F)ccc3[C@@H]2Nc2ncnc3[nH]c(C(F)(F)F)cc23)CCO1]

or a pharmaceutically acceptable salt thereof.

### C. COMPOUNDS

In various embodiments, disclosed are compounds useful in the disclosed methods. It is understood that a disclosed compound can be provided by the disclosed methods.

In various embodiments, the disclosed compounds are useful as intermediates in the synthesis of N-(3-substituted-chroman-4-yl)-7H-pyrrolo[2,3-d]pyrimidin-4-amine compounds useful as PINK1 modulators.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Structure

In some embodiments, the present disclosure provides compounds having a structure represented by a formula:

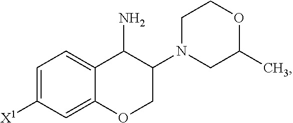

wherein X¹ is halogen, or a salt thereof.

In some further embodiments, the compound has a structure represented by a formula:

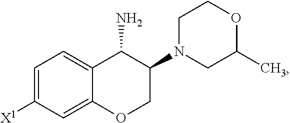

or a salt thereof.

In some further embodiments, the compound has a structure represented by a formula:

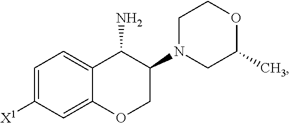

or a salt thereof.

In some further embodiments, the compound is:

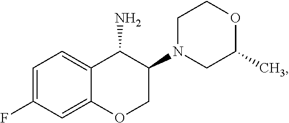 [SMILES: C[C@@H]1CN([C@H]2COc3cc(F)ccc3[C@@H]2N)CCO1]

or a salt thereof.

In some embodiments, the present disclosure provides compounds having a structure represented by a formula:

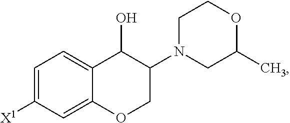

wherein X¹ is halogen, or a salt thereof.

In some further embodiments, the compound is:

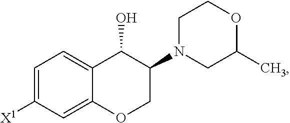

or a salt thereof.

In some further embodiments, the compound is:

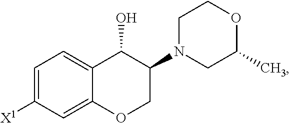

or a salt thereof.

In some further embodiments, the compound is:

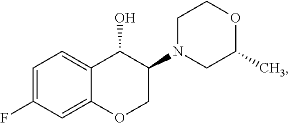 [SMILES: C[C@@H]1CN([C@H]2COc3cc(F)ccc3[C@@H]2O)CCO1]

or a salt thereof.

In some embodiments, the present disclosure provides compounds having a structure represented by a formula:

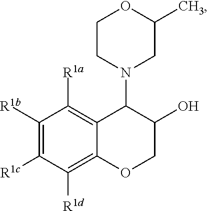

wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, provided that at least one of R^{1a}, R^{1b}, R^{1c}, and R^{1d} is not hydrogen, or a salt thereof.

In some further embodiments, the compound has a structure represented by a formula:

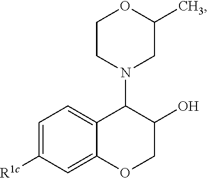

or a salt thereof.

In some further embodiments, the compound has a structure represented by a formula:

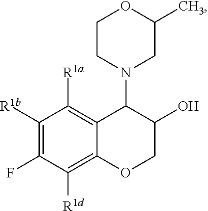

or a salt thereof.

In some further embodiments, the compound has a structure represented by a formula:

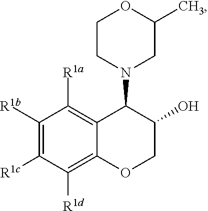

or a salt thereof.

In some further embodiments, the compound has a structure represented by a formula:

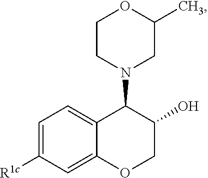

or a salt thereof.

In some further embodiments, the compound has a structure represented by a formula:

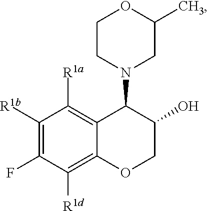

or a salt thereof.

In some further embodiments, the compound has a structure represented by a formula:

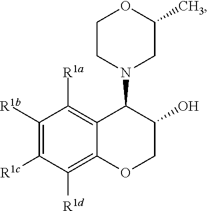

or a salt thereof.

In some further embodiments, the compound has a structure represented by a formula:

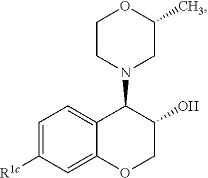

or a salt thereof.

In some further embodiments, the compound has a structure represented by a formula:

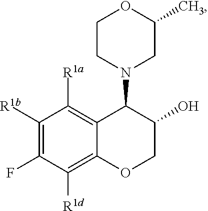

or a salt thereof.

In some further embodiments, the compound is:

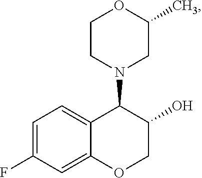 [SMILES: C[C@@H]1CN([C@@H]2c3ccc(F)cc3OC[C@H]2O)CCO1]

or a salt thereof.

In some embodiments, the present disclosure provides compounds having a structure represented by a formula:

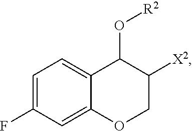

wherein X² is a halogen; and wherein R² is —C(O)(C1-C4 alkyl), or a salt thereof.

In some further embodiments, the compound is:

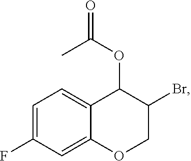 [SMILES: CC(=O)OC1c2ccc(F)cc2OCC1Br]

or a salt thereof.

In some further embodiments, the compound is selected from:

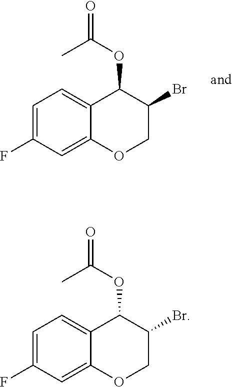 [SMILES: CC(=O)O[C@@H]1c2ccc(F)cc2OC[C@@H]1Br ; CC(=O)O[C@H]1c2ccc(F)cc2OC[C@H]1Br]

or a salt thereof.

In some embodiments, the present disclosure provides compounds having a structure:

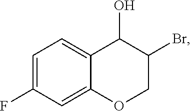 [SMILES: OC1c2ccc(F)cc2OCC1Br]

or a salt thereof.

In some embodiments, the present disclosure provides compounds having a structure represented by a formula:

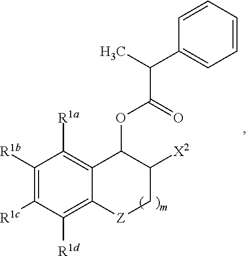

wherein m is 0 or 1; wherein X² is a halogen; wherein Z is selected from —CH₂—, —O—, and —NH—; and wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a salt thereof.

In some further embodiments, the compound has a structure represented by a formula:

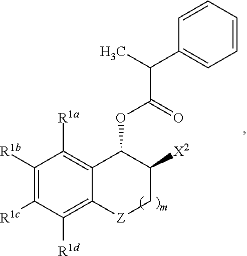

or a salt thereof.

In some further embodiments, the compound has a structure represented by a formula:

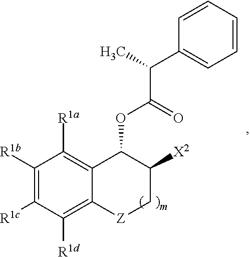

or a salt thereof.

In some further embodiments, the compound has a structure represented by a formula:

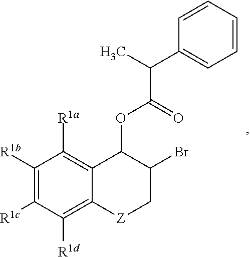

or a salt thereof.

In some further embodiments, the compound has a structure represented by a formula:

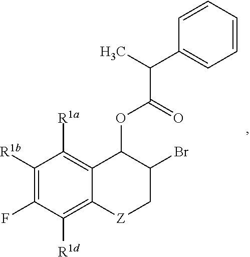

or a salt thereof.

In some embodiments, the present disclosure provides compounds having a structure represented by a formula:

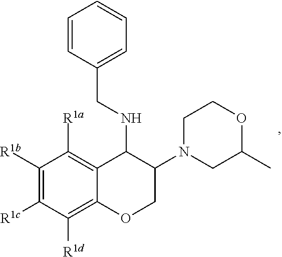

or a salt thereof, wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, provided that at least one of R^{1a}, R^{1b}, R^{1c}, and R^{1d} is not hydrogen, or a salt thereof.

In some embodiments, the present disclosure provides compounds having a structure represented by a formula:

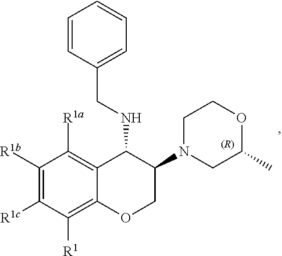

or a salt thereof, wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, provided that at least one of R^{1a}, R^{1b}, R^{1c}, and R^{1d} is not hydrogen, or a salt thereof.

In some further embodiments, the compound has a structure represented by a formula:

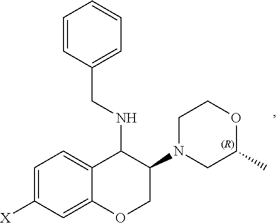

or a salt thereof, wherein X is a halogen.

In some further embodiments, the compound has a structure represented by a formula:

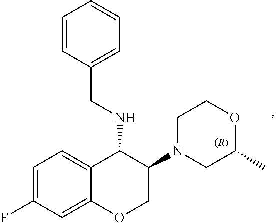

or a salt thereof.

In some further embodiments, the compound is:

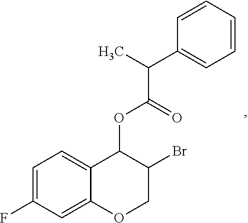 [SMILES: CC(C(=O)OC1c2ccc(F)cc2OCC1Br)c1ccccc1]

or a salt thereof.

In some further embodiments, the compound is:

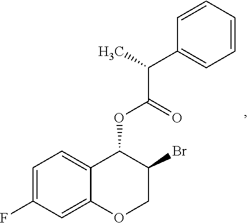 [SMILES: C[C@@H](C(=O)O[C@H]1c2ccc(F)cc2OC[C@@H]1Br)c1ccccc1]

or a salt thereof.

Specific examples of compounds are provided in the EXAMPLES section and are included herein. Pharmaceutically acceptable salts as well as the neutral forms of these compounds are also included.

a. X¹ Groups

In some embodiments, X¹ is a halogen. In a further embodiment, X¹ is selected from fluoro, chloro and bromo. In a still further embodiment, X¹ is selected from fluoro and chloro. In yet a further aspect, X¹ is selected from chloro and bromo. In an even further embodiment, X¹ is iodo. In a still further embodiment, X¹ is bromo. In yet a further embodiment, X¹ is chloro. In an even further embodiment, X¹ is fluoro.

b. X² and X^{2′} Groups

In some embodiments, X² is a halogen. In a further embodiment, X² is selected from fluoro, chloro and bromo. In a still further embodiment, X² is selected from fluoro and chloro. In yet a further aspect, X² is selected from chloro and bromo. In an even further embodiment, X² is iodo. In a still further embodiment, X² is bromo. In yet a further embodiment, X² is chloro. In an even further embodiment, X² is fluoro.

In some embodiments, each of X² and X²′ is independently halogen. In a further embodiment, each of X² and X²′ is independently selected from fluoro, chloro and bromo. In a still further embodiment, each of X² and X²′ is independently selected from fluoro and chloro. In yet a further aspect, each of X² and X²′ is independently selected from chloro and bromo. In an even further embodiment, each of X² and X²′ is iodo. In a still further embodiment, each of X² and X²′ is bromo. In yet a further embodiment, each of X² and X²′ is chloro. In an even further embodiment, each of X² and X²′ is fluoro.

h. R² Groups

In some embodiments, R² is —C(O)(C1-C4 alkyl) or a residue of a chiral auxiliary group.

In some embodiments, R² is —C(O)(C1-C4 alkyl). In a further embodiment, R² is selected from —C(O)CH₃, —C(O)CH₂CH₃, —C(O)CH(CH₃)₂, and —C(O)CH₂CH₂CH₃. In a still further embodiment, R² is selected from —C(O)CH₃ and —C(O)CH₂CH₃. In yet a further embodiment, R² is —C(O)CH₃.

In some embodiments, R² is a residue of a chiral auxiliary group. Examples of chiral auxiliary agents include, but are not limited to, (R)-2-phenylpropanoic acid, (1R)-(+)-campanic aid, (R)-(+)-2-methyl-2-propanesulfinamide, (S)-(−)-1-phenylethylamine, (1R,2S)-(+)-pseudoephedrin, (R)-(+)-1-phenylethylamine, (1R,2S)-(−)-ephedrine, (−)-nicotine, (1S, 2R)-(+)-norephedrine, (S)-(−)-2-methyl-2-propanesulfinamide, (S)-4-benzyl-2-oxazolidinone, (1R,2S)-(−)-ephedrine hydrochloride, (R)-4-benzyl-2-oxazolidinone, (1R,2R)-(−)-pseudoephedrine, (1R,2S)-(−)-N-methylephedrine, (1S,2R)-(+)-ephedrine hydrochloride, (S)-(−)-4-isopropyl-2-oxazolidinone, (1S)-(−)-2,10-camphorsultam, (S)-(+)-4-phenyl-2-oxazolidinone, L(−)-malic acid, (−)-quinine, (S)-(−)-3-boc-2,2-dimethyloxazolidine-4-carboxaldehyde, (1S,2R)-(+)-N-methylephedrine, (R)-(−)-4-phenyl-2-oxazolidinone, (−)-shikimic acid, (S)-(+)-4-benzyl-3-propionyl-2-oxazolidinone, (R)-4-benzylthiazolidine-2-thione, (S)-4-isopropylthiazolidine-2-thione, (1S,2S)-(+)-norpseudoephedrine, (R)-(−)-mandelic acid, D-(−)-N-methylglucamine, (2S,3S)-(+)-di-o-benzoyltartaric acid, (4R,5S)-(+)-4-methyl-5-phenyl-2-oxazolidinone, (1S)-(+)-camphor-10-sulfonic acid, (S)-4-(4-aminobenzyl)-2(1H)-oxazolidinone, (R)-4-isopropylthiazolidine-2-thione, (R)-(+)-3-boc-2,3-dimethyloxazolidine-4-carboxaldehyde, methyl (R)-(+)-3-boc-2,2-dimethyl-4-oxazolidinecarboxylate, methyl(S)-(−)-3-boc-2,2-dimethyl-4-oxazolidinecarboxylate, (S)-4-benzyloxazolidine-2-thione, (R)-4-phenylthiazolidine-2-thione, (R)-4-benzyloxazolidine-2-thione, (R)-(+)-4-benzyl-5,5-dimethyl-2-oxazolidinone, (S)-4-methyl-2-oxazolidinone, (S)-(+)-3-acetyl-4-benzyl-2-oxazolidinone, (4S,5R)-(−)-cis-4,5-diphenyl-2-oxazolidinone, (4R,5S)-(+)-cis-4,5-diphenyl-2-oxazolidinone, (+)-cinchonine, (2R,3R)-(−)-di-o-4-toluoyl-L-tartaric acid, (−)-quinic acid, (S)-(−)-2-methyl-2-propanesulfinamide solution, (S)-4-benzylthiazolidine-2-thione, (S)-(+)-S-methyl-S-phenylsulfoximine, (+)-N,N-diethylnorephedrine hydrochloride, (R)-(+)-4-isopropyl-5,5-diphenyl-2-oxazolidinone, (R)-(+)-4-tert-butyl-2-oxazolidinone, (S)-(+)-3-(benzyloxycarbonyl)-5-oxo-4-oxazolidineacetic acid, (S)-(+)-4-isopropyl-3-propionyl-2-oxazolidinone, (R)-4-benzyl-3-chloroacetyl-2-oxazolidinone, (R)-(+)-5,5-diphenyl-4-methyl-2-oxazolidinone, (S)-4-tert-butyl-2-oxazolidinone, (S)-(−)-4-isopropyl-2-oxazolidinethione, (R)-(−)-4-benzyl-3-propionyl-2-oxazoldinone, (R)-(+)-3-(benzyloxycarbonyl)-4-oxazolidinecarboxylic acid, (4S,5R)-(−)-4-methyl-5-phenyl-2-oxazolidinone, (S)-(−)-3-(benzyloxycarbonyl)-4-oxazolidinecarboxylic acid, (S)-(−)-4-isopropyl-5,5-dimethyl-2-oxazolidinone, (R)-(+)-4-isopropyl-5,5-dimethyl-2-oxazolidinone, (3aS-cis)-(−)-3,3a,8,8a-tetrahydro-2H-inden[1,2-d]oxazol-2-one, (S)-(−)-4-benzyl-5,5-dimethyl-2-oxazolidinone, (S)-(+)-3-(benzyloxycarbonyl)-5-oxo-4-oxazolidinepropionic acid, (S)-(+)-5,5-dimethyl-4-phenyl-2-oxazolidinone, (S)-(−)-5,5-diphenyl-4-(phenylmethyl)-2-oxazolidinone, (R)-(+)-4-(hydroxymethyl)-2-oxazolidinone benzoate, (4S)-4-(chloromethyl)-3-[(1R)-1-phenylethyl]-2-oxazolidinone, and (5R)-(hydroxymethyl-3-[(1R)-1-phenylethyl]-2-oxazolidinone. In a further embodiment, the chiral auxiliary agent is (R)-2-phenylpropanoic acid.

In some embodiments, the residue of the chiral auxiliary group is:

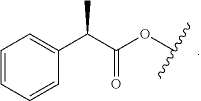 [SMILES: *OC(=O)[C@H](C)c1ccccc1]

### 2. Example Compounds

In some embodiments, a compound can be present as the following structure:

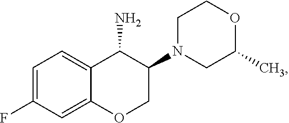 [SMILES: C[C@@H]1CN([C@H]2COc3cc(F)ccc3[C@@H]2N)CCO1]

or a pharmaceutically acceptable salt thereof.

In some embodiments, a compound can be present as the following structure:

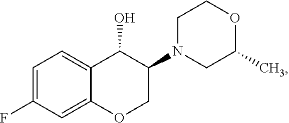 [SMILES: C[C@@H]1CN([C@H]2COc3cc(F)ccc3[C@@H]2O)CCO1]

or a pharmaceutically acceptable salt thereof.

In some embodiments, a compound can be present as the following structure:

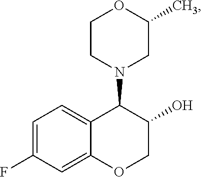 [SMILES: C[C@@H]1CN([C@@H]2c3ccc(F)cc3OC[C@H]2O)CCO1]

or a pharmaceutically acceptable salt thereof.

In some embodiments, a compound can be present as the following structure:

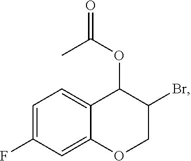 [SMILES: CC(=O)OC1c2ccc(F)cc2OCC1Br]

or a pharmaceutically acceptable salt thereof.

In some embodiments, a compound can be present as one or more of the following structures:

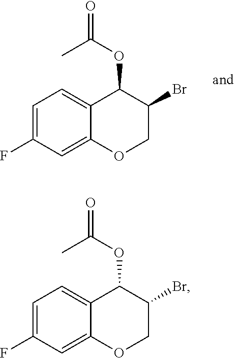 [SMILES: CC(=O)O[C@@H]1c2ccc(F)cc2OC[C@@H]1Br ; CC(=O)O[C@H]1c2ccc(F)cc2OC[C@H]1Br]

or a pharmaceutically acceptable salt thereof.

In some embodiments, a compound can be present as the following structure:

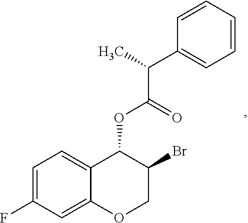 [SMILES: C[C@@H](C(=O)O[C@H]1c2ccc(F)cc2OC[C@@H]1Br)c1ccccc1]

or a pharmaceutically acceptable salt thereof.

### D. ADDITIONAL COMPOUNDS

In various embodiments, disclosed are compounds useful in the disclosed methods. It is understood that a disclosed compound can be provided by the disclosed methods.

In various embodiments, the disclosed compounds are useful as intermediates in the synthesis of N-(3-substituted-chroman-4-yl)-7H-pyrrolo[2,3-d]pyrimidin-4-amine compounds useful as PINK1 modulators.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Structure

In some embodiments, the present disclosure provides alcohols having a structure represented by a formula:

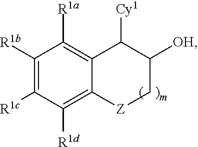

wherein m is 0 or 1; wherein Z is selected from —CH₂—, —O—, and —NH—; wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein Cy¹ is a 3- to 10-membered nitrogen-linked heterocycle substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a salt thereof.

In some further embodiments, the alcohol has a structure represented by a formula:

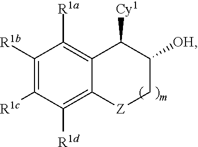

or a salt thereof.

In some further embodiments, the alcohol has a structure represented by a formula:

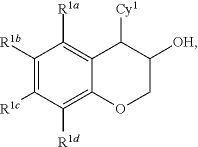

or a salt thereof.

In some further embodiments, the alcohol has a structure represented by a formula:

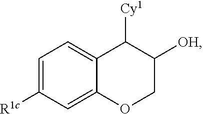

wherein R^{1c} is a non-hydrogen group, or a salt thereof.

In some further embodiments, the alcohol has a structure represented by a formula:

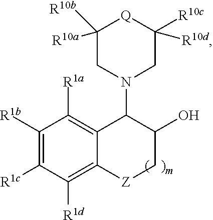

wherein Q is selected form —CH₂—, —O—, and —NR¹¹; wherein R¹¹, when present, is selected from hydrogen and C1-C4 alkyl; and wherein each of R^{10a}, R^{10b}, R^{10c}, and R^{10d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a salt thereof.

In some further embodiments, the alcohol has a structure represented by a formula:

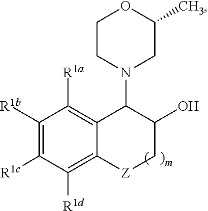

or a salt thereof.

In some further embodiments, the alcohol has a structure represented by a formula:

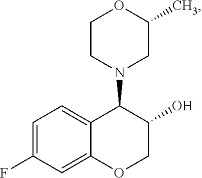 [SMILES: C[C@@H]1CN([C@@H]2c3ccc(F)cc3OC[C@H]2O)CCO1]

or a salt thereof.

In some embodiments, the present disclosure provides chromanamine derivatives having a structure represented by a formula:

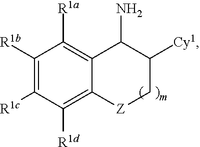

wherein m is 0 or 1; wherein Z is selected from —CH₂—, —O—, and —NH—; wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein Cy¹ is a 3- to 10-membered nitrogen-linked heterocycle substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a salt thereof.

In some further embodiments, the chromanamine derivative has a structure represented by a formula:

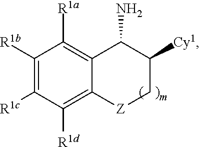

or a salt thereof.

In some further embodiments, the chromanamine derivative has a structure represented by a formula:

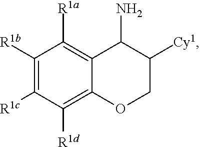

or a salt thereof.

In some further embodiments, the chromanamine derivative has a structure represented by a formula:

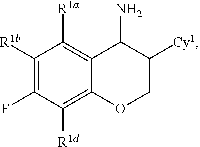

or a salt thereof.

In some further embodiments, the chromanamine derivative has a structure represented by a formula:

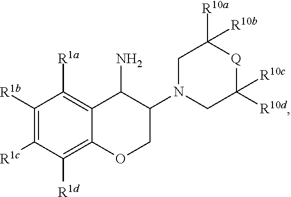

or a salt thereof.

In some further embodiments, wherein the chromanamine derivative has a structure represented by a formula:

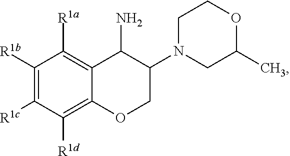

or a salt thereof.

In some further embodiments, the chromanamine derivative has a structure represented by a formula:

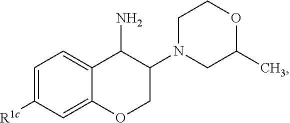

wherein R^{1c} is a non-hydrogen group, or a salt thereof.

In some further embodiments, the chromanamine derivative has a structure represented by a formula:

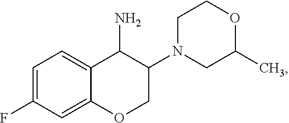 [SMILES: CC1CN(C2COc3cc(F)ccc3C2N)CCO1]

or a salt thereof.

In some further embodiments, the chromanamine derivative has a structure represented by a formula:

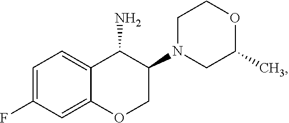 [SMILES: C[C@@H]1CN([C@H]2COc3cc(F)ccc3[C@@H]2N)CCO1]

or a salt thereof.

In some embodiments, the present disclosure provides chromane derivatives having a structure represented by a formula:

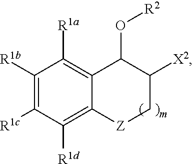

wherein m is 0 or 1; wherein X² is a halogen; and wherein Z is selected from —CH₂—, —O—, and —NH—; wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein R² is —C(O)(C1-C4 alkyl) or a residue of a chiral auxiliary group; and or a salt thereof.

In some further embodiments, the chromane derivative has a structure represented by a formula:

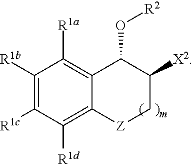

In some further embodiments, the chromane derivative has a structure represented by a formula:

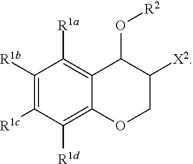

In some further embodiments, the chromane derivative has a structure represented by a formula:

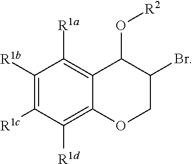

In some further embodiments, the chromane derivative has a structure represented by a formula:

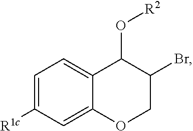

wherein R^{1c} is a non-hydrogen group.

In some further embodiments, the chromane derivative has a structure:

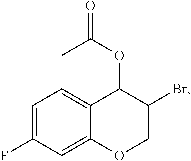 [SMILES: CC(=O)OC1c2ccc(F)cc2OCC1Br]

or a salt thereof.

In some further embodiments, the chromane derivative has a structure represented by a formula:

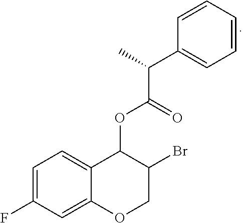 [SMILES: C[C@@H](C(=O)OC1c2ccc(F)cc2OCC1Br)c1ccccc1]

In some further embodiments, the chromane derivative has a structure represented by a formula:

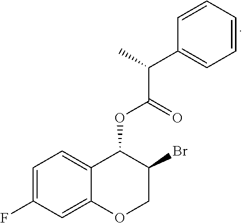 [SMILES: C[C@@H](C(=O)O[C@H]1c2ccc(F)cc2OC[C@@H]1Br)c1ccccc1]

In some embodiments, the present disclosure provides cyclic amines having a structure represented by a formula:

Cy¹-H,
wherein Cy¹ is a 3- to 10-membered nitrogen-linked heterocycle substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a salt thereof.

In some further embodiments, the cyclic amine has a structure represented by a formula:

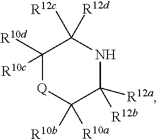

wherein Q is selected form —CH₂—, —O—, and —NR¹¹—; wherein R¹¹, when present, is selected from hydrogen and C1-C4 alkyl; wherein each of R^{10a}, R^{10b}, R^{10c}, and R^{10d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein each of R^{12a}, R^{12b}, R^{12c}, and R^{12d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino.

In some further embodiments, the cyclic amine has a structure represented by a formula:

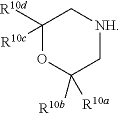

In some further embodiments, the cyclic amine has a structure represented by a formula:

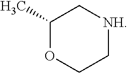

In some embodiments, the present disclosure provides halo-chromanol derivatives having a structure represented by a formula:

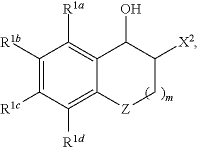

wherein m is 0 or 1; wherein X² is a halogen; wherein Z is selected from —CH₂—, —O—, and —NH—; and wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a salt thereof.

In some further embodiments, the halo-chromanol derivative has a structure:

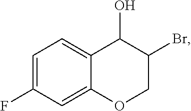 [SMILES: OC1c2ccc(F)cc2OCC1Br]

or a salt thereof.

In some embodiments, the present disclosure provides compounds having a structure:

R²—R¹³,
wherein R¹³ is selected from —OH, —O(C1-C4 alkyl), and —O(C2-C4 alkenyl), or a salt thereof.

In some further embodiments, the compound has a structure:

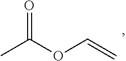

or a salt thereof.

In some embodiments, the present disclosure provides chromene derivatives having a structure represented by a formula:

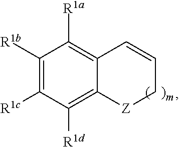

wherein m is 0 or 1; wherein Z is selected from —CH₂—, —O—, and —NH—; and wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a salt thereof.

In some embodiments, the present disclosure provides chromanol derivatives having a structure represented by a formula:

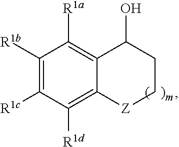

wherein m is 0 or 1; wherein Z is selected from —CH₂—, —O—, and —NH—; and wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a salt thereof.

In some embodiments, the present disclosure provides chromanol derivatives having a structure represented by a formula:

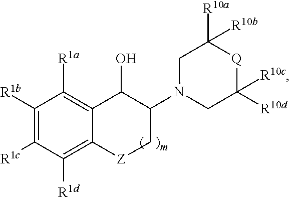

wherein m is 0 or 1; wherein Q is selected form —CH₂—, —O—, and —NR¹¹; wherein R¹¹, when present, is selected from hydrogen and C1-C4 alkyl; wherein Z is selected from —CH₂—, —O—, and —NH—; wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein each of R^{10a}, R^{10b}, R^{10c}, and R^{10d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a salt thereof.

In some embodiments, the present disclosure provides chromanone derivatives having a structure represented by a formula:

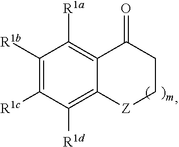

wherein m is 0 or 1; wherein Z is selected from —CH₂—, —O—, and —NH—; and wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; or a salt thereof.

In some embodiments, the present disclosure provides pyrrolopyrimidines having a structure represented by a formula:

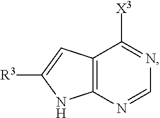

wherein X³ is a halogen; and wherein R³ is selected from C1-C6 haloalkyl, C1-C6 haloalkoxy, C1-C6 halohydroxy alkyl, and an unsubstituted 3- to 6-membered cycloalkyl, or a salt thereof.

In some embodiments, the present disclosure provides chromenone derivatives having a structure represented by a formula:

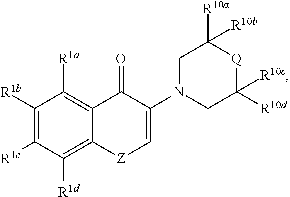

wherein Q is selected form —CH₂—, —O—, and —NR¹¹—; wherein R¹¹, when present, is selected from hydrogen and C1-C4 alkyl; wherein Z is selected from —CH₂—, —O—, and —NH—;
wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein each of R^{10a}, R^{10b}, R^{10c}, and R^{10d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a salt thereof.

In some further embodiments, the chromenone derivative has a structure represented by a formula:

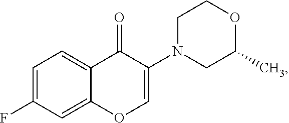 [SMILES: C[C@@H]1CN(c2coc3cc(F)ccc3c2=O)CCO1]

or a salt thereof.

In some embodiments, the present disclosure provides di-halides having a structure represented by a formula:

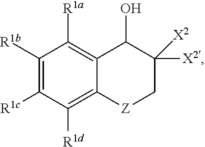

wherein each of X² and X^{2′} is independently halogen, or a salt thereof.

In some further embodiments, the di-halide has a structure represented by a formula:

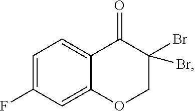 [SMILES: O=C1c2ccc(F)cc2OCC1(Br)Br]

or a salt thereof.

In some embodiments, the present disclosure provides chromanone derivatives having a structure represented by a formula:

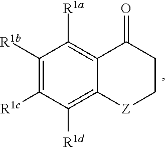

or a salt thereof.

In some further embodiments, the chromanone derivative has a structure represented by a formula:

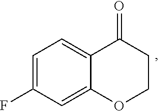 [SMILES: O=C1CCOc2cc(F)ccc21]

or a salt thereof.

In some embodiments, the present disclosure provides chromanone derivatives having a structure represented by a formula:

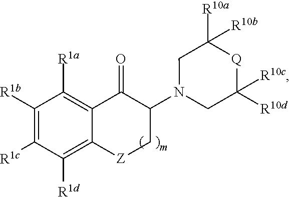

wherein m is 0 or 1; wherein Q is selected form —CH₂—, —O—, and —NR¹¹; wherein R¹¹, when present, is selected from hydrogen and C1-C4 alkyl; wherein Z is selected from —CH₂—, —O—, and —NH—; wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein each of R^{10a}, R^{10b}, R^{10c}, and R^{10d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a salt thereof.

In some further embodiments, the chromanone derivative has a structure represented by a formula:

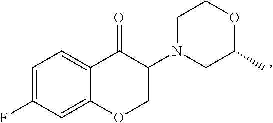 [SMILES: C[C@@H]1CN(C2COc3cc(F)ccc3C2=O)CCO1]

or a salt thereof.

In some embodiments, the present disclosure provides halides having a structure represented by a formula:

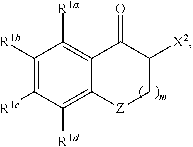

wherein X² is halogen, or a salt thereof.

In some further embodiments, the halide has a structure represented by a formula:

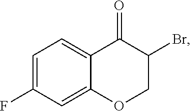 [SMILES: O=C1c2ccc(F)cc2OCC1Br]

or a salt thereof.
   a. R^{12A}, R^{12B}, R^{12C}, and R^{12D} Groups

In some embodiments, each of R^{12a}, R^{12b}, R^{12c}, and R^{12d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, each of R^{12a}, R^{12b}, R^{12c}, and R^{12d} is independently selected from hydrogen, F, —Cl, —CN, —NH₂, —OH, —NO₂, methyl, ethyl, n-propyl, isopropyl, ethenyl, propenyl, —CH₂F, —CH₂CH₂F, —CH(CH₃) CH₂F, —CH₂CH₂CH₂F, —CH₂Cl, —CH₂CH₂Cl, —CH(CH₃) CH₂Cl, —CH₂CH₂CH₂Cl, —CH₂CN, —CH₂CH₂CN, —CH(CH₃) CH₂CN, —CH₂CH₂CH₂CN, —CH₂OH, —CH₂CH₂OH, —CH(CH₃) CH₂OH, —CH₂CH₂CH₂OH, methoxy, ethoxy, n-propoxy, isopropoxy, —OCF₃, —OCHF₂, —OCH₂F, —OCH₂CH₂F, —OCH(CH₃) CH₂F, —OCH₂CH₂CH₂F, —OCCl₃, —OCHCl₂, —OCH₂Cl, —OCH₂CH₂Cl, —OCH(CH₃) CH₂Cl, —OCH₂CH₂CH₂Cl, —NHCH₃, —NHCH₂CH₃, —NHCH(CH₃) CH₃, —NHCH₂CH₂CH₃, —N(CH₃)₂, —N(CH₃) CH₂CH₃, —N(CH₃) CH(CH₃) CH₃, and —N(CH₃) CH₂CH₂CH₃. In still further embodiments, each of R^{12a}, R^{12b}, R^{12c}, and R^{12d} is independently selected from hydrogen, F, —Cl, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, methoxy, ethoxy, —OCF₃, —OCHF₂, —OCH₂F, —OCH₂CH₂F, —OCCl₃, —OCHCl₂, —OCH₂Cl, —OCH₂CH₂Cl, —NHCH₃, —NHCH₂CH₃, —N(CH₃)₂, and —N(CH₃) CH₂CH₃. In yet further embodiments, each of R^{12a}, R^{12b}, R^{12c}, and R^{12d} is independently selected from hydrogen, F, —Cl, —CN, —NH₂, —OH, —NO₂, methyl, —CH₂F, —CH₂Cl, —CH₂CN, —CH₂OH, methoxy, —OCF₃, —OCHF₂, —OCH₂F, —OCCl₃, —OCHCl₂, —OCH₂Cl, —NHCH₃, and —N(CH₃)₂.

In further embodiments, each of R^{12a}, R^{12b}, R^{12c}, and R^{12d} is hydrogen.

In various embodiments, each of R^{12a}, R^{12b}, R^{12c}, and R^{12d} is independently selected from hydrogen, halogen, —CN, —NH₂, —OH, —NO₂, C1-C4 hydroxyalkyl, C1-C4 haloalkoxy, C1-C4 alkoxy. In further embodiments, each of R^{12a}, R^{12b}, R^{12c}, and R^{12d} is independently selected from hydrogen, F, —Cl, —CN, —NH₂, —OH, —NO₂, —CH₂OH, —CH₂CH₂OH, —CH(CH₃) CH₂OH, —CH₂CH₂CH₂OH, methoxy, ethoxy, n-propoxy, isopropoxy, —OCF₃, —OCHF₂, —OCH₂F, —OCH₂CH₂F, —OCH(CH₃) CH₂F, —OCH₂CH₂CH₂F, —OCCl₃, —OCHCl₂, —OCH₂Cl, —OCH₂CH₂Cl, —OCH(CH₃) CH₂Cl, and —OCH₂CH₂CH₂Cl. In still further embodiments, each of R^{12a}, R^{12b}, R^{12c}, and R^{12d} is independently selected from hydrogen, F, —Cl, —CN, —NH₂, —OH, —NO₂, —CH₂OH, —CH₂CH₂OH, methoxy, ethoxy, —OCF₃, —OCHF₂, —OCH₂F, —OCH₂CH₂F, —OCCl₃, —OCHCl₂, —OCH₂Cl, and —OCH₂CH₂Cl. In yet further embodiments, each of R^{12a}, R^{12b}, R^{12c}, and R^{12d} is independently selected from hydrogen, F, —Cl, —CN, —NH₂, —OH, —NO₂, —CH₂OH, methoxy, —OCF₃, —OCHF₂, —OCH₂F, —OCCl₃, —OCHCl₂, and —OCH₂Cl.

In various embodiments, each of R^{12a}, R^{12b}, R^{12c}, and R^{12d} is independently selected from hydrogen, halogen, —CN, —NH₂, —OH, —NO₂, C1-C4 alkylamino, and (C1-C4)(C1-C4) dialkylamino. In further embodiments, each of R^{12a}, R^{12b}, R^{12c}, and R^{12d} is independently selected from hydrogen, F, —Cl, —CN, —NH₂, —OH, —NO₂, —NHCH₃, —NHCH₂CH₃, —NHCH(CH₃) CH₃, —NHCH₂CH₂CH₃, —N(CH₃)₂, —N(CH₃) CH₂CH₃, —N(CH₃) CH(CH₃) CH₃, and —N(CH₃) CH₂CH₂CH₃. In still further embodiments, each of R^{12a}, R^{12b}, R^{12c}, and R^{12d} is independently selected from hydrogen, F, —Cl, —CN, —NH₂, —OH, —NO₂, —NHCH₃, —NHCH₂CH₃, —N(CH₃)₂, and —N(CH₃) CH₂CH₃. In yet further embodiments, each of R^{12a}, R^{12b}, R^{12c}, and R^{12d} is independently selected from hydrogen, F, —Cl, —CN, —NH₂, —OH, —NO₂, —NHCH₃, and —N(CH₃)₂.

In various embodiments, each of R^{12a}, R^{12b}, R^{12c}, and R^{12d} is independently selected from hydrogen, halogen, —CN, —NH₂, —OH, —NO₂, C1-C4 haloalkyl, and C1-C4 cyanoalkyl. In further embodiments, each of R^{12a}, R^{12b}, R^{12c}, and R^{12d} is independently selected from hydrogen, F, —Cl, —CN, —NH₂, —OH, —NO₂, —CH₂F, —CH₂CH₂F, —CH(CH₃) CH₂F, —CH₂CH₂CH₂F, —CH₂Cl, —CH₂CH₂Cl, —CH(CH₃) CH₂Cl, —CH₂CH₂CH₂Cl, —CH₂CN, —CH₂CH₂CN, —CH(CH₃) CH₂CN, and —CH₂CH₂CH₂CN. In still further embodiments, each of R^{12a}, R^{12b}, R^{12c}, and R^{12d} is independently selected from hydrogen, F, —Cl, —CN, —NH₂, —OH, —NO₂, —CH₂F, —CH₂CH₂F, —CH₂Cl, —CH₂CH₂Cl, —CH₂CN, and —CH₂CH₂CN. In yet further embodiments, each of R^{12a}, R^{12b}, R^{12c}, and R^{12d} is independently selected from hydrogen, F, —Cl, —CN, —NH₂, —OH, —NO₂, —CH₂F, —CH₂Cl, and —CH₂CN.

In various embodiments, each of R^{12a}, R^{12b}, R^{12c}, and R^{12d} is independently selected from hydrogen, halogen, —CN, —NH₂, —OH, —NO₂, C1-C4 alkyl, and C2-C4 alkenyl. In further embodiments, each of R^{12a}, R^{12b}, R^{12c}, and R^{12d} is independently selected from hydrogen, F, —Cl, —CN, —NH₂, —OH, —NO₂, methyl, ethyl, n-propyl, isopropyl, ethenyl, and propenyl. In still further embodiments, each of R^{12a}, R^{12b}, R^{12c}, and R^{12d} is independently selected from hydrogen, F, —Cl, —CN, —NH₂, —OH, —NO₂, methyl, ethyl, and ethenyl. In yet further embodiments, each of R^{12a}, R^{12b}, R^{12c}, and R^{12d} is independently selected from hydrogen, F, —Cl, —CN, —NH₂, —OH, —NO₂, and methyl.

In various embodiments, each of R^{12a}, R^{12b}, R^{12c}, and R^{12d} is independently selected from hydrogen and C1-C4 alkyl. In further embodiments, each of R^{12a}, R^{12b}, R^{12c}, and R^{12d} is independently selected from hydrogen, methyl, ethyl, n-propyl, and isopropyl. In still further embodiments, each of R^{12a}, R^{12b}, R^{12c}, and R^{12d} is independently selected from hydrogen, methyl, and ethyl. In yet further embodiments, each of R^{12a}, R^{12b}, R^{12c}, and R^{12d} is independently selected from hydrogen and methyl.

In various embodiments, each of R^{12a}, R^{12b}, R^{12c}, and R^{12d} is independently selected from hydrogen and halogen. In further embodiments, each of R^{12a}, R^{12b}, R^{12c}, and R^{12d} is independently selected from hydrogen, —F, —Cl, and —Br. In still further embodiments, each of R^{12a}, R^{12b}, R^{12c}, and R^{12d} is independently selected from hydrogen, —F, and —Cl. In yet further embodiments, each of R^{12a}, R^{12b}, R^{12c}, and R^{12d} is independently selected from hydrogen and —Cl. In still further embodiments, each of R^{12a}, R^{12b}, R^{12c}, and R^{12d} is independently selected from hydrogen and —F.

In some embodiments, each of R^{12a}, R^{12b}, R^{12c}, and R^{12d} is independently hydrogen, halogen, or C1-C4 alkyl. In further embodiments, each of R^{12a}, R^{12b}, R^{12c}, and R^{12d} is independently hydrogen, —F, —Cl, —Br, methyl, ethyl, n-propyl, or isopropyl. In still further embodiments, each of R^{12a}, R^{12b}, R^{12c}, and R^{12d} is independently hydrogen, —F, —Cl, methyl, and ethyl. In yet further embodiments, each of R^{12a}, R^{12b}, R^{12c}, and R^{12d} is independently hydrogen, —F, and methyl.

b. R¹³ Groups

In some embodiments, R¹³ is selected from —OH, —O(C1-C4 alkyl), and —O(C2-C4 alkenyl). In a further embodiment, R¹³ is selected from —OH, —C(O)CH₃, —C(O)CH₂CH₃, —C(O)CH(CH₃)₂, —C(O)CH₂CH₂CH₃, —C(O)CH═CH₂, —C(O)CH(CH₃)═CH₂, —C(O)CH₂CH═CH₂, and —C(O)CH═CHCH₃. In a still further embodiment, R¹³ is selected from —OH, —C(O)CH₃, —C(O)CH₂CH₃, —C(O)CH═CH₂, and —C(O)CH(CH₃)═CH₂. In yet a further embodiment, R¹³ is selected from —OH and —C(O)CH₃.

In various embodiments, R¹³ is selected from —OH and —O(C1-C4 alkyl). In a further embodiment, R¹³ is selected from —OH, —C(O)CH₃, —C(O)CH₂CH₃, —C(O)CH(CH₃)₂, and —C(O)CH₂CH₂CH₃. In a still further embodiment, R¹³ is selected from —OH, —C(O)CH₃, and —C(O)CH₂CH₃. In yet a further embodiment, R¹³ is selected from —OH and —C(O)CH₃.

In various embodiments, R¹³ is —O(C1-C4 alkyl). In a further embodiment, R¹³ is selected from —C(O)CH₃, —C(O)CH₂CH₃, —C(O)CH(CH₃)₂, and —C(O)CH₂CH₂CH₃. In a still further embodiment, R¹³ is selected from —C(O)CH₃ and —C(O)CH₂CH₃. In yet a further embodiment, R¹³ is —C(O)CH₃.

In various embodiments, R¹³ is selected from —OH and —O(C2-C4 alkenyl). In a further embodiment, R¹³ is selected from —OH, —C(O)CH═CH₂, —C(O)CH(CH₃)═CH₂, —C(O)CH₂CH═CH₂, and —C(O)CH═CHCH₃. In a still further embodiment, R¹³ is selected from —OH, —C(O)CH═CH₂, and —C(O)CH(CH₃)═CH₂.

In various embodiments, R¹³ is —O(C2-C4 alkenyl). In a further embodiment, R¹³ is selected from —C(O)CH═CH₂, —C(O)CH(CH₃)═CH₂, —C(O)CH₂CH═CH₂, and —C(O)CH═CHCH₃. In a still further embodiment, R¹³ is selected from —C(O)CH═CH₂ and —C(O)CH(CH₃)═CH₂.

In various embodiments, R¹³ is —OH.

### 2. Example Compounds

In some embodiments, a compound can be present as the following structure:

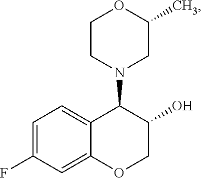 [SMILES: C[C@@H]1CN([C@@H]2c3ccc(F)cc3OC[C@H]2O)CCO1]

or a pharmaceutically acceptable salt thereof.

In some embodiments, a compound can be present as the following structure:

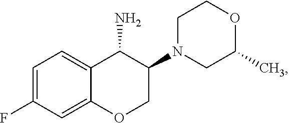 [SMILES: C[C@@H]1CN([C@H]2COc3cc(F)ccc3[C@@H]2N)CCO1]

or a pharmaceutically acceptable salt thereof.

In some embodiments, a compound can be present as the following structure:

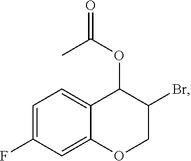 [SMILES: CC(=O)OC1c2ccc(F)cc2OCC1Br]

or a pharmaceutically acceptable salt thereof.

In some embodiments, a compound can be present as the following structure:

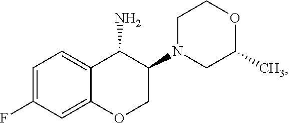 [SMILES: C[C@@H]1CN([C@H]2COc3cc(F)ccc3[C@@H]2N)CCO1]

or a pharmaceutically acceptable salt thereof.

In some embodiments, a compound can be present as the following structure:

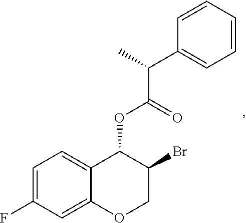 [SMILES: C[C@@H](C(=O)O[C@H]1c2ccc(F)cc2OC[C@@H]1Br)c1ccccc1]

or a pharmaceutically acceptable salt thereof.

In some embodiments, a compound can be present as the following structure:

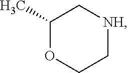

or a pharmaceutically acceptable salt thereof.

In some embodiments, a compound can be present as the following structure:

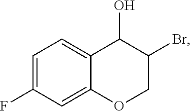 [SMILES: OC1c2ccc(F)cc2OCC1Br]

or a pharmaceutically acceptable salt thereof.

In some embodiments, a compound can be present as the following structure:

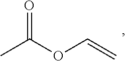

or a pharmaceutically acceptable salt thereof.

In some embodiments, a compound can be present as the following structure:

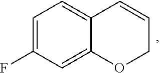 [SMILES: Fc1ccc2c(c1)OCC=C2]

or a pharmaceutically acceptable salt thereof.

In some embodiments, a compound can be present as the following structure:

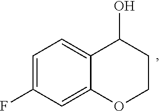 [SMILES: OC1CCOc2cc(F)ccc21]

or a pharmaceutically acceptable salt thereof.

In some embodiments, a compound can be present as the following structure:

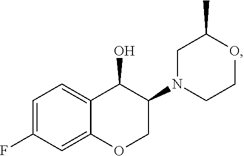 [SMILES: C[C@@H]1CN([C@H]2COc3cc(F)ccc3[C@H]2O)CCO1]

or a pharmaceutically acceptable salt thereof.

In some embodiments, a compound can be present as the following structure:

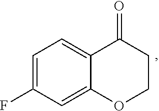 [SMILES: O=C1CCOc2cc(F)ccc21]

or a pharmaceutically acceptable salt thereof.

In some embodiments, a compound can be present as the following structure:

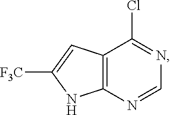 [SMILES: FC(F)(F)c1cc2c(Cl)ncnc2[nH]1]

or a pharmaceutically acceptable salt thereof.

In some embodiments, the present disclosure provides compounds having a structure represented by a formula:

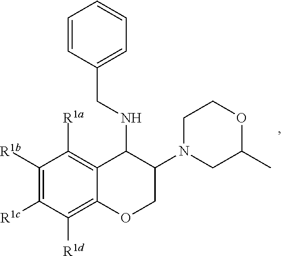

or a salt thereof, wherein each of Ra, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, provided that at least one of R^{1a}, R^{1b}, R^{1c}, and R^{1d} is not hydrogen, or a salt thereof.

In some embodiments, a compound can be present as the following structure:

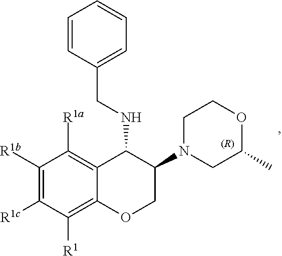

or a salt thereof, wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, provided that at least one of R^{1a}, R^{1b}, R^{1c}, and R^{1d} is not hydrogen, or a salt thereof.

In some embodiments, a compound can be present as the following structure:

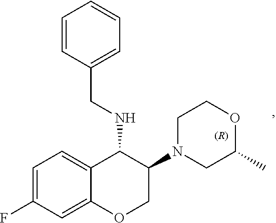

or a salt thereof.

In some embodiments, a compound can be present as the following structure:

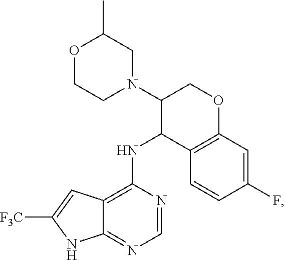 [SMILES: CC1CN(C2COc3cc(F)ccc3C2Nc2ncnc3[nH]c(C(F)(F)F)cc23)CCO1]

or a pharmaceutically acceptable salt thereof.

In some embodiments, a compound can be present as the following structure:

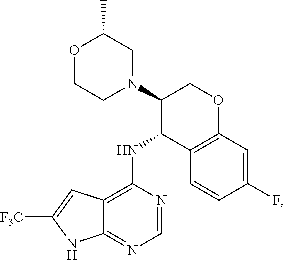 [SMILES: C[C@@H]1CN([C@H]2COc3cc(F)ccc3[C@@H]2Nc2ncnc3[nH]c(C(F)(F)F)cc23)CCO1]

or a pharmaceutically acceptable salt thereof.

In some embodiments, a compound can be present as the following structure:

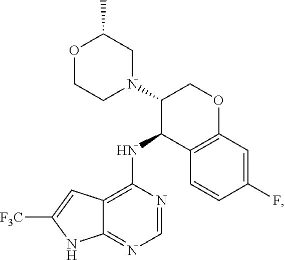 [SMILES: C[C@@H]1CN([C@@H]2COc3cc(F)ccc3[C@H]2Nc2ncnc3[nH]c(C(F)(F)F)cc23)CCO1]

or a pharmaceutically acceptable salt thereof. In some embodiments, the disclosure relates to a pharmaceutical composition comprising: (i) a therapeutically effective amount of a compound chosen from one of:

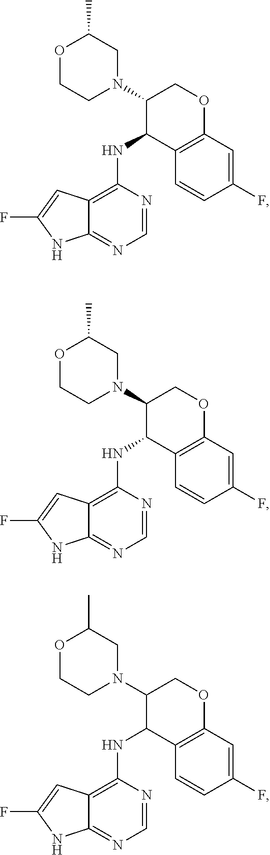 [SMILES: C[C@@H]1CN([C@@H]2COc3cc(F)ccc3[C@H]2Nc2ncnc3[nH]c(F)cc23)CCO1 ; C[C@@H]1CN([C@H]2COc3cc(F)ccc3[C@@H]2Nc2ncnc3[nH]c(F)cc23)CCO1 ; CC1CN(C2COc3cc(F)ccc3C2Nc2ncnc3[nH]c(F)cc23)CCO1]

or a salt of any of the foregoing; and (ii) a pharmaceutically acceptable carrier. In some embodiments, the disclosure relates to a pharmaceutical composition comprising: (i) a therapeutically effective amount of a compound chosen from one of:

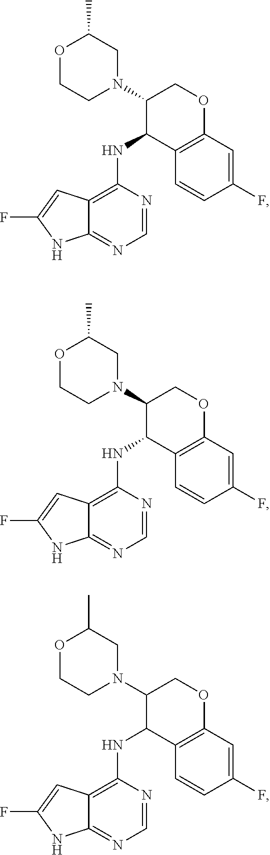 [SMILES: C[C@@H]1CN([C@@H]2COc3cc(F)ccc3[C@H]2Nc2ncnc3[nH]c(F)cc23)CCO1 ; C[C@@H]1CN([C@H]2COc3cc(F)ccc3[C@@H]2Nc2ncnc3[nH]c(F)cc23)CCO1 ; CC1CN(C2COc3cc(F)ccc3C2Nc2ncnc3[nH]c(F)cc23)CCO1]

or a salt of any of the foregoing; and (ii) a pharmaceutically acceptable carrier; wherein the pharmaceutical composition is free of Compound 6, Compound 7 and/or Compound 8 (as identified in the Examples). In some embodiments, compositions comprise one or a combination of compounds made by the process of synthesis steps disclosed herein.

### E. METHODS OF MAKING THE COMPOUNDS

In some embodiments, provided herein are methods comprising: (a) activating an alcohol having a structure represented by a formula:

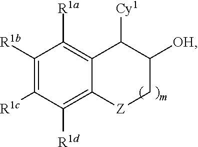

wherein m is 0 or 1; wherein Z is selected from —CH₂—, —O—, and —NH—; wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein Cy¹ is a 3- to 10-membered nitrogen-linked heterocycle substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a salt thereof, thereby forming an activated alcohol; and (b) displacing the activated alcohol via addition of an amine, thereby providing a chromanamine derivative having a structure represented by a formula:

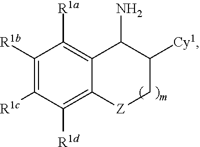

or a salt thereof.

In some further embodiments, the alcohol is activated by a tosylate or a mesylate.

In some further embodiments, the activating step is in the presence of a base. In a further embodiment, the base is an amine base. In a still further embodiment, the base is triethylamine or N,N-diisopropylethylamine (DIPEA).

In some further embodiments, the activating step is in an aprotic solvent. In a further embodiment, the polar solvent is dichloromethane.

In some further embodiments, the activating step is at a temperature of from about −10 °C to about 10 °C. In a further embodiment, the activating step is at a temperature of from about −10 °C to about 8 °C, about −10 °C to about 6 °C, about −10 °C to about 4 °C, about −10 °C to about 2 °C, about −10 °C to about 0 °C, about −10 °C to about −2 °C, about −10 °C to about −4 °C, about −10 °C to about −6 °C, about −10 °C to about −8 °C, 8 about −8 °C to about 8 °C, about −6 °C to about 8 °C, about −4 °C to about 8 °C, about −2 °C to about 8 °C, about 0 °C to about 8 °C, about 2 °C to about 8 °C, about 4 °C to about 8 °C, about 6 °C to about 8 °C, about −8 °C to about 6 °C, about −6 °C to about 4 °C, about −4 °C to about 2 °C, or about −2 °C to about 0 °C. In a still further embodiment, the activating step is at a temperature of about 0 °C.

In some further embodiments, the activating step is for a time period of from about 1.0 hour to about 3.0 hours. In a further embodiment, the activating step is for a time period of from about 1.0 hour to about 2.5 hours, about 1.0 hour to about 2.0 hours, about 1.0 hour to about 1.5 hours, about 1.5 hour to about 3.0 hours, about 2.0 hour to about 3.0 hours, about 2.5 hour to about 3.0 hours, or about 1.5 hour to about 2.5 hours. In a still further embodiment, the activating step is for a time period of about 1.5 hours.

In some further embodiments, the activating step is with stirring.

In some further embodiments, the alcohol has a structure represented by a formula:

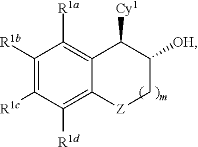

or a salt thereof.

In some further embodiments, the alcohol has a structure represented by a formula:

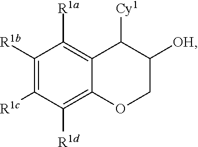

or a salt thereof.

In some further embodiments, the alcohol has a structure represented by a formula:

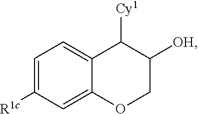

wherein R^{1c} is a non-hydrogen group, or a salt thereof.

In some further embodiments, the alcohol has a structure represented by a formula:

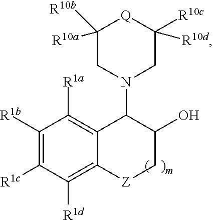

wherein Q is selected form —CH₂—, —O—, and —NR¹¹; wherein R¹¹, when present, is selected from hydrogen and C1-C4 alkyl; and wherein each of R^{10a}, R^{10b}, R^{10c}, and R^{10d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a salt thereof.

In some further embodiments, the alcohol has a structure represented by a formula:

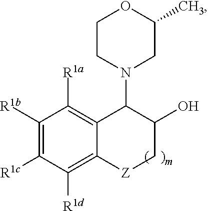

or a salt thereof.

In some further embodiments, the alcohol has a structure represented by a formula:

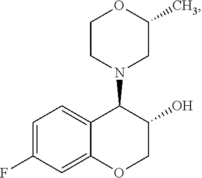 [SMILES: C[C@@H]1CN([C@@H]2c3ccc(F)cc3OC[C@H]2O)CCO1]

or a salt thereof.

In some embodiments, the displacing step is performed at an elevated temperature. In a further embodiment, the elevated temperature is of from about 35 °C to about 55 °C. In a still further embodiment, the elevated temperature is of from about 35 °C to about 50 °C, about 35 °C to about 45 °C, about 35 °C to about 40 °C, about 40 °C to about 55 °C, about 45 °C to about 55 °C, about 50 °C to about 55 °C, or about 40 °C to about 50 °C. In yet a further embodiment, the elevated temperature is about 45 °C.

In some embodiments, the elevated temperature is maintained for a time period of from about 1 hour to about 3 hours. In a further embodiment, the elevated temperature is maintained for a time period of from about 1 hour to about 2.5 hours, about 1 hour to about 2 hours, about 1 hour to about 1.5 hours, about 1.5 hour to about 3 hours, about 2 hour to about 3 hours, about 2.5 hour to about 3 hours, or about 1.5 hour to about 2.5 hours. In a still further embodiment, the elevated temperature is maintained for a time period of about 2 hours.

In some embodiments, the amine is ammonia. In a further embodiment, the amine is (2,4-dimethyoxyphenyl) methanamine, benzophenone imine, or benzylamine.

In some embodiments, the amine is a protected amine. In a further embodiment, the method further comprises deprotecting the protected amine. In a still further embodiment, deprotecting is via the addition of an acid (e.g., trifluoroacetic acid, hydrochloric acid). In yet a further aspect, deprotecting is via hydrogenation.

In some embodiments, m is 1.

In some embodiments, Z is —O—.

In some embodiments, at least one of R^{1a}, R^{1b}, R^{1c}, and R^{1d} is a non-hydrogen group. In a further embodiment, at least one of R^{1a}, R^{1b}, R^{1c}, and R^{1d} is halogen. In a still further embodiment, at least one of Ra, R^{1b}, R^{1c}, and R^{1d} is fluoro.

In some embodiments, R^{1c} is a non-hydrogen group. In a further embodiment, R^{1c} is halogen. In a still further embodiment, R^{1c} is fluoro.

In some embodiments, Cy¹ is selected from pyrrolidinyl, imidazolidinyl, piperidinyl, piperazinyl, and morpholinyl. In a still further embodiment, Cy¹ is a structure represented by a formula:

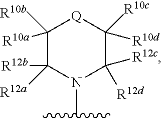

wherein Q is selected form —CH₂—, —O—, and —NR¹¹—; wherein R¹¹, when present, is selected from hydrogen and C1-C4 alkyl; wherein each of R^{10a}, R^{10b}, R^{10c}, and R^{10d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein each of R^{12a}, R^{12b}, R^{12c}, and R^{12d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino.

In some embodiments, Q is —O—.

In some embodiments, each of R^{12a}, R^{12b}, R^{12c}, and R^{12d} is hydrogen.

In some embodiments, Cy¹ is a structure represented by a formula:

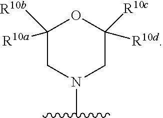

In some embodiments, Cy¹ is a structure represented by a formula:

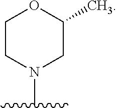 [SMILES: *N1CCO[C@H](C)C1]

In some embodiments, the method further comprises epoxidizing a chromane derivative having a structure represented by a formula:

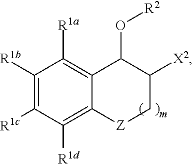

wherein X² is a halogen; and wherein R² is —C(O)(C1-C4 alkyl) or a residue of a chiral auxiliary group, or a salt thereof, and reacting the epoxide with a cyclic amine having a structure represented by a formula:

Cy¹-H,
thereby providing the alcohol.

In some embodiments, R² is —C(O)CH₃.

In some embodiments, epoxidizing is in the presence of a base. In a further embodiment, the base is sodium methoxide.

In some embodiments, epoxidizing is in the presence of an aprotic solvent. In a further embodiment, the aprotic solvent is diethyl ether.

In some embodiments, the chromane derivative has a structure:

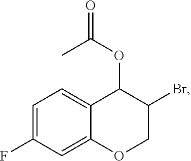 [SMILES: CC(=O)OC1c2ccc(F)cc2OCC1Br]

or a salt thereof.

In some embodiments, the method further comprises coupling a halo-chromanol derivative having a structure represented by a formula:

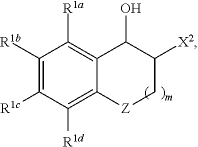

or a salt thereof, and either a chiral auxiliary agent or a compound having a structure:

R²—R¹³,
wherein R¹³ is selected from —OH, —O(C1-C4 alkyl), and —O(C2-C4 alkenyl), thereby providing the chromane derivative.

In some embodiments, the halo-chromanol derivative is coupled to the chiral auxiliary agent. Examples of chiral auxiliary agents include, but are not limited to, (R)-2-phenylpropanoic acid, (1R)-(+)-campanic aid, (R)-(+)-2-methyl-2-propanesulfinamide, (S)-(−)-1-phenylethylamine, (1R,2S)-(+)-pseudoephedrin, (R)-(+)-1-phenylethylamine, (1R,2S)-(−)-ephedrine, (−)-nicotine, (1S,2R)-(+)-norephedrine, (S)-(−)-2-methyl-2-propanesulfinamide, (S)-4-benzyl-2-oxazolidinone, (1R,2S)-(−)-ephedrine hydrochloride, (R)-4-benzyl-2-oxazolidinone, (1R,2R)-(−)-pseudoephedrine, (1R,2S)-(−)-N-methylephedrine, (1S,2R)-(+)-ephedrine hydrochloride, (S)-(−)-4-isopropyl-2-oxazolidinone, (1S)-(−)-2,10-camphorsultam, (S)-(+)-4-phenyl-2-oxazolidinone, L(−)-malic acid, (−)-quinine, (S)-(−)-3-boc-2,2-dimethyloxazolidine-4-carboxaldehyde, (1S,2R)-(+)-N-methylephedrine, (R)-(−)-4-phenyl-2-oxazolidinone, (−)-shikimic acid, (S)-(+)-4-benzyl-3-propionyl-2-oxazolidinone, (R)-4-benzylthiazolidine-2-thione, (S)-4-isopropylthiazolidine-2-thione, (1S,2S)-(+)-norpseudoephedrine, (R)-(−)-mandelic acid, D-(−)-N-methylglucamine, (2S,3S)-(+)-di-o-benzoyltartaric acid, (4R,5S)-(+)-4-methyl-5-phenyl-2-oxazolidinone, (1S)-(+)-camphor-10-sulfonic acid, (S)-4-(4-aminobenzyl)-2(1H)-oxazolidinone, (R)-4-isopropylthiazolidine-2-thione, (R)-(+)-3-boc-2,3-dimethyloxazolidine-4-carboxaldehyde, methyl (R)-(+)-3-boc-2,2-dimethyl-4-oxazolidinecarboxylate, methyl(S)-(−)-3-boc-2,2-dimethyl-4-oxazolidinecarboxylate, (S)-4-benzyloxazolidine-2-thione, (R)-4-phenylthiazolidine-2-thione, (R)-4-benzyloxazolidine-2-thione, (R)-(+)-4-benzyl-5,5-dimethyl-2-oxazolidinone, (S)-4-methyl-2-oxazolidinone, (S)-(+)-3-acetyl-4-benzyl-2-oxazolidinone, (4S,5R)-(−)-cis-4,5-diphenyl-2-oxazolidinone, (4R,5S)-(+)-cis-4,5-diphenyl-2-oxazolidinone, (+)-cinchonine, (2R,3R)-(−)-di-o-4-toluoyl-L-tartaric acid, (−)-quinic acid, (S)-(−)-2-methyl-2-propanesulfinamide solution, (S)-4-benzylthiazolidine-2-thione, (S)-(+)-S-methyl-S-phenylsulfoximine, (+)-N,N-diethylnorephedrine hydrochloride, (R)-(+)-4-isopropyl-5,5-diphenyl-2-oxazolidinone, (R)-(+)-4-tert-butyl-2-oxazolidinone, (S)-(+)-3-(benzyloxycarbonyl)-5-oxo-4-oxazolidineacetic acid, (S)-(+)-4-isopropyl-3-propionyl-2-oxazolidinone, (R)-4-benzyl-3-chloroacetyl-2-oxazolidinone, (R)-(+)-5,5-diphenyl-4-methyl-2-oxazolidinone, (S)-4-tert-butyl-2-oxazolidinone, (S)-(−)-4-isopropyl-2-oxazolidinethione, (R)-(−)-4-benzyl-3-propionyl-2-oxazoldinone, (R)-(+)-3-(benzyloxycarbonyl)-4-oxazolidinecarboxylic acid, (4S,5R)-(−)-4-methyl-5-phenyl-2-oxazolidinone, (S)-(−)-3-(benzyloxycarbonyl)-4-oxazolidinecarboxylic acid, (S)-(−)-4-isopropyl-5,5-dimethyl-2-oxazolidinone, (R)-(+)-4-isopropyl-5,5-dimethyl-2-oxazolidinone, (3aS-cis)-(−)-3,3a,8,8a-tetrahydro-2H-inden[1,2-d]oxazol-2-one, (S)-(−)-4-benzyl-5,5-dimethyl-2-oxazolidinone, (S)-(+)-3-(benzyloxycarbonyl)-5-oxo-4-oxazolidinepropionic acid, (S)-(+)-5,5-dimethyl-4-phenyl-2-oxazolidinone, (S)-(−)-5,5-diphenyl-4-(phenylmethyl)-2-oxazolidinone, (R)-(+)-4-(hydroxymethyl)-2-oxazolidinone benzoate, (4S)-4-(chloromethyl)-3-[(1R)-1-phenylethyl]-2-oxazolidinone, and (5R)-(hydroxymethyl-3-[(1R)-1-phenylethyl]-2-oxazolidinone. In a further embodiment, the chiral auxiliary agent is (R)-2-phenylpropanoic acid.

In some embodiments, the coupling step is performed in the presence of a coupling agent. Examples of coupling agents include, but are not limited to, dicyclohexyl carbodiimide (DCC), 1-ethyl-3-(3-dimethylaminopropyl) carbodiimide (EDC), and diisopropylcarbodiimide (DIC). In a further embodiment, the coupling agent is DCC.

In some embodiments, the coupling step is performed in the presence of an activating agent. Examples of activating agents include, but are not limited to, pyridine, triethylamine, and 4-dimethylaminopyridine (DMAP). In a further embodiment, the activating agent is DMAP.

In some embodiments, the coupling step is performed in a polar solvent. Examples of polar solvents include, but are not limited to, dichloromethane, methyl tert-butyl ether (MTBE), cyclopentyl methyl ether (CPME), actic anhydride, and acetonitrile.

In some embodiments, the halo-chromanol derivative is coupled to the compound having a structure:

R²—R¹³

In some embodiments, R¹³ is —O(C2-C4 alkenyl).

In some embodiments, the halo-chromanol derivative is coupled to a compound having a structure:

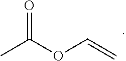

In some embodiments, coupling is in the presence of an enzyme. In a further embodiment, the enzyme is Novozyme 435 or Novozyme 51032. In a still further embodiment, the enzyme is Novozyme 435.

In some embodiments, coupling is in the presence of an aprotic solvent. In a further embodiment, the aprotic solvent is methyl tert-butyl ether (MTBE).

In some embodiments, coupling is at an elevated temperature. In a further embodiment, the elevated temperature is at about 30 °C.

In some embodiments, the halo-chromanol derivative has a structure:

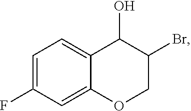 [SMILES: OC1c2ccc(F)cc2OCC1Br]

or a salt thereof.

In some embodiments, the method further comprises reacting a chromene derivative having a structure represented by a formula:

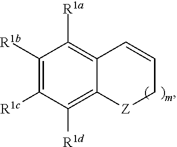

with a halogenating agent in a protic solvent, thereby providing the halo-chromanol derivative.

In some embodiments, the halogen source is N-bromosuccinimide (NBS).

In some embodiments, the protic solvent is an alcohol. In a further embodiment, the protic solvent is water.

In some embodiments, the method further comprises dehydrating a chromanol derivative having a structure represented by a formula:

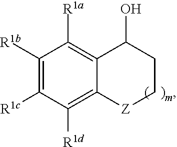

thereby providing the chromene derivative.

In some embodiments, dehydrating is via reaction with an acid. In a further embodiment, the acid is 4-methylbenzenesulfonic acid.

In some embodiments, the method further comprises reducing a chromanone derivative having a structure represented by a formula:

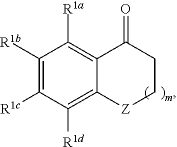

thereby providing the chromanol derivative.

In some embodiments, reducing is via addition of a reducing agent. In a further embodiment, the reducing agent is sodium borohydride.

In some embodiments, the method further comprises coupling the chromanamine derivative and a pyrrolopyrimidine having a structure represented by a formula:

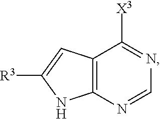

wherein X³ is a halogen; and wherein R³ is selected from C1-C6 haloalkyl, C1-C6 haloalkoxy, C1-C6 halohydroxy alkyl, and an unsubstituted 3- to 6-membered cycloalkyl, or a salt thereof, thereby providing a pyrrolopyridine having a structure represented by a formula:

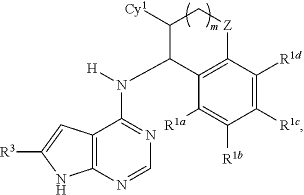

or a pharmaceutically acceptable salt thereof.

In some embodiments, the method further comprises coupling the chromanamine derivative and a pyrrolopyrimidine having a structure represented by a formula:

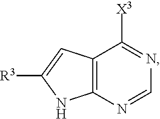

wherein X³ is a halogen; and wherein R³ is selected from C1-C6 haloalkyl, C1-C6 haloalkoxy, C1-C6 halohydroxy alkyl, and an unsubstituted 3- to 6-membered cycloalkyl, or a salt thereof, thereby providing a pyrrolopyridine having a structure represented by a formula:

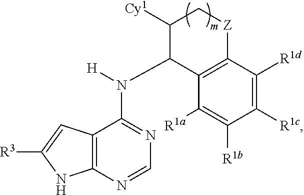

or a pharmaceutically acceptable salt thereof.

In some embodiments, coupling is in the presence of a base. In a further embodiment, the base is an amine base. Examples of amine bases include, but are not limited to, diethylamine, triethylamine, diisopropylamine, and diisopropylethylamine.

In some embodiments, coupling is in a protic solvent. In a further embodiment, the protic solvent is an alcohol. In a still further embodiment, the alcohol is n-butanol.

In some embodiments, coupling is at a temperature of from about 150 °C to about 190 °C. In a further embodiment, coupling is at a temperature of from about 150 °C to about 180 °C, about 150 °C to about 170 °C, about 150 °C to about 160 °C, about 160 °C to about 190 °C, about 170 °C to about 190 °C, about 180 °C to about 190 °C, about 160 °C to about 180 °C, or about 165 °C to about 175 °C. In a still further embodiment, coupling is at a temperature of about 170 °C.

In some embodiments, the chromanamine derivative has a structure represented by a formula:

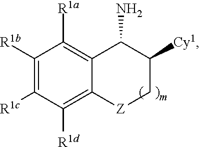

or a salt thereof.

In some embodiments, the chromanamine derivative has a structure represented by a

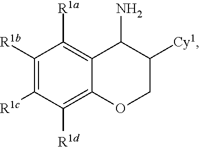

or a salt thereof.

In some embodiments, the chromanamine derivative has a structure represented by a formula:

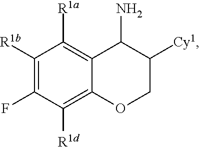

or a salt thereof.

In some embodiments, the chromanamine derivative has a structure represented by a formula:

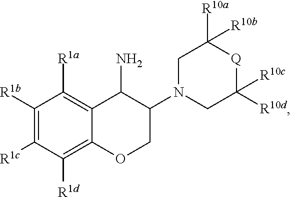

or a salt thereof.

In some embodiments, the chromanamine derivative has a structure represented by a formula:

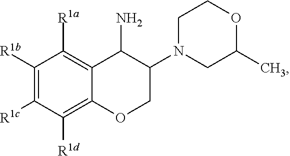

or a salt thereof.

In some embodiments, the chromanamine derivative has a structure represented by a formula:

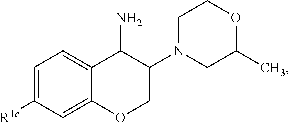

wherein R^{1c} is a non-hydrogen group, or a salt thereof.

In some embodiments, the chromanamine derivative has a structure represented by a formula:

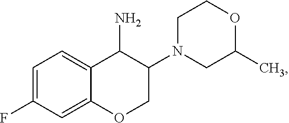 [SMILES: CC1CN(C2COc3cc(F)ccc3C2N)CCO1]

or a salt thereof.

In some embodiments, the chromanamine derivative has a structure represented by a formula:

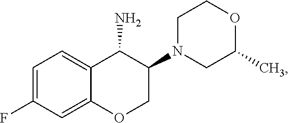 [SMILES: C[C@@H]1CN([C@H]2COc3cc(F)ccc3[C@@H]2N)CCO1]

or a salt thereof.

In some embodiment, provided herein are methods comprising: (a) epoxidizing a chromane derivative having a structure represented by a formula:

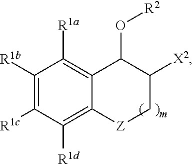

wherein m is 0 or 1; wherein X² is a halogen; and wherein Z is selected from —CH₂—, —O—, and —NH—; wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein R² is —C(O)(C1-C4 alkyl) or a residue of a chiral auxiliary group, or a salt thereof; and (b) reacting the epoxide with a cyclic amine having a structure represented by a formula:

Cy¹-H,
wherein Cy¹ is a 3- to 10-membered nitrogen-linked heterocycle substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, thereby making an alcohol having a structure represented by a formula:

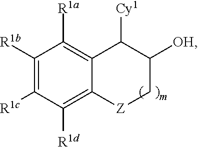

or a salt thereof.

In some embodiments, the epoxidation step is performed at room temperature.

In some embodiments, the epoxidation step is for a time period of from about 2 hours to about 8 hours. In a further embodiment, the epoxidation step is for a time period of from about 2 hours to about 7 hours, about 2 hours to about 6 hours, about 2 hours to about 5 hours, about 2 hours to about 4 hours, about 2 hours to about 3 hours, about 3 hours to about 8 hours, about 4 hours to about 8 hours, about 5 hours to about 8 hours, about 6 hours to about 8 hours, about 7 hours to about 8 hours, about 3 hours to about 7 hours, about 4 hours to about 6 hours, or about 4.5 hours to about 5.5 hours. In a still further embodiment, the epoxidation step is for a time period of about 4 hours.

In some embodiments, the epoxidation step is performed in an aprotic solvent. In a further embodiment, the aprotic solvent is diethyl ether.

In some embodiments, the epoxide is formed via addition of a base. In a further embodiment, the base is sodium methoxide.

In some embodiments, X² is bromo.

In some embodiments, R² is the residue of the chiral auxiliary group. Examples of chiral auxiliary agents include, but are not limited to, (R)-2-phenylpropanoic acid, (1R)-(+)-campanic aid, (R)-(+)-2-methyl-2-propanesulfinamide, (S)-(−)-1-phenylethylamine, (1R,2S)-(+)-pseudoephedrin, (R)-(+)-1-phenylethylamine, (1R,2S)-(−)-ephedrine, (−)-nicotine, (1S, 2R)-(+)-norephedrine, (S)-(−)-2-methyl-2-propanesulfinamide, (S)-4-benzyl-2-oxazolidinone, (1R,2S)-(−)-ephedrine hydrochloride, (R)-4-benzyl-2-oxazolidinone, (1R,2R)-(−)-pseudoephedrine, (1R,2S)-(−)-N-methylephedrine, (1S,2R)-(+)-ephedrine hydrochloride, (S)-(−)-4-isopropyl-2-oxazolidinone, (1S)-(−)-2,10-camphorsultam, (S)-(+)-4-phenyl-2-oxazolidinone, L(−)-malic acid, (−)-quinine, (S)-(−)-3-boc-2,2-dimethyloxazolidine-4-carboxaldehyde, (1S,2R)-(+)-N-methylephedrine, (R)-(−)-4-phenyl-2-oxazolidinone, (−)-shikimic acid, (S)-(+)-4-benzyl-3-propionyl-2-oxazolidinone, (R)-4-benzylthiazolidine-2-thione, (S)-4-isopropylthiazolidine-2-thione, (1S,2S)-(+)-norpseudoephedrine, (R)-(−)-mandelic acid, D-(−)-N-methylglucamine, (2S,3S)-(+)-di-o-benzoyltartaric acid, (4R,5S)-(+)-4-methyl-5-phenyl-2-oxazolidinone, (1S)-(+)-camphor-10-sulfonic acid, (S)-4-(4-aminobenzyl)-2(1H)-oxazolidinone, (R)-4-isopropylthiazolidine-2-thione, (R)-(+)-3-boc-2,3-dimethyloxazolidine-4-carboxaldehyde, methyl (R)-(+)-3-boc-2,2-dimethyl-4-oxazolidinecarboxylate, methyl(S)-(−)-3-boc-2,2-dimethyl-4-oxazolidinecarboxylate, (S)-4-benzyloxazolidine-2-thione, (R)-4-phenylthiazolidine-2-thione, (R)-4-benzyloxazolidine-2-thione, (R)-(+)-4-benzyl-5,5-dimethyl-2-oxazolidinone, (S)-4-methyl-2-oxazolidinone, (S)-(+)-3-acetyl-4-benzyl-2-oxazolidinone, (4S,5R)-(−)-cis-4,5-diphenyl-2-oxazolidinone, (4R,5S)-(+)-cis-4,5-diphenyl-2-oxazolidinone, (+)-cinchonine, (2R,3R)-(−)-di-o-4-toluoyl-L-tartaric acid, (−)-quinic acid, (S)-(−)-2-methyl-2-propanesulfinamide solution, (S)-4-benzylthiazolidine-2-thione, (S)-(+)-S-methyl-S-phenylsulfoximine, (+)-N,N-diethylnorephedrine hydrochloride, (R)-(+)-4-isopropyl-5,5-diphenyl-2-oxazolidinone, (R)-(+)-4-tert-butyl-2-oxazolidinone, (S)-(+)-3-(benzyloxycarbonyl)-5-oxo-4-oxazolidineacetic acid, (S)-(+)-4-isopropyl-3-propionyl-2-oxazolidinone, (R)-4-benzyl-3-chloroacetyl-2-oxazolidinone, (R)-(+)-5,5-diphenyl-4-methyl-2-oxazolidinone, (S)-4-tert-butyl-2-oxazolidinone, (S)-(−)-4-isopropyl-2-oxazolidinethione, (R)-(−)-4-benzyl-3-propionyl-2-oxazoldinone, (R)-(+)-3-(benzyloxycarbonyl)-4-oxazolidinecarboxylic acid, (4S,5R)-(−)-4-methyl-5-phenyl-2-oxazolidinone, (S)-(−)-3-(benzyloxycarbonyl)-4-oxazolidinecarboxylic acid, (S)-(−)-4-isopropyl-5,5-dimethyl-2-oxazolidinone, (R)-(+)-4-isopropyl-5,5-dimethyl-2-oxazolidinone, (3aS-cis)-(−)-3,3a,8,8a-tetrahydro-2H-inden[1,2-d]oxazol-2-one, (S)-(−)-4-benzyl-5,5-dimethyl-2-oxazolidinone, (S)-(+)-3-(benzyloxycarbonyl)-5-oxo-4-oxazolidinepropionic acid, (S)-(+)-5,5-dimethyl-4-phenyl-2-oxazolidinone, (S)-(−)-5,5-diphenyl-4-(phenylmethyl)-2-oxazolidinone, (R)-(+)-4-(hydroxymethyl)-2-oxazolidinone benzoate, (4S)-4-(chloromethyl)-3-[(1R)-1-phenylethyl]-2-oxazolidinone, and (5R)-(hydroxymethyl-3-[(1R)-1-phenylethyl]-2-oxazolidinone. In a further embodiment, the chiral auxiliary agent is (R)-2-phenylpropanoic acid.

In some embodiments, the residue of the chiral auxiliary group is:

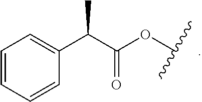 [SMILES: *OC(=O)[C@H](C)c1ccccc1]

In some embodiments, R² is —C(O)(C1-C4 alkyl). In a further embodiment, R² is —C(O)CH₃.

In some embodiments, the chromane derivative has a structure represented by a formula:

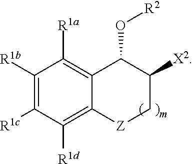

In some embodiments, the chromane derivative has a structure represented by a formula:

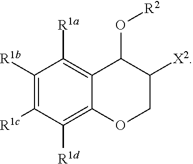

In some embodiments, the chromane derivative has a structure represented by a formula:

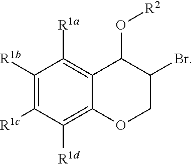

In some embodiments, the chromane derivative has a structure represented by a

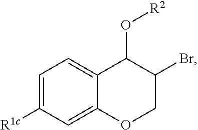

wherein R^{1c} is a non-hydrogen group.

In some embodiments, the chromane derivative has a structure represented by a formula:

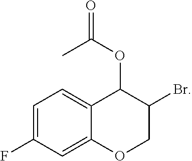 [SMILES: CC(=O)OC1c2ccc(F)cc2OCC1Br]

In some embodiments, the chromane derivative has a structure represented by a formula:

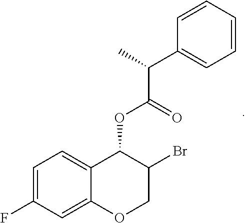 [SMILES: C[C@@H](C(=O)O[C@H]1c2ccc(F)cc2OCC1Br)c1ccccc1]

In some embodiments, the chromane derivative has a structure represented by a formula:

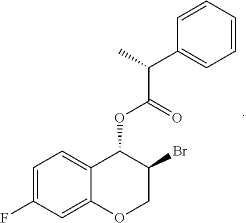 [SMILES: C[C@@H](C(=O)O[C@H]1c2ccc(F)cc2OC[C@@H]1Br)c1ccccc1]

In some embodiments, Cy¹ is selected from a pyrrolidinyl, imidazolidinyl, piperidinyl, piperazinyl, and morpholinyl ring, and is substituted with 0, 1, 2, 3, 4, 5, 6, 7, or 8 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino.

In some embodiments, the cyclic amine has a structure represented by a formula:

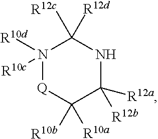

wherein Q is selected form —CH₂—, —O—, and —NR¹¹—; wherein R¹¹, when present, is selected from hydrogen and C1-C4 alkyl; wherein each of R^{10a}, R^{10b}, R^{10c}, and R^{10d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein each of R^{12a}, R^{12b}, R^{12c}, and R^{12d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino.

In some embodiments, Q is —O—.

In some embodiments, each of R^{12a}, R^{12b}, R^{12c}, and R^{12d} is hydrogen.

In some embodiments, the cyclic amine has a structure represented by a formula:

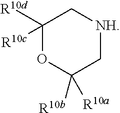

In some embodiments, the cyclic amine has a structure represented by a formula:

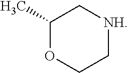

In some embodiments, the method further comprises coupling a halo-chromanol derivative having a structure represented by a formula:

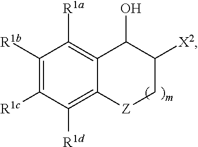

or a salt thereof, and either a chiral auxiliary agent or a compound having a structure:

R²—R¹³,
wherein R¹³ is selected from —OH, —O(C1-C4 alkyl), and —O(C2-C4 alkenyl), thereby providing the chromane derivative.

In some embodiments, the method further comprises reacting a chromene derivative having a structure represented by a formula:

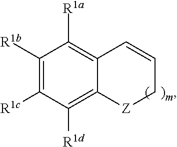

and a halogen source in a protic solvent, thereby providing the halo-chromanol derivative.

In some embodiments, the method further comprises dehydrating a chromanol derivative having a structure represented by a formula:

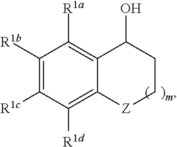

thereby providing the chromene derivative.

In some embodiments, the method further comprises reducing a chromanone derivative having a structure represented by a formula:

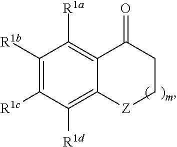

thereby providing the chromanol derivative.

In some embodiments, the method further comprises activating the alcohol, thereby forming an activated alcohol, and displacing the activated alcohol via addition of an amine, thereby providing a chromanamine derivative having a structure represented by a formula:

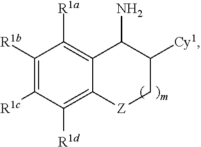

or a salt thereof.

In some embodiments, the method further comprises coupling the chromanamine derivative and a pyrrolopyrimidine having a structure represented by a formula:

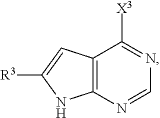

wherein X³ is a halogen; and wherein R³ is selected from C1-C6 haloalkyl, C1-C6 haloalkoxy, C1-C6 halohydroxy alkyl, and an unsubstituted 3- to 6-membered cycloalkyl, or a salt thereof, thereby providing a pyrrolopyridine having a structure represented by a formula:

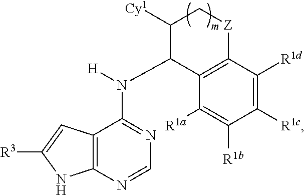

or a pharmaceutically acceptable salt thereof.

In some embodiments, the alcohol has a structure represented by a formula:

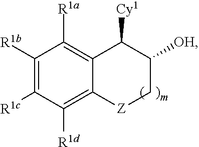

or a salt thereof.

In some embodiments, the alcohol has a structure represented by a formula:

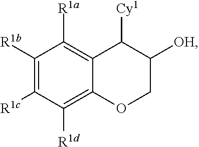

or a salt thereof.

In some embodiments, the alcohol has a structure represented by a formula:

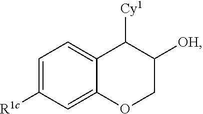

wherein R^{1c} is a non-hydrogen group, or a salt thereof.

In some embodiments, the alcohol has a structure represented by a formula:

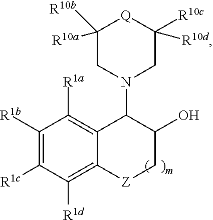

wherein Q is selected form —CH₂—, —O—, and —NR¹¹; wherein R¹¹, when present, is selected from hydrogen and C1-C4 alkyl; and wherein each of R^{10a}, R^{10b}, R^{10c}, and R^{10d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a salt thereof.

In some embodiments, the alcohol has a structure represented by a formula:

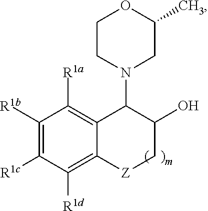

or a salt thereof.

In some embodiments, the alcohol has a structure represented by a formula:

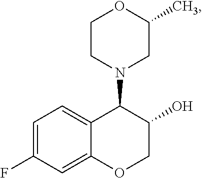 [SMILES: C[C@@H]1CN([C@@H]2c3ccc(F)cc3OC[C@H]2O)CCO1]

or a salt thereof.

In some embodiments, provided herein are methods comprising the mesylation of the alcohol of paragraphs [0470]-[0475] to obtain a structure represented by formula:

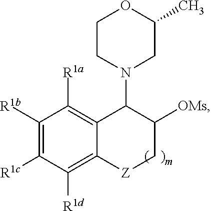

or a salt thereof.

In some embodiments, the mesylation structure is represented by formula:

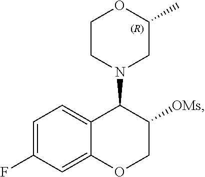

or a salt thereof.

In some embodiments, provided herein are methods comprising reacting a mesylate structure (such as, those presented in paragraphs and/or [0477]) with a benzylamine to obtain a benzylamine structure represented by formula:

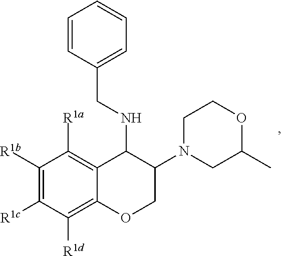

or a salt thereof, wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, provided that at least one of R^{1a}, R^{1b}, R^{1c}, and R^{1d} is not hydrogen, or a salt thereof.

In some embodiments, the benzylamine structure is represented by formula:

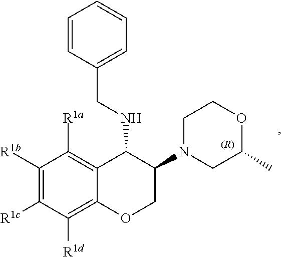

or a salt thereof, wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, provided that at least one of R^{1a}, R^{1b}, R^{1c}, and R^{1d} is not hydrogen, or a salt thereof.

In some embodiments, the benzylamine structure is represented by formula:

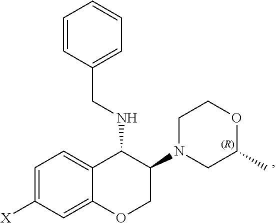

or a salt thereof, wherein X is a halogen.

In some embodiments, the benzylamine structure represented by formula:

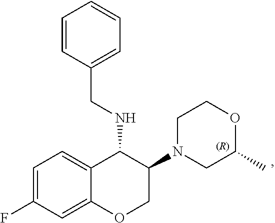

or a salt thereof.

In some embodiments, the benzylamine structure may be reduced to obtain the chromanamine derivative having a structure represented by formula:

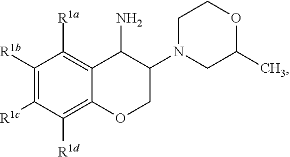

or a salt thereof.

In some embodiments, the chromanamine derivative has a structure represented by the following formula:

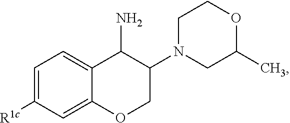

wherein R^{1c} is a non-hydrogen group, or a salt thereof.

In some embodiments, the chromanamine derivative has a structure represented by formula:

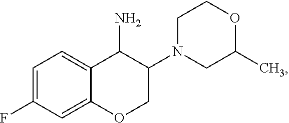 [SMILES: CC1CN(C2COc3cc(F)ccc3C2N)CCO1]

or a salt thereof.

In some embodiments, the chromanamine derivative has a structure represented by formula:

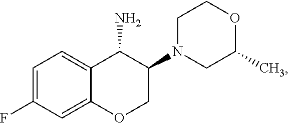 [SMILES: C[C@@H]1CN([C@H]2COc3cc(F)ccc3[C@@H]2N)CCO1]

or a salt thereof.

In some embodiments, provided herein are methods comprising activating a chromanol derivative (e.g., from the alcohol of paragraphs [0470]-[0475]) having a structure represented by a formula:

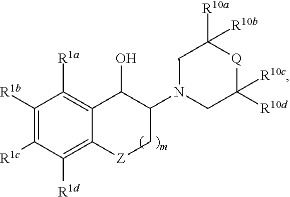

wherein m is 0 or 1; wherein Q is selected form —CH₂—, —O—, and —NR¹¹; wherein R¹¹, when present, is selected from hydrogen and C1-C4 alkyl; wherein Z is selected from —CH₂—, —O—, and —NH—; wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein each of R^{10a}, R^{10b}, R^{10c}, and R 10d is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a salt thereof, thereby forming an activated alcohol, and displacing the activated alcohol via addition of an amine, thereby providing a chromanamine derivative having a structure represented by a formula:

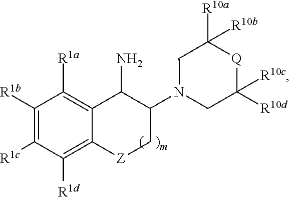

or a salt thereof.

In some embodiments, the method further comprises reducing a chromenone derivative having a structure represented by a formula:

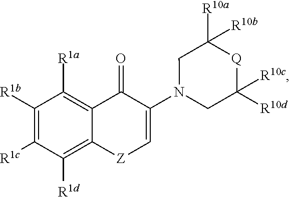

or a salt thereof, thereby providing the chromanol derivative. In some embodiments, reducing is via dynamic kinetic resolution.

In some embodiments, the chromenone derivative has a structure represented by a

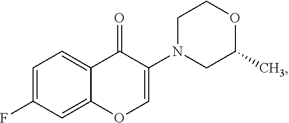 [SMILES: C[C@@H]1CN(c2coc3cc(F)ccc3c2=O)CCO1]

or a salt thereof.

In some embodiments, the method further comprises reacting a di-halide having a structure represented by a formula:

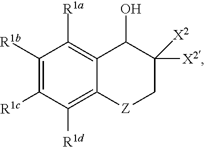

wherein each of X² and X^{2′} is independently halogen, or a salt thereof, and a cyclic amine having a structure represented by a formula:

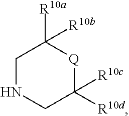

or a salt thereof, and eliminating a halide group, thereby providing the chromenone derivative.

In some embodiments, the di-halide has a structure represented by a formula:

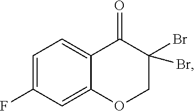 [SMILES: O=C1c2ccc(F)cc2OCC1(Br)Br]

or a salt thereof.

In some embodiments, the method further comprises di-halogenating a chromanone derivative having a structure represented by a formula:

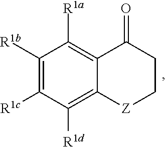

or a salt thereof, thereby providing the di-halide.

In some embodiments, the chromanone derivative has a structure represented by a

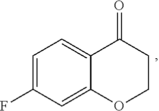 [SMILES: O=C1CCOc2cc(F)ccc21]

or a salt thereof.

In some embodiments, the method further comprises coupling the chromanamine derivative and a pyrrolopyrimidine having a structure represented by a formula:

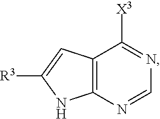

wherein X³ is a halogen; and wherein R³ is selected from C1-C6 haloalkyl, C1-C6 haloalkoxy, C1-C6 halohydroxy alkyl, and an unsubstituted 3- to 6-membered cycloalkyl, or a salt thereof, thereby providing a pyrrolopyridine having a structure represented by a formula:

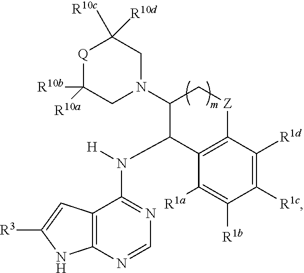

or a pharmaceutically acceptable salt thereof.

In some embodiments, the method further comprises reducing a chromanone derivative having a structure represented by a formula:

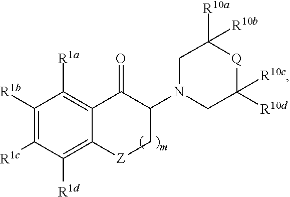

or a salt thereof, thereby providing the chromanol derivative.

In some embodiments, the chromanone derivative has a structure represented by a formula:

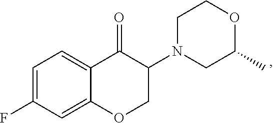 [SMILES: C[C@@H]1CN(C2COc3cc(F)ccc3C2=O)CCO1]

or a salt thereof.

In some embodiments, the method further comprises reacting a halide having a structure represented by a formula:

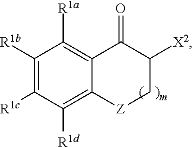

wherein X² is a halogen, or a salt thereof, and a cyclic amine having a structure represented by a formula:

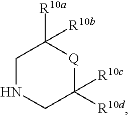

or a salt thereof, thereby providing the chromanone derivative.

In some embodiments, the halide has a structure represented by a formula:

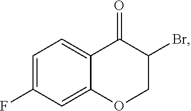 [SMILES: O=C1c2ccc(F)cc2OCC1Br]

or a salt thereof.

In some embodiments, the method further comprises coupling the chromanamine derivative and a pyrrolopyrimidine having a structure represented by a formula:

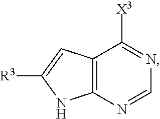

wherein X³ is a halogen; and wherein R³ is selected from C1-C6 haloalkyl, C1-C6 haloalkoxy, C1-C6 halohydroxy alkyl, and an unsubstituted 3- to 6-membered cycloalkyl, or a salt thereof, thereby providing a pyrrolopyridine having a structure represented by a formula:

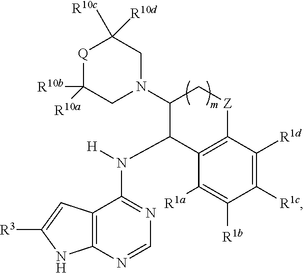

or a pharmaceutically acceptable salt thereof.

In some embodiments, the method provides a pyrrolopyridine having a structure:

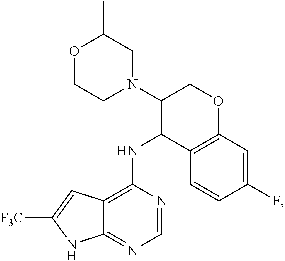 [SMILES: CC1CN(C2COc3cc(F)ccc3C2Nc2ncnc3[nH]c(C(F)(F)F)cc23)CCO1]

or a pharmaceutically acceptable salt thereof.

In some embodiments, the method provides a pyrrolopyridine having a structure:

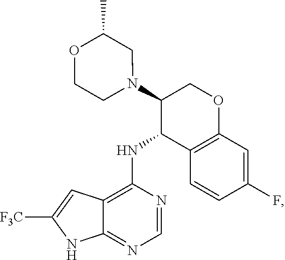 [SMILES: C[C@@H]1CN([C@H]2COc3cc(F)ccc3[C@@H]2Nc2ncnc3[nH]c(C(F)(F)F)cc23)CCO1]

or a pharmaceutically acceptable salt thereof.

In some embodiments, the method provides a pyrrolopyridine having a structure:

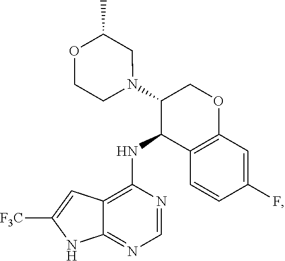 [SMILES: C[C@@H]1CN([C@@H]2COc3cc(F)ccc3[C@H]2Nc2ncnc3[nH]c(C(F)(F)F)cc23)CCO1]

or a pharmaceutically acceptable salt thereof.

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, as described and exemplified below. In certain specific examples, the disclosed compounds can be prepared by Routes I-VII, as described and exemplified below. The following examples are provided so that the invention might be more fully understood, are illustrative only, and should not be construed as limiting.

### 1. Route I

In some embodiments, N-(3-substituted-chroman-4-yl)-7H-pyrrolo[2,3-d]pyrimidin-4-amine compounds can be prepared as shown below.

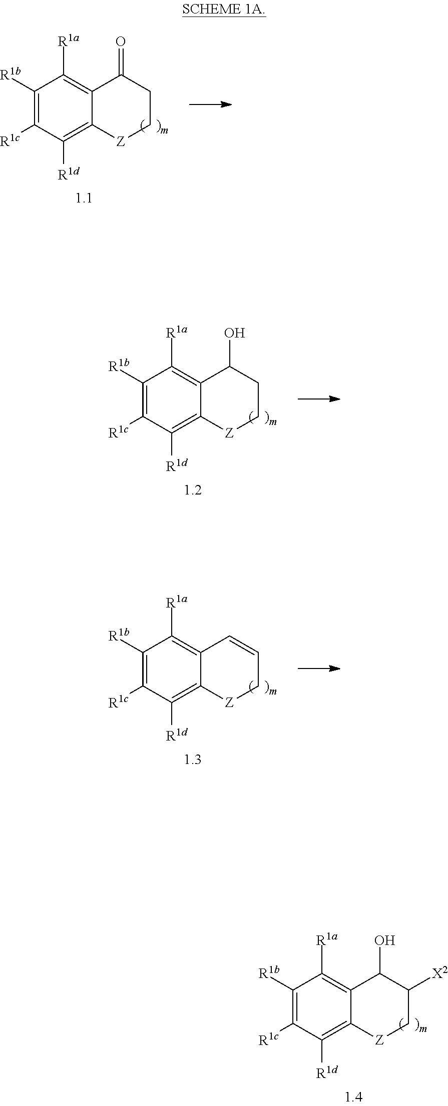

Compounds are represented in generic form, with substituents as noted in compound descriptions elsewhere herein. A more specific example is set forth below.

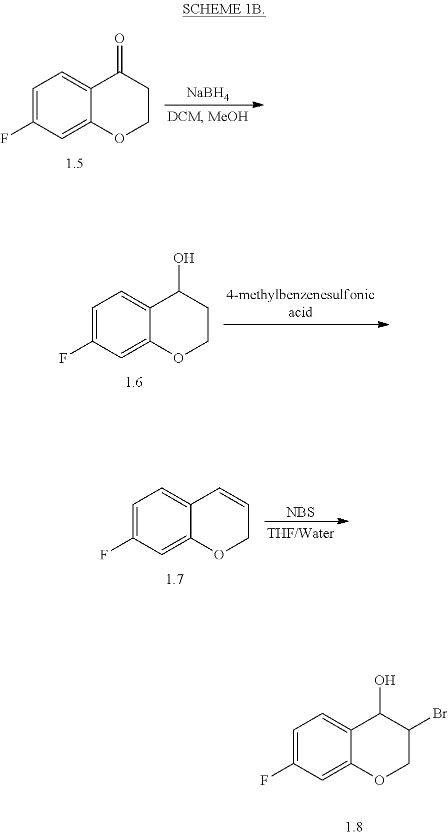

In some embodiments, compounds of type 1.8, and similar compounds, can be prepared according to reaction Scheme 1B above. Thus, compounds of type 1.5 can be prepared by reduction of an appropriate ketone, e.g., 1.5 as shown above. Appropriate ketones are commercially available or prepared by methods known to one skilled in the art. The reduction is carried out in the presence of an appropriate reducing agent, e.g., sodium borohydride, in an appropriate solvent, e.g., dichloromethane (DCM), in an appropriate solvent, e.g., methanol (MeOH). Compounds of type 1.7 can be prepared by dehydration of an appropriate alcohol, e.g., 1.6 as shown above. The dehydration is carried out in the presence of an appropriate acid, e.g., 4-methylbenzenesulfonic acid. Compounds of type 1.8 can be prepared by halohydrin formation from an appropriate alkene, e.g., 1.17 as shown above. The halohydrin formation is carried out in the presence of an appropriate halide source, e.g., N-bromosuccinimide (NBS), an appropriate hydroxide source, e.g., water, in an appropriate solvent, e.g., tetrahydrofuran (THF). As can be appreciated by one skilled in the art, the above reaction provides an example of a generalized approach wherein compounds similar in structure to the specific reactants above (compounds similar to compounds of type 1.1, 1.2, and 1.3), can be substituted in the reaction to provide compounds similar to Formula 1.4.

### 2. Route 2

In some embodiments, N-(3-substituted-chroman-4-yl)-7H-pyrrolo[2,3-d]pyrimidin-4-amine compounds can be prepared as shown below.

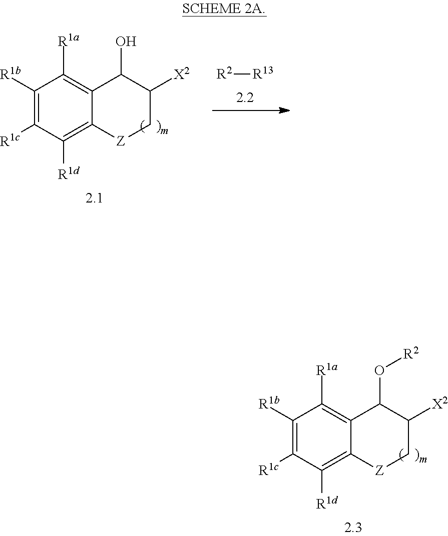

Compounds are represented in generic form, with substituents as noted in compound descriptions elsewhere herein. More specific examples are set forth below.

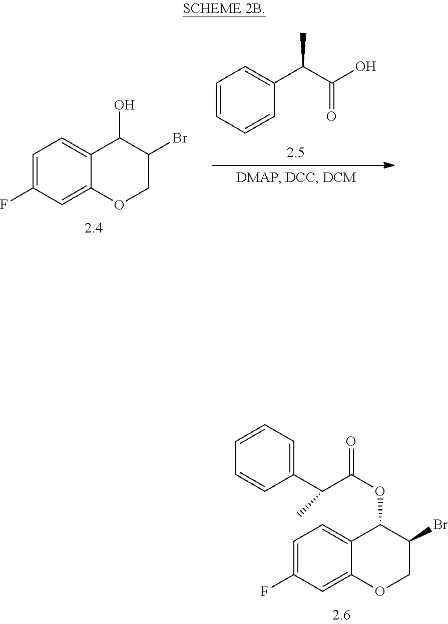

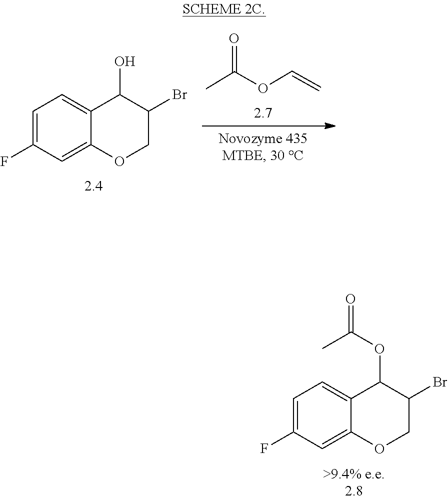

In some embodiments, compounds of type 2.6 and 2.8, and similar compounds, can be prepared according to reaction Scheme 2B and Scheme 2C above, respectively. Thus, compounds of type 2.6 and 2.8 can be prepared by a coupling reaction between an appropriate halohydrin, e.g., 2.4 as shown above, and an appropriate carboxylate, e.g., 2.5 and 2.6 as shown above. Appropriate carboxylates are commercially available or prepared by methods known to one skilled in the art. The coupling reaction is carried out in the presence of an appropriate coupling agent, e.g., N,N′-dicyclohexylcarbodiimide (DCC), and an appropriate activating agent, e.g., 4-dimethylaminopyridine (DMAP), in an appropriate solvent, dichloromethane (DCM). Alternatively, the coupling reaction is carried out in the presence of an appropriate enzyme, e.g., Novozyme 435, in an appropriate solvent, e.g., methyl tert-butyl ether (MTBE), at an appropriate temperature, e.g., 30 °C. As can be appreciated by one skilled in the art, the above reaction provides an example of a generalized approach wherein compounds similar in structure to the specific reactants above (compounds similar to compounds of type 2.1 and 2.2), can be substituted in the reaction to provide compounds similar to Formula 2.3.

### 3. Route 3

In some embodiments, N-(3-substituted-chroman-4-yl)-7H-pyrrolo[2,3-d]pyrimidin-4-amine compounds can be prepared as shown below.

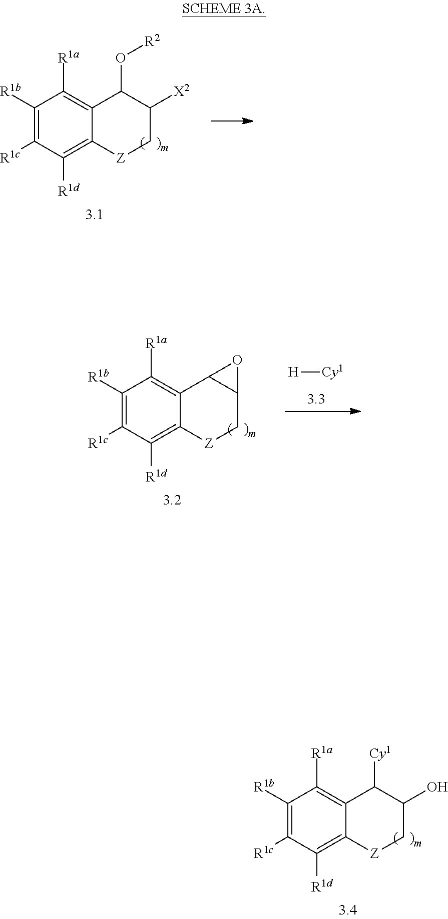

Compounds are represented in generic form, with substituents as noted in compound descriptions elsewhere herein. A more specific example is set forth below.

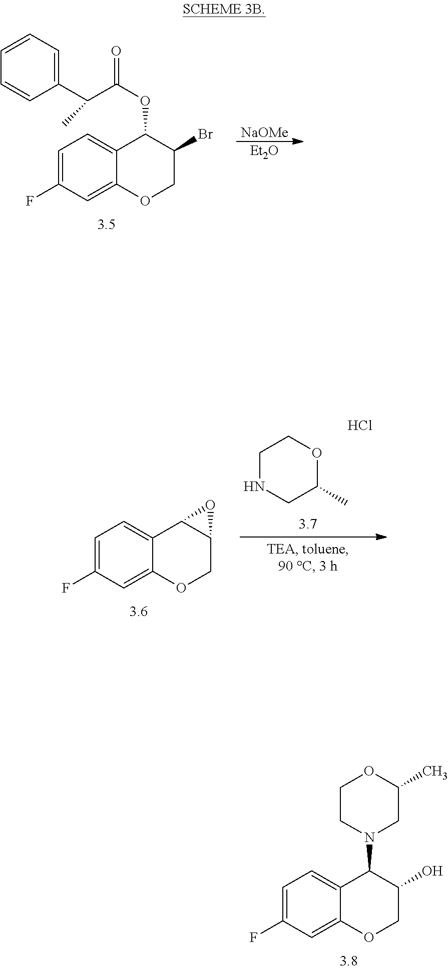

In some embodiments, compounds of type 3.8, and similar compounds, can be prepared according to reaction Scheme 3B above. Thus, compounds of type 3.6 can be prepared by epoxidation of an appropriate halide, e.g., 3.5 as shown above. The epoxidation reaction is carried out in the presence of an appropriate base, e.g., sodium methoxide, in an appropriate solvent, e.g., diethyl ether. Compounds of type 3.8 can be prepared by addition of an appropriate cyclic amine, e.g., 3.7 as shown above. Appropriate cyclic amines are commercially available or prepared by methods known to those of skill in the art. The addition is carried out in the presence of an appropriate base, e.g., triethylamine, in an appropriate solvent, e.g., toluene, at an appropriate temperature, e.g., 90 °C, for an appropriate period of time, e.g., 3 hours. As can be appreciated by one skilled in the art, the above reaction provides an example of a generalized approach wherein compounds similar in structure to the specific reactants above (compounds similar to compounds of type 3.1, 3.2, and 3.3), can be substituted in the reaction to provide compounds similar to Formula 3.4.

### 4. Route 4

In some embodiments, N-(3-substituted-chroman-4-yl)-7H-pyrrolo[2,3-d]pyrimidin-4-amine compounds can be prepared as shown below.

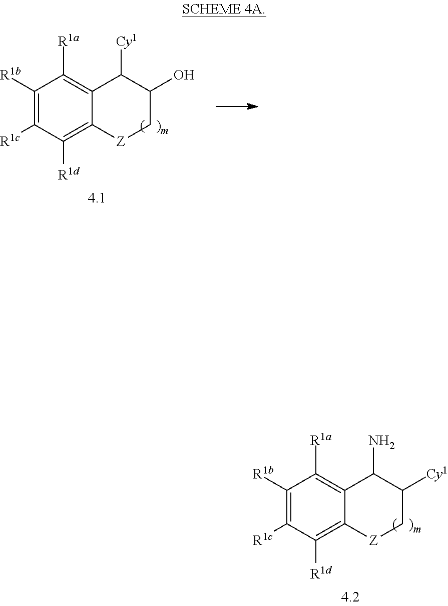

Compounds are represented in generic form, with substituents as noted in compound descriptions elsewhere herein. A more specific example is set forth below.

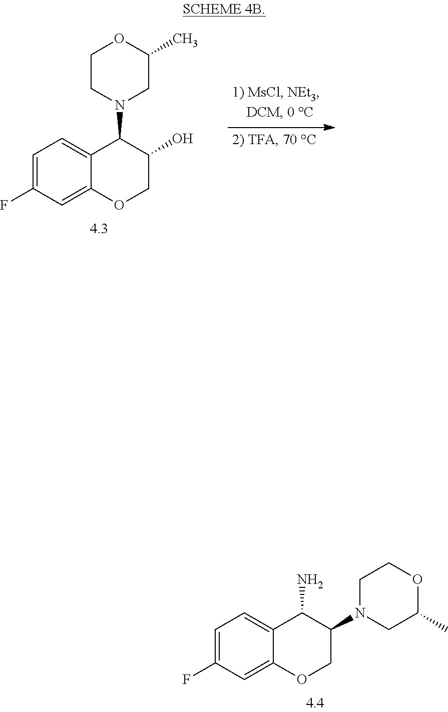 [SMILES: C[C@@H]1CN([C@@H]2c3ccc(F)cc3OC[C@H]2O)CCO1 ; C[C@@H]1CN([C@H]2COc3cc(F)ccc3[C@@H]2N)CCO1]

In some embodiments, compounds of type 4.4, and similar compounds, can be prepared according to reaction Scheme 4B above. Thus, compounds of type 4.4 can be prepared by rearrangement of an appropriate chromanol derivative, e.g., 4.3 as shown above. The rearrangement is carried out via addition of an appropriate activating agent, e.g., mesyl chloride, in an appropriate base, e.g., triethylamine, in an appropriate solvent, e.g., dichloromethane, at an appropriate temperature, e.g., 0 °C, followed by addition of an appropriate acid, e.g., trifluoroacetic acid, at an appropriate temperature, e.g., 70 °C. As can be appreciated by one skilled in the art, the above reaction provides an example of a generalized approach wherein compounds similar in structure to the specific reactants above (compounds similar to compounds of type 4.1), can be substituted in the reaction to provide compounds similar to Formula 4.2.

### 5. Route 5

In some embodiments, N-(3-substituted-chroman-4-yl)-7H-pyrrolo[2,3-d]pyrimidin-4-amine compounds can be prepared as shown below.

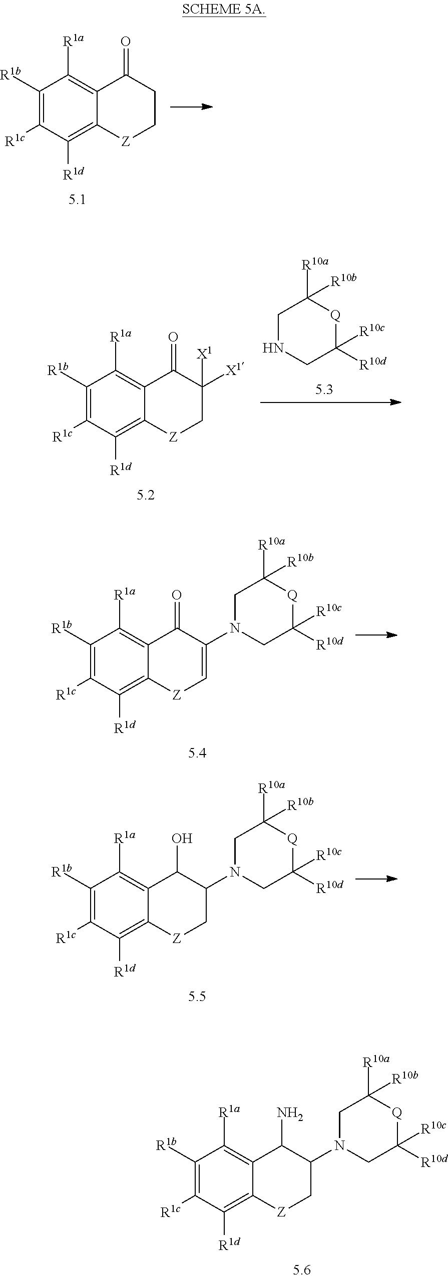

Compounds are represented in generic form, with substituents as noted in compound descriptions elsewhere herein. A more specific example is set forth below.

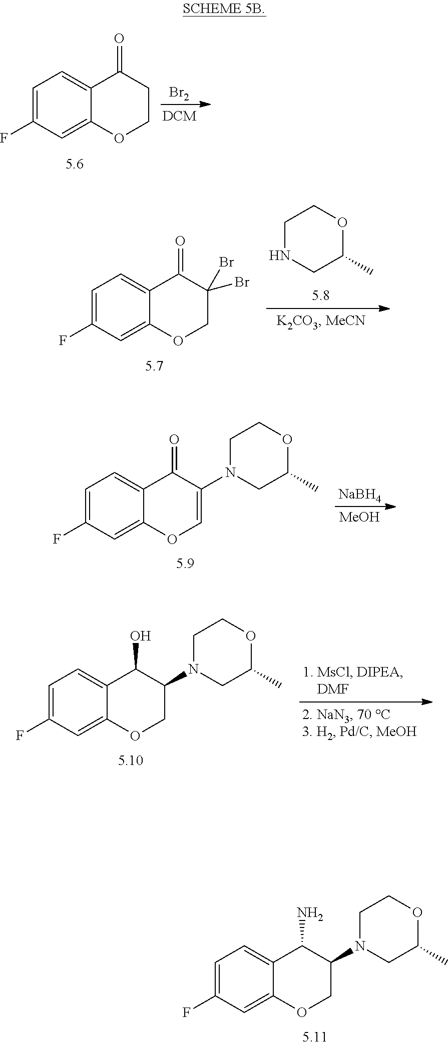

In some embodiments, compounds of type 5.10, and similar compounds, can be prepared according to reaction Scheme 5B above. Thus, compounds of type 5.7 can be prepared by halogenation of an appropriate tetrahydropyranone, e.g., 5.6 as shown above. Appropriate tetrahydropyranones are commercially available or prepared by methods known to one of skill in the art. The halogenation is carried out in the presence of an appropriate halide source, e.g., bromine, in an appropriate solvent, e.g., dichloromethane (DCM). Compounds of type 5.8 can be prepared by elimination of an appropriate halide, e.g., 5.7 as shown above, followed by substitution with an appropriate amine, e.g., 5.8 as shown above. Appropriate amines are commercially available or prepared by methods known to one of skill in the art. The elimination/substitution reaction is carried out in the presence of an appropriate base, e.g., potassium carbonate, in an appropriate solvent, e.g., acetonitrile. Compounds of type 5.9 can be prepared by reduction of an appropriate a, β-unsaturated ketone, e.g., 5.8 as shown above. The reduction is carried out in the presence of an appropriate reducing agent, e.g., sodium borohydride, in an appropriate solvent, e.g., methanol. See, e.g., Venkati et al. (2002) Synthetic Communications 32(14): 2227-2235. Compounds of type 5.10 can be prepared by a nucleophilic substitution reaction of an appropriate hydroxy analog, e.g., 5.9 as shown above. The nucleophilic substitution reaction is carried out by converting the hydroxyl group to a leaving group using, for example, mesyl chloride, in the presence of an appropriate base, e.g., diisopropylethylamine, in an appropriate solvent, e.g., dimethylformamide, followed by displacement and inversion of the stereocenter using an appropriate nucleophile, e.g., sodium azide, at an appropriate temperature, e.g., 70 °C. The azide can then be reduced using an appropriate reducing agent, e.g., hydrogen gas, in the presence of an appropriate catalyst, e.g., palladium on carbon, in an appropriate solvent, e.g., methanol. As can be appreciated by one skilled in the art, the above reaction provides an example of a generalized approach wherein compounds similar in structure to the specific reactants above (compounds similar to compounds of type 5.1, 5.2, 5.3, 5.4, and 5.5), can be substituted in the reaction to provide N-(3-substituted-chroman-4-yl)-7H-pyrrolo[2,3-d]pyrimidin-4-amine compounds similar to Formula 5.6.

### 6. Route 6

In some embodiments, N-(3-substituted-chroman-4-yl)-7H-pyrrolo[2,3-d]pyrimidin-4-amine compounds can be prepared as shown below.

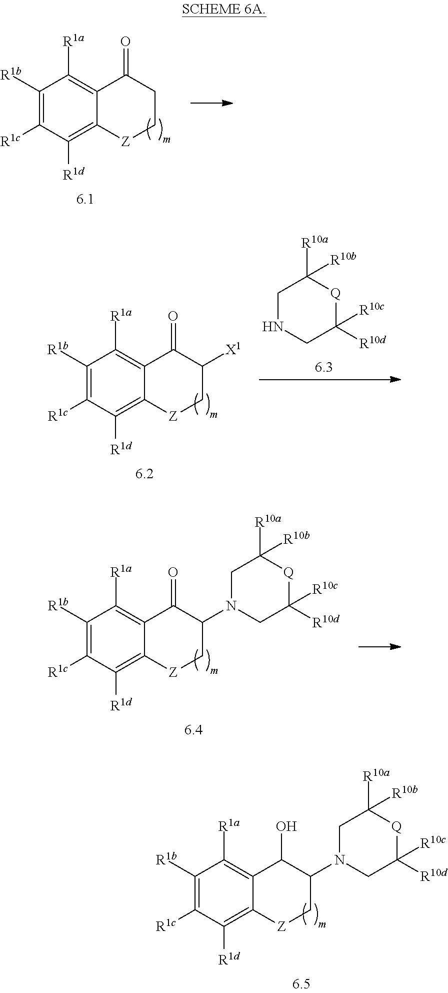

Compounds are represented in generic form, with substituents as noted in compound descriptions elsewhere herein. A more specific example is set forth below.

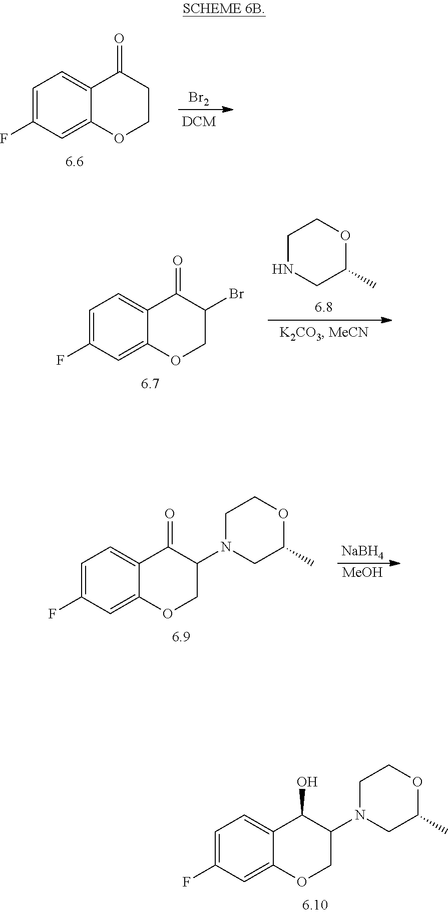

In some embodiments, compounds of type 6.9, and similar compounds, can be prepared according to reaction Scheme 6B above. Thus, compounds of type 6.7 can be prepared by halogenation of an appropriate tetrahydropyranone, e.g., 6.6 as shown above. Appropriate tetrahydropyranones are commercially available or prepared by methods known to one of skill in the art. The halogenation is carried out in the presence of an appropriate halide source, e.g., bromine, in an appropriate solvent, e.g., dichloromethane (DCM). Compounds of type 6.9 can be prepared by a substitution reaction between an appropriate halide, e.g., 6.7 as shown above, and an appropriate amine, e.g., 6.8 as shown above. Appropriate amines are commercially available or prepared by methods known to one of skill in the art. The substitution reaction is carried out in the presence of an appropriate base, e.g., potassium carbonate, in an appropriate solvent, e.g., acetonitrile. Compounds of type 6.10 can be prepared by reduction of an appropriate ketone, e.g., 5.9 as shown above. The reduction is carried out in the presence of an appropriate reducing agent, e.g., sodium borohydride, in an appropriate solvent, e.g., methanol. See, e.g., Venkati et al. (2002) Synthetic Communications 32(14): 2227-2235. As can be appreciated by one skilled in the art, the above reaction provides an example of a generalized approach wherein compounds similar in structure to the specific reactants above (compounds similar to compounds of type 6.1, 6.2, 6.3, and 6.4), can be substituted in the reaction to provide N-(3-substituted-chroman-4-yl)-7H-pyrrolo[2,3-d]pyrimidin-4-amine compounds similar to Formula 6.5.

### 7. Route 7

In some embodiments, N-(3-substituted-chroman-4-yl)-7H-pyrrolo[2,3-d]pyrimidin-4-amine compounds can be prepared as shown below.

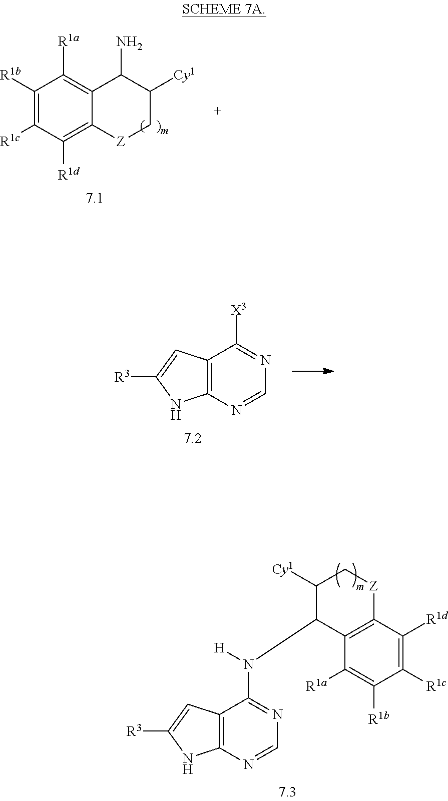

Compounds are represented in generic form, with substituents as noted in compound descriptions elsewhere herein. A more specific example is set forth below.

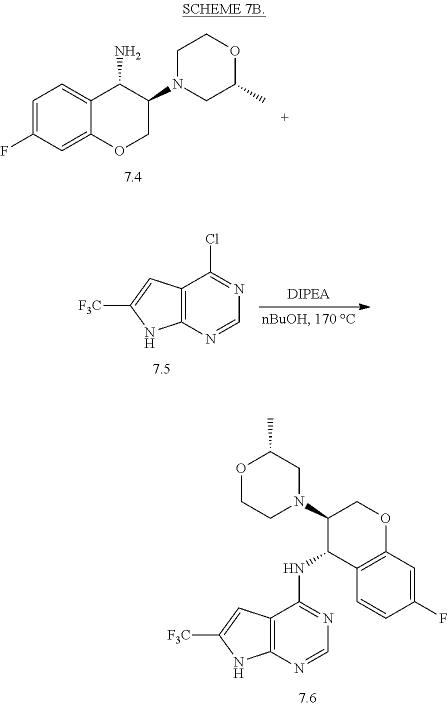 [SMILES: C[C@@H]1CN([C@H]2COc3cc(F)ccc3[C@@H]2N)CCO1 ; FC(F)(F)c1cc2c(Cl)ncnc2[nH]1 ; C[C@@H]1CN([C@H]2COc3cc(F)ccc3[C@@H]2Nc2ncnc3[nH]c(C(F)(F)F)cc23)CCO1]

In some embodiments, compounds of type 7.6, and similar compounds, can be prepared according to reaction Scheme 7B above. Thus, compounds of type 7.6 can be prepared by a coupling reaction between an appropriate amine, e.g., 7.4 as shown above, and an appropriate aryl halide, e.g., 7.5 as shown above. The coupling reaction is carried out in the presence of an appropriate base, e.g., diisopropylethylamine (DIPEA), in an appropriate solvent, e.g., n-butanol, at an appropriate temperature, e.g., 170 °C. As can be appreciated by one skilled in the art, the above reaction provides an example of a generalized approach wherein compounds similar in structure to the specific reactants above (compounds similar to compounds of type 7.1 and 7.2), can be substituted in the reaction to provide N-(3-substituted-chroman-4-yl)-7H-pyrrolo[2,3-d]pyrimidin-4-amine compounds similar to Formula 7.3.

Compounds and compositions described herein are generally useful for modulating the activity of PINK1. In some embodiments, the compounds and compositions described herein inhibit the activity of PINK1. The disclosure relates to a method of modulating PINK1 comprising contacting a cell expressing PINK1 with one or a plurality of compositions or pharmaceutical compositions disclosed herein. In some embodiments, the step of contacting comprising contacting for a time period sufficient to associate and to modulate the biological effect of PINK1 in the cell. In some embodiments the time period is about 5 minutes, 10 minute, 20 minutes, 30 minutes, 1 hour, 2, 3, 4, 5, 6, 7, 8, 9, 10, 11, 12, 16, or about 18 hours or more. In some embodiments, the cell is positioned in vitro. In some embodiments, the cell is within a subject, such as a human or dog.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### F. PHARMACEUTICAL COMPOSITIONS

In some embodiments, the present disclosure provides a pharmaceutical composition comprising a compound described herein and one or more pharmaceutically acceptable carriers or excipients.

In some embodiments, the present disclosure provides a pharmaceutical composition comprising a compound being prepared by a method described herein (e.g., a N-(3-substituted-chroman-4-yl)-7H-pyrrolo[2,3-d]pyrimidin-4-amine) and one or more pharmaceutically acceptable carriers or excipients.

In some embodiments, the present disclosure provides a pharmaceutical composition comprising an effective amount of a compound having a structure:

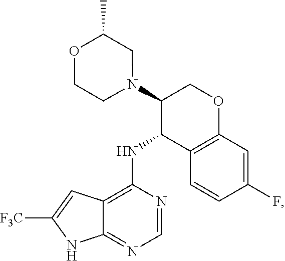 [SMILES: C[C@@H]1CN([C@H]2COc3cc(F)ccc3[C@@H]2Nc2ncnc3[nH]c(C(F)(F)F)cc23)CCO1]

or a pharmaceutically acceptable salt thereof, and a pharmaceutically acceptable carrier, wherein the compound has an enantiomeric purity of at least about 80%, at least about 85%, at least about 90%, at least about 95%, at least about 99%, or greater than 99%. In some embodiments, the compound can be provided in percent enantiomeric excess (e.e.). Thus, in various embodiments, the enantiomeric excess of the desired enantiomer of the disclosed pyrrolopyrimidine compounds is at least about 50%, at least about 60%, at least about 70%, at least about 75%, at least about 80%, at least about 85%, at least about 90%, at least about 95%, at least about 98%, or at least about 99%.

In some embodiments, the present disclosure provides a pharmaceutical composition comprising an effective amount of a compound of formula:

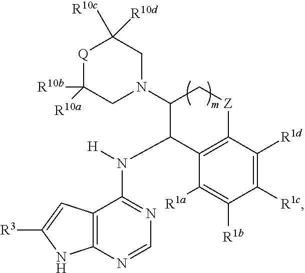

wherein m is 0 or 1; wherein Q is selected form —CH₂—, —O—, and —NR¹¹; wherein R¹¹, when present, is selected from hydrogen and C1-C4 alkyl; wherein Z is selected from —CH₂—, —O—, and —NH—; wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; wherein R³ is selected from C1-C6 haloalkyl, C1-C6 haloalkoxy, C1-C6 halohydroxy alkyl, and an unsubstituted 3- to 6-membered cycloalkyl; and wherein each of R^{10a}, R^{10b}, R^{10c}, and R^{10d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a salt thereof, wherein the compound has an enantiomeric purity of at least about 80%, at least about 85%, at least about 90%, at least about 95%, at least about 99%, or greater than 99%. In some embodiments, the compound can be provided in percent enantiomeric excess (e.e.). Thus, in various embodiments, the enantiomeric excess of the desired enantiomer of the disclosed pyrrolopyrimidine compounds is at least about 50%, at least about 60%, at least about 70%, at least about 75%, at least about 80%, at least about 85%, at least about 90%, at least about 95%, at least about 98%, or at least about 99%.

Pharmaceutically acceptable salts of the compounds are conventional acid-addition salts or base-addition salts that retain the biological effectiveness and properties of the compounds and are formed from suitable non-toxic organic or inorganic acids or organic or inorganic bases. Exemplary acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Example base-addition salts include those derived from ammonium, potassium, sodium and, quaternary ammonium hydroxides, such as for example, tetramethylammonium hydroxide. Chemical modification of a pharmaceutical compound into a salt is a known technique to obtain improved physical and chemical stability, hygroscopicity, flowability and solubility of compounds. See, e.g., H. Ansel et. al., Pharmaceutical Dosage Forms and Drug Delivery Systems (6th Ed. 1995) at pp. 196 and 1456-1457.

The pharmaceutical compositions comprise the compounds prepared by the methods disclosed herein with or in a pharmaceutically acceptable carrier. A pharmaceutically acceptable carrier refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. The compounds can be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

In further embodiments, the pharmaceutical composition is administered to a mammal. In still further embodiments, the mammal is a human. In an even further embodiment, the human is a patient.

In further embodiments, the pharmaceutical composition is administered following identification of the mammal in need of treatment of a disorder associated with PINK1 kinase activity. In still further embodiments, the mammal has been diagnosed with a need for treatment of a disorder associated with PINK1 kinase activity prior to the administering step.

In various embodiments, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

The pharmaceutical compositions typically include one or more compounds described herein, which can be used in combination with one another and/or with other active agents known to be useful in treating a disease associated neurodegeneration (e.g., Parkinson's disease, such as levodopa or amantadine, dopamine agonists (e.g., bromocriptine, pergolide, pramipexole, ropinirole, piribedil, cabergoline, apomorphine, lisuride, tetrabenazine), MAO-B modulators (e.g., selegiline or rasagiline), amantadine, anticholinergics, antipsychotics (e.g., clozapine, haloperidol), cholinesterase modulators, modafinil, or non-steroidal anti-inflammatory drugs), or with adjunctive agents that may not be effective alone, but may contribute to the efficacy of the active agent. The pharmaceutical compositions may also include one or more active agents known to be useful in treating a cardiomyopathy such as Angiotensin Converting Enzyme Modulators (e.g., Enalipril, Lisinopril), Angiotensin Receptor Blockers (e.g., Losartan, Valsartan), Beta Blockers (e.g., Lopressor, Toprol-XL), Digoxin, or Diuretics (e.g., Lasixdisease associated neurodegeneration, such as those described above.

The pharmaceutical compositions comprise may, additionally or alternatively, include one or more active agent selected from “cognitive enhancing drugs,” such as those that improve impaired human cognitive abilities of the brain (namely, thinking, learning, and memory). Cognitive enhancing drugs may work by altering the availability of neurochemicals (e.g., neurotransmitters, enzymes, and hormones), by improving oxygen supply, by stimulating nerve growth, or by inhibiting nerve damage. Examples of cognitive enhancing drugs include a compound that increases the activity of acetylcholine such as, but not limited to, an acetylcholine receptor agonist (e.g., a nicotinic α-7 receptor agonist or allosteric modulator, an α4β2 nicotinic receptor agonist or allosteric modulators), an acetylcholinesterase inhibitor (e.g., donepezil, rivastigmine, and galantamine), a butyrylcholinesterase inhibitor, an N-methyl-D-aspartate (NMDA) receptor antagonist (e.g., memantine), an activity-dependent neuroprotective protein (ADNP) agonist, a serotonin 5-HT1A receptor agonist (e.g., xaliproden), a 5-HT4 receptor agonist, a 5-HT6 receptor antagonist, a serotonin 1A receptor antagonist, a histamine H3 receptor antagonist, a calpain inhibitor, a vascular endothelial growth factor (VEGF) protein or agonist, a trophic growth factor, an anti-apoptotic compound, an AMPA-type glutamate receptor activator, a L-type or N-type calcium channel blocker or modulator, a potassium channel blocker, a hypoxia inducible factor (HIF) activator, a HIF prolyl 4-hydroxylase inhibitor, an antiinflammatory agent, an inhibitor of amyloid Aβ peptide or amyloid plaque, an inhibitor of tau hyperphosphorylation, a phosphodiesterase 5 inhibitor (e.g., tadalafil, sildenafil), a phosphodiesterase 4 inhibitor, a monoamine oxidase inhibitor, or pharmaceutically acceptable salt thereof. Specific examples of such cognitive enhancing drugs include, but are not limited to, cholinesterase inhibitors such as donepezil (Aricept®), rivastigmine (Exelon®), galanthamine (Reminyl®), N-methyl-D-aspartate antagonists such as memantine (Namenda®). At least one cognitive enhancing drug can be administered simultaneously with the antibodies of the present invention or sequentially with the antibodies of the present invention (and in any order) including those agents currently recognized, or in the future being recognized, as useful to treat the disease or condition being treated by an antibody of the present invention). Additionally, it is believed that the combinations described herein may have additive or synergistic effects when used in the above described treatment. The additional agent also can be an agent that imparts a beneficial attribute to the therapeutic composition, e.g., an agent that affects the viscosity of the composition.

In some embodiments, co-administration includes administering one active agent within about 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In embodiments, co-administration can be accomplished by co-formulation, i.e., preparing a single pharmaceutical composition including both active agents. In other embodiments, the active agents can be formulated separately. In embodiments, the active and/or adjunctive agents may be linked or conjugated to one another. In embodiments, the compounds described herein may be combined with treatments for neurodegeneration such as surgery. In some embodiments, the compounds described herein may be combined with treatments for cardiomyopathy such as surgery.

In certain embodiments, provided is a method by administering any of the pharmaceutical compositions disclosed herein, wherein the pharmaceutical composition may include two or more compounds disclosed herein. In some cases, one of the compounds in the pharmaceutical compositions is one of the above disclosed active agents known to be useful in treating a disease associated neurodegeneration. In further embodiments, provided is a method of treatment comprising manufacturing and/or synthesizing one or more of compound disclosed herein (e.g., a compound, including final compounds (such as, the pyrrolopyridine compounds disclosed herein), intermediate compounds disclosed herein, and salts thereof); and administering any of the pharmaceutical compositions disclosed herein, wherein the pharmaceutical composition may include one or more of the compounds disclosed herein.

The choice of carrier will be determined in part by the particular method used to administer the composition. Accordingly, there is a wide variety of suitable formulations of the pharmaceutical composition of the present invention. The following formulations for oral, aerosol, parenteral, subcutaneous, intravenous, intraarterial, intramuscular, intraperitoneal, intrathecal, rectal, and vaginal administration are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granule; (c) powders; (d) suspensions in an appropriate liquid; and (e) suitable emulsions. Liquid formulations may include diluents, such as water, cyclodextrin, dimethyl sulfoxide and alcohols, for example, ethanol, benzyl alcohol, propylene glycol, glycerin, and the polyethylene alcohols including polyethylene glycol, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, the addition to the active ingredient in an inert base, such as gelatin and glycerin, or sucrose and acadia, emulsions, and gels containing, in addition to the active ingredient, such carriers as are known in the art.

The compounds prepared by the methods of the present disclosure, alone or in combination with other suitable components, can be made into aerosol formulations to be administered via inhalation. These aerosol formulations can be placed into pressurized acceptable propellants, such as dichlorodifluoromethane, propane, and nitrogen. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils that can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl β-aminopropionates, and 2-alkylimidazoline quaternary ammonium salts, and (e) mixtures thereof.

The parenteral formulations typically contain from about 0.5% to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

For parenteral administration of the pharmaceutical composition (including subcutaneous, intradermal, intravenous, intramuscular, and intraperitoneal), the pharmaceutical composition may be an aqueous or an oil-based composition. Aqueous compositions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based compositions (e.g., solutions or suspensions) may further comprise sesame, peanut, olive oil, or mineral oil. The pharmaceutical compositions may be presented in unit-dose or multi-dose containers, for example sealed ampoules and vials, and may be stored in a freeze-dried (lyophilized) condition requiring only the addition of the sterile liquid carried, for example, water for injections, immediately prior to use. Extemporaneous injection solutions and suspensions may be prepared from sterile powders, granules, and tablets.

Pharmaceutically acceptable excipients are also well-known to those who are skilled in the art. The choice of excipient will be determined in part by the particular compound, as well as by the particular method used to administer the composition. Accordingly, there is a wide variety of suitable formulations of the pharmaceutical composition of the present disclosure. The following methods and excipients are merely exemplary and are in no way limiting. The pharmaceutically acceptable excipients preferably do not interfere with the action of the active ingredients and do not cause adverse side-effects. Suitable carriers and excipients include solvents such as water, alcohol, and propylene glycol, solid absorbants and diluents, surface active agents, suspending agent, tableting binders, lubricants, flavors, and coloring agents.

Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, and combinations of two or more thereof.

In a further embodiment, the excipient may be a filler. Examples of fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, sorbitol, or a combination thereof.

In still another embodiment, the excipient may be a buffering agent. Non-limiting examples of suitable buffering agents include, but are not limited to, phosphates, carbonates, citrates, tris buffers, and buffered saline salts (e.g., Tris buffered saline or phosphate buffered saline). In various embodiments, the excipient may be a pH modifier. By way of non-limiting example, the pH modifying agent may be sodium carbonate, sodium bicarbonate, sodium citrate, citric acid, and/or phosphoric acid. In a further embodiment, the excipient may be a disintegrant. The disintegrant may be non-effervescent or effervescent. Examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of effervescent disintegrants include sodium bicarbonate in combination with citric acid and sodium bicarbonate in combination with tartaric acid.

In another alternate embodiment, the excipient may be a preservative. Non-limiting examples of suitable preservatives include antioxidants, such as BHA, BHT, vitamin A, vitamin C, vitamin E, or retinyl pa Imitate, citric acid, sodium citrate; chelators such as EDTA or EGTA; and antimicrobials, such as parabens, chlorobutanol, or phenol.

In a further embodiment, the excipient may be a lubricant. Nonlimiting examples of suitable lubricants include minerals such as talc or silica; and fats such as vegetable stearin, magnesium stearate, or stearic acid.

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

In still a further embodiment, the excipient may comprise a coloring agent. Suitable color additives include, but are not limited to, food, drug and cosmetic colors (FD&C), drug and cosmetic colors (D&C), or external drug and cosmetic colors (Ext. D&C).

The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutical carriers for injectable compositions are well known to those of ordinary skill in the art. See Pharmaceutics and Pharmacy Practice, J. B. Lippincott Co., Philadelphia, PA, Banker and Chalmers, Eds., 238-250 (1982) and ASHP Handbook on Injectable Drugs, Toissel, 4^{th} ed., 622-630 (1986).

Formulations suitable for topical administration include lozenges comprising the active ingredient in a flavor, usually sucrose and acacia or tragacanth; pastilles comprising the active ingredient in an inert base, such as gelatin and glycerin, or sucrose and acacia; and mouthwashes comprising the active ingredient in a suitable liquid carrier; as well as creams, emulsions, and gels containing, in addition to the active ingredient, such carriers as are known in the art.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

One skilled in the art will appreciate that suitable methods of exogenously administering a compound of the present disclosure to an animal are available, and, although more than one route can be used to administer a particular compound, a particular route can provide a more immediate and more effective reaction than another route.

As regards these applications, the present method includes the administration to an animal, particularly a mammal, and more particularly a human, of a therapeutically effective amount of the compound effective in the treatment (e.g., prophylactic or therapeutic) of a disorder associated with PINK1 kinase activity. The method also includes the administration of a therapeutically effect amount of the compound for the treatment of patient having a predisposition for being afflicted with a disorder associated with PINK1 kinase activity. The dose administered to an animal, particularly a human, in the context of the present invention should be sufficient to affect a therapeutic response in the animal over a reasonable timeframe. One skilled in the art will recognize that dosage will depend upon a variety of factors including the condition of the animal, the body weight of the animal, as well as the severity and stage of the disorder.

The total amount of the compound of the present disclosure administered in a typical treatment is preferably from about 1 mg/kg to about 100 mg/kg of body weight for mice, and from about 10 mg/kg to about 50 mg/kg of body weight, and from about 20 mg/kg to about 40 mg/kg of body weight for humans per daily dose. This total amount is typically, but not necessarily, administered as a series of smaller doses over a period of about one time per day to about three times per day for about 24 months, and over a period of twice per day for about 12 months.

The one or more compound(s) (e.g., a pyrrolopyridine compound disclosed herein, an intermediate compound disclosed herein, and/or a salt hereof) of the present disclosure may be administered in an amount from about 1 μg/kg to about 500 mg/kg. In some embodiments, a compound disclosed herein may be administered in an amount (e.g., present in the pharmaceutical composition in an amount) from about 1 mg/kg to about 500 mg/kg, about 1 to about 450 mg/kg, about 1 to about 400 mg/kg, about 1 to about 350 mg/kg, about 1 to about 300 mg/kg, about 1 to about 250 mg/kg, about 1 to about 200 mg/kg, about 1 to about 150 mg/kg, about 1 to about 125 mg/kg, about 1 to about 100 mg/kg, about 1 to about 80 mg/kg, about 1 to about 60 mg/kg, about 1 to about 50 mg/kg, about 1 to about 40 mg/kg, about 1 to about 30 mg/kg, about 1 to about 20 mg/kg, about 1 to about 10 mg/kg, about 1 to about 5 mg/kg; from about 20 to about 500 mg/kg, about 20 to about 450 mg/kg, about 20 to about 400 mg/kg, about 20 to about 350 mg/kg, about 20 to about 300 mg/kg, about 20 to about 250 mg/kg, about 20 to about 200 mg/kg, about 20 to about 150 mg/kg, about 20 to about 125 mg/kg, about 20 to about 100 mg/kg, about 20 to about 80 mg/kg, about 20 to about 60 mg/kg, about 20 to about 50 mg/kg; from about 60 to about 500 mg/kg, about 60 to about 450 mg/kg, about 60 to about 400 mg/kg, about 60 to about 350 mg/kg, about 60 to about 300 mg/kg, about 60 to about 250 mg/kg, about 60 to about 200 mg/kg, about 60 to about 150 mg/kg, about 60 to about 125 mg/kg, about 60 to about 100 mg/kg; from about 120 to about 500 mg/kg, about 120 to about 450 mg/kg, about 120 to about 400 mg/kg, about 120 to about 350 mg/kg, about 120 to about 300 mg/kg, about 120 to about 250 mg/kg, about 120 to about 200 mg/kg; from about 180 to about 500 mg/kg, about 180 to about 450 mg/kg, about 180 to about 400 mg/kg, about 180 to about 350 mg/kg, about 180 to about 300 mg/kg, about 180 to about 250 mg/kg; from about 240 to about 500 mg/kg, about 240 to about 450 mg/kg, about 240 to about 400 mg/kg, about 240 to about 350 mg/kg, about 240 to about 300 mg/kg; from about 300 to about 500 mg/kg, about 300 to about 450 mg/kg, about 300 to about 400 mg/kg, about 300 to about 350 mg/kg; from about 360 to about 500 mg/kg, about 360 to about 450 mg/kg, about 360 to about 400 mg/kg, about 400 to about 500 mg/kg, about 450 to about 500 mg/kg, or any range or subrange thereof. In any of the above, kg is a kilograms of a subject administered the any of the pharmaceutical compositions or compositions disclosed herein.

In additional embodiments, a compound disclosed herein may be administered in an amount (e.g., present in the pharmaceutical composition in an amount) from about 1 mg to about 500 mg, about 1 to about 450 mg, about 1 to about 400 mg, about 1 to about 350 mg, about 1 to about 300 mg, about 1 to about 250 mg, about 1 to about 200 mg, about 1 to about 150 mg, about 1 to about 125 mg, about 1 to about 100 mg, about 1 to about 80 mg, about 1 to about 60 mg, about 1 to about 50 mg, about 1 to about 40 mg, about 1 to about 30 mg, about 1 to about 20 mg, about 1 to about 10 mg, about 1 to about 5 mg; from about 20 to about 500 mg, about 20 to about 450 mg, about 20 to about 400 mg, about 20 to about 350 mg, about 20 to about 300 mg, about 20 to about 250 mg, about 20 to about 200 mg, about 20 to about 150 mg, about 20 to about 125 mg, about 20 to about 100 mg, about 20 to about 80 mg, about 20 to about 60 mg, about 20 to about 50 mg; from about 60 to about 500 mg, about 60 to about 450 mg, about 60 to about 400 mg, about 60 to about 350 mg, about 60 to about 300 mg, about 60 to about 250 mg, about 60 to about 200 mg, about 60 to about 150 mg, about 60 to about 125 mg, about 60 to about 100 mg; from about 120 to about 500 mg, about 120 to about 450 mg, about 120 to about 400 mg, about 120 to about 350 mg, about 120 to about 300 mg, about 120 to about 250 mg, about 120 to about 200 mg; from about 180 to about 500 mg, about 180 to about 450 mg, about 180 to about 400 mg, about 180 to about 350 mg, about 180 to about 300 mg, about 180 to about 250 mg; from about 240 to about 500 mg, about 240 to about 450 mg, about 240 to about 400 mg, about 240 to about 350 mg, about 240 to about 300 mg; from about 300 to about 500 mg, about 300 to about 450 mg, about 300 to about 400 mg, about 300 to about 350 mg; from about 360 to about 500 mg, about 360 to about 450 mg, about 360 to about 400 mg, about 400 to about 500 mg, about 450 to about 500 mg, or any range or subrange thereof.

In further embodiments, a compound disclosed herein may be administered in an amount (e.g., present in the pharmaceutical composition in an amount) from about 1 μg/kg to about 1 mg/kg, about 1 to about 900 μg/kg, about 1 to about 850 μg/kg, about 1 to about 800 μg/kg, about 1 to about 750 μg/kg, about 1 to about 700 μg/kg, about 1 to about 650 μg/kg, about 1 to about 600 μg/kg, about 1 to about 550 μg/kg, about 1 to about 500 μg/kg, about 1 to about 450 μg/kg, about 1 to about 400 μg/kg, about 1 to about 350 μg/kg, about 1 to about 300 μg/kg, about 1 to about 250 μg/kg, about 1 to about 200 μg/kg, about 1 to about 150 μg/kg, about 1 to about 100 μg/kg, about 1 to about 50 μg/kg, about 1 to about 30 μg/kg, about 1 to about 20 μg/kg, about 1 to about 100 μg/kg; from about 50 μg/kg to about 1 mg/kg, about 50 to about 900 μg/kg, about 50 to about 850 μg/kg, about 50 to about 800 μg/kg, about 50 to about 750 μg/kg, about 50 to about 700 μg/kg, about 50 to about 650 μg/kg, about 50 to about 600 μg/kg, about 50 to about 550 μg/kg, about 50 to about 500 μg/kg, about 50 to about 450 μg/kg, about 50 to about 400 μg/kg, about 50 to about 350 μg/kg, about 50 to about 300 μg/kg, about 50 to about 250 g/kg, about 50 to about 200 μg/kg, about 50 to about 150 μg/kg, about 50 to about 100 μg/kg; from about 100 μg/kg to about 1 mg/kg, about 100 to about 900 μg/kg, about 100 to about 850 μg/kg, about 100 to about 800 μg/kg, about 100 to about 750 μg/kg, about 100 to about 700 μg/kg, about 100 to about 650 μg/kg, about 100 to about 600 μg/kg, about 100 to about 550 μg/kg, about 100 to about 500 μg/kg, about 100 to about 450 μg/kg, about 100 to about 400 μg/kg, about 100 to about 350 μg/kg, about 100 to about 300 μg/kg, about 100 to about 250 μg/kg, about 100 to about 200 μg/kg, about 100 to about 150 μg/kg; from about 150 μg/kg to about 1 mg/kg, about 150 to about 900 μg/kg, about 150 to about 850 μg/kg, about 150 to about 800 μg/kg, about 150 to about 750 μg/kg, about 150 to about 700 μg/kg, about 150 to about 650 μg/kg, about 150 to about 600 μg/kg, about 150 to about 550 μg/kg, about 150 to about 500 μg/kg, about 150 to about 450 μg/kg, about 150 to about 400 μg/kg, about 150 to about 350 μg/kg, about 150 to about 300 g/kg, about 150 to about 250 μg/kg; from about 200 μg/kg to about 1 mg/kg, about 200 to about 900 μg/kg, about 200 to about 850 μg/kg, about 200 to about 800 μg/kg, about 200 to about 750 μg/kg, about 200 to about 700 μg/kg, about 200 to about 650 μg/kg, about 200 to about 600 μg/kg, about 200 to about 550 μg/kg, about 200 to about 500 μg/kg, about 200 to about 450 μg/kg, about 200 to about 400 μg/kg, about 200 to about 350 μg/kg, about 200 to about 300 μg/kg, about 200 to about 250 μg/kg; from about 300 μg/kg to about 1 mg/kg, about 300 to about 900 μg/kg, about 300 to about 850 μg/kg, about 300 to about 800 μg/kg, about 300 to about 750 μg/kg, about 300 to about 700 μg/kg, about 300 to about 650 μg/kg, about 300 to about 600 μg/kg, about 300 to about 550 μg/kg, about 300 to about 500 μg/kg, about 300 to about 450 μg/kg, about 300 to about 400 μg/kg; from about 400 μg/kg to about 1 mg/kg, about 400 to about 900 μg/kg, about 400 to about 850 μg/kg, about 400 to about 800 μg/kg, about 400 to about 750 μg/kg, about 400 to about 700 μg/kg, about 400 to about 650 μg/kg, about 400 to about 600 μg/kg, about 400 to about 550 μg/kg, about 400 to about 500 μg/kg; from about 500 μg/kg to about 1 mg/kg, about 500 to about 900 μg/kg, about 500 to about 850 μg/kg, about 500 to about 800 μg/kg, about 500 to about 750 μg/kg, about 500 to about 700 μg/kg, about 500 to about 650 μg/kg, about 500 to about 600 μg/kg; from about 600 μg/kg to about 1 mg/kg, about 600 to about 900 μg/kg, about 600 to about 850 μg/kg, about 600 to about 800 μg/kg, about 600 to about 750 μg/kg, about 600 to about 700 μg/kg, about 600 to about 650 μg/kg, about 600 to about 600 μg/kg; from about 700 μg/kg to about 1 mg/kg, about 700 to about 900 μg/kg, about 700 to about 850 μg/kg, about 700 to about 800 μg/kg; from about 800 μg/kg to about 1 mg/kg, about 800 to about 900 μg/kg, about 800 to about 850 μg/kg, about 90 μg/kg to about 1 mg/kg, or any range or subrange thereof. The use of kg above is kilograms of a subject to whom the composition or pharmaceutical composition is administered.

In yet further embodiments, a compound disclosed herein may be administered in an amount (e.g., present in the pharmaceutical composition in an amount) from about 1 μg to about 1 mg, about 1 to about 900 μg, about 1 to about 850 μg, about 1 to about 800 μg, about 1 to about 750 μg, about 1 to about 700 μg, about 1 to about 650 μg, about 1 to about 600 μg, about 1 to about 550 μg, about 1 to about 500 μg, about 1 to about 450 μg, about 1 to about 400 μg, about 1 to about 350 μg, about 1 to about 300 μg, about 1 to about 250 μg, about 1 to about 200 μg, about 1 to about 150 μg, about 1 to about 100 μg, about 1 to about 50 μg, about 1 to about 30 μg, about 1 to about 20 μg, about 1 to about 100 μg; from about 50 μg to about 1 mg, about 50 to about 900 μg, about 50 to about 850 μg, about 50 to about 800 μg, about 50 to about 750 μg, about 50 to about 700 μg, about 50 to about 650 μg, about 50 to about 600 μg, about 50 to about 550 μg, about 50 to about 500 μg, about 50 to about 450 μg, about 50 to about 400 μg, about 50 to about 350 μg, about 50 to about 300 μg, about 50 to about 250 μg, about 50 to about 200 μg, about 50 to about 150 μg, about 50 to about 100 μg; from about 100 μg to about 1 mg, about 100 to about 900 μg, about 100 to about 850 μg, about 100 to about 800 μg, about 100 to about 750 μg, about 100 to about 700 μg, about 100 to about 650 μg, about 100 to about 600 μg, about 100 to about 550 μg, about 100 to about 500 μg, about 100 to about 450 μg, about 100 to about 400 μg, about 100 to about 350 μg, about 100 to about 300 μg, about 100 to about 250 μg, about 100 to about 200 μg, about 100 to about 150 μg; from about 150 μg to about 1 mg, about 150 to about 900 μg, about 150 to about 850 μg, about 150 to about 800 μg, about 150 to about 750 μg, about 150 to about 700 μg, about 150 to about 650 μg, about 150 to about 600 μg, about 150 to about 550 μg, about 150 to about 500 μg, about 150 to about 450 μg, about 150 to about 400 μg, about 150 to about 350 μg, about 150 to about 300 μg, about 150 to about 250 μg; from about 200 μg to about 1 mg, about 200 to about 900 μg, about 200 to about 850 μg, about 200 to about 800 μg, about 200 to about 750 μg, about 200 to about 700 μg, about 200 to about 650 μg, about 200 to about 600 μg, about 200 to about 550 μg, about 200 to about 500 μg, about 200 to about 450 μg, about 200 to about 400 μg, about 200 to about 350 μg, about 200 to about 300 μg, about 200 to about 250 μg; from about 300 μg to about 1 mg, about 300 to about 900 μg, about 300 to about 850 μg, about 300 to about 800 μg, about 300 to about 750 μg, about 300 to about 700 μg, about 300 to about 650 μg, about 300 to about 600 μg, about 300 to about 550 μg, about 300 to about 500 μg, about 300 to about 450 μg, about 300 to about 400 μg; from about 400 μg to about 1 mg, about 400 to about 900 μg, about 400 to about 850 μg, about 400 to about 800 μg, about 400 to about 750 μg, about 400 to about 700 μg, about 400 to about 650 μg, about 400 to about 600 μg, about 400 to about 550 μg, about 400 to about 500 μg; from about 500 μg to about 1 mg, about 500 to about 900 μg, about 500 to about 850 μg, about 500 to about 800 μg, about 500 to about 750 μg, about 500 to about 700 μg, about 500 to about 650 μg, about 500 to about 600 μg; from about 600 μg to about 1 mg, about 600 to about 900 μg, about 600 to about 850 μg, about 600 to about 800 μg, about 600 to about 750 μg, about 600 to about 700 μg, about 600 to about 650 μg, about 600 to about 600 μg; from about 700 μg to about 1 mg, about 700 to about 900 μg, about 700 to about 850 μg, about 700 to about 800 μg; from about 800 μg to about 1 mg, about 800 to about 900 μg, about 800 to about 850 μg, about 90 μg to about 1 mg, or any range or subrange thereof.

The pyrrolopyridine compound(s) of the present disclosure may be administered in an amount of 1 μM or more, 2 μM or more, about 5 μM or more, about 10 μM or more, about 14 μM or more, about 18 UM or more, about 22 μM or more, about 25 μM or more, about 28 μM or more, about 31 μM or more, about 35 μM or more, about 40 μM or more, or about 45 μM or more. For example, the amount of pyrrolopyridine compound(s) of the present disclosure administered in a dose may be from about 1 to about 45 μM. In some embodiments, the amount of pyrrolopyridine compound(s) of the present disclosure administered in a dose is from about 1 to about 45 μM, about 1 to about 40 μM, about 1 to about 35 μM, about 1 to about 32 μM, about 1 to about 29 μM, about 1 to about 26 μM, about 1 to about 23 μM, about 1 to about 20 μM, about 1 to about 17 μM, about 1 to about 12 μM, about 1 to about 7 μM; from about 5 to about 45 μM, about 5 to about 40 μM, about 5 to about 35 μM, about 5 to about 32 μM, about 5 to about 29 μM, about 5 to about 26 μM, about 5 to about 23 μM, about 5 to about 20 μM, about 5 to about 17 μM, about 5 to about 12 μM; from about 9 to about 45 μM, about 9 to about 40 μM, about 9 to about 35 μM, about 9 to about 32 μM, about 9 to about 29 μM, about 9 to about 26 μM, about 9 to about 23 μM, about 9 to about 20 μM, about 9 to about 17 μM; from about 13 to about 45 μM, about 13 to about 40 μM, about 13 to about 35 μM, about 13 to about 32 μM, about 13 to about 29 μM, about 13 to about 26 μM, about 13 to about 23 μM, about 13 to about 20 μM; from about 17 to about 45 μM, about 17 to about 40 μM, about 17 to about 35 μM, about 17 to about 32 μM, about 17 to about 29 μM, about 17 to about 26 μM, about 17 to about 23 μM, about 17 to about 20 μM; from about 21 to about 45 μM, about 21 to about 40 μM, about 21 to about 35 μM, about 21 to about 32 μM, about 21 to about 29 μM, about 21 to about 26 μM, about 21 to about 23 μM; from about 23 to about 45 μM, about 23 to about 40 μM, about 23 to about 35 μM, about 23 to about 32 μM, about 23 to about 29 μM, about 23 to about 26 μM; from about 25 to about 45 μM, about 25 to about 40 μM, about 25 to about 3 μM, about 25 to about 32 μM, about 25 to about 29 μM, about 25 to about 26 μM; from about 27 to about 45 μM, about 27 to about 40 μM, about 27 to about 35 μM, about 27 to about 32 μM, about 27 to about 29 μM; from about 29 to about 45 μM, about 29 to about 40 μM, about 29 to about 35 μM, about 29 to about 32 μM; from about 32 to about 45 μM, about 32 to about 40 μM, about 32 to about 35 μM; from about 35 to about 45 μM, about 35 to about 4 μM, about 39 to about 45 μM, or any range or subrange thereof.

The size of the dose also will be determined by the route, timing, and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

In certain embodiments, a composition described herein is formulated for administration to a patient in need of such composition. Compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the compositions described herein may be aqueous or oleaginous suspension. These suspensions may be formulated according to techniques known in the art using suitable dispersing or wetting agents and suspending agents.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

A compound described herein can be administered alone or can be coadministered with an additional therapeutic agent. Thus, the preparations can also be combined, when desired, with other active substances (e.g., to reduce metabolic degradation). Additional therapeutic agents include, but are not limited to, other active agents known to be useful in treating a disease associated neurodegeneration (e.g., Parkinson's disease such as levodopa), dopamine agonists (e.g., bromocriptine, pergolide, pramipexole, ropinirole, piribedil, cabergoline, apomorphine, lisuride), MAO-B modulators (e.g., selegiline or rasagiline), amantadine, anticholinergics, antipsychotics (e.g., clozapine), cholinesterase modulators, modafinil, or non-steroidal anti-inflammatory drugs), Angiotensin Converting Enzyme Modulators (e.g., Enalipril, Lisinopril), Angiotensin Receptor Blockers (e.g., Losartan, Valsartan), Beta Blockers (e.g., Lopressor, Toprol-XL), Digoxin, or Diuretics.

In some embodiments, the compounds described herein can be delivered in a vesicle, in particular a liposome (see, Langer, Science, 1990, 249, 1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses)n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy) propyl)-N,N,N-trimethyl ammonium chloride, 1, 1 ‘-dioctadecyl-3,3,3’,3′-tetramethylindocarbocyanine perchloarate, 3,3′-deheptyloxacarbocyanine iodide, 1,T-dedodecyl-3,3,3′,3′-tetramethylindocarbocyanine perchloarate, 1,T-dioleyl-3,3,3′,3′-tetramethylindo carbocyanine methanesulfonate, N-4-(delinoleylaminostyryl)-N-methylpyridinium iodide, or 1,1,dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons. Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetro nitrile, alcohols, dimethylformamide, tetrahydrofuran, or combinations thereof.

The liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar liposomes.

Suitable compositions include, but are not limited to, oral non-absorbed compositions. Suitable compositions also include, but are not limited to saline, water, cyclodextrin solutions, and buffered solutions of pH 3-9.

The compounds described herein, or pharmaceutically acceptable salts thereof, can be formulated with numerous excipients including, but not limited to, purified water, propylene glycol, PEG 400, glycerin, DMA, ethanol, benzyl alcohol, citric acid/sodium citrate (pH3), citric acid/sodium citrate (pH5), tris(hydroxymethyl)amino methane HCl (pH7.0), 0.9% saline, 1.2% saline, acetate, aspartate, benzenesulfonate, benzoate, besylate, bicarbonate, bitartrate, bromide, camsylate, carbonate, chloride, citrate, decanoate, edetate, esylate, fumarate, gluceptate, gluconate, glutamate, glycolate, hexanoate, hydroxynaphthoate, iodide, isethionate, lactate, lactobionate, malate, maleate, mandelate, mesylate, methylsulfate, mucate, napsylate, nitrate, octanoate, oleate, pamoate, pantothenate, phosphate, polygalacturonate, propionate, salicylate, stearate, succinate, sulfate, tartrate, teoclate, tosylate, and any combination thereof. In some embodiments, excipient is chosen from propylene glycol, purified water, and glycerin.

In some embodiments, the formulation can be lyophilized to a solid and reconstituted with, for example, water prior to use.

When administered to a mammal (e.g., to an animal for veterinary use or to a human for clinical use) the compounds can be administered in isolated form.

When administered to a human, the compounds can be sterile. Water is a suitable carrier when the compound of Formula I is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The compositions described herein can take the form of a solution, suspension, emulsion, tablet, pill, pellet, capsule, capsule containing a liquid, powder, sustained-release formulation, suppository, aerosol, spray, or any other form suitable for use. Examples of suitable pharmaceutical carriers are described in Remington's Pharmaceutical Sciences, A. R. Gennaro (Editor) Mack Publishing Co.

In some embodiments, the compounds are formulated in accordance with routine procedures as a pharmaceutical composition adapted for administration to humans. Typically, compounds are solutions in sterile isotonic aqueous buffer. Where necessary, the compositions can also include a solubilizing agent. Compositions for intravenous administration may optionally include a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound is to be administered by infusion, it can be dispensed, for example, with an infusion bottle containing sterile pharmaceutical grade water or saline. Where the compound is administered by injection, an ampoule of sterile water for injection or saline can be provided so that the ingredients may be mixed prior to administration.

The pharmaceutical compositions can be in unit dosage form. In such form, the composition can be divided into unit doses containing appropriate quantities of the active component. The unit dosage form can be a packaged preparation, the package containing discrete quantities of the preparations, for example, packeted tablets, capsules, and powders in vials or ampules. The unit dosage form can also be a capsule, cachet, or tablet itself, or it can be the appropriate number of any of these packaged forms.

In some embodiments, a composition of the present disclosure is in the form of a liquid wherein the active agent is present in solution, in suspension, as an emulsion, or as a solution/suspension. In some embodiments, the liquid composition is in the form of a gel. In other embodiments, the liquid composition is aqueous. In other embodiments, the composition is in the form of an ointment.

In some embodiments, the composition is in the form of a solid article. For example, in some embodiments, the ophthalmic composition is a solid article that can be inserted in a suitable location in the eye, such as between the eye and eyelid or in the conjunctival sac, where it releases the active agent as described, for example, U.S. Pat. Nos. 3,863,633; 3,867,519; 3,868,445; 3,960,150; 3,963,025; 4,186,184; 4,303,637; 5,443,505; and 5,869,079. Release from such an article is usually to the cornea, either via the lacrimal fluid that bathes the surface of the cornea, or directly to the cornea itself, with which the solid article is generally in intimate contact. Solid articles suitable for implantation in the eye in such fashion are generally composed primarily of polymers and can be bioerodible or non-bioerodible. Bioerodible polymers that can be used in the preparation of ocular implants carrying one or more of the compounds described herein in accordance with the present disclosure include, but are not limited to, aliphatic polyesters such as polymers and copolymers of poly(glycolide), poly(lactide), poly(epsilon-caprolactone), poly-(hydroxybutyrate) and poly(hydroxyvalerate), polyamino acids, polyorthoesters, polyanhydrides, aliphatic polycarbonates and polyether lactones. Suitable non-bioerodible polymers include silicone elastomers.

The compositions described herein can contain preservatives. Suitable preservatives include, but are not limited to, mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof; phenoxyethanol; chlorophenoxyethanol; phenoxypropanol; chlorobutanol; chlorocresol; phenylethyl alcohol; disodium EDTA; and sorbic acid and salts thereof.

It is understood that the disclosed compositions can be prepared from the disclosed compounds. It is also understood that the disclosed compositions can be employed in the disclosed methods of using.

### G. EXAMPLES

Representative examples of the disclosed compounds are illustrated in the following non-limiting methods, schemes, and examples.

### 1. General Experimental Method

General starting materials used were obtained from commercial sources or prepared in other examples, unless otherwise noted. All temperatures are in degrees Celsius (° C) and are uncorrected. Reagent grade chemicals and anhydrous solvent were purchased from commercial sources and unless otherwise mentioned, were used without further purification. The names of the products were determined using the naming software included in Biovia electronic lab notebook. Silica gel chromatography was performed on Teledyne Isco instruments using pre-packaged disposable SiO₂ stationary phase columns with eluent flow rate range of 15 to 200 mL/min, UV detection (254 and 280 nm). Reverse phase preparative HPLC was carried out using C18 columns, UV detection (214 and 254 nm) eluting with gradients of MeCN in H₂O (0.03% (NH₄)₂CO₃/0.375% NH₄OH, high pH) or MeCN in H₂O (0.1% HCOOH, low pH). The analytical HPLC chromatograms were performed using an Agilent 1100 series instrument with DAD detector (190 nm to 300 nm). The mass spectra were recorded with a Waters Micromass ZQ detector at 130 °C. The mass spectrometer was equipped with an electrospray ion source (ESI) operated in a positive ion mode and was set to scan between m/z 150-750 with a scan time of 0.3 s. Products and intermediates were analyzed by HPLC/MS on a Gemini-NX (5 μM, 2.0×30 mm) using a high pH buffer gradient of 5% to 100% of MeCN in H₂O (0.03% (NH₄)₂CO₃/0.375% NH₄OH) over 2.5 min at 1.8 mL/min for a 3.5 min run (B05) and EVO C18 (5 μM, 3.0×50 mm) using a low pH buffer gradient of 5% to 100% of MeCN in H₂O (0.1% HCOOH) over 2.5 min at 2.2 mL/min for a 3.5 min run (A05). The ¹H NMR spectra were recorded on a Bruker UltraShield 500 MHz/54 mm instrument (BZH 43/500/70B, D221/54-3209). The chemical shifts are referenced to solvent peaks, which in ¹H NMR appear at 7.26 ppm for CDCl₃, 2.50 for DMSO-d6, and 3.31 ppm for CD₃OD.

The following abbreviations have the indicated meanings:
aq aqueous;
(Bpin)₂ bis(pinacolato)diboron;
Comins' reagent N-bis(trifluoromethanesulfonimide);
DBDMH 1,3-dibromo-5,5-dimethylhydantoin
DMF N,N-dimethyl formamide;
DMSO dimethyl sulfoxide;
Et₂O diethyl ether;
EtOAc ethyl acetate;
EtOH ethanol;
eq. or equiv. equivalent
h hour(s);
HPLC high performance liquid chromatography;
LCMS liquid chromatography mass spectrometry
LiHMDS lithium bis(trimethylsilyl)amide
MeOH methanol;
m minute(s);
MS mass spectrometry
NaHMDS sodium bis(trimethylsilyl)amide
NMP N-methylpyrrolidone
NMR nuclear magnetic resonance;
23 °C room temperature;
sat. saturated;
SFC supercritical fluid chromatography;
THF tetrahydrofuran;
OTI trifluoromethanesulfonate;

### 2. Synthesis of N-((3R,4S)-7-fluoro-3-((R)-2-methylmorpholino)chroman-4-yl)-6-(trifluoromethyl)-7H-pyrrolo[2,3-d]pyrimidin-4-amine

A complete synthesis of N-((3R,4S)-7-fluoro-3-((R)-2-methylmorpholino)chroman-4-yl)-6-(trifluoromethyl)-7H-pyrrolo[2,3-d]pyrimidin-4-amine is shown in Scheme 1 below.

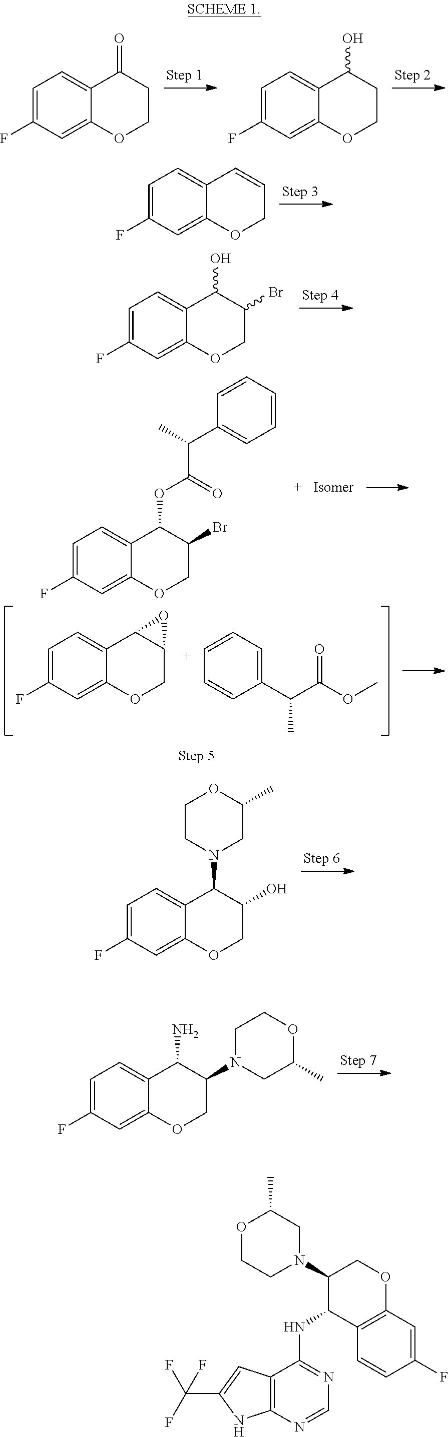

### a. Step 1—Reduction of 7-Fluorochroman-4-one

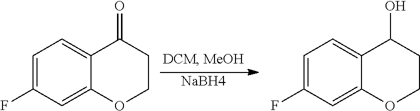

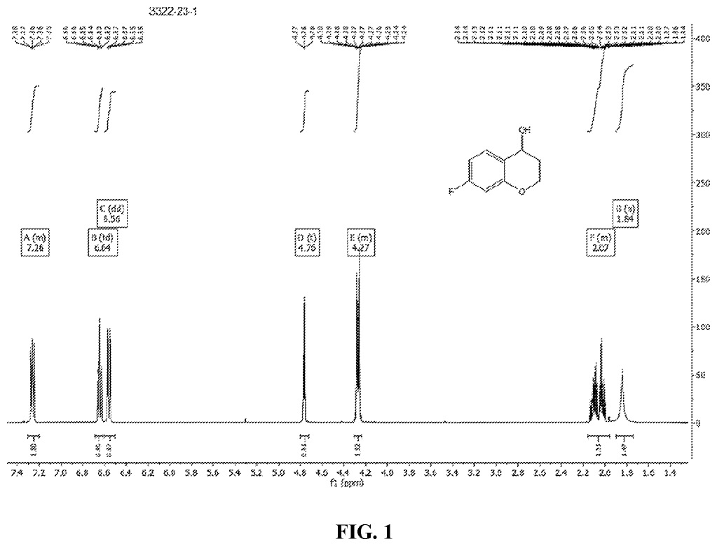

The reduction of 7-fluorochroman-4-one was successfully conducted on >100 g scale multiple times following the described procedure below. Briefly, NaBH₄ (11.95 g, 0.5 eq, 315.95 mmol) was added portionwise over 25 minutes to a solution of 7-fluorochroman-4-one (104.99 g, 1 eq, 631.90 mmol) in DCM (470 mL) and methanol (89.08 g, 112 mL, 4.4 Eq, 2.7804 mol) at 0-10 °C and stirred for 30 minutes at 0-10 °C. The reaction was allowed to warm to r.t. and stirred for a further 3 hours. The reaction was cooled to 0-10 °C and HCl (12.67 g, 347.54 mL, 1 molar, 0.55 eq, 347.54 mmol) was added over 10 minutes, stirred for 10 minutes, allowed to warm to r.t., and stirred for 30 minutes. The two layers were separated and the aqueous was extracted with DCM (3×200 mL). The combined organics were washed with water (200 mL) dried (MgSO₄), filtered, and the filtrate was concentrated to dryness in vacuo to give 7-fluorochroman-4-ol (114.23 g, 0.65 mol, 100%, 95% purity) as a white solid. ¹H NMR ¹H NMR (500 MHz, CDCl₃) δ 7.30-7.20 (m, 1H), 6.64 (td, J=8.4, 2.6 Hz, 1H), 6.56 (dd, J=10.3, 2.5 Hz, 1H), 4.76 (t, J=4.0 Hz, 1H), 4.30-4.24 (m, 2H), 2.16-1.96 (m, 2H), 1.84 (s, 1H). See FIG. 1.

### i. Step 2—Dehydration of 7-Fluorochroman-4-ol

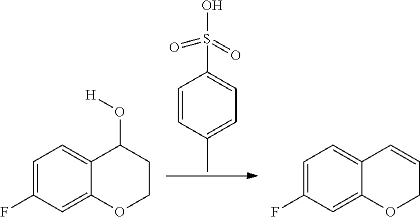

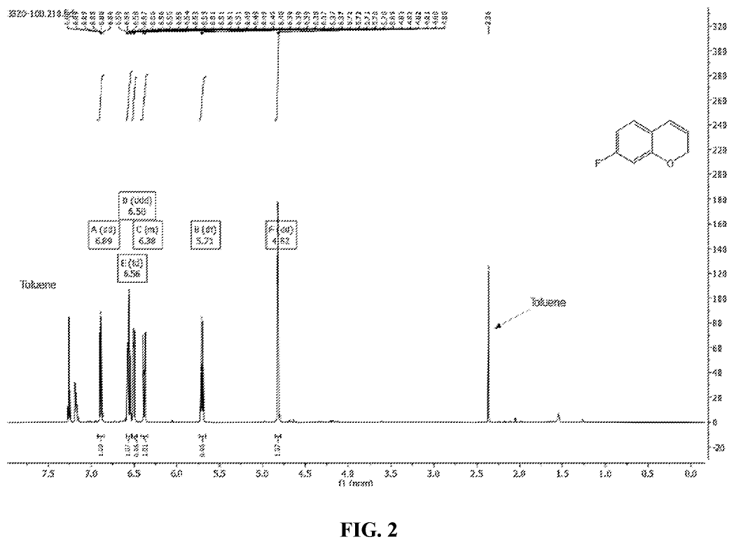

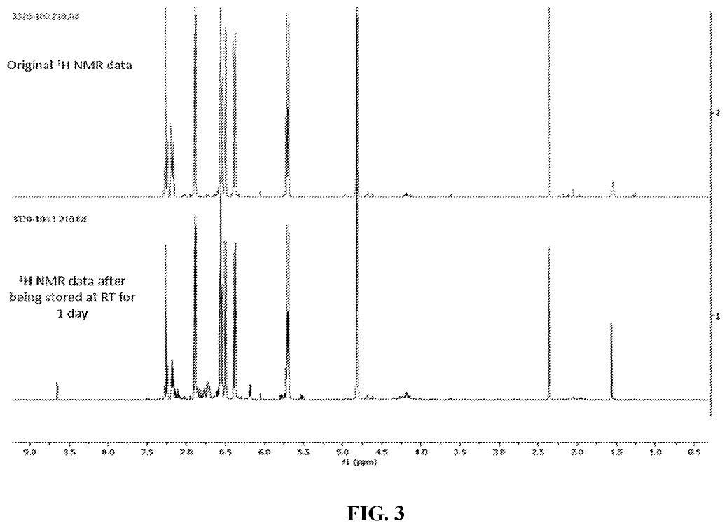

The dehydration of 7-fluorochroman-4-ol was successfully conducted on a >100 g scale multiple times following the procedure described below. Without wishing to be bound by theory, it is advised to immediately progress the isolated product in downstreaming processing as there are indications that the product is unstable to storage. Briefly, 4-methylbenzenesulfonic acid, monohydrate (1.6749 g, 0.0125 eq, 8.8053 mmol) was added to a stirring solution of 7-fluorochroman-4-ol (120.88 g, 98 wt %, 1 eq, 704.42 mmol) in toluene (1200 mL) at 50 °C and the reaction was heated to reflux with attached Dean-Stark apparatus and stirred for 2.5 hours. The cool reaction mixture was washed with water (2×500 mL), brine (400 mL), dried over MgSO₄, filtered, and the filtrate was concentrated in vacuum to afford 7-fluoro-2H-chromene (108.9 g, 0.62 mol, 89%, 86% purity) as a yellow oil. ¹H NMR (500 MHz, Chloroform-d) δ 6.89 (dd, J=8.3, 6.5 Hz, 1H), 6.58-6.54 (m, 1H), 6.50 (ddd, J=10.0, 2.5, 0.7 Hz, 1H), 6.40-6.37 (m, 1H), 5.73-5.68 (m, 1H), 4.82 (dd, J=3.6, 1.9 Hz, 2H)—see FIG. 2 for ¹H NMR analysis of isolated 7-fluoro-2H-chromene and FIG. 3 for ¹H NMR overlay showing degradation after being stored for 24 hours at RT. See FIG. 2. See also FIG. 3, which contains a ¹H NMR overlay illustrating the degradation observed after storage for 24 hours at room temperature.

### j. Step 3—Formation of 3-Bromo-7-fluorochroman-4-ol

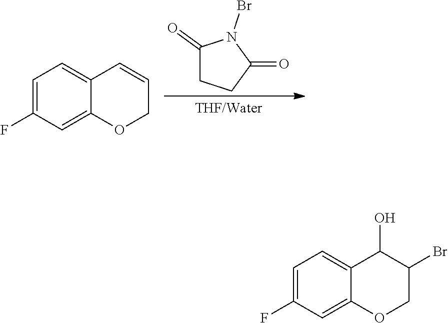 [SMILES: Fc1ccc2c(c1)OCC=C2 ; O=C1CCC(=O)N1Br ; OC1c2ccc(F)cc2OCC1Br]

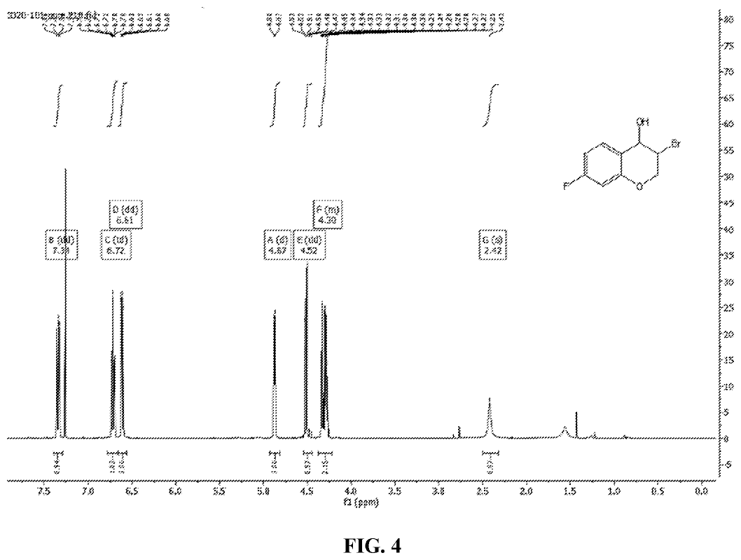

The formation of 3-bromo-7-fluorochroman-4-ol was successfully conducted on >100 g scale following the procedure below. 1-Bromopyrrolidine-2,5-dione (116.9 g, 1.0 eq, 656.6 mol) was added to a stirring solution of 7-fluoro-2H-chromene (108.9 g, 86 wt %, 0.95 eq, 623.7 mmol) in THE (900 mL) and water (450 mL) at 0 °C. Upon complete addition the reaction was allowed to warm to 22 °C and stirred for 18 hours. The reaction mixture was diluted with water (1000 mL), MTBE (1000 mL) was added, and the reaction stirred for 5 minutes. The combined organic layers were washed with water (3×500 mL), 20% brine (500 mL), dried over magnesium sulfate, filtered, and concentrated in vacuo. An emulsion formed during each wash so the solution was allowed to settle for 30 minutes prior to separation. This was recrystallized from cyclohexane (700 mL) and isohexane (300 mL) to afford 3-bromo-7-fluorochroman-4-ol (133.56 g, 0.51 mol, 78%, 95% purity) as a brown solid. ¹H NMR (500 MHz, Chloroform-d) δ 7.34 (dd, J=8.6, 6.4 Hz, 1H), 6.75-6.68 (m, 1H), 6.61 (dd, J=10.0, 2.5 Hz, 1H), 4.87 (d, J=5.1 Hz, 1H), 4.51 (dd, J=11.7, 2.3 Hz, 1H), 4.36-4.23 (m, 2H). See FIG. 4.

### k. Step 4—Formation of (3S,48)-3-Bromo-7-fluorochroman-4-yl (R)-2-phenlpropanoate

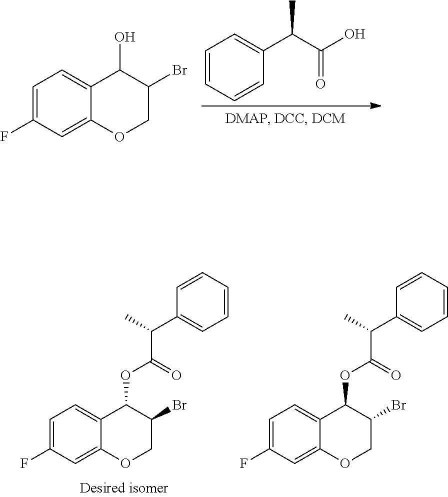

MAP (77.564 g, 1.1 eq, 634.88 mmol) and DCC (131.00 g, 1.1 Eq, 634.88 mmol) were slowly added to a mixture of 3-bromo-7-fluorochroman-4-ol (150.10 g, 95 wt %, 1 eq, 577.17 mmol) and (R)-2-phenylpropanoic acid (95.347 g, 1.1 eq, 634.88 mmol) in DCM (1700 mL) at 0 °C under nitrogen. The reaction was then stirred at 22 °C for 3 hours and filtered through a pad of Celite. The filtrate was concentrated under vacuum to afford an orange oil to give 1:1 mixture of diastereomers.

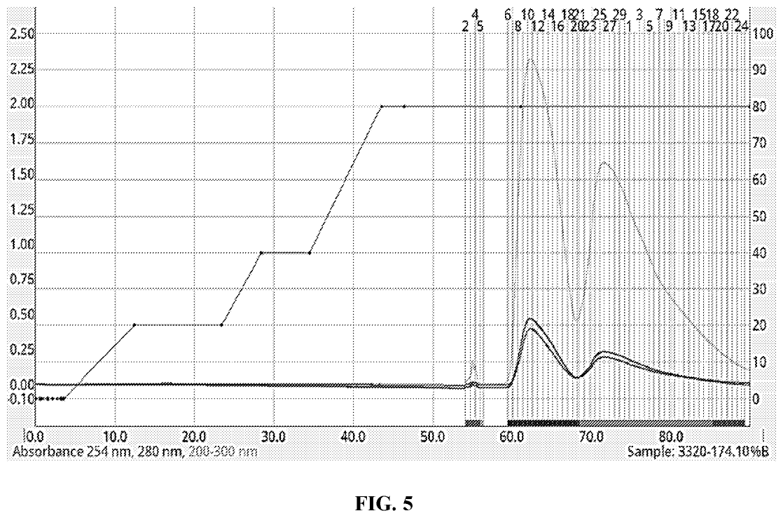

The mixture was purified batchwise by silica gel chromatography. In a typical run, 18 g of crude was pre-adsorbed onto silica (36 g). This was purified using pre-packed 330 g silica cartridges using the parameters in Table 1 below. An exemplary chromatography trace is illustrated in FIG. 5.

TABLE 1

| | % Solvent A | % Solvent B |
|---|---|---|
| Time (min) | (Isohexane) | (3% Et₂O/isohexane) |
| 0 | 100 | 0 |
| 5 | 100 | 0 |
| 12 | 80 | 20 |
| 24 | 80 | 20 |
| 29 | 60 | 40 |
| 34 | 60 | 40 |
| 43 | 20 | 80 |
| 43 onwards | 20 | 80 |

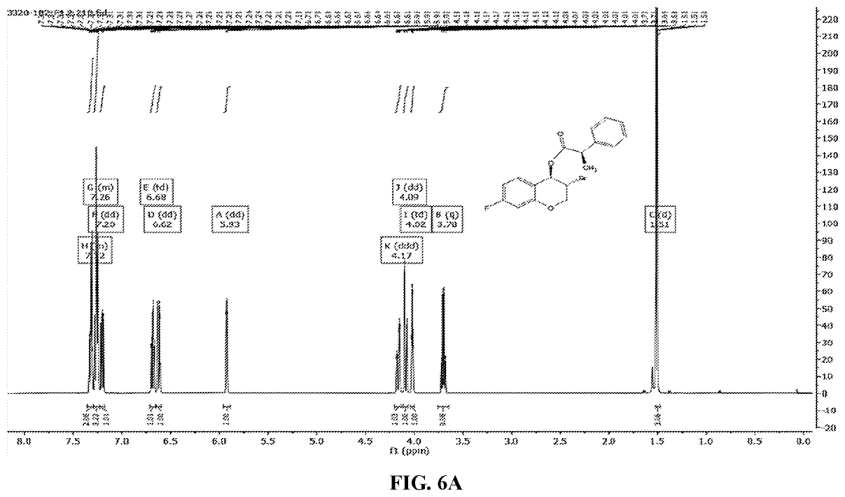

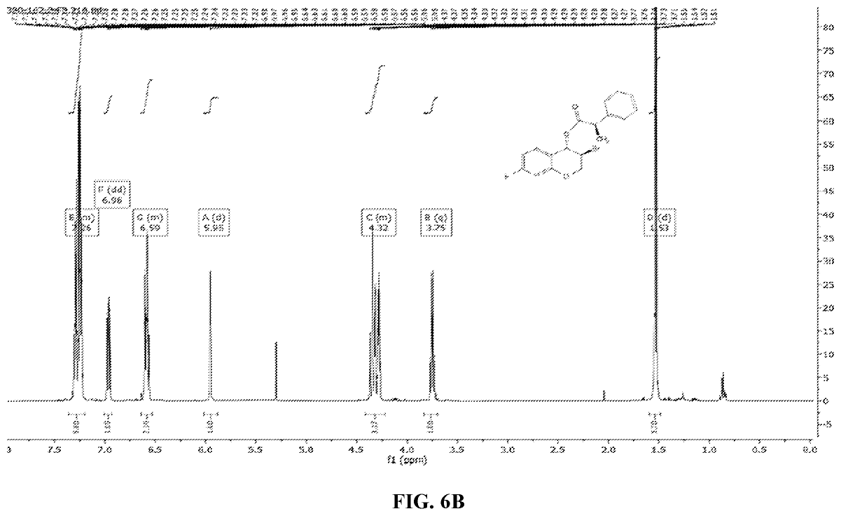

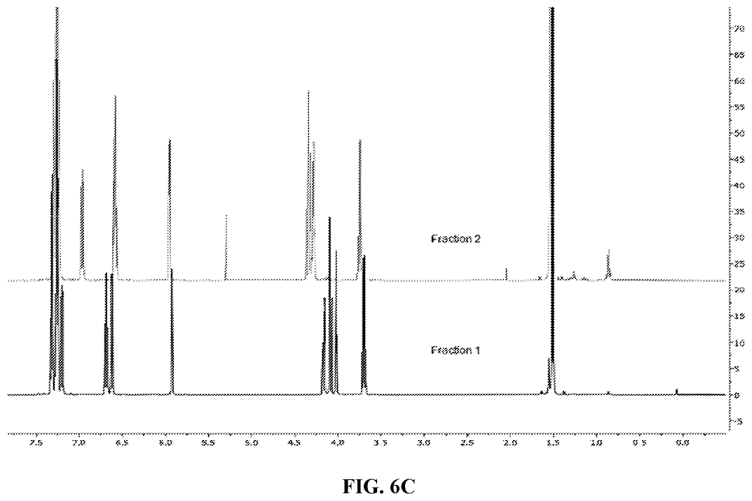

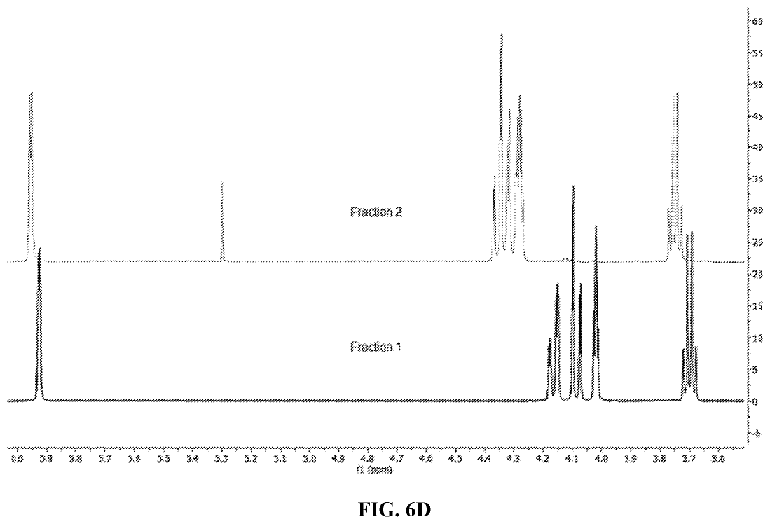

The first eluting isomer afforded undesired (3R,4R)-3-bromo-7-fluorochroman-4-yl (R)-2-phenylpropanoate (84 g, 0.22 mol, 38%) as a white solid. ¹H NMR (500 MHz, CDCl₃) δ 7.36-7.29 (m, 2H), 7.29-7.23 (m, 3H), 7.21 (dd, J=8.6, 6.3 Hz, 1H), 6.72-6.65 (m, 1H), 6.62 (dd, J=10.0, 2.6 Hz, 1H), 5.93 (dd, J=3.5, 1.4 Hz, 1H), 4.17 (ddd, J=12.5, 3.3, 1.4 Hz, 1H), 4.09 (dd, J=12.4, 2.0 Hz, 1H), 4.06-3.98 (m, 1H), 3.70 (q, J=7.1 Hz, 1H), 1.51 (d, J=7.2 Hz, 3H). See FIG. 6A.

The second eluting product afforded desired product (3S,4S)-3-bromo-7-fluorochroman-4-yl (R)-2-phenylpropanoate (86 g, 0.23 mol, 39%) as a colourless oil. ¹H NMR (500 MHz, Chloroform-c) δ 7.35-7.19 (m, 5H), 6.98 (dd, J=8.5, 6.3 Hz, 1H), 6.65-6.55 (m, 2H), 5.97 (d, J=3.8 Hz, 1H), 4.41-4.26 (m, 3H), 3.76 (q, J=7.2 Hz, 1H), 1.54 (d, J=7.2 Hz, 3H). See FIG. 6B.

A ¹H NMR overlay of both isomers is shown in FIG. 6C (full spectrum) and FIG. 6D (expanded).

### 1. Step 5—Synthesis of (3S,4R)-7-fluoro-4-((R)-2-methylmorpholino)chroman-3-ol

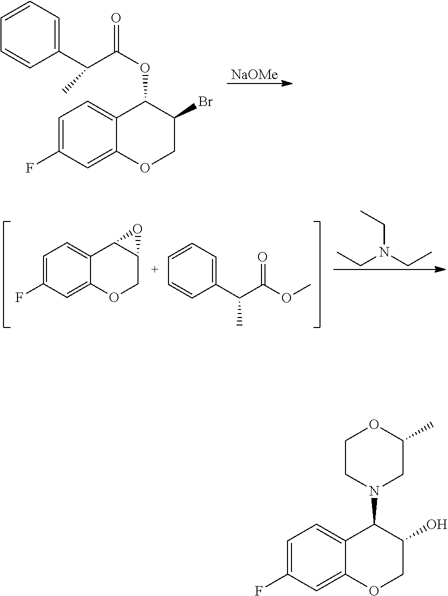

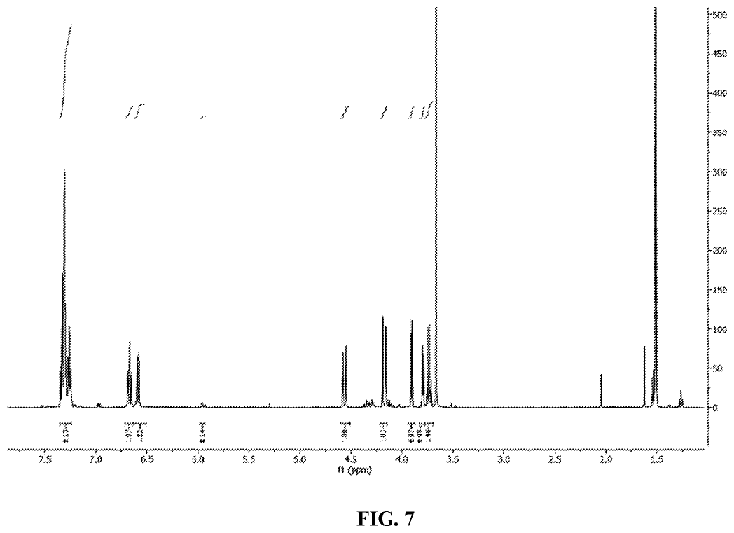

To a stirred solution of (3S,4S)-3-bromo-7-fluorochroman-4-yl (R)-2-phenylpropanoate (40.0 g, 95 wt %, 1 eq, 100 mmol) in anhydrous diethyl ether (130 mL) under nitrogen was added sodium methanolate (13.5 g, 2.5 eq, 251 mmol). The reaction was stirred at 25 °C for 4 hours. This was partitioned between cold water (100 mL) and EtOAc (20 mL). The aqueous phase was extracted with DCM (50 mL). The combined organics were dried (MgSO₄) and concentrated in vacuo to afford (1aR,7bS)-5-fluoro-1a,7b-dihydro-2H-oxireno[2,3-c]chromene (38.97 g, 94 mmol, 94%, 40% purity) as a yellow oil. Without wishing to be bound by theory, it is recommend to store the oil at 0 °C under nitrogen atmosphere. ¹H NMR (500 MHz, Chloroform-d) δ 7.39-7.21 (m, 1H), 6.99-6.91 (m, 1H), 6.87-6.82 (m, 1H), 4.56 (dd, J=12.6, 1.3 Hz, 1H), 4.15 (dd, J=12.7, 0.6 Hz, 1H), 3.92 (d, J=4.5 Hz, 1H), 3.83-3.79 (m, 1H). See FIG. 7.

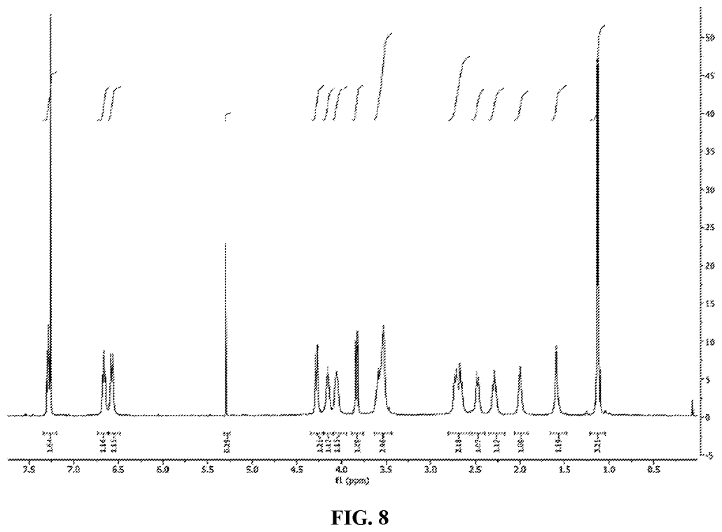

A solution of (1aR,7bS)-5-fluoro-1a,7b-dihydro-2H-oxireno[2,3-c]chromene (38.97 g, 40 wt % 0.95 eq, 93.82 mmol), (R)-2-methylmorpholine hydrochloride (13.59 g, 1 eq, 98.76 mmol) and triethylamine (14.99 g, 20.6 mL, 1.5 eq, 148.1 mmol) in toluene (10 mL) was heated to 90 °C for 3 hours. The cooled reaction mixture was partitioned between water (100 mL) and EtOAc (50 mL). The aqueous phase was then extracted with DCM (60 mL). The combined organics were dried (MgSO₄), filtered, and the filtrate concentrated in vacuo. The crude product was spilt in two portions and purified by silica gel chromatography eluting with 0-5% MeOH/DCM to afford (3S,4R)-7-fluoro-4-((R)-2-methylmorpholino)chroman-3-ol (23.10 g, 81 mmol, 87%, 94% purity) as a dark yellow oil. 1H NMR (500 MHz, CDCl₃) δ 7.34-7.19 (m, 1H), 6.66 (br.s, 1H), 6.57 (br.d, J=10.1 Hz, 1H), 4.28 (d, J=11.2 Hz, 1H), 4.15 (br.s, 1H), 4.06 (br.s, 1H), 3.83 (d, J=11.1, 1H), 3.66-3.45 (m, 2H), 2.80-2.57 (m, 2H), 2.48 (br.s, 1H), 2.32-2.25 (m, 1H), 2.00 (br.s, 1H), 1.59 (br.s, 1H), 1.13 (d, J=6.3 Hz, 3H). See FIG. 8.

### m. Step 6—Synthesis of (3R,4J)-7-fluoro-3-((R)-2-methylmorpholino)chroman-4-amine

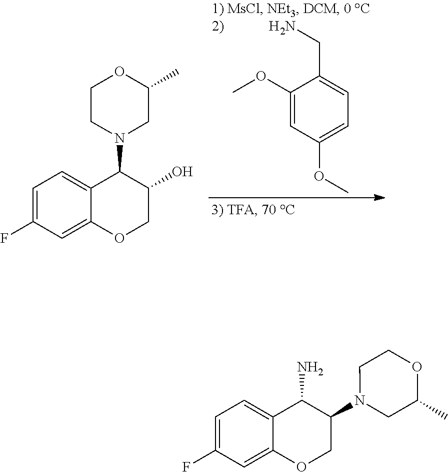

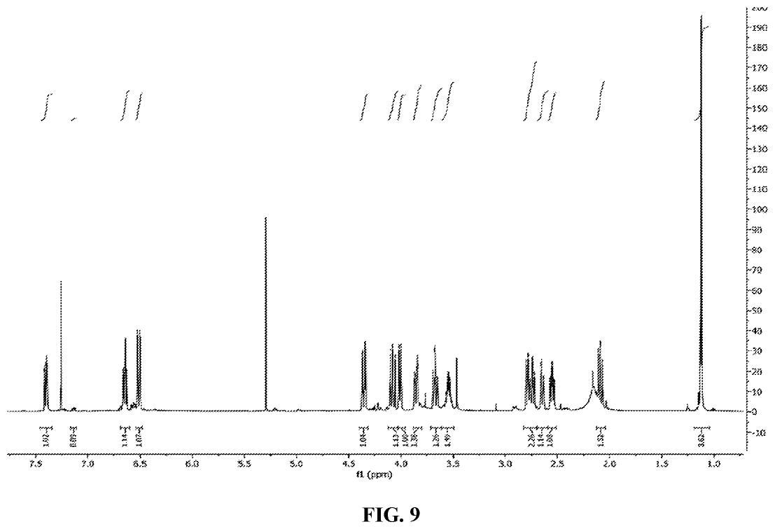

Methanesulfonyl chloride (11.17 g, 7.54 mL, 1.2 eq, 97.48 mmol) was added to a mixture of (3S,4R)-7-fluoro-4-((R)-2-methylmorpholino)chroman-3-ol (23.10 g, 94% wt, 1 eq, 81.23 mmol) and triethylamine (17.26 g, 23.8 mL, 2.1 Eq, 170.6 mmol) in anhydrous DCM (350 mL) at 0 °C under nitrogen. The reaction was stirred for 1.5 hours. (2,4-dimethoxyphenyl)methanamine (16.30 g, 14.64 mL, 1.2 Eq, 97.48 mmol) was added, and the reaction was heated to 45 °C for 2 hours. This was cooled to 22 °C and washed with water (350 mL). The aqueous phase was extracted with DCM (200 mL). The combined organics were dried (MgSO₄) and concentrated in vacuo to afford the crude intermediate diamine. This crude was dissolved in TFA (100 mL) and heated to 70 °C for 1 hour. The reaction was cooled to 0 °C and basified with 12M NaOH (pH >12). The aqueous phase was extracted with 1:3 MeOH:DCM (2×200 mL). The combined organics were dried (MgSO₄), filtered through celite, and concentrated in vacuo. The crude product was spilt in two portions and purified by silica gel chromatography eluting with 0-10%, MeOH/DCM to afford (3R,4S)-7-fluoro-3-((R)-2-methylmorpholino)chroman-4-amine (20.1 g, 59 mmol, 72%, 78% purity) as a dark yellow oil. ¹H NMR (500 MHz, CDCl3) δ 7.44-7.36 (m, 1H), 6.68-6.60 (m, 1H), 6.51 (d, J=10.2 Hz, 1H), 4.36 (dd, J=11.2, 3.1 Hz, 1H), 4.13-4.05 (m, 1H), 4.01 (d, J=7.6 Hz, 1H), 3.86 (d, J=11.2 Hz, 1H), 3.68 (td, J=11.1, 2.7 Hz, 1H), 3.58-3.49 (m, 1H), 2.80-2.73 (m, 2H), 2.68-2.61 (m, 1H), 2.57-5.48 (m, 1H), 2.09 (dd, J=10.9, 9.6 Hz, 2H), 1.13 (d, J=6.2 Hz, 3H) (exchangeable proton not observed). See FIG. 9.

### n. Step 7—Synthesis of N-((3R,4S)-7-fluoro-3-((R)-2-methylmorpholino)chroman-4-yl)-6-(trifluoromethyl)-7H-pyrrolo[2,3-d]pyrimidin-4-amine

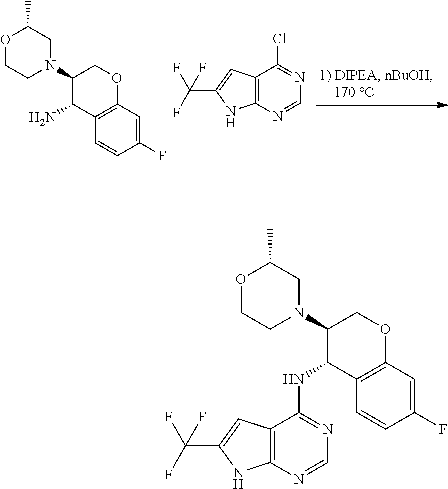

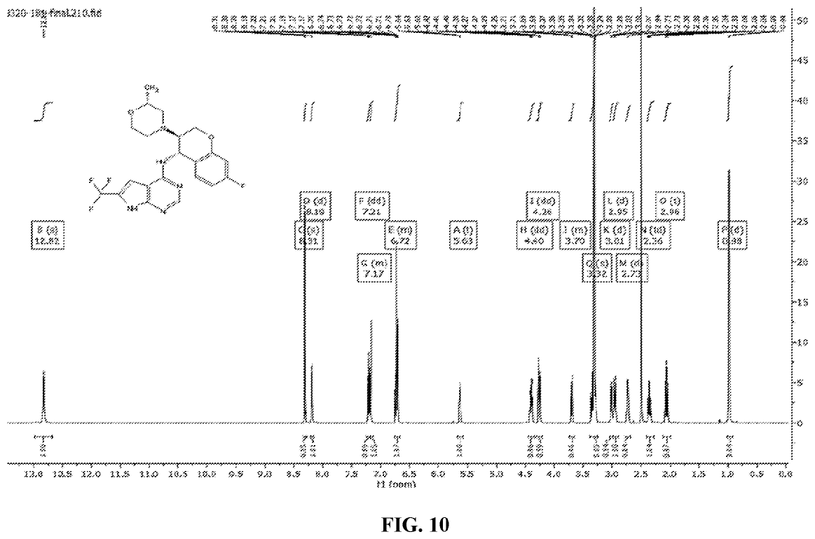

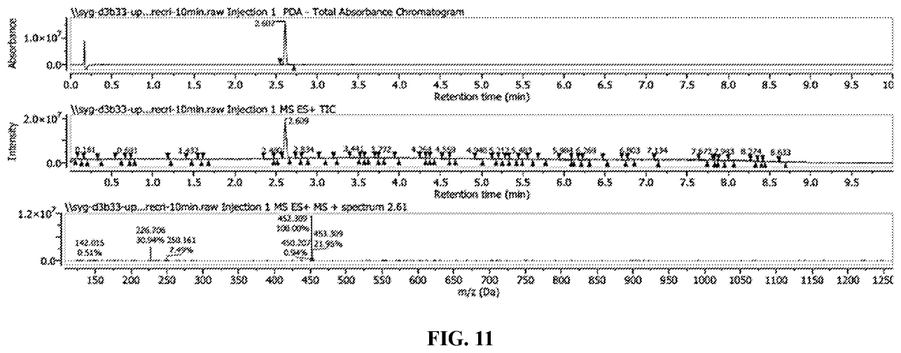

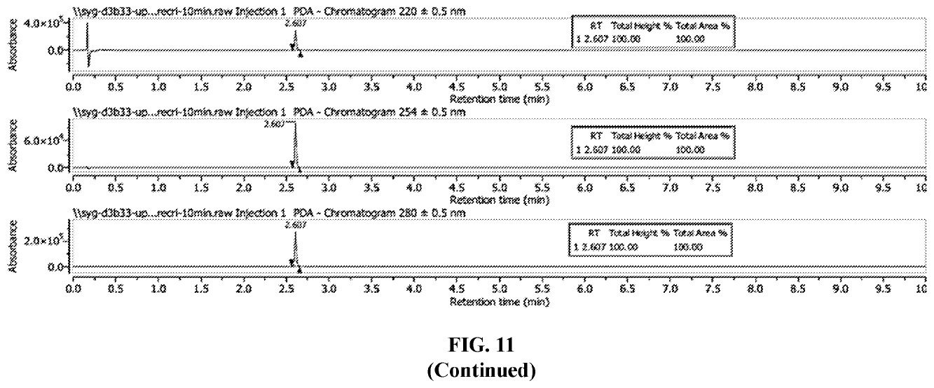

(3R,4S)-7-fluoro-3-((R)-2-methylmorpholino)chroman-4-amine (17.0 g, 78% wt, 1.1 eq, 49.6 mmol), 4-chloro-6-(trifluoromethyl)-7H-pyrrolo[2,3-d]pyrimidine (10.0 g, 1 eq, 45.1 mmol) and DIPEA (17.5 g, 23.6 mL, 3 eq, 135 mmol) in nBuOH (85 mL) was heated to 170 °C in a sealed tube for 8 hours. The reaction was cooled to room temperature and partitioned between DCM (100 mL) and water (100 mL). The organic phase was separated, washed with water (100 mL) and brine (150 mL). The organic phase was dried (MgSO₄), filtered, and concentrated in vacuo to a brown gum. The crude material was dissolved in DCM (32 mL), seeded with trace quantities of the desired product, and agitated. The resultant solid was collected via filtration to afford N-((3R,4S)-7-fluoro-3-((R)-2-methylmorpholino)chroman-4-yl)-6-(trifluoromethyl)-7H-pyrrolo[2,3-d]pyrimidin-4-amine (14.97 g, 33 mmol, 73%, 99% purity) as a white solid. ¹H NMR (500 MHz, DMSO) δ 12.82 (s, 1H), 8.31 (s, 1H), 8.19 (d, J=8.1 Hz, 1H), 7.21 (dd, J=8.3, 6.7 Hz, 1H), 7.18-7.13 (m, 1H), 6.75-6.67 (m, 2H), 5.63 (t, J=6.7 Hz, 1H), 4.40 (dd, J=11.9, 5.4 Hz, 1H), 4.26 (dd, J=11.9, 2.5 Hz, 1H), 3.74-3.65 (m, 1H), 3.37-3.24 (m, 2H), 3.01 (d, J=11.5 Hz, 1H), 2.95 (d, J=11.0 Hz, 1H), 2.73 (d, J=4.9 Hz, 1H), 2.36 (td, J=11.5, 3.1 Hz, 1H), 2.06 (t, J=10.4 Hz, 1H), 0.98 (d, J=6.3 Hz, 3H). See FIG. 10. LCMS traces of the isolated product are shown in FIG. 11.

A further crop of material was isolated by concentrating the liquors and purifying by silica gel chromatography (eluting with 0-25%, 3:1 EtOAc:EtOH/isohexane) to afford N-((3R,4S)-7-fluoro-3-((R)-2-methylmorpholino)chroman-4-yl)-6-(trifluoromethyl)-7H-pyrrolo[2,3-d]pyrimidin-4-amine (1.77 g, 3.8 mmol, 8.5%, 98% purity) as a yellow solid. This was purified by recrystallization in DCM. ¹H NMR analysis was consistent with FIG. 10.

### 3. Alternative Prophetic Synthesis of (3R,4S)-7-Fluoro-3-((R)-2-methylmorpholino)chroman-4-amine

An alternative synthesis of intermediate (3R,4S)-7-fluoro-3-((R)-2-methylmorpholino)chroman-4-amine is shown in Scheme 2.

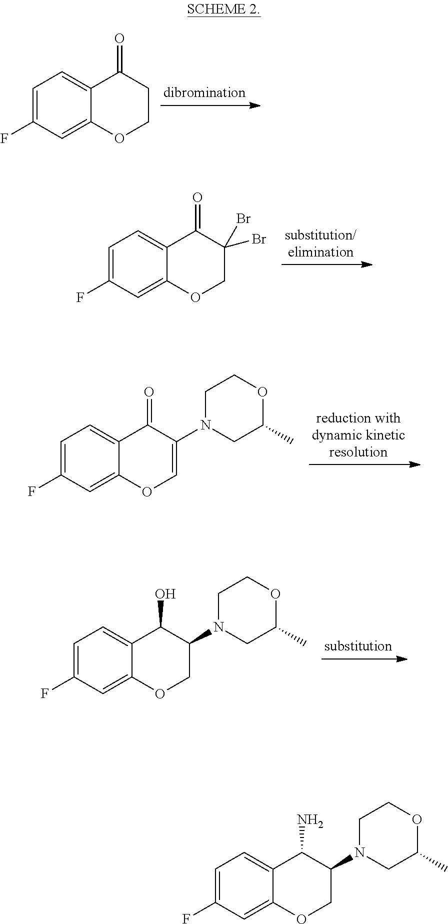 [SMILES: O=C1CCOc2cc(F)ccc21 ; O=C1c2ccc(F)cc2OCC1(Br)Br ; C[C@@H]1CN(c2coc3cc(F)ccc3c2=O)CCO1 ; C[C@@H]1CN([C@H]2COc3cc(F)ccc3[C@H]2O)CCO1 ; C[C@@H]1CN([C@H]2COc3cc(F)ccc3[C@@H]2N)CCO1]

### 4. Alternative Prophetic Synthesis of 7-Fluoro-3-((R)-2-methylmorpholino)chroman-4-one

An alternative synthesis of intermediate 7-fluoro-3-((R)-2-methylmorpholino)chroman-4-one is shown in Scheme 3.

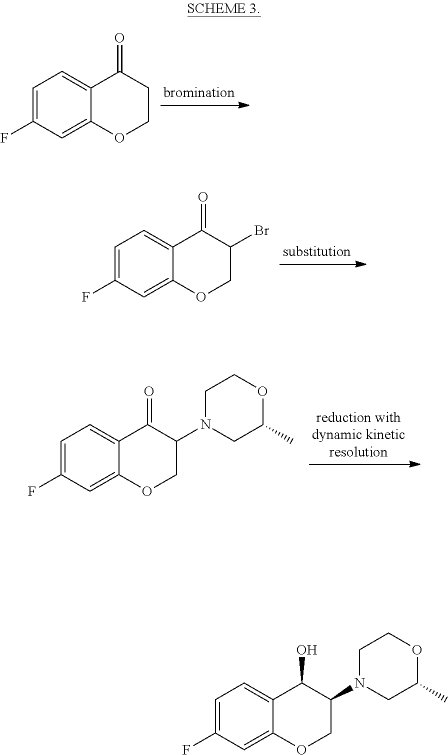 [SMILES: O=C1CCOc2cc(F)ccc21 ; O=C1c2ccc(F)cc2OCC1Br ; C[C@@H]1CN(C2COc3cc(F)ccc3C2=O)CCO1 ; C[C@@H]1CN([C@H]2COc3cc(F)ccc3[C@H]2O)CCO1]

### 5. Preparation of Racemic-trans 3-Bromo-7-fluorochroman-4-yl Acetate

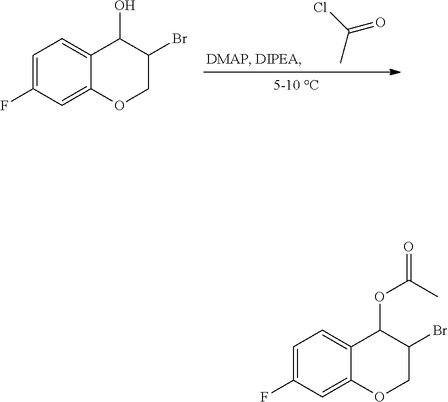

Trans-racemic bromohydrin was used to make the trans-racemic acetate as an analytical marker using the procedure described below. An analytical SFC method was developed, which could separate both isomers of the starting bromohydrin and both isomers of the acetate product.

Briefly, acetyl chloride (572 mg, 518 μL, 1.2 eq, 7.29 mmol) was added portion-wise to a stirring solution of 3-bromo-7-fluorochroman-4-ol (1.50 g, 1 eq, 6.07 mmol), N,N-dimethylpyridin-4-amine (37.1 mg, 0.05 eq, 304 μmol) and DIPEA (1.18 g, 1.59 mL, 1.5 Eq, 9.11 mmol) in DCM (15.0 mL) at 5-10 °C over 10 minutes. The reaction was allowed to warm to ambient temperature and stirred for 30 minutes. The reaction was diluted with DCM (10 mL) and washed with water (2×15 mL). The combined aqueous was extracted with DCM (15 mL) and the combined organics were washed with water (15 mL). The DCM was passed through a phase separator cartridge and adsorbed onto silica gel and concentrated under reduced pressure. The crude product was purified by chromatography on silica gel (40 g cartridge, 0-20% EtOAc/isohexane) to afford 3-bromo-7-fluorochroman-4-yl acetate (1.33 g, 4.5 mmol, 74%, 98% purity) as a clear colourless oil that crystallised upon standing. ¹H NMR (500 MHz, DMSO) δ 7.37 (dd, J=8.4, 6.6 Hz, 1H), 6.87-6.79 (m, 2H), 5.81 (d, J=3.1 Hz, 1H), 4.74 (q, J=2.7 Hz, 1H), 4.41 (d, J=2.4 Hz, 2H), 2.06 (s, 3H). ¹⁹F NMR (471 MHz, DMSO) δ −110.06.

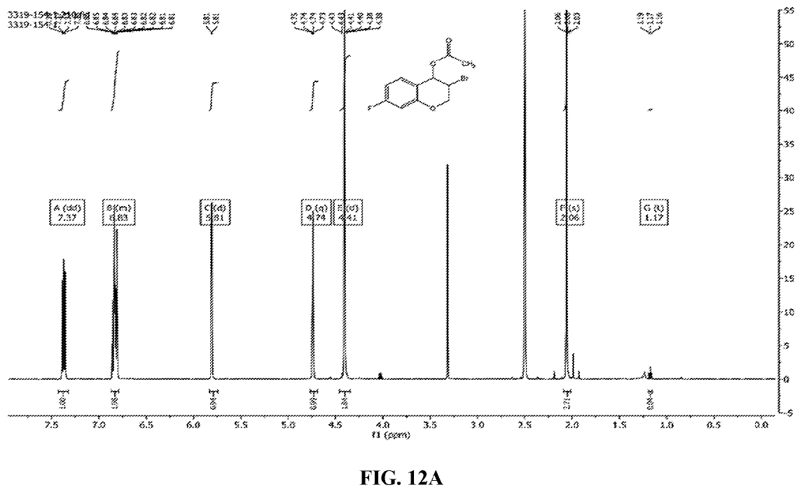

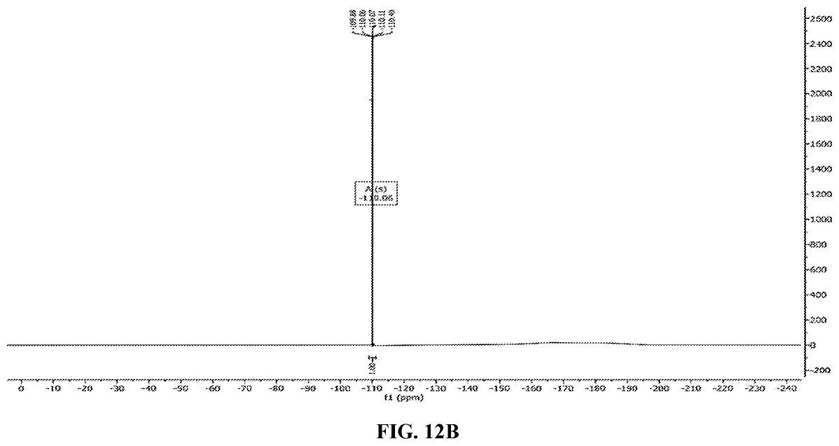

A ¹H NMR and ¹³C NMR of the isolated product is shown in FIG. 12A and FIG. 12B.

The product was analysed by UPLC (CSH C18 Column, 130 ∀, 1.7 μm, 2.1 mm×30 mm, 3 min method, 0.1% Formic acid, 2-100% MeCN/water) (1.57 min, 98.5% purity 210-400 nm). UPLC traces of the isolated product are shown in FIG. 12C.

### 6. Alternative to Step 6—Synthesis of (3R,4S)-7-fluoro-3-((R)-2-methylmorpholino)chroman-4-amine

An alternative synthesis of intermediate (3R,4S)-7-fluoro-3-((R)-2-methylmorpholino)chroman-4-amine is shown in Scheme 8. The synthesis of intermediate (3R,4S)-7-fluoro-3-((R)-2-methylmorpholino)chroman-4-amine according to Scheme 8 can be used in conjunction with and/or as a replacement step of one or more of the synthesis schemes disclosed herein.

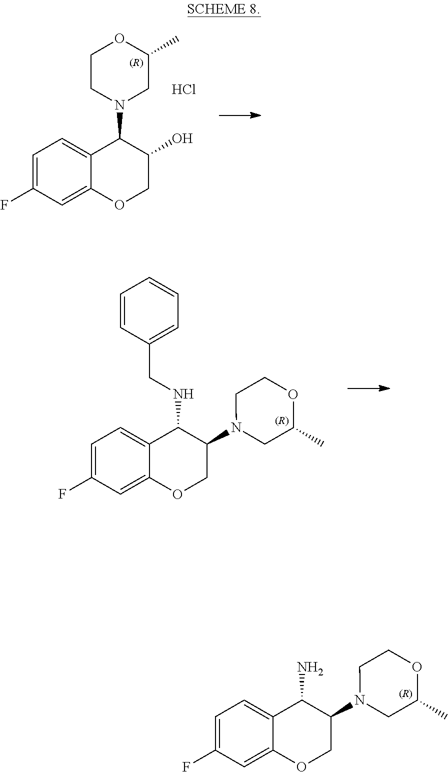

Scheme 8 can be achieved by the following synthesis scheme.

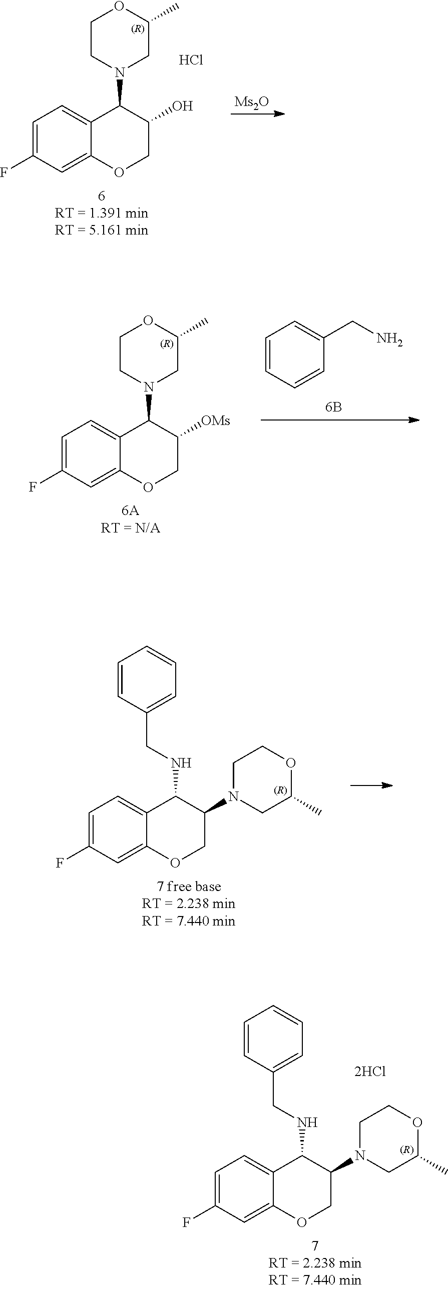

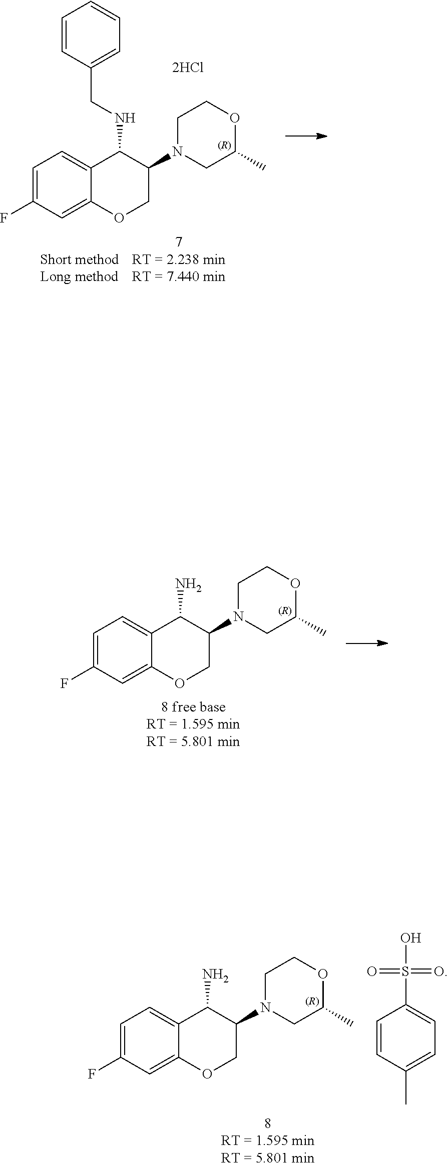

Compounds 6-8, which are discussed in this example of an alternative to Step 6, refer to the compounds identified as Compounds 6-8 in Scheme 8.

As discussed herein, Compound 6 may be in the form of a pharmaceutically acceptable salt thereof. For instance, the Compound 6 may be in the form of a chloride salt. In certain embodiments, however, Compound 6 is not in the form of a salt and is in a free form. Compound 6 and/or a pharmaceutically acceptable salt thereof may undergo mesylation of a hydroxyl to obtain Compound 6A. In certain embodiments, mesylation may occur by way of employing ethanesulfonic anhydride (Ms₂O).

Compound 6A may be in the form of a pharmaceutically acceptable salt thereof, such as a chloride salt. Compound 7 may be obtained by way of reacting Compound 6A may with a benzylamine. Additionally or alternatively, in certain embodiments, the formation of Compound 7 from Compound 6 and/or Compound 6A is free of trifluoroacetic acid (TFA). In certain embodiments, the formation of Compound 7 from Compound 6 and/or Compound 6A is free of 1,4-dimethoxybenzylamine.

Compound 7 may be in a free form or in the form of a pharmaceutically acceptable salt thereof. For example, Compound 7 may be a pharmaceutically acceptable chloride salt. In certain embodiments, a pharmaceutically acceptable salt of Compound 7 may be obtained while impurities, such as an aziridine, may be isolated and/or removed. Compound 7 may be reduced using hydrogen from an acid, such as HCOONH₄ and/or HCOOK, to obtain Compound 8 ((3R,4S)-7-fluoro-3-((R)-2-methylmorpholino)chroman-4-amine). Additionally or alternatively, Compound 8 may be formed from Compound 7 using a hydrogenator.

Compound 8 may be in a free form or in the form of a pharmaceutically acceptable salt thereof. In certain embodiments, the pharmaceutically acceptable salt of Compound 8 may be a TsOH salt. In some embodiments, a pharmaceutically acceptable salt of Compound 8 may be obtained while impurities are isolated and/or removed.

### 7. Chiral Analytical Analysis

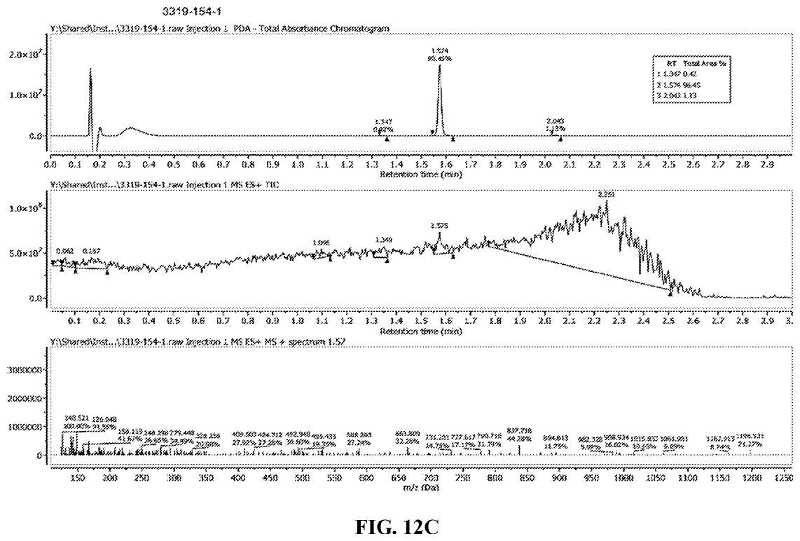

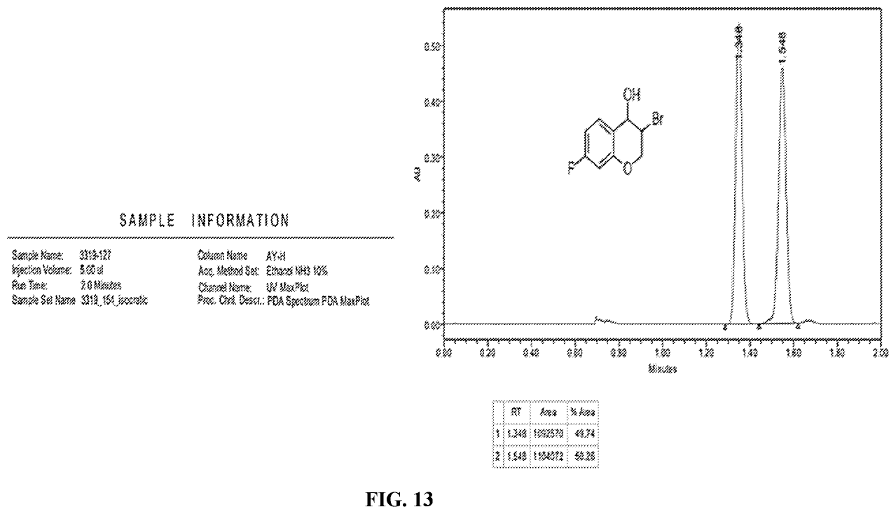

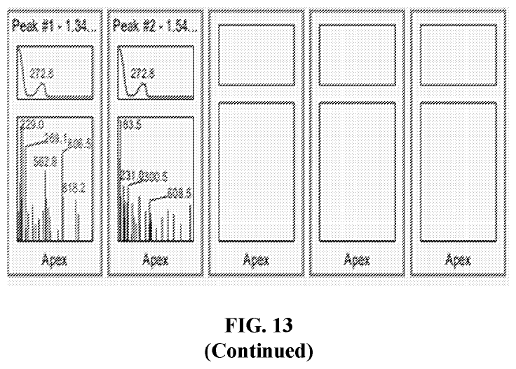

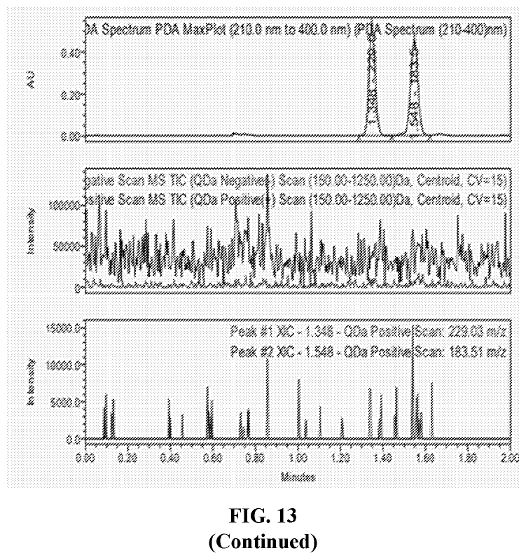

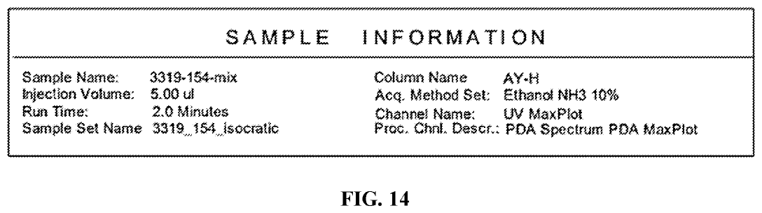

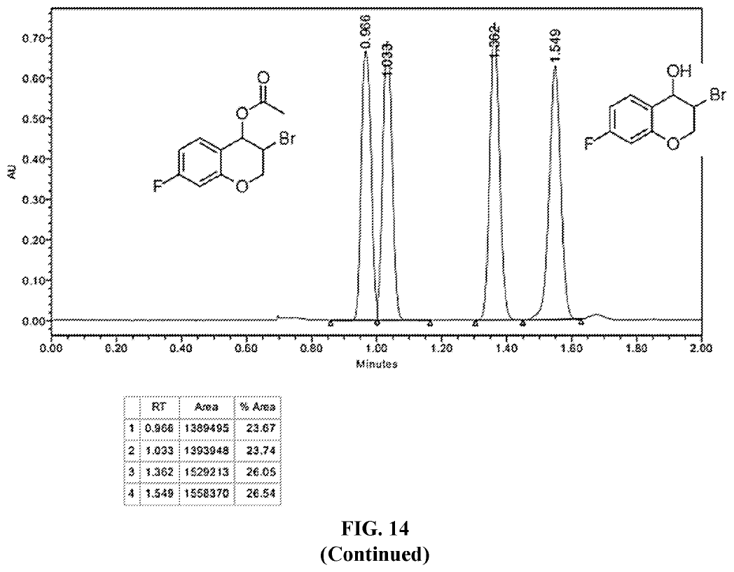

A supercritical fluid chromatography (SFC) method was developed to differentiate the racemic bromohydrin and the acylated product using an AY-H column (4.6×250 mm, 5 μM) with mobile phase 90% CO₂ and 10% EtOH (no modifier). The flow rate was 4 mL/min at a UV detection of 220-400 nm. The run time was 2 mins. BPR 120 bar. See Table 2, FIG. 13, and FIG. 14.

TABLE 2
Bromohydrin

| | |
|---|---|
| Retention time, Isomer 1 | 1.348 min |
| Retention time, Isomer 2 | 1.548 min |

Acylated product

| | |
|---|---|
| Retention time, Isomer 1 | 0.966 min |
| Retention time, Isomer 2 | 1.033 min |

### 8. Preparation of Racemic-trans 6-Fluoro-((R)-2-methylmorpholino-chroman-3-ol

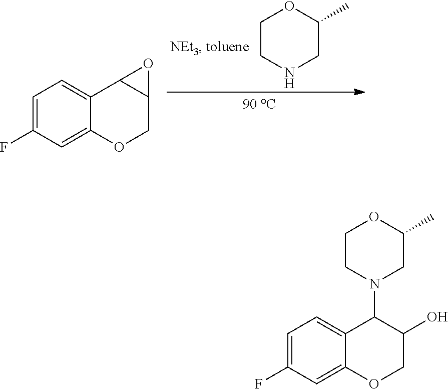

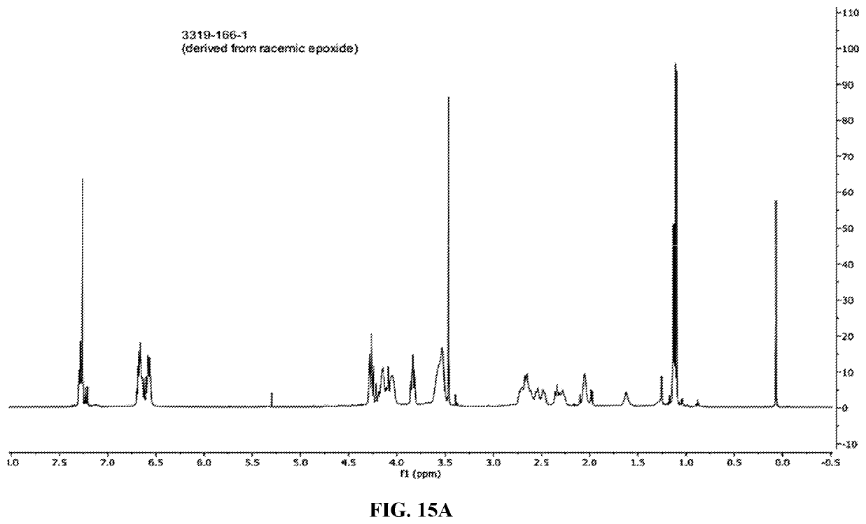

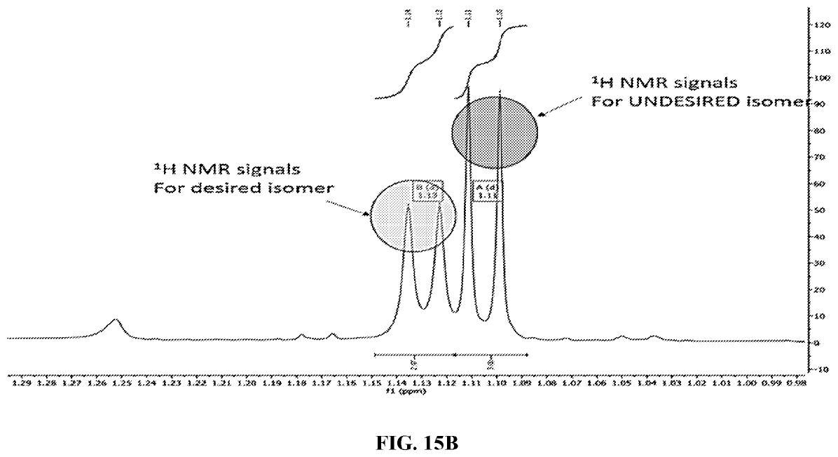

A mixture of 5-fluoro-1a,7b-dihydro-2H-oxireno[2,3-c]chromene (27.1 mg, 0.95 eq, 163 mol), (R)-2-methylmorpholine HCl (23.6 mg, 1 eq, 172 μmol) and triethylamine (26.1 mg, 35.9 μL, 1.5 eq, 258 μmol) in toluene (0.75 mL) was heated to 90 °C for 4.5 hr. The cool reaction mixture was partitioned between water (10 mL) and EtOAc (10 mL). The aqueous phase was then extracted with EtOAc (10 mL) and the combined organics were washed with water (2×10 mL). The organic was dried over Na₂SO₄ then concentrated under vacuum. The crude product purified by chromatography on silica gel (12 g cartridge, 0-5 MeOH/DCM) to afford 7-fluoro-4-((R)-2-methylmorpholino)chroman-3-ol (10.0 mg, 37.4 μmol, 23.0%) as a dark yellow oil. A ¹H NMR of the product is shown in FIG. 15A (full spectra) and FIG. 15B (expanded).

### 9. Enzymatic Hydrolysis

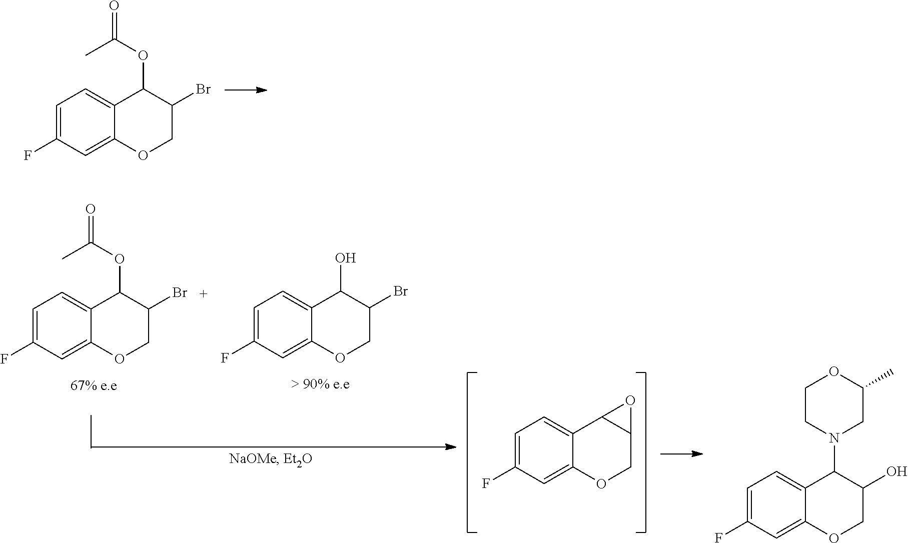

Novozyme 51032 (200 mg) was added to a stirring emulsion of 3-bromo-7-fluorochroman-4-yl acetate (200 mg, 1 eq, 692 μmol), THE (0.8 mL) and potassium phosphate buffer (2.00 mL). The mixture was heated to 30 °C for 66 hours. The cool reaction was diluted with water (10 mL) and extracted with MTBE (2×10 mL). The combined organics were washed with water (2×10 mL), dried (MgSO₄) and pre-adsorbed directly onto silica. The crude product was purified by chromatography on silica gel (24 g cartridge, 0-25 EtOAc/isohexane) to afford 3-bromo-7-fluorochroman-4-yl acetate (95.0 mg, 329 μmol, 47.5%) as a clear colourless gum (3319-160-A), and 3-bromo-7-fluorochroman-4-ol (50.0 mg, 202 μmol, 29.3%) as a clear oil which crystallised upon standing (3319-160-B).

3319-160-A, 3-Bromo-7-fluorochroman-4-yl acetate. ¹H NMR (500 MHz, DMSO) δ 7.42-7.32 (m, 1H), 6.86-6.78 (m, 2H), 5.81 (d, J=3.1 Hz, 1H), 4.74 (dt, J=3.1, 2.5 Hz, 1H), 4.41 (d, J=2.3 Hz, 2H), 2.06 (s, 3H).

3319-160-B, 3-Bromo-7-fluorochroman-4-ol. ¹H NMR (500 MHz, DMSO) δ 7.34 (dd, J=8.6, 6.8 Hz, 1H), 6.79 (td, J=8.6, 2.6 Hz, 1H), 6.72 (dd, J=10.5, 2.6 Hz, 1H), 6.19 (d, J=5.8 Hz, 1H), 4.65-4.61 (m, 1H), 4.50-4.44 (m, 2H), 4.34-4.28 (m, 1H).

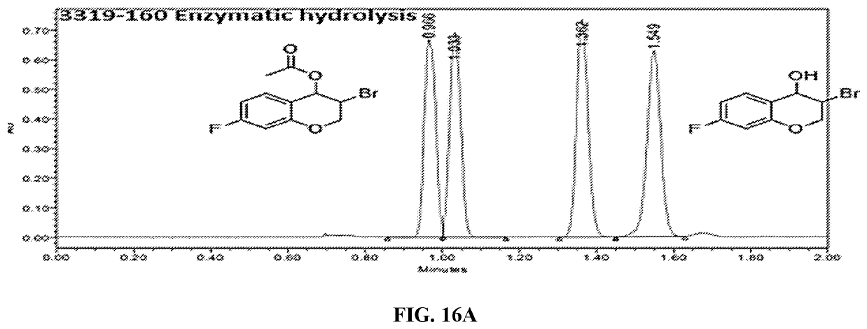

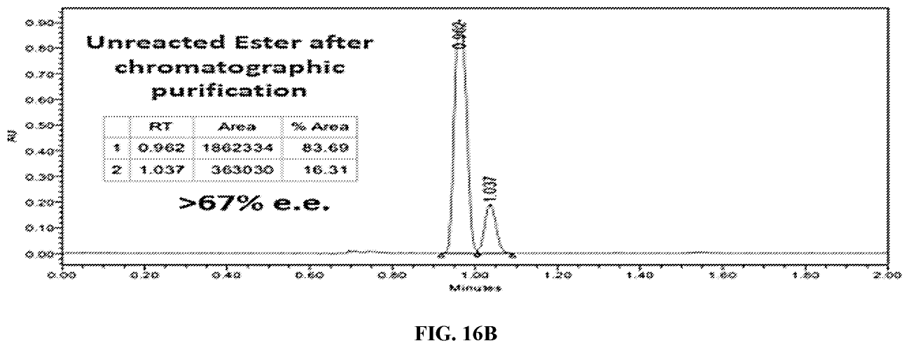

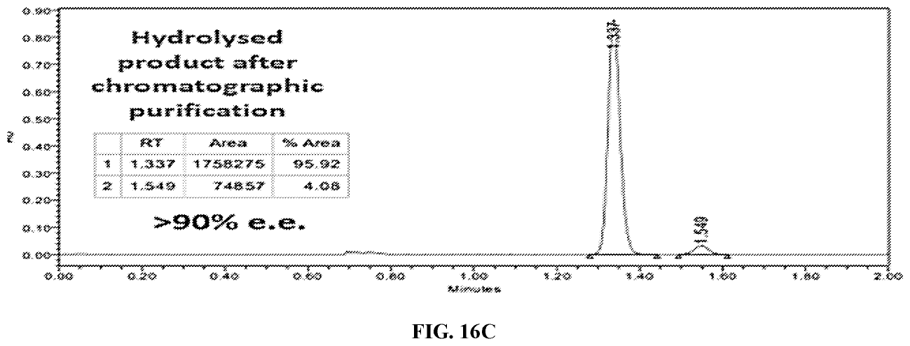

3319-160-A and 3319-160-B were dissolved in methanol to 1 mg/mL and analysed by SFC using an AY-H column (4.6×250 mm, 5 μm) with mobile phase 90% CO₂ and 10% EtOH. The flow rate was 4 mL/min at a UV detection of 220-400 nm. The run time was 2 mins. See FIG. 16A-C.

The enriched, unreacted, ester (67% e.e.) obtained in the previous step was progressed using the following procedure: To a stirred solution of (3S,4S)-3-bromo-7-fluorochroman-4-yl acetate (84.0 mg, 1 eq, 291 μmol, 67% e.e.) in dry diethyl ether (1.00 mL) under nitrogen was added carefully sodium methanolate (31.4 mg, 2.0 eq, 581 μmol). The reaction was stirred at 25 °C for 1 h 30 min. This was diluted with diethyl ether (5 mL), washed with water (5 mL). The aqueous was extracted with diethyl ether (2×5 mL). The organic layers were combined, dried over MgSO4 and concentrated under vacuum to afford 5-fluoro-1a,7b-dihydro-2H-oxireno[2,3-c]chromene (47.0 mg, 283 μmol, 97.4%) as a white solid which was used without further purification.

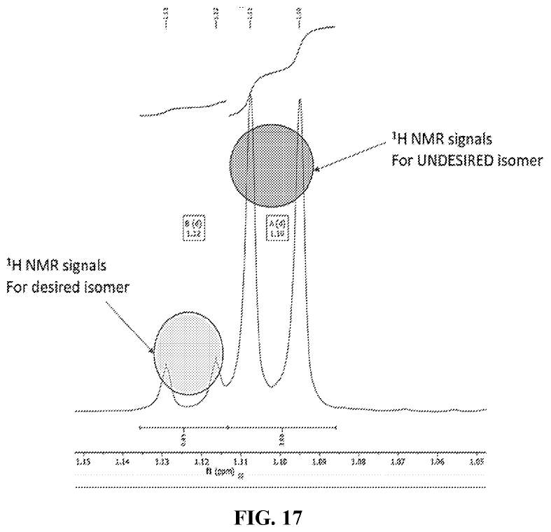

A mixture of (1aR,7bS)-5-fluoro-1a,7b-dihydro-2H-oxireno[2,3-c]chromene (46.0 mg, 0.95 eq, 277 μmol), (R)-2-methylmorpholine, HCl (40.1 mg, 1 eq, 291 μmol) and triethylamine (44.2 mg, 60.9 μL, 1.5 eq, 437 μmol) in toluene (0.75 mL) was heated to 90 °C for 4.5 hr. The reaction mixture was partitioned between water (10 mL) and EtOAc (10 mL). The aqueous phase was then extracted with EtOAc (10 mL) and the combined organics were washed with water (2×10 mL). The organic was dried over Na2SO4 then concentrated under vacuum. The crude product purified by chromatography on silica gel (12 g cartridge, 0-5 MeOH/DCM) to afford (3S,4R)-7-fluoro-4-((R)-2-methylmorpholino)chroman-3-ol as a dark yellow oil. ¹H NMR analysis of this indicates that the major product is the undesired derivative (approx. 67% e.e.). See FIG. 17.

### 10. Enzymatic Transesterification

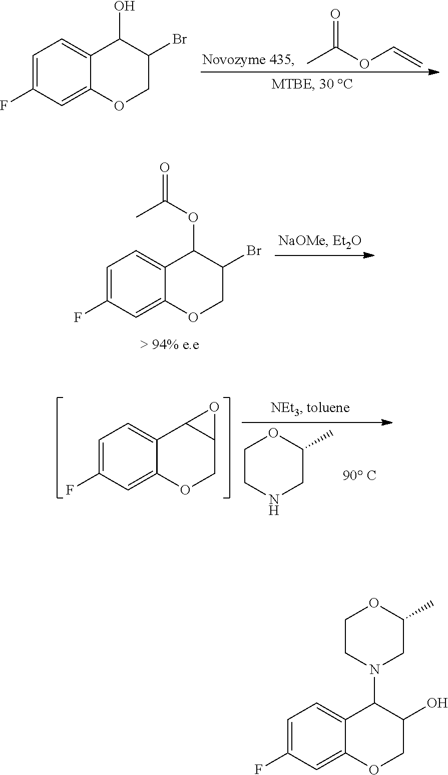

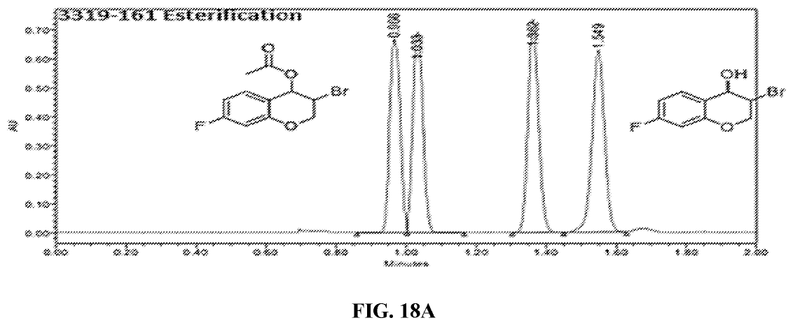

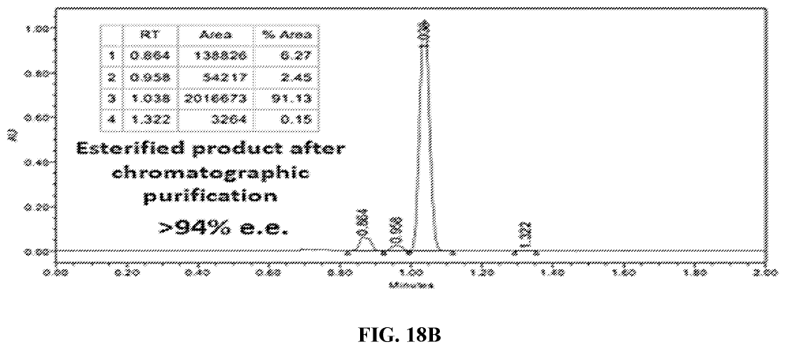

A mixture of vinyl acetate (83.6 mg, 89.5 μL, 1.2 eq, 971 μmol), Novozyme 435 (200 mg) and 3-bromo-7-fluorochroman-4-ol (200 mg, 1 eq, 810 μmol) in MTBE (4.00 mL) was stirred at 30 °C for 66 hours. The cool reaction was diluted with water (10 mL) and extracted with MTBE (2×10 mL). The combined organics were washed with water (2×10 mL), dried (MgSO4) and pre-adsorbed directly onto silica. The crude product was purified by chromatography on silica gel (24 g cartridge, 0-25 EtOAc/isohexane) to afford 3-bromo-7-fluorochroman-4-yl acetate (27.0 mg, 93.4 μmol, 11.5%) as a clear oil which crystallised upon standing (3319-161-A). ¹H NMR (500 MHz, DMSO) δ 7.37 (dd, J=8.4, 6.6 Hz, 1H), 6.88-6.80 (m, 2H), 5.81 (d, J=3.1 Hz, 1H), 4.74 (q, J=2.6 Hz, 1H), 4.41 (d, J=2.4 Hz, 2H), 2.06 (s, 3H). 3319-161-A was dissolved in methanol to 1 mg/mL and analysed by SFC. See FIG. 18A and FIG. 18B.

The optimal technique and conditions are SFC using an AY-H (4.6×250 mm, 5 μm) with mobile phase 90% CO₂ and 10% EtOH. The flow rate was 4 mL/min at a UV detection of 220-400 nm. The run time was 2 mins.

To a stirred solution of 3-bromo-7-fluorochroman-4-yl acetate (27.0 mg, 93% Wt, 1 eq, 86.9 μmol) in dry diethyl ether (1.00 mL) under nitrogen was added carefully sodium methanolate (9.38 mg, 2.0 eq, 174 μmol). The reaction was stirred at 25 °C overnight. This was diluted with diethyl ether (5 mL), washed with water (5 mL). The aqueous was extracted with diethyl ether (2×5 mL). The organic layers were combined, dried over MgSO₄ and concentrated under vacuum to afford 5-fluoro-1a, 7b-dihydro-2H-oxireno[2,3-c]chromene (7.00 mg, 42.1 μmol, 48.5%) as a white solid, which was used without further purification.

A mixture of (1aR,7bS)-5-fluoro-1a, 7b-dihydro-2H-oxireno[2,3-c]chromene (7.00 mg, 0.95 eq, 42.1 μmol), (R)-2-methylmorpholine, HCl (6.10 mg, 1 eq, 44.3 μmol) and triethylamine (6.73 mg, 9.27 μL, 1.5 eq, 66.5 μmol) in toluene (0.75 mL) was heated to 90 °C for 4.5 hr. The reaction mixture was partitioned between water (10 mL) and EtOAc (10 mL). The aqueous phase was then extracted with EtOAc (10 mL) and the combined organics were washed with water (2×10 mL). The organic was dried over Na₂SO₄ then concentrated under vacuum. See FIG. 18.

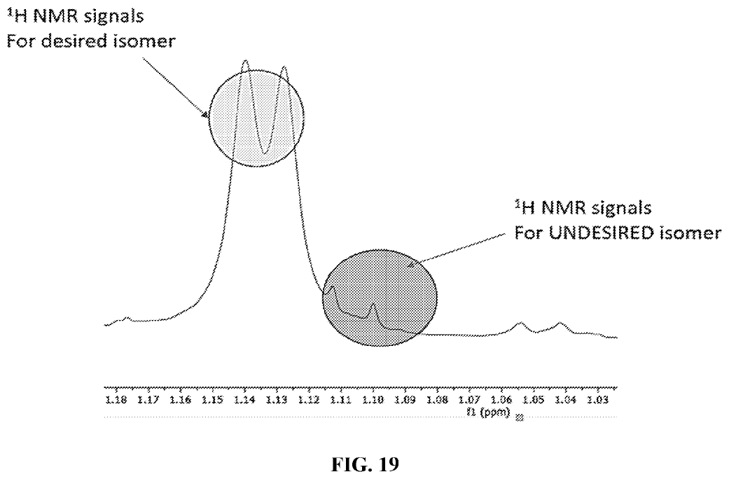

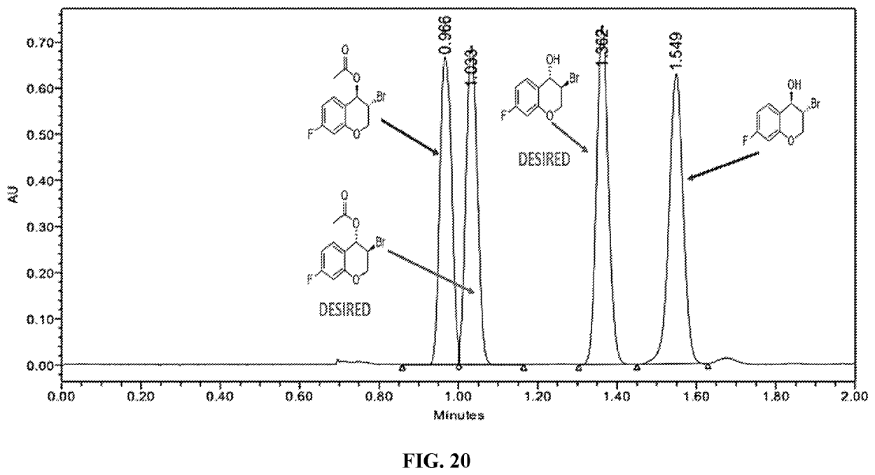

Based on these data, stereochemistry has been assigned as shown in FIG. 19.

### 11. Biological Methods

a. Human mt-Keima Mitophagy Assay

HeLa cells expressing mt-Keima and YFP-Parkin (HeLa MKYP) were plated at 10,000 cells/well in 96 well plates along with Mitokinin compounds at various concentrations. Following 16 hrs of incubation, cells were treated with 1 μM FCCP/oligomycin for 6 hours, then analyzed via FACS for the presence of mitochondria in lysosomes (as determined by an emissions spectrum shift from the pH-sensitive mt-Keima tag). Additionally, cells treated with 25 μM Mitokinin compound but not treated with FCCP/oligomycin were analyzed for cytotoxicity.

b. In Vitro Mitotoxicity (glucose/galactose) Assay

Briefly, SKOV3 cells (ATCC HTB-77) were plated in DMEM (Corning 10-013-CV) medium containing 25 mM glucose or DMEM with no glucose (Gibco 11966-025) supplemented with 10 mM galactose at 9,000 cells/well in 96 well plates (Corning costar 3610). 24 hrs later, compounds, in DMSO, were added at various concentrations to a final 0.1% DMSO for all wells. Following 20 hours of incubation with compound, all wells had their growth medium replaced with DMEM containing 25 mM of glucose followed by the addition of Promega CellTiter-Glo® reagent as per manufacturer's instructions. After a 15 minute incubation cellular ATP levels were read out using a Promega GloMax® DiscoverMicroplate Reader.

The ratio of relative CellTiter-Glo values from cells grown in galactose to cells grown in glucose was determined (gal/glc ratio). The gal/glc ratio vs concentration is then graphed, and non-linear regression is used to find the highest concentration of MTK compound at which gal/glc>0.80 (the “highest mito-safe dose”, or IC₂₀). The Mito-Safety Index in Table 2 is equal to the highest mito-safe dose divided by the mt-Keima EC₅₀ dose.

c. Evaluation of an Exemplary, Non-Limiting Compound for PINK1 Kinase Activity

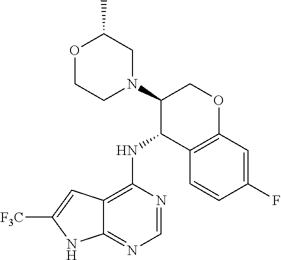 [SMILES: C[C@@H]1CN([C@H]2COc3cc(F)ccc3[C@@H]2Nc2ncnc3[nH]c(C(F)(F)F)cc23)CCO1]

The above compound was prepared using the synthetic methods detailed herein, and evaluated for PINK1 activity. See Table 3. Additional exemplary PINK1 modulators for which the synthetic methods described herein can be used are described in WO 2021/168446

### INCORPORATED BY REFERENCE

TABLE 3

| | | Highest | | |
|---|---|---|---|---|
| | | safe dose | Highest | |
| | Max % | (cytotoxicity; | safe dose | Mito-Safety |
| Mitophagy | death | no FO; safe | (μM) | Index |
| induction | with | means DAPI +< | (mitotox or | (highest safe |
| EC₅₀ (μM) | 1 μM FO | 20%) (μM) | cytotox) | dose/EC₅₀) |
| 0.13 | 29.6 | 21.9 | 17.3 | 135.44 |

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### 12. Another Exemplary Synthesis of N-((3R,4S)-7-fluoro-3-((R)-2-methylmorpholino)chroman-4-yl)-6-(trifluoromethyl)-7H-pyrrolo[2,3-d]pyrimidin-4-amine

Another non-limiting, exemplary complete synthesis of N-((3R,4S)-7-fluoro-3-((R)-2-methylmorpholino)chroman-4-yl)-6-(trifluoromethyl)-7H-pyrrolo[2,3-d]pyrimidin-4-amine is shown in Scheme 9.

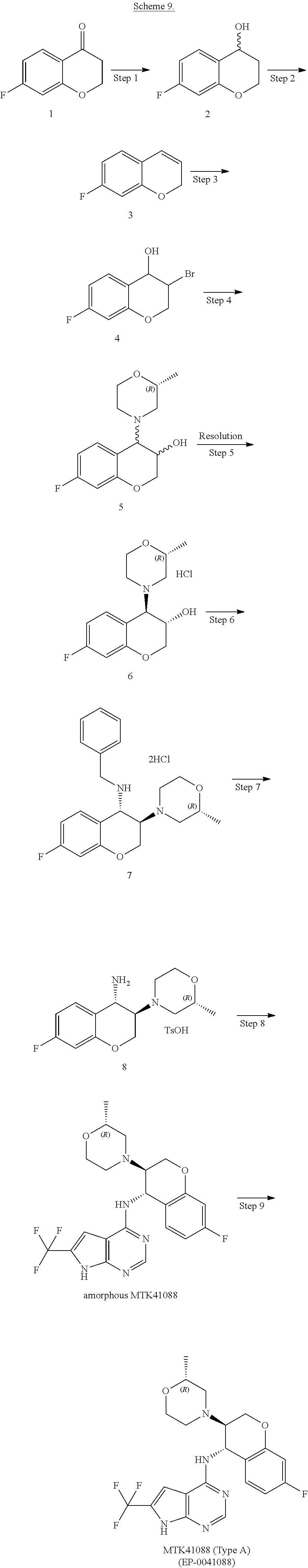

### A. GENERAL ANALYTIC METHODS

¹H NMR spectra were recorded on Bruker Fourier 400/300 MHz and TMS was used as an internal standard.

LC-MS was taken on a quadruple Mass Spectrometer on Agilent LC/MSD 1200 Series (Column: ODS 2000 (50× 4.6 mm, 5 μm) operating in ES (+) or (−) ionization mode; T=30 °C; flow rate=1.5 mL/min; detected wavelength: 214 nm.

### b. LIST OF ABBREVIATIONS

The following table provides a list of abbreviations for this Example.

| | |
|---|---|
| aq | Aqueous |
| °C | Celsius |
| Equiv. | Equivalent |
| THF | tetrahydrofuran |
| NaCl | sodium chloride |
| Na₂SO₄ | sodium sulfate |
| EtOAc | Ethyl acetate |
| DCM | Dichloromethane |
| MeOH | Methanol |
| NBS | N-Bromosuccinimide |
| NaOH | Sodium hydroxide |
| TEA | Triethylamine |
| NaHCO₃ | Sodium bicarbonate |
| ACN | Acetonitrile |
| n-BuOH | n-Butanol |
| DIPEA | N,N-diisopropylethylamine |
| NaHSO₄ | Sodium bisulfate |
| NaBH₄ | Sodium borohydride |
| TsOH•H₂O | p-toluenesulfonic acid monohydrate |
| K₂CO₃ | Potassium carbonate |
| Ms₂O | Methanesulfonic anhydrate |
| MTBE | Methyl tertbutyl ether |
| EtOH | Ethanol |
| NaHSO₄ | Sodium bisulfate |

### c. STEP 1 SYNTHESIS OF COMPOUND 2

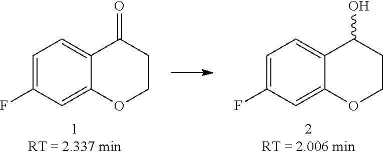 [SMILES: O=C1CCOc2cc(F)ccc21 ; OC1CCOc2cc(F)ccc21]

### Impurities

An impurity was identified (Impurity 1-1), which appeared to be a MeOH residual. The MeOH residual come from the reagent used and may affect the following step to generate impurity. In this example, the MeOH residual was not specifically controlled and/or reduced.

### Observations/Process Changes/Deviations

According to HPLC of aqueous layer, some product was lost in the aqueous layer. The product exhibited good solubility in water. Accordingly, the volume of water (aqueous washing) was reduced from 5 v/w to 1 v/w to minimize the loss, but some product in aqueous layer was still observed.

Another potential reason for the loss in aqueous layer is the large amount of MeOH in aqueous phase. In certain prophetic embodiments, a concentration step may be completed first to remove most of MeOH and being starting the quenching and the extraction. For example, some acetone may be added to quench the reaction before the work up to fully decompose the potential boronic ester or prolong the aging time after the quenching.

A toluene solution was then used to promotone the next step directly. However, one impurity increased a lot. The proposed structure of this impurity is list below. We think the impurity (RT=3.937 min) generated at step 2 was caused by MeOH residual in compound 2 toluene solution. It suggests to perform a solvent swap by azeotrope with fresh toluene to reduce the MeOH residual in crude compound 2 solution before used to next step.

Probable structure of the impurity

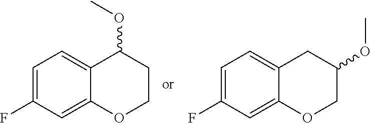

### Batch Summary

| | Input, | Amount | | | | |
|---|---|---|---|---|---|---|
| Lot No. | SM | Product* | Yield | Assay | Purity | Remarks |
| 21099- | 5.84 kg | N/A | 100.00% | N/A | 98.84% | The |
| 006 | | | | | | weight of |
| | | | | | | toluene |
| | | | | | | solution |
| | | | | | | wasn't |
| | | | | | | recorded |
| 21099- | 7.60 kg | 61.02 kg | 100.68% | 12.15% | 98.72% | N/A |
| 020 | | | | | | |
| 21596- | 6.50 kg | 49.95 kg | 101.74% | 13.40% | 98.37% | N/A |
| 001 | | | | | | |

Note:
*toluene solution was used into the next step directly as 100% yield.

### Typical Operation Procedure (E02344-21099-020)

| No. | Raw-material | Amount | | Mol. wt. | Mol | Molar-ratio | |
|---|---|---|---|---|---|---|---|
| 1 | Compound 1 | 7.60 | kg | 166.15 | 45.74 mol | 1.00 | equiv. |
| 2 | Toluene | 114.00 | L | N/A | N/A | 15.00 | v/w |
| 3 | NaBH₄ | 865.20 | g | 37.83 | 22.87 mol | 0.50 | equiv. |
| 4 | MeOH | 11.72 | kg | 32.04 | 365.92 mol | 8.00 | equiv. |
| 5 | Citric acid | 2.28 | kg | N/A | N/A | 0.30 | w/w |
| | monohydrate | | | | | | |
| 6 | NaCl | 9.20 | kg | N/A | N/A | 1.20 | w/w |
| 7 | NaHCO₃ | 0.30 | kg | N/A | N/A | 0.04 | w/w |

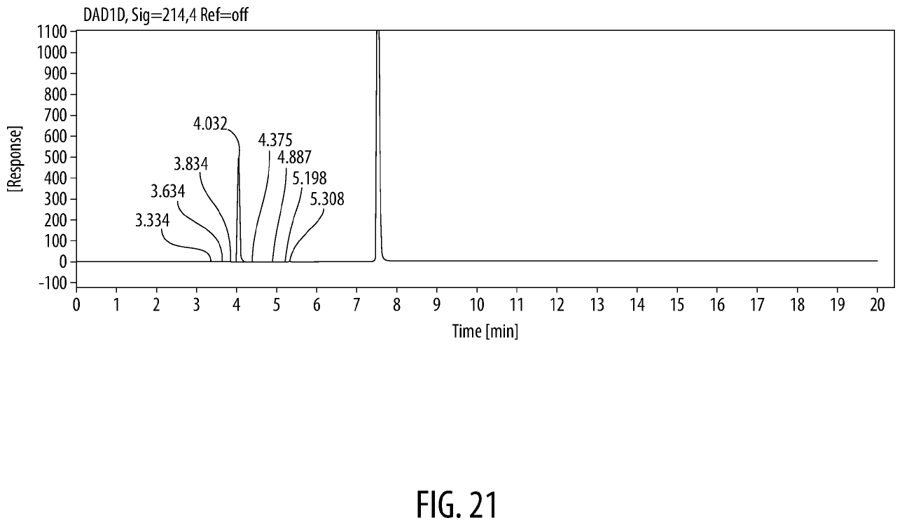

The following steps were conducted.
1. Charge toluene (38.00 L, 5.00 v/w) into a 100 L reactor at room temperature (“r.t.”) under N₂.
2. Charge compound 1 (7.60 kg, 1.00 equiv.) into reactor at r.t. under N₂.
3. Cool to a temperature of about 0 to about 10 °C under N₂.
4. Charge NaBH₄ (865.20 g, 0.50 equiv.) by portions into the solution at about 0 to about 10 °C under N₂.
5. Charge MeOH (11.72 kg, 8.00 equiv.) dropwise into the reactor at about 0 to about 10 °C under N₂. (exothermic).
6. Agitate the solution at about 0 to about 10 °C for 0.5 hr.
7. IPC: HPLC.
8. Charge citric acid monohydrate (2.28 kg, 0.30 w/w) and NaCl (6.90 kg, 0.90 w/w) in H₂O (22.80 L, 3.00 v/w) dropwise into the solution at 0~20 °C under N₂.
9. Warm up to about 10 to about 20 °C.
10. Agitate for 10 min at about 10 to about 20 °C.
11. Stand and Separate the mixture.
12. Extract the aqueous layer with toluene (38.00 L, 5.00 v/w).
13. IPC: aqueous layer HPLC (some product was lost in mother liquor).
14. Extract the aqueous layer with toluene (38.00 L, 5.00 v/w).
15. IPC: aqueous layer HPLC.
16. Wash the combined organic layer with 5% NaHCO₃ aqueous (6.00 L, 1.00 v/w).
   Note: NaHCO₃ (0.30 kg, 0.04 w/w).
17. IPC: aqueous layer (pH=8~9, some product was lost in mother liquor).
18. Wash the combined organic layer with H₂O (7.00 L, 1.00 v/w).
19. IPC: aqueous layer (pH=7~8).
20. Wash the combined organic layer with brine (9.00 L, 1.00 v/w).
   Note: NaCl (2.30 kg, 0.30 w/w).
21. IPC: aqueous layer (pH=7~8).
   Note: Get 22.81 kg of combined aqueous layer (assay: 0.24%), lost about 54.7 g in the aqueous layer.
22. Concentrate the filtrate under vacuum at a temperature of 55 °C to left (70.00 L, 10.00 v/w) to remove MeOH.
23. IPC: GC (almost no MeOH was left).
24. Determined the weight of solution: 61.02 kg, assay: 12.15%, yield: 100.68%, purity: 98.72%. HPLC of Compound 2 is shown in FIG. 21.

### d. Step 2 Synthesis of Compound 3

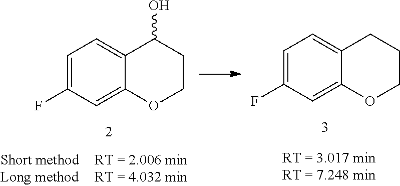

### Impurities

An impurity (Impurity 2-1) was identified during this step. Impurity 2-1 may have the following potential structure:

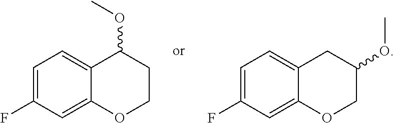

This impurity is believed to be generated by MeOH residual in starting material (compound 2). It will not react further in the following steps and was purged by step 4. This impurity could be controlled by reducing the MeOH lever in crude compound 2 toluene solution.

### Observations/Process Changes/Deviations

It is believed that the HPLC method may not be sufficient for separating toluene and compound 3. Thus, the following method was used.
1. TsOH·H₂O was charged into the reaction mixture at a temperature of about 80 to about 95 °C to keep the reaction less violent and avoid potential run away.
2. A lower yield and low purity on batch 21596-003 was observed. Without necessarily limiting the synthesis procedures, it is believed that the lower yield may be caused by extra MeOH residual in compound 2 toluene solution. The crude compound 3 with lower purity could be used in the next step, with the impurity removed at step 4 after the purification.
3. In certain prophetic embodiments, the MeOH residual may be controlled before being used for this reaction.
4. In this Example, the extraction organic layer was concentrated via drying to afford yellow oil. This operation may, in some instances, not be suitable for further scale up. In certain prophetic embodiments, the crude compound 3 may be isolated as THF solution.

### Batch Summary

| | Input, | Amount | | | | |
|---|---|---|---|---|---|---|
| Lot No. | SM | Product | Yield | Assay | Purity | Remarks |
| 21099- | Toluene | 5.00 kg | 94.70%* | N/A | 92.22% | Yield over |
| 010 | solution | | | | | 2 steps |
| | from | | | | | |
| | 21099-006 | | | | | |
| 21099- | Toluene | 7.51 kg | 74.92% | 66.10% | 98.21% | Yield over |
| 022 | solution | | | | | 2 steps |
| | from | | | | | |
| | 21099-020 | | | | | |
| 21596- | Toluene | 6.62 kg | 61.60% | 55.54% | 72.40% | Yield over |
| 003 | solution | | | | | 2 steps |
| | from | | | | | |
| | 21596-001 | | | | | |

*yield wasn't corrected by assay

### Typical Operation Procedure (E02344-21099-010)

| No. | Raw-material | Amount | | Mol. wt. | Mol | Molar-ratio | |
|---|---|---|---|---|---|---|---|
| 1 | Toluene solution | 5.91 kg | | 168.16 | 35.14 mol | 1.0 | equiv. |
| | | from 21099-006* | | | | | |
| 2 | TsOH•H₂O | 83.57 | g | 190.21 | 0.44 mol | 1.25% | equiv. |
| 3 | NaHCO₃ | 1.50 | kg | N/A | N/A | 0.25 | w/w |
| 4 | Na₂SO₄ | 5.91 | kg | N/A | N/A | 1.00 | w/w |
| 5 | Toluene | 5.91 | L | N/A | N/A | 1.00 | v/w |

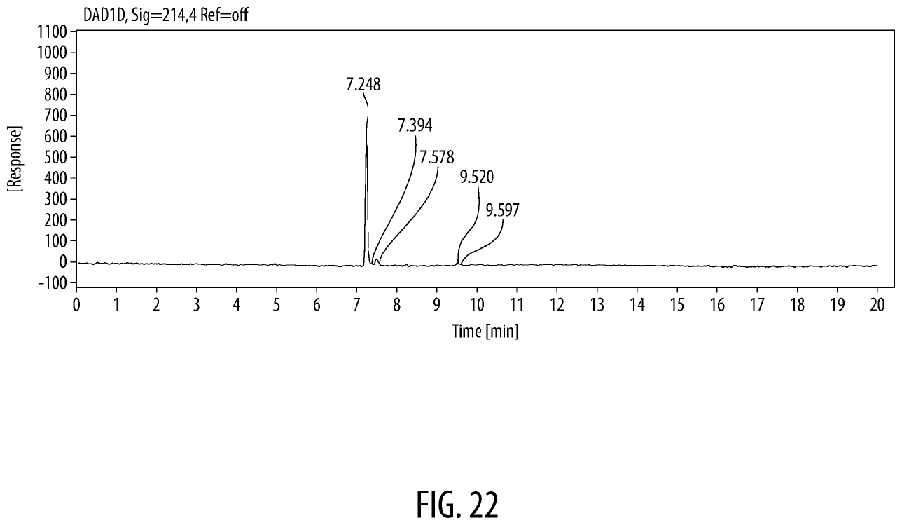

The following steps were performed:
1. Charge the compound 2 solution (about 50.00 L) into a 100 L reactor at r.t. under N₂.
2. Heat up to a temperature of about 90 to about 100 degree.
3. Charge TsOH·H₂O (83.57 g, 1.25% equiv.) into reactor at a temperature of 95 degree under N₂.
   Note: Without being limited to any particular theory, it is believed that if TsOH. H₂O was added at r.t., more impurity will be observed. So, it was added at a temperature of about 90 to about 100 degree, for which the reaction went smoothly. It should be noticed the addition at high temperature in one portion has potential of runaway.
4. Heat up to reflux with Dean-Stark to remove water over 1 to about 2 hr.
5. IPC: short method 1 hr HPLC; long method of 1.5 hr HPLC.
6. Cool down to a temperature of about 20 to about 25 degree.
7. Wash the mixture with 5% NaHCO₃ aqueous (30.00 L, 5.00 v/w).
   Note: NaHCO₃ (1.5 kg, 0.25 w/w).
8. IPC: pH=8~9, no product was lost in aqueous layer.
9. Wash the combined organic layer with H₂O (30.00 L, 5.00 v/w).
10. IPC: aqueous layer pH=7~8.
11. Dry the organic layer with anhydrous Na₂SO₄ (5.91 kg, 1.00 w/w) for 1 hr.
12. Filter the suspension and rinse the cake with toluene (5.91 L, 1.00 v/w).
13. Concentrate the filtrate under vacuum at 60 degree to obtain the product as yellow oil.
14. Determine the weight: 5.0 kg, purity: 92.22%, yield: 94.7% over 2 steps HPLC spectra for Compound 3 (see FIG. 22).

### e. Step 3 Synthesis of Compound 4

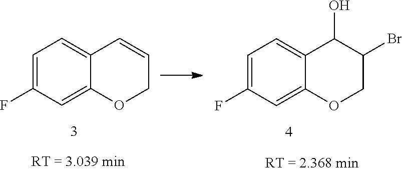 [SMILES: Fc1ccc2c(c1)OCC=C2 ; OC1c2ccc(F)cc2OCC1Br]

### Impurities:

An impurity (Impurity 3-1) was identified during this step of the Example. Impurity 3-1 was a succinimide having the following structure:

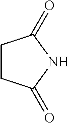

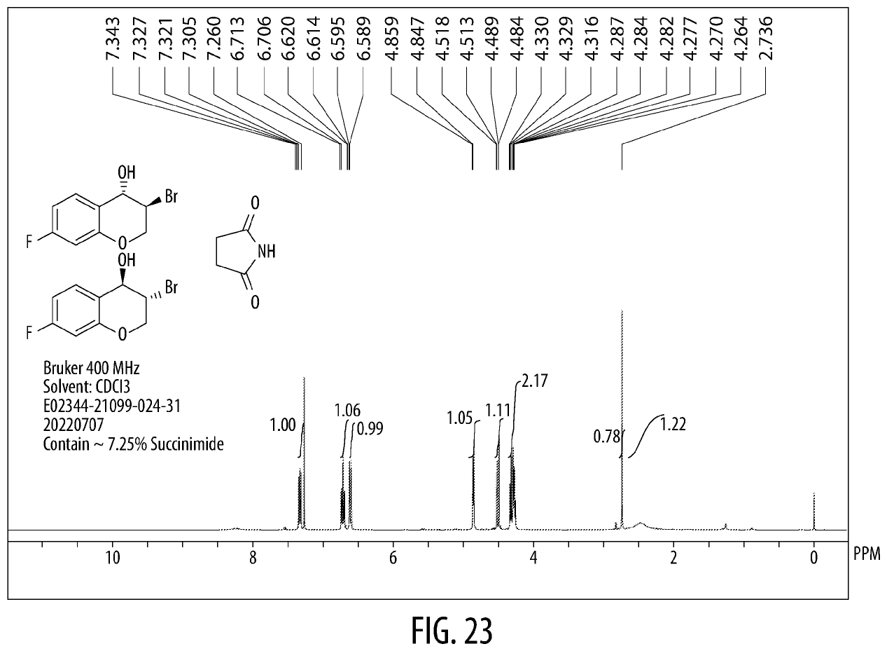

¹H NMR of crude Compound 3 can be seen in FIG. 23.

7.25% succinimide was detected by HNMR in isolated crude Compound 3. This byproduct may affect one or more of the following step and potentially form new impurities (e.g., open the ethylene oxide). In certain prophetic embodiments, the method of synthesizing Compound 4 includes controlling the lever in crude Compound 4 to reduce production of the succinimide byproduct. For example, the succinimide byproduct may be partially removed by slurring with water. However, purging may in certain cases not be effective because the slurring process is heterogeneous.

### Observations/Process Changes/Deviations

As it is believed that excessive NBS may produce many impurities. Usage of NBS was calculated according the assay of Compound 3, and it was added by portions.

Without being limited to any theory, it is believed that the temperature may be important for controlling impurities. A high reaction temperature at this stage is believed to generate more impurities. Thus, the temperature was controlled to a temperature of 10 °C for this stage.

Some succinimide byproduct was detected in the isolated crude product. Preferably, the succinimide byproduct is purged as much as possible by slurring with water.

### Batch Summary

| | Input, | Amount | | | | |
|---|---|---|---|---|---|---|
| Lot No. | SM | Product | Yield | Assay | Purity | Remarks |
| 21099-012 | 5.00 kg* | 6.03 kg | 73.27% | N/A | 92.60% | The yield |
| | | | | | | wasn't |
| | | | | | | corrected |
| 21099-024 | 7.99 kg | 8.30 kg | 86.92% | 91.00% | 94.13% | N/A |
| | Assay: | | | | | |
| | 66.10% | | | | | |
| 21625-002 | 6.62 kg | 1.60 kg | 73.00% | 91.32% | 92.36% | Purified |
| | Assay: | 2.80 kg | | 83.73% | 87.40% | by column |
| | 55.54% | | | | | |

*Assay wasn't tested.

### Typical Operation Procedure (E02344-21099-024)

| | Raw- | | | Mol. | | Molar- | |
|---|---|---|---|---|---|---|---|
| No. | material | Amount | | wt. | Mol | ratio | |
| 1 | Compound 3 | 5.28 | kg | 150.15 | 35.16 mol | 1.0 | equiv. |
| 2 | THF | 48.00 | L | N/A | N/A | 9.00 | v/w |
| 3 | H₂O | 24.00 | L | N/A | N/A | 4.50 | v/w |
| 4 | NBS | 8.76 | kg | 177.98 | 49.22 mol | 1.40 | equiv. |
| 5 | MTBE | 30.00 | L | N/A | N/A | 6.00 | v/w |
| 6 | NaCl | 5.70 | kg | N/A | N/A | 1.11 | w/w |
| 7 | n-hexane | 135.00 | L | N/A | N/A | 27.00 | v/w |
| 8 | Na₂SO₄ | 5.00 | kg | N/A | N/A | 1.00 | w/w |

The following steps were conducted.
1. Charge THF (48.00 L, 9.00 v/w) into a 100 L reactor at r.t. under N₂.
2. Charge Compound 3 (7.99 kg, 1.00 equiv., assay: 66.10%, 5.28 kg by assay) into reactor at r.t. under N₂.
3. Charge H₂O (24.00 L, 4.50 v/w) into the reactor at r.t. under N₂.
4. Cool down to a temperature of 10 °C.
5. Charge NBS (6.26 kg, 1.00 equiv.) into reactor at a temperature of 10 °C under N₂.
6. IPC: 1 hr HPLC.
7. Charge NBS (2.50 kg, 0.40 equiv.) into reactor at a temperature of 10 °C under N₂.
   Note: the amount of additional NBS is based on the IPC result.
8. IPC: 1 hr HPLC.
9. Extract the mixture with MTBE (25.00 L, 5.00 v/w).
   Note: emulsification and difficult to phase cut.
10. Charge 20% NaCl aqueous (10.00 L, 2.00 v/w) into the mixture.
   Note: NaCl (0.20 kg, 0.04 w/w).
11. Agitate, stand, and separate.
12. IPC: aqueous no produce loss.
13. Wash the organic layer with 5% NaCl aqueous (25.00 L*2, 5.00 v/w*2).
   Note: NaCl (2.50 kg, 0.50 w/w).
14. IPC: aqueous no produce loss.
15. Wash the organic layer with 20% NaCl aqueous (15.00 L, 3.00 v/w).
   Note: NaCl (3.00 kg, 0.57 w/w).
16. Dry the organic layer with Na₂SO₄ anhydrous (5.00 kg, 1.00 w/w).
17. Filter and rinse the cake with MTBE (5.00 L, 1.00 v/w).
18. Concentrate the filtrate under vacuum at a temperature of 60 °C to left 15 L solvent.
19. Charge n-hexane (50.00 L, 10.00 v/w) into the residue.
   Note: solid was precipitated.
20. Concentrate the filtrate under vacuum at a temperature of 60 °C to left solvent (15.00 L).
21. Charge n-hexane (75.00 L, 15.00 v/w) into the residue.
22. IPC: GC (RT-9.67 min, MTBE; RT=10.02 min, n-hexane).
23. Cool down to a temperature of 0 °C.
24. Filter the suspension and rinse the cake with n-hexane (10.00 L, 2.00 v/w).
25. IPC: filtrate HPLC.
   Note: weight: 59.44 kg, assay: 0.59%, 350.70 g compound 4 of product was lost in mother liquor.
26. Dry the solid under vacuum at a temperature of 60 °C to obtain product as off-white solid (9.95 kg).
27. Determine weight: 9.95 kg, assay: 87.01%, yield: 99.63%, purity: 91.95%. Perform short method HPLC, LCMS, and ¹HNMR.
   Note: the product of step 26 contained about 7.25% Succinimide
28. W1=9.95 kg.
29. Slurry the solid with water (50.00 L, 5.00 v/w1) at a temperature of 50 °C for 2 hr.
30. Cool to a temperature of about 20 to 30 °C.
31. Filter the suspension and rinse the cake with water (5.00 L, 0.2 v/w1).
32. IPC: filtrate HPLC.
33. Slurry the solid with water (50.00 L, 5.00 v/w1) at r.t. for overnight.
34. Filter the suspension and rinse the cake with water (10.00 L, 1.00 v/w).
35. IPC: filtrate HPLC.
   Note: weight: 70.34 kg, assay: 0.22%, about 155 g of Compound 4 was lost in the aqueous phase.
36. Dry the solid under vacuum at a temperature of 60 °C to constant weight.
37. Determine weight: 8.30 kg, assay: 91.00%, yield: 86.92%, purity: 94.13%, KF: 0.13%.

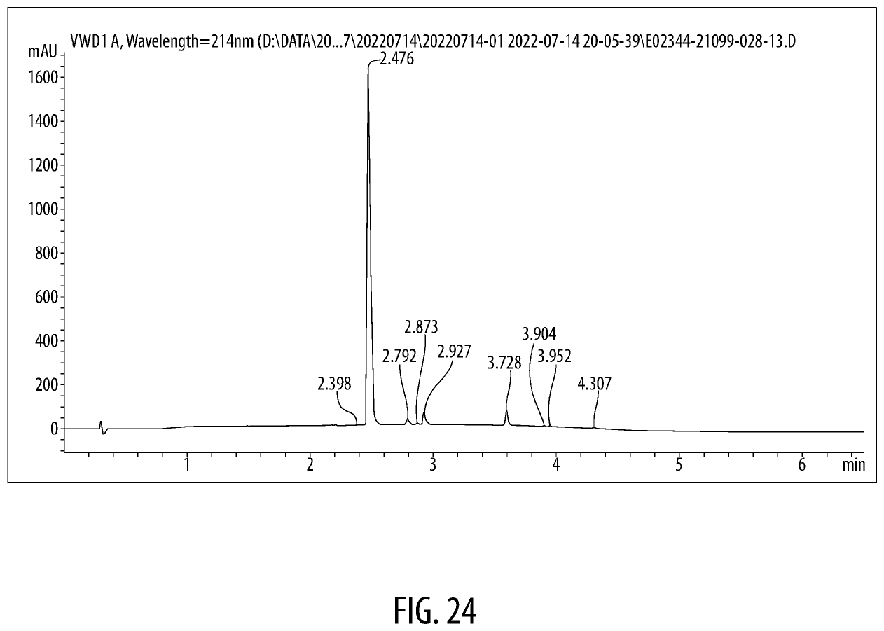

The HPLC spectra of Compound 4 is shown in FIG. 24.

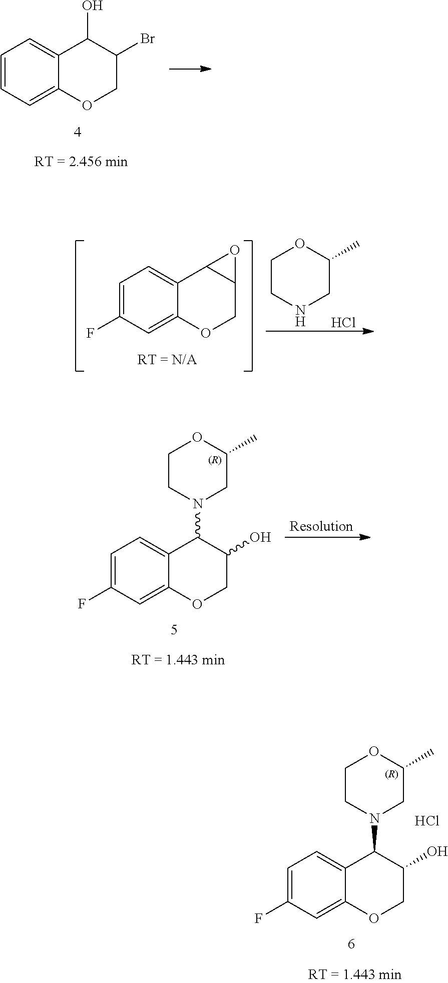

### Impurities

A new impurity-1 (RT=1.977 min) was observed. While the structure of this impurity was not identified, it was determined that the new impurity can be purged in the next step.

An undesired isomer was identified. The undesired isomer had the following structure:

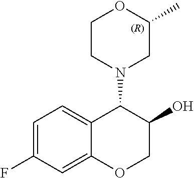

Compound 6 was also identified. After reaction the two isomers were formed as 1/1 ratio. The undesired isomer was controlled by resolution.

### Observations/Process Changes/Deviations

It was determined that the intermediate (ethylene oxide) was very active and was not well detected by HPLC. Instead, the hydrolyzed dihydroxyl material was observed by HPLC.

Considering the instability of ethylene oxide intermediate, anhydrous acetonitrile/K₂CO₃ system was used to generate it in situ, which was then converted to Compound 5 directly without any work up. The isolated crude Compound 5 was an oily material. It was used in the next step directly to chemical resolution without purification.

After resolution the chemical purity of Compound 6 was significantly increased with most impurities being purged. It was determined that the undesired isomer was more crystalline than the target isomer. (+)-Dibenzoyl-D-tartaric aid, L(−)-camphorsulfonic acid, (−)-di-p-toluoyl-L-tartaric acid, (2s, 3s)-2,3-dihydroxysuccinic acid and D-camphorsulfonic acid were screened to resolute compound 5 at eight different solvents. All positive conditions promoted the undesired isomer salt to precipitate out as a solid.

Finally, L-(−)-camphorsulfonic acid was used as the resolution reagent to precipitate out the majority of the undesired isomer. The mother liquor enriched with Compound 6 (right isomer) was converted to HCl salt and was crystallized to upgrade the chiral purity to 98% ee.

### Batch Summary

### Step 4

| Lot No. | Input, 4 | Product, 5 | Yield | Assay | Purity | Remarks |
|---|---|---|---|---|---|---|
| 21099-015 | 5.95 kg | 5.34 kg | 82.92% | N/A | 98.98% | N/A |
| 21099-032 | 7.24 kg | 7.33 kg | Quan. | N/A | 95.64% | N/A |
| | Assay: | | | | | |
| | 91.00% | | | | | |
| 21596-006 | 1.60 kg | 3.86 kg | 92.48% | N/A | 99.11% | N/A |
| | Assay: | | | | | |
| | 91.30% | | | | | |
| | 2.80 kg | | | | | |
| | Assay: | | | | | |
| | 83.73% | | | | | |

### Sten 5

| | Input, | Product, | | | | |
|---|---|---|---|---|---|---|
| Lot No. | 5 | 6 | Yield* | Assay | Purity | Remarks |
| 21099-018 | 5.24 kg | 7.35 kg | 32.35% | 73.88%* | 97.60% | Chiral |
| 21099-034 | 7.33 kg | | | | | Purity: |
| | | | | | | 100.00% |
| 21596-008 | 3.86 kg | 1.91 kg | 35.57% | 95.00% | 99.80% | Chiral |
| | | | | | | purity: |
| | | | | | | 99.52% |

*yield is overall-yield of two steps.

### Typical Operation Procedure (E60523-21596-006 and E60523-21596-008)

| No. | Raw-material | Amount | | Mol. wt. | Mol | Molar-ratio | |
|---|---|---|---|---|---|---|---|
| 1 | Compound 4 | 3.81 | kg | 247.06 | 15.42 mol | 1.00 | equiv. |
| 2 | Acetonitrile | 50.00 | L | N/A | N/A | 12.50 | v/w |
| 3 | K₂CO₃ | 5.32 | kg | 138.21 | 38.49 mol | 2.50 | equiv. |
| 4 | Compound 4A | 2.54 | kg | 137.61 | 18.46 mol | 1.20 | equiv. |
| 5 | NaHSO₄ | 2.78 | kg | 120.06 | 23.16 mol | 1.50 | equiv. |
| 6 | MTBE | 52.00 | L | N/A | N/A | 13.00 | v/w |
| 7 | NaOH | 1.10 | kg | 40.00 | 27.50 mol | 1.80 | equiv. |

Resolution step

| | | | | | | | |
|---|---|---|---|---|---|---|---|
| 1 | Compound 5 | 3.86 | kg | 267.30 | 14.44 mol | 0.94 | equiv. |
| 2 | Acetone | 46.00 | L | N/A | N/A | 12.07 | v/w |
| 3 | L-(−)- | 3.58 | kg | 232.30 | 15.41 mol | 1.00 | equiv. |
| | Camphorsulfonic acid | | | | | | |
| 4 | NaOH | 463.00 | g | 40.00 | 11.58 mol | 0.75 | equiv. |
| 5 | MTBE | 23.00 | L | N/A | N/A | 6.02 | v/w |
| 6 | Na₂SO₄ | 1.92 | kg | N/A | N/A | 0.50 | w/w |
| 7 | i-PrOAc | 11.00 | L | N/A | N/A | 2.87 | v/w |
| 8 | i-PrOH | 5.00 | L | N/A | N/A | 1.30 | v/w |
| 9 | AcCl | 730.00 | g | 78.50 | 9.30 mol | 0.60 | equiv. |

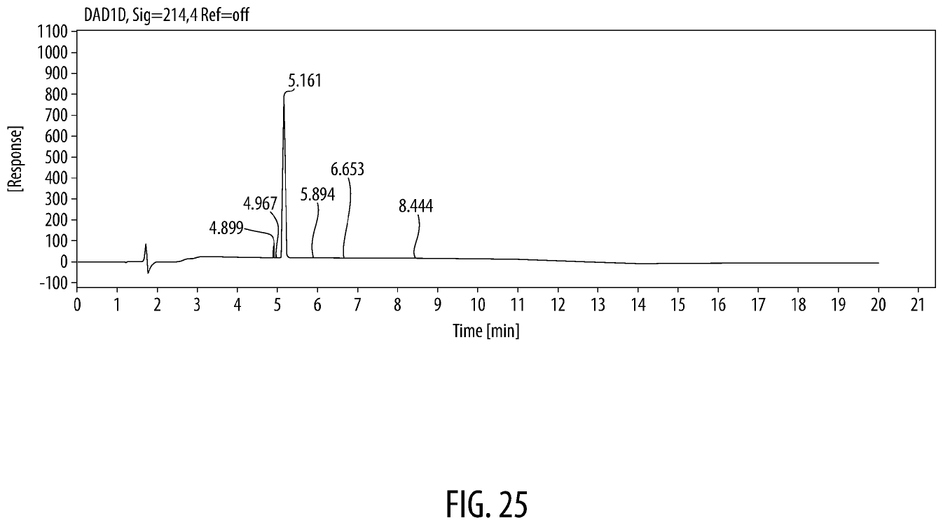

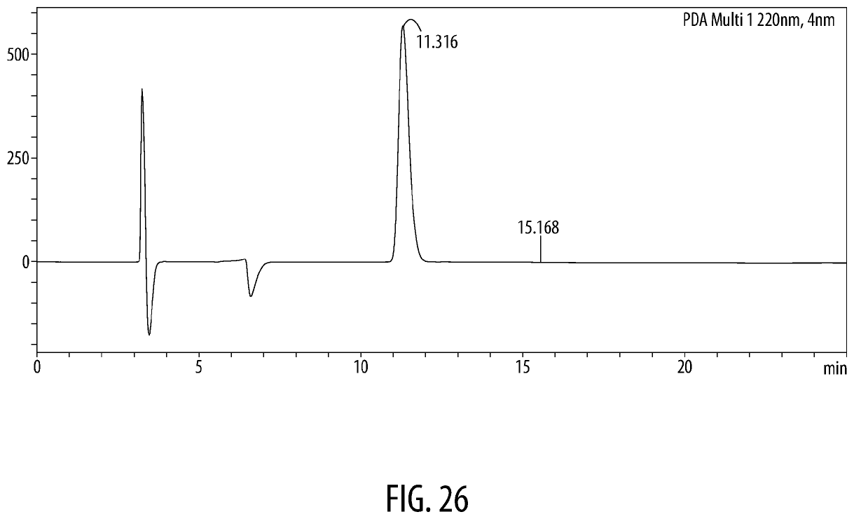

The following steps were conducted.
1. Charge acetonitrile (40.00 L, 10.00 v/w) into a 100 L reactor at r.t. under N₂.
2. Charge Compound 4 (3.81 kg by assay, 1.00 equiv.) into the reactor at r.t. under N₂.
   Note: weight1: 1.60 kg, assay: 91.30%; weight2: 2.80 kg, assay: 83.73%
3. Agitate to obtain a clear solution.
4. Charge K₂CO₃ (4.26 kg, 2.00 equiv.) by one portion into the reactor at r.t. under N₂.
5. Heat to a temperature of 65 °C.
6. Agitate at a temperature of 65 °C over 8 hr.
7. IPC: 3.1% SM was left.
8. Cool to r.t.
9. Charge K₂CO₃ (1.06 kg, 0.5 equiv.) by one portion into the reactor at r.t. under N₂.
10. IPC: 1 hr HPLC.
11. Cool to a temperature of about 20 to about 30 °C.
12. Charge Compound 4A (2.54 kg, 1.20 equiv.) by one portion into the reactor at a temperature of about 20 to about 30 °C under N₂.
13. Heat to a temperature of 80 °C.
14. Agitate for at a temperature of 80 °C overnight.
15. IPC: HPLC.
16. Cool to r.t.
17. Filter the suspension and rinse the cake with ACN (10.00 L, 2.50 v/w).
18. Concentrate the filtrate under vacuum at 60 °C to dryness.
19. Crude product weight: 5.30 kg.
20. Charge MTBE (20.00 L, 5.00 v/w) into the residue to get red solution.
21. Wash solution with NaHSO₄ aqueous (20.00 L, 5.00 v/w) at a temperature of about 10 to about 20 °C.
   Note: NaHSO₄ (1.85 kg, 1.00 equiv.); exothermic obviously
22. Wash the organic layer with NaHSO₄ aqueous (20.00 L, 5.00 v/w).
   Note: NaHSO₄ (0.93 kg, 0.50 equiv.).
23. Combine the aqueous layer from step 17 and step 18.
24. IPC: HPLC of organic layer showed no product was lost, but did show impurities.
25. Charge fresh MTBE (20.00 L, 5.00 v/w) into the resulting combined aqueous layer.
26. Charge NaOH (1.10 kg, 1.80 equiv.) in H₂O (18.00 L, 3.00 v/w) dropwise into the combined aqueous layer to pH=10~11 at a temperature of about 10 toa bout 20 °C.
   Note: exothermic obviously.
27. Agitate, stand, and separate the mixture.
28. Extract the aqueous layer with MTBE (12.00 L, 3.00 v/w).
29. Wash the combined organic layer with H₂O (2*30.00 L, 2*5.00 v/w).
30. Concentrate the organic layer under vacuum at a temperature of about 50 to about 60 °C to constant weight.
31. Weight: 3.86 kg, Purity: 99.11%, Yield: 92.48%; then HPLC.
Resolution: all material was calculated by weight of compound 4 (w=3.81 kg by assay, 15.42 mol).
1. Charge crude compound 5 (3.86 kg, 1.00 equiv.) into a 100 L reactor.
2. Charge acetone (20.00 L, 5.25 v/w) into the reactor to get clear solution.
3. Heat up to a temperature of 50 °C.
4. Charge the solution of L-(−)-Camphorsulfonic acid (3.58 kg, 1.00 equiv.) in acetone (20.00 L, 5.25 v/w) dropwise into the reactor at 50~55 °C.
5. Agitate at a temperature of about 50 to about 55 °C for 45 min at reflux under N₂.
6. Cool to a temperature of about 0 to about 5 °C under N₂.
7. Filter the suspension and rinse the cake with acetone (6.00 L, 1.57 v/w).
8. IPC: solid Chiral HPLC. Filtrate Chiral HPLC.
9. Concentrate the filtrate under vacuum at a temperature of about 55 to about 60 °C to left solvent (1.00~2.00 v/w).
10. Charge H₂O (12.00 L, 3.15 v/w) into the reactor.
11. Concentrate the mixture under vacuum at a temperature of about 50 to about 60 °C to left solvent (3.00 v/w).
12. Cool to a temperature of about 0 to about 10 °C.
13. Charge NaOH (463.00 g, 0.75 equiv.) in H₂O (8.00 L, 2.10 v/w) dropwise into the reactor at 0~10 °C.
   Note: pH=13.
14. Extract the mixture with MTBE (2*10.00 L, 2*2.62 v/w).
15. Wash the combined organic layer with H₂O (15.00 L, 3.94 v/w).
16. IPC: pH=8~9.
17. Dry the organic layer with Na₂SO₄ (1.92 kg, 0.50 w/w).
18. Filter the suspension and rinse the cake with MTBE (3.00 L, 0.78 v/w1).
19. Concentrate the filtrate under vacuum at 50~60 °C to dryness.
20. Charge i-PrOAc (6.00 L, 1.57 v/w) into the residue.
21. Concentrate the solution under vacuum at a temperature of about 50 to about 60 °C to dryness.
22. Charge i-PrOAc (4.00 L, 1.04 v/w) into the residue to get clear solution A.
23. Charge i-PrOH (4.00 L, 1.04 v/w) into reactor 2.
24. Cool the reactor 2 to a temperature of about 0 to about 10 °C under N₂.
25. Charge AcCl (730.00 g, 0.60 equiv.) dropwise into the reactor 2 at a temperature of about 0 to about 10 °C over 1 hr under N₂.
26. Agitate the reactor 2 at a temperature of about 0 to about 10 °C for 4 hr under N₂.
27. Charge solution A from Step 22 dropwise into reactor 2 at a temperature of about 0 to about 10 °C over 1 hr under N₂.
   Note: clear solution.
28. Charge seed of compound 6 (0.50 g, 0.013% w/w) into reactor 2 at a temperature of about 0 to about 10 °C under N₂.
29. Agitate reactor 2 at a temperature of about 0 to about 10 °C for about 12 to about 16 hr under N₂.
30. Filter the suspension and rinse the cake with i-PrOH/i-PrOAc (v/v=1/1, 2.00 L, 0.52 v/w).
31. IPC: solid chiral HPLC.
   Note: mother liquor weight 9.07 kg, assay 1.3%, 118 g compound 6 was lost in mother liquor.
32. Dry solid under vacuum at a temperature of about 50 to about 60 °C to constant weight.
33. Determine weight: 1.91 kg, chiral Purity: 99.52%, yield: 35.57%, assay: 95.00%, purity: 99.80%, and HPLC. The HPLC spectra for Compound 6 can been seen in FIG. 25. The chiral HPLC spectra for Compound 6 can bee seen in FIG. 26.

### g. Step 6 Synthesis of Compound 7

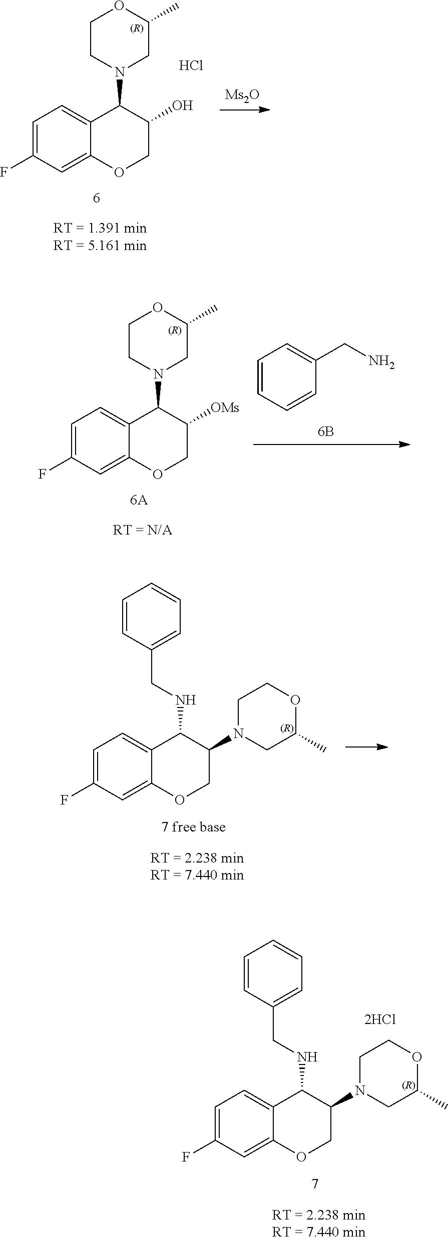

### Impurities

An impurity (Intermediate 6A) was identified having the following structure:

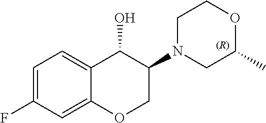

Intermediate 6A was determined to be very unstable under basic condition. With extra base, Intermediate 6A converted to aziridine quickly. The aziridine is very active and could be monitored by HPLC.

A potential impurity (Potential Impurity 6-1) was identified having the following structure:

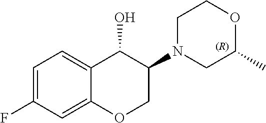

Potential Impurity 6-1 may be formed with moisture. This potential impurity may be purged after the purification by salt formation. As it wasn't observed at isolated Compound 7, it wasn't isolated and characterized during this Example.

### Observations/Process Changes/Deviations

Benzylamine was used in this step instead of 2,4-dimethoxybenzylamine because following cleavage using TFA is very harsh and yield an unclean reaction(s). By replacing with benzylamine, using hydrogenation to do the deprotection should in certain embodiments yield a clean reaction.

Attempts were made to use ammonia to replace 2,4-dimethoxybenzylamine because it theoretically would directly afford desired Compound 8 without any deprotection step. Literature reports some similar reaction worked well using ammonia. But it was determined that using ammonia yielded a messy reaction with undesired impurities. The major impurity is from the competition reaction by water and only a limited amount desired product was observed.

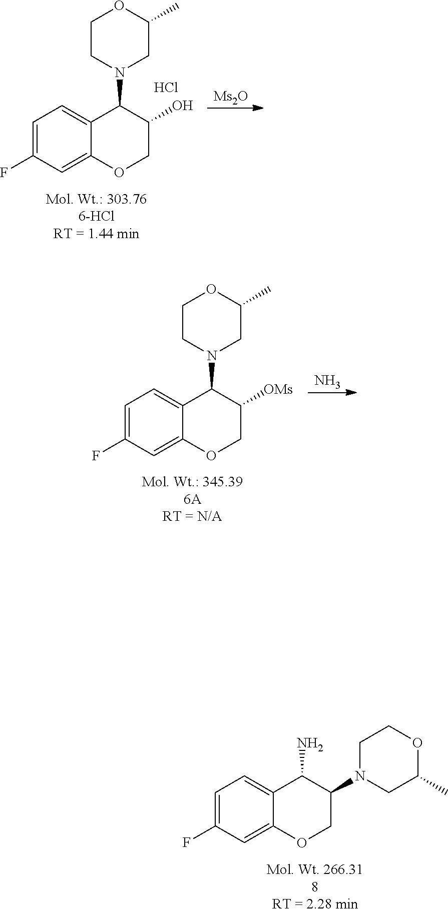

| Batch No. | SM, 6 | Conditions | IPC | Product, 8 |
|---|---|---|---|---|
| 21108-040 | 5.00 g | DCM (5.0 v/w) | 0 °C~r.t | messy |
| | | Ms₂O (1.7 equiv.) | (PDF: E60523- | |
| | | TEA (3.6 equiv.) | 21108-040-20) | |
| | | NH₃ flow | | |
| 21108-041 | 5.00 g | DCM (5 v/w) | 0 °C~r.t | messy |
| | | Ms₂O (1.7 equiv.) | (E60523-21108- | |
| | | TEA (3.6 equiv.) | 041-15) | |
| | | NH₃ flow | | |
| 21109-043 | 2.37 g | DCM (5 v/w) | 0 °C~reflux | messy |
| | | Ms₂O (1.7 equiv.) | (PDF: E60523- | |
| | | TEA (6.6 equiv.) | 21109-043-21) | |
| | | NH₄Cl (3.0 | | |
| | | equiv.) | | |
| 21108-049 | 3.04 g | DCM (10 v/w) | 0~40 °C | No |
| | | Ms₂O (1.3 equiv.) | (PDF: E60523- | reaction |
| | | TEA (2.7 equiv.) | 21108-049-19) | |
| | | HCOONH₄(3.0 | | |
| | | equiv.) | | |

It was difficult to yield Compound 7 free base in a solid form, which made the isolation and purification quite difficult. It was determined that the HCl salt formed of Compound 7 was a good white solid. Thus, Compound 7 was converted to a HCl salt in this Example. Through the salt formation, Compound 7 can be isolated with high purity.

It was found some batches couldn't be used for the next hydrogenation step directly. The material was slurried with THF/i-PrOAc twice again and then was determined to be suitable for the next step.

The intermediate was determined to be very active and hard to monitor by HPLC. Thus, TLC was used to track the intermediate.

Ms₂O was also not very stable at room temperature and became less potent with prolonged storage time. It is important to use fresh and qualified Ms₂O.

Without being limited to any particular theory, the equiv. of TEA is believed to be an important parameter. Less TEA may promote incomplete conversion while excess may lead the active intermediate easier to decompose. Its equiv. is associated with the equiv. of Ms₂O used.

### Batch Summary

| | Input, | Amount | | | | |
|---|---|---|---|---|---|---|
| Lot No. | SM | Product | Yield | Assay | Purity | Remarks |
| 21108-032 | 67.68 g | 9.06 kg | 98.09% | 79.36% | 95.06% | N/A |
| | Assay: | | | | | |
| | 73.88% | | | | | |
| 21108-033 | 6.95 kg | | | | | |
| | Assay: | | | | | |
| | 73.88% | | | | | |
| 21596-010 | 1.91 kg | 2.50 kg | 80.24% | 82.3% | 98.33% | N/A |
| | Assay: | | | | | |
| | 95.00% | | | | | |

### Typical Operation Procedure (E60523-21596-010)

| | Raw- | | | Mol. | | Molar- | |
|---|---|---|---|---|---|---|---|
| No. | material | Amount | | wt. | Mol | ratio | |
| 1 | Compound 6 | 1.81 | kg | 303.76 | 5.97 mol | 1.00 | equiv. |
| 2 | DCM | 32.00 | L | N/A | N/A | 17.00 | v/w |
| 3 | Ms₂O | 1.45 | kg | 174.19 | 8.32 mol | 1.40 | equiv. |
| 4 | TEA | 1.756 | kg | 101.19 | 17.35 mol | 2.91 | equiv. |
| 5 | Benzylamine | 1.28 | kg | 107.15 | 11.95 mol | 2.00 | equiv. |
| | (compound | | | | | | |
| | 6B) | | | | | | |
| 6 | Citric acid | 0.91 | kg | N/A | N/A | 0.50 | w/w |
| | monohydrate | | | | | | |
| 7 | NaCl | 2.53 | kg | N/A | N/A | 1.40 | w/w |
| 8 | NaOH | 0.78 | kg | N/A | N/A | 0.15 | w/w |
| 9 | Na₂SO₄ | 5.18 | kg | N/A | N/A | 2.72 | w/w |
| 10 | IPA | 3.62 | L | N/A | N/A | 2.00 | v/w |
| 11 | AcCl | 935.50 | g | 78.50 | 11.92 mol | 2.00 | equiv. |
| 12 | THF | 19.00 | L | N/A | N/A | 9.50 | v/w |
| 13 | i-PrOAc | 11.00 | L | N/A | N/A | 5.50 | v/w |

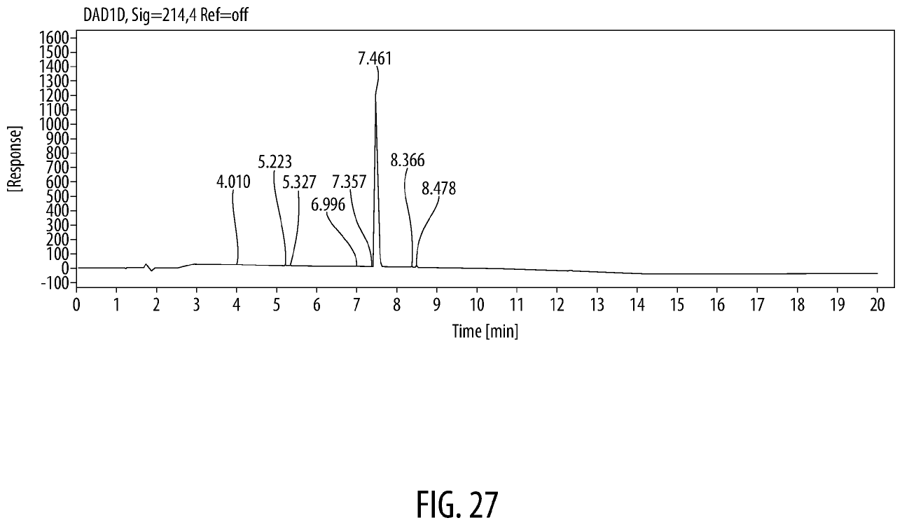

The following steps were conducted.
1. Charge DCM (18.00 L, 10.00 v/w) into a 100 L reactor at r.t. under N₂.
2. Charge compound 6 (1.91 kg, assay: 95.00%, 1.00 equiv.) into the reactor at r.t. under N₂.
3. Cool to a temperature of about 0 to about 10 °C under N₂.
4. Charge Ms₂O (1.35 kg, 1.30 equiv.) dropwise into the reactor at a temperature of about 0 to about 10 °C under N₂.
5. Charge TEA (1.63 kg, 2.70 equiv.) in DCM (10.00 L, 5.00 v/w) dropwise into the reactor under N₂ a temperature of about 0 to about 10 °C.
6. Agitate at a temperature of about 0 to about 10 °C for 60 min under N₂.
7. IPC: TLC some SM was remained
8. Charge Ms₂O (100.00 g, 0.10 equiv.) dropwise into the reactor at a temperature of about 0 to about 10 °C under N₂.
9. Charge TEA (126.00 g, 0.21 equiv.) into the reactor under N₂ at a temperature of about 0 to about 10 °C.
10. Agitate at a temperature of about 0 to about 10 °C for 60 min under N₂.
11. IPC: TLC SM was consumed almost completely. (DCM/EtOAc=2/1).
12. Charge compound 6B (1.28 kg, 2.00 equiv.) into the reactor at a temperature of about 0 to about 10 °C.
   Note: The mixture became turbid in 3-4 hours, and then turn to a clear solution after 5 hr.
13. Agitate for overnight at a temperature of about 20 to about 30 °C under N₂.
14. IPC: 18 hr HPLC the reaction was done
15. Cool to a temperature of about 0 to about 10 °C.
16. Charge 10% citric acid monohydrate aqueous (10.00 L, 5.00 v/w) dropwise into the reactor at a temperature of about 0 to about 10 °C.
   Note: citric acid monohydrate (0.91 kg, 0.50 w/w).
17. Agitate, stand and phase cut.
18. Wash the organic layer with brine (10.00 L, 5.00 v/w).
   Note: NaCl (2.53 kg, 1.40 w/w).
19. Wash the organic layer with 5% NaOH aqueous (15.00 L, 3.00 v/w).
   Note: NaOH (0.78 kg, 0.15 w/w).
20. Wash the combined organic layer with H₂O (2*25.00 L, 2*5.00 v/w).
21. Dry the organic layer with Na₂SO₄ (5.18 kg, 2.72 w/w).
22. Filter the suspension and rinse the cake with DCM (4.00 L, 2.00 v/w).
23. Concentrate the filtrate under vacuum at a temperature of about 50 to about 60 °C to left solvent (1~2 v/w) to obtain solution A.
24. Charge IPA (3.62 L, 2.00 v/w) into the reactor 2 at a temperature of 0 to 10 °C.
25. Charge AcCl (935.50 g, 2.00 equiv.) dropwise into the reactor 2 at a temperature of 0 to 10 °C under N₂.
26. Agitate for 4 hr at a temperature of 0 to 10 °C to obtain a solution B under N₂.
27. Charge solution A from step 23 dropwise into the solution B in reactor 2 at a temperature of 0 to 10 °C under N₂.
28. Agitate for overnight at a temperature of about 0 to about 5 °C under N₂.
29. Concentrate the filtrate under vacuum at a temperature of about 50 to about 60 °C to left solvent (1~2 v/w).
30. Charge THF (10.00 L, 5.00 v/w) into the reactor.
31. Concentrate the filtrate under vacuum at a temperature of about 50 to about 60 °C to left solvent (1~2 v/w).
32. Charge THF (8.00 L, 4.00 v/w) into the reactor.
33. Charge i-PrOAc (10.00 L, 5.00 v/w) into the reactor.
34. Agitate at r.t for 2~3 days to obtain white suspension.
   Note: the prolonged agitation time is to ensure a fine suspension is formed otherwise some large lumps may presented and affect the purging ability.
35. Filter the suspension and rinse the cake with i-PrOAc/THF (v/v=1/1, 2.00 L, 1.00 v/w).
36. Dry the solid under vacuum at 50~60 °C to constant weight.
37. Determine weight: 2.50 kg, assay: 82.30%, purity: 98.33%, yield: 80.24%, and HPLC (see FIG. 27).

### h. Step 7 Synthesis of Compound 8

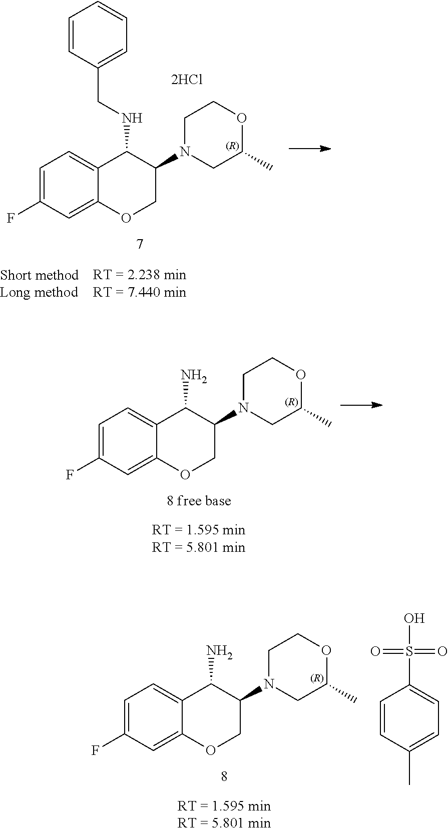

### Impurities

An impurity (Impurity 7-1) having the following structure was identified:

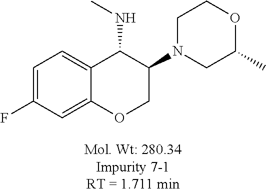 [SMILES: CN[C@H]1c2ccc(F)cc2OC[C@@H]1N1CCO[C@H](C)C1]

Impurity 7-1 was detected when using HCOONH₄ or HCOOK reduction condition. Further study showed it could be generated from starting material (Compound 7), but is not produced from Compound 8.

Another impurity (Impurity 7-2) having the following structure was identified:

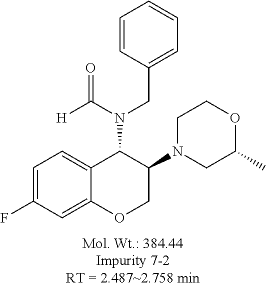 [SMILES: C[C@@H]1CN([C@H]2COc3cc(F)ccc3[C@@H]2N(C=O)Cc2ccccc2)CCO1]

Impurity 7-2 was also detected when using HCOONH₄ or HCOOK reduction condition.

### Observations/Process Changes/Deviations

HCOONH₄ and HCOOK, as hydrogen sources, were screened with different solvent. Two major impurities, Impurity 7-1 and Impurity 7-2, were formed. The lever of those two impurities is believed to be strongly connected with reaction speed. Without being limited to any particular theory, it is believed that when the reaction was not fast, the two impurities were generated. In certain embodiments, this reduction method may be used as it avoids the usage of hydrogenator.

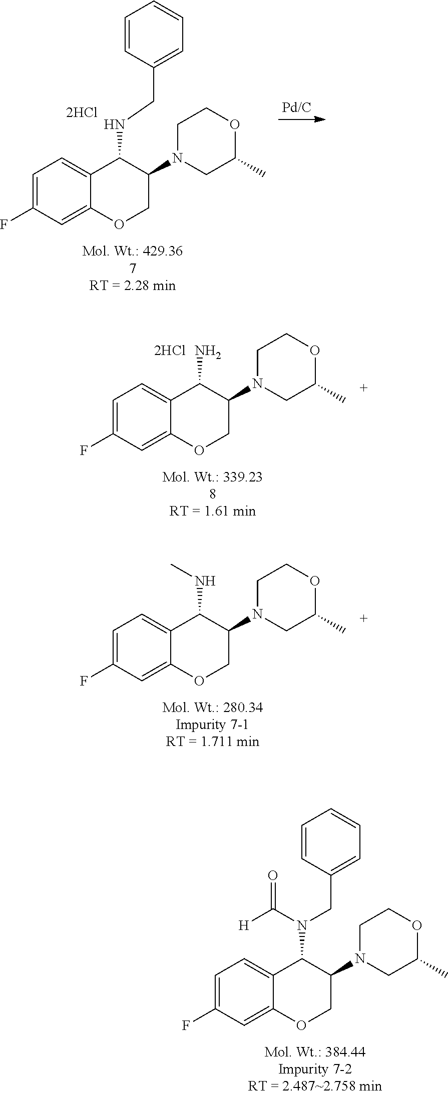 [SMILES: C[C@@H]1CN([C@H]2COc3cc(F)ccc3[C@@H]2NCc2ccccc2)CCO1 ; C[C@@H]1CN([C@H]2COc3cc(F)ccc3[C@@H]2N)CCO1 ; CN[C@H]1c2ccc(F)cc2OC[C@@H]1N1CCO[C@H](C)C1 ; C[C@@H]1CN([C@H]2COc3cc(F)ccc3[C@@H]2N(C=O)Cc2ccccc2)CCO1]

| Batch | | | | | |
|---|---|---|---|---|---|
| No. | SM, 7 | Conditions | IPC | | Product, 8 |
| 21239- | 1.0 g | HCOOK (5.5 equiv.) | (PDF: E60523- | | Better |
| 018 | | IPA (3.3 v/w) | 21329-018-18) | | Using HCOOK instead of |
| | | H₂O (3.5 equiv.) | | | HCOONH₄ |
| | | 10% Pd/C (0.1 w/w) | | | at r.t. |
| | | r.t | | | |
| 21239- | 1.0 g | HCOOK (5.5 equiv.) | (PDF: E60523- | | No product was formed |
| 019 | | IPA (3.3 v/w) | 21329-019-13) | | The impurity 7-2 was |
| | | H₂O (3.5 equiv.) | | | detected |
| | | 10% Pd/C (0.1 w/w) | | | Using HCOOK instead of |
| | | reflux | | | HCOONH₄ |
| | | | | | at reflux. |
| 21239- | 1.0 g | MeOH (10 v/w) | 2 hr | 16 hr | Better |
| 020 | | HCOOK (5.5 equiv.) | (PDF: | (PDF: | Charge Compound 7 and |
| | | 10% Pd/C (0.1 w/w) | E60523- | E60523- | Pd/C into the reactor, agitate |
| | | r.t | 21329- | 21329- | for 0.5 hr, then charge |
| | | | 020-12) | 020-14) | HCOOK into the reactor |
| 21239- | 1.0 g | Acetic Acid (10 v/w) | 2 hr | 16 hr | No product was formed |
| 022 | | H₂O (1.5 v/w) | (PDF: | (PDF: | The impurity 7-2 was |
| | | HCOOK (5.5 equiv.) | E60523- | E60523- | detected |
| | | 10% Pd/C (0.1 w/w) | 21329- | 21329- | Using AcOH as solvent |
| | | r.t | 022-14) | 022-16) | |
| 21239- | 1.0 g | MeOH (10 v/w) | 3.5 hr | 17.5 hr | Good |
| 023 | | H₂O (1.5 v/w) | (PDF: | (PDF: | Increase HCOOK from 5.5 |
| | | HCOOK (11 equiv.) | E60523- | E60523- | equiv. to 11 equiv. |
| | | 10% Pd/C (0.1 w/w) | 21329- | 21329- | Charge HCOOK in H₂O |
| | | r.t | 023-14) | 023-16-) | dropwise into the reactor in |
| | | | | | 30 minutes |
| 21239- | 1.0 g | MeOH (15 v/w) | 1.5 hr | 19.5 hr | Good |
| 024 | | H₂O (10 v/w) | (PDF: | (PDF: | Charge HCOOK in H₂O |
| | | HCOOK (11 equiv.) | E60523- | E60523- | into the reactor in short time |
| | | 10% Pd/C (0.1 w/w) | 21329- | 21329- | |
| | | r.t | 024-13) | 024-20) | |
| 21239- | 1.0 g | MeOH (15 v/w) | 0 hr | 16 hr | No product was formed |
| 025 | | H₂O (10 v/w) | (PDF: | (PDF: | The impurity 7-2 was |
| | | HCOOK (11 equiv.) | E60523- | E60523- | detected |
| | | 10% Pd/C (0.1 w/w) | 21329- | 21329- | Charge HCOOK in H₂O |
| | | r.t | 025-16) | 025-19) | dropwise into the reactor in |
| | | | | | 3.5 hr |

The hydrogenation reaction worked well with Pd/C as catalyst and hydrogen as the reducing reagent.

A salt form of Compound 8 was studied. TsOH salt form of Compound 8 was selected for ease of preparation and non-hydroscopic nature. The isolated TsOH salt of Compound 8 was determined to be suitable for the last coupling step without free base process. Meanwhile the salt formation further purges impurities in the free base and ensures the impurity control for the last GMP step.

### Batch Summary

| | Input, | Amount | | | | | |
|---|---|---|---|---|---|---|---|
| Lot No. | SM | Product | | Yield | Assay | Purity | Remarks |
| 21384- | 1.22 kg | 1.34 | kg* | 99.32% | 94.88% | 98.97% | N/A |
| 005 | Assay: | | | | | | |
| | 84.67% | | | | | | |
| 21384- | 1.22 kg | | | | | | N/A |
| 006 | Assay: | | | | | | |
| | 84.67% | | | | | | |
| 21596- | 1.21 kg | 4.58 | kg** | 90.57% | N/A | 99.70% | N/A |
| 015 | Assay: | | | | | | |
| | 84.26% | | | | | | |
| 21596- | 1.21 kg | | | | | | N/A |
| 016 | Assay: | | | | | | |
| | 84.26% | | | | | | |
| 21596- | 1.21 kg | | | | | | N/A |
| 017 | Assay: | | | | | | |
| | 84.26% | | | | | | |
| 21596- | 1.20 kg | | | | | | N/A |
| 018 | Assay: | | | | | | |
| | 82.30% | | | | | | |
| 21596- | 1.10 kg | | | | | | N/A |
| 019 | Assay: | | | | | | |
| | 82.30% | | | | | | |

Note:
*1.34 kg was isolated as compound 8-free base (oil), and the free base was used into the next step directly.
**4.58 kg was isolated as TsOH salt, it was used as GMP raw material.

### Typical Operation Procedure (E60523-21596-018)

| 1 | Raw-material | Amount | | Mol. wt. | Mol | Molar-ratio | |
|---|---|---|---|---|---|---|---|
| 1 | Compound 7 | 987.60 g (by | | 429.36 | 2.30 mol | 1.00 | equiv. |
| 2 | IPA | 4.00 | L | N/A | N/A | 4.00 | v/w |
| 3 | H₂O | 4.00 | L | N/A | N/A | 4.00 | v/w |
| 4 | 10% Pd/C | 100.00 | g | N/A | N/A | 0.10 | w/w |
| 5 | IPA | 6.50 | L | N/A | N/A | 1.00 | v/w1 |
| 6 | NaOH | 1235.00 | g | 40.00 | 30.87 mol | 2.00 | equiv. |
| 7 | DCM | 55.25 | L | N/A | N/A | 8.50 | v/w1 |
| 8 | Na₂SO₄ | 6.50 | kg | N/A | N/A | 1.00 | w/w1 |
| 9 | Crude compound 8 free | 2.79 | kg | 266.31 | 10.48 mol | 1.00 | equiv. |
| 10 | MTBE | 33.50 | L | | | 12.00 | v/w2 |
| 11 | TsOH•H₂O | 2.00 | kg | 190.22 | 10.51 mol | 1.00 | equiv. |

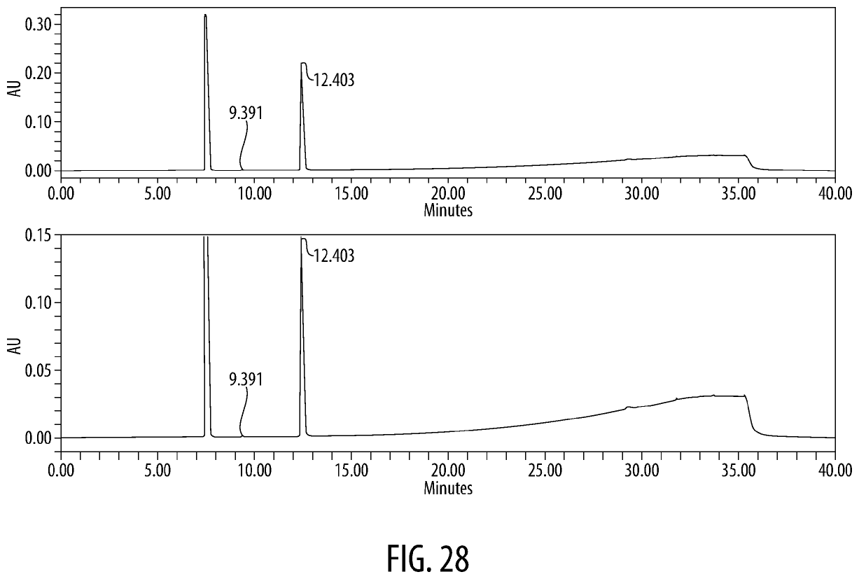

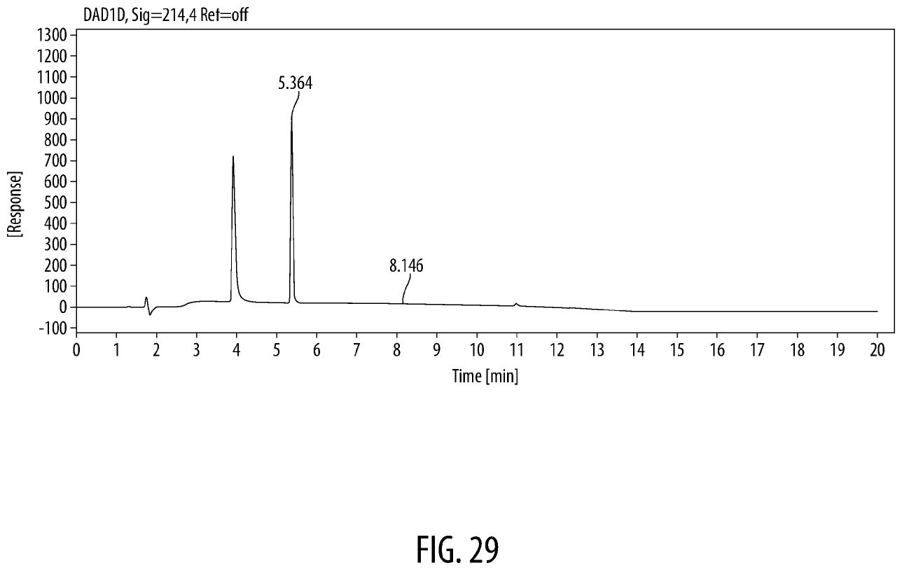

The following steps were conducted:
1. Charge IPA (3.50 L, 3.50 v/w) into a 10 L reactor at r.t.
2. Charge H₂O (3.50 L, 3.50 v/w) into the reactor at r.t.
3. Charge Compound 7 (987.60 g by assay, 1.00 equiv.) into the reactor at r.t.
   Note: weight of crude: 1.20 kg, assay: 82.30%.
4. Charge 10% Pd/C (100.00 g, 0.1 w/w) into the reactor.
5. Exchange inner atmosphere of reactor with H₂ bag.
6. Agitate at a temperature of 30 °C under H₂ bag until SM<1%.
7. Combine the reaction mixture with 21596-015/016/017/018/019 to filter.
   w1=6.50 kg from this step.
8. Filter the suspension and rinse the cake with i-PrOH/H₂O (v/v=1/1, 13.00 L, 2.00 v/w1).
9. Concentrate the filtrate under vacuum at a temperature of 55 °C to remove i-PrOH.
10. Cool the residue to a temperature of about 0 to about 10 °C.
11. Charge NaOH (1235.00 g, 2.0 equiv.) in H₂O (6.50 L, 1.0 v/w1) dropwise into the reactor at a temperature of about 0 to about 10 °C to adjust pH to 12~13.
12. Extract the mixture with DCM (32.50 L, 5.00 v/w1; 16.25 L, 2.50 v/w1).
13. IPC showed there is no loss in aqueous layer by HPLC.
14. Wash the organic layer with H₂O (2*16.25 L, 2*2.50 v/w1).
15. IPC showed there is no loss in aqueous layer by HPLC.
16. Dry the organic layer with Na₂SO₄ anhydrous (1.00 w/w1, 6.50 kg).
17. Filter the suspension and rinse the cake with DCM (6.50 L, 1.00 v/w1).
18. Concentrate the filtrate under vacuum at a temperature of 55 °C to constant weight (2.79 kg).
   w2=2.79 kg from this step (weight of crude compound 8-free base).
19. Charge MTBE (28.00 L, 10.00 v/w2) into the reactor at r.t. under N₂.
20. Charge Compound 8 (2.79 kg, 1.00 equiv. of crude compound 8-free base) into the reactor.
21. Charge TsOH·H₂O (2.0 kg, 1.0 equiv. of crude Compound 8-free base) into the reactor in portion.
22. Agitate for a period of overnight at a temperature of about 20 to 30 °C.
23. Filter the suspension and rinse the cake with MTBE (5.50 L, 2.00 v/w2).
24. IPC: filtrate HPLC.
25. Dry the solid under vacuum at a temperature of 55 °C over 24 hr to constant weight.
26. Determine weight: 4.58 kg, yield: 98.35%, Yield: 90.57%, and HPLC. FIG. 28 shows a representative HPLC spectra for Compound 8 of Scheme 9, at different scales. FIG. 29 shows a HPLC spectra for a higher purity sample of Compound 8 of Scheme 9.

### i. Step 8 Synthesis of Crude MTK410088 (Crude EP-0041088)

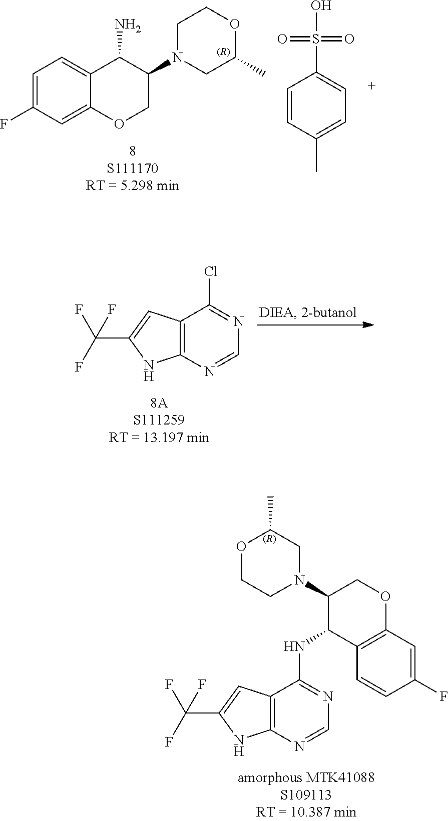

### Impurities

Unconsumed starting materials were identified. For instance, Compound 8 and Compound 8A, which are shown below, were found with the final compound (MTK41088). Compound 8 and Compound 8A could be purged by slurry with DCM.

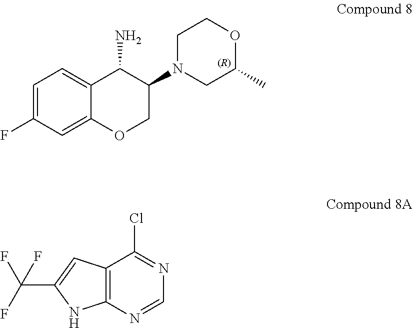

### Observations/Process Changes/Deviations

n-BuOH, 2-BuOH, and t-BuOH as reaction solvents were evaluated, the result showing a high temperature could improve the reaction speed, but the purity of the reaction mixture may be lower than if a low temperature was used. For this Example, 2-BuOH was employed as the reaction solvent.

DIPEA, DBU, TEA, DABCO and K₂CO₃ were evaluated for use as a base. Positive result was obtained when DIPEA was used as the base. The unconsumed compound 8 could be purged by workup completely.

The crude product was slurried with DCM to purge impurities as amorphous material. The isolated amorphous material typically contained some DCM residual. It was difficult to remove by drying, but could be removed by slurring with water. However, the amorphous material will be slowly transformed to crystalline compound with prolonged reaction time.

### Batch Summary

| | Input, | | Amount | | | | |
|---|---|---|---|---|---|---|---|
| Lot No. | SM | | Product | Yield | Assay | Purity | Remarks |
| 15808-050 | 1.30 | kg* | 1.38 kg | 66.03% | N/A | 99.02% | ~0.37% |
| | | | | | | | DCM |
| S109113C- | 3.90 | kg** | 3.53 kg | 87.92% | N/A | 100.00% | 1049 ppm |
| 2201 | | | | | | | DCM |

*free base;
**TsOH salt

### Typical Operation Procedure (S109113C-2201)

| | Raw- | | | Mol. | | Molar- | |
|---|---|---|---|---|---|---|---|
| No. | material | Amount | | wt. | Mol | ratio | |
| 1 | Compound 8 | 3.90 | kg | 438.51 | 8.89 mol | 1.00 | equiv. |
| 2 | n-BuOH | 1.15 | L | N/A | N/A | 9.0 | v/w |
| 3 | Compound | 2.00 | kg | 221.57 | 9.03 mol | 1.00 | equiv. |
| | 8A | | | | | | |
| 4 | DIPEA | 4.60 | kg | 129.24 | 35.59 mol | 4.00 | equiv. |
| 5 | i-PrOAc | 45.43 | kg | N/A | N/A | 11.35 | w/w |
| 6 | NaCl | 2.60 | kg | N/A | N/A | 0.66 | w/w |
| 7 | DCM | 167.48 | kg | N/A | N/A | 41.72 | w/w |

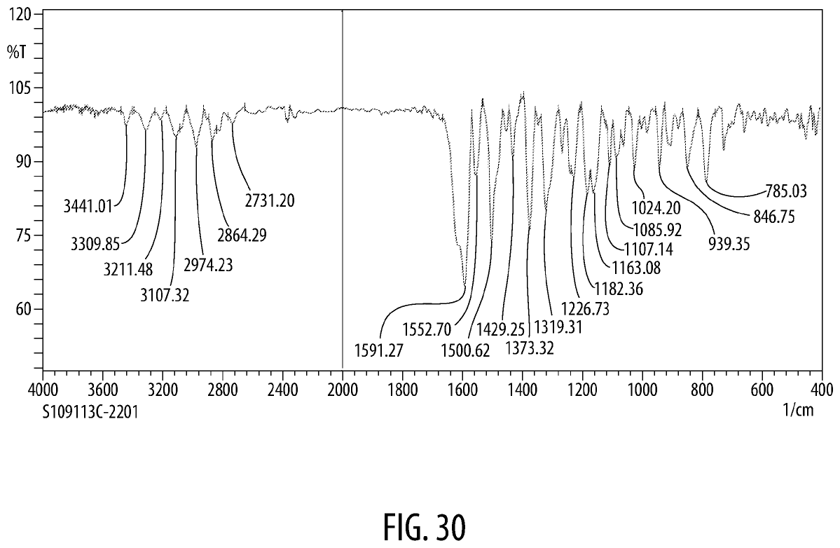

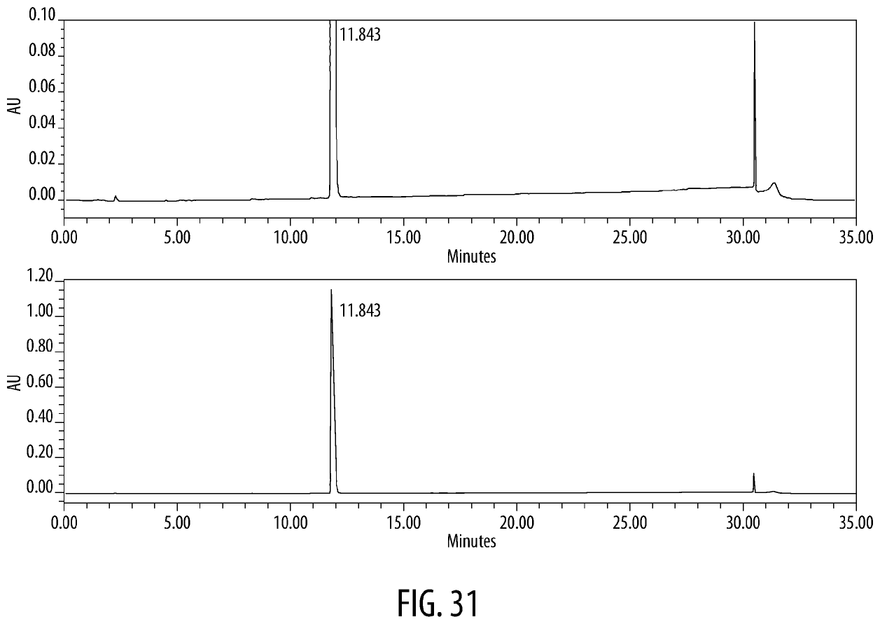

The following steps were performed:
1. Charge Compound 8 (3.90 kg, 1.00 equiv., 1.00 w/w) into the reactor at r.t. under N₂.
2. Charge Compound 8A (2.00 kg, 1.00 equiv., 0.51 w/w) into the reactor.
3. Charge 2-BuOH (28.00 kg, 7.27 w/w) into the reactor.
4. Charge DIPEA (4.60 kg, 4.00 equiv., 1.18 w/w) into the reactor.
5. Heat to reflux (at temperature of about 95 to about 105 °C) under N₂.
6. Agitate for 72 hr at a temperature of about 95 to about 105 °C under N₂.
7. IPC1: S111170/(S111170+S109113)=1.2%<3.0%.
8. Cool to a temperature of about 45 to about 55 °C under N₂.
9. Concentrate the reaction mixture under vacuum at a temperature of about 50 to about 60 °C to left solvent (11.70 L, 3.00 v/w).
10. Cool to at a temperature of about 20 to about 30 °C under N₂.
11. Charge i-PrOAc (35.26 kg, 8.73 w/w) into the reactor at a temperature of about 20 to about 30 °C under N₂.
12. Charge H₂O (40.12 kg, 10.00 w/w) into the reactor at a temperature of about 20 to about 30 °C under N₂.
13. Agitate, stand, and phase separate at a temperature at about 20 to about 30 °C under N₂.
14. Extracted the aqueous with i-PrOAc (10.17 kg, 2.62 w/w) at a temperature of about 20 to about 30 °C under N₂.
15. Wash the organic layer with NaCl aqueous (13.50 kg, 3.33 w/w) at a temperature of about 20 to about 30 °C under N₂.
   Note: NaCl (1.30 kg, 0.33 w/w), H₂O (12.20 kg, 3.00 w/w).
16. Wash the organic layer with NaCl aqueous (13.50 kg, 3.33 w/w) at a temperature of about 20 to about 30 °C under N₂.
   Note: NaCl (1.30 kg, 0.33 w/w), H₂O (12.20 kg, 3.00 w/w).
17. Wash the organic layer with NaCl aqueous (13.41 kg, 3.33 w/w) at a temperature of about 20 to about 30 °C under N₂.
   Note: NaCl (1.33 kg, 0.33 w/w), H₂O (12.08 kg, 3.00 w/w).
18. Concentrate the organic layer under vacuum at a temperature of about 45 to about 55 °C to left the residual (3.90~7.80 L, 1.00~2.00 v/w).
19. Charge DCM (26.65 kg, 6.62 w/w) into the reactor.
20. Concentrate the organic layer under vacuum at a temperature of about 45 to about 55 °C to left the residual (3.90~7.80 L, 1.00~2.00 v/w).
   Note: solid precipitation.
21. Charge DCM (26.65 kg, 6.62 w/w) into the reactor.
22. Concentrate the organic layer under vacuum at a temperature of about 45 to about 55 °C to left the residual (3.90~7.80 L, 1.00~2.00 v/w).
   Note: solid precipitation.
23. Charge DCM (26.65 kg, 6.62 w/w) into the reactor.
24. Concentrate the organic layer under vacuum at a temperature of about 45 to about 55 °C to left the residual (11.70~15.60 L, 3.00~4.00 v/w).
   Note: solid precipitation.
25. Cool to a temperature of about 0 to about 10 °C under N₂.
26. IPC2-1: i-PrOAc/(i-PrOAc+DCM)=20.42%>10.0%, unqualified.
27. Charge DCM (26.65 kg, 6.62 w/w) into the reactor.
28. Concentrate the organic layer under vacuum at a temperature of about 45 to about 55 °C to left the residual (3.90~7.80 L, 1.00~2.00 v/w).
   Note: solid precipitation.
29. Charge DCM (26.65 kg, 6.62 w/w) into the reactor.
30. Concentrate the organic layer under vacuum at a temperature of about 45 to about 55 °C to left the residual (11.70~15.60 L, 3.00~4.00 v/w).
   Note: solid precipitation.
31. Cool to a temperature of about 0 to about 10 °C under N₂.
32. IPC2-2: i-PrOAc/(i-PrOAc+DCM)=16.51%>10.0%, unqualified.
33. Agitate at a temperature of about 0 to about 10 °C for 5 hr under N₂.
34. Filter the suspension and rinse the cake with DCM (5.20 kg, 1.33 w/w).
   Note: wet weight of solid: 5.71 kg; weight of filtrate: 6.60 kg, assay: 1.26%, 83.16 g (2.07%) was lost in mother liquor.
35. Slurry the solid with DCM (21.00 kg, 5.30 w/w).
36. Heat to reflux (35~40 °C) under N₂.
37. Agitate at a temperature of about 35 to about 40 °C for 1 hr.
38. Cool to a temperature of about 0 to about 10 °C under N₂.
39. Agitate at a temperature of about 0 to about 10 °C for 2 hr under N₂.
40. Filter the suspension and rinse the cake with DCM (8.03 kg, 1.99 w/w).
   Note: wet weight of solid: 5.69 kg; weight of filtrate: 24.13 kg, assay: 0.11%, 26.54 g (0.66%) was lost in mother liquor.
41. IPC3: purity: 100.00%, qualified (unknown impurity NMT 0.15%; total impurity NMT 2.0%).
42. Dry the solid under vacuum at a temperature of about 50 to about 60 °C to constant weight.
43. Determine weight of off-white solid: 3.53 kg, yield: 87.92%, purity: 100.00% DCM residual: 1049 ppm. FIG. 30 shows a representative IR spectra for the amorphous end product of Scheme 9. FIG. 31 shows a representative HPLC spectra at different scales for the amorphous end product of Scheme 9.

### j. Step 9 Synthesis of MTK410088

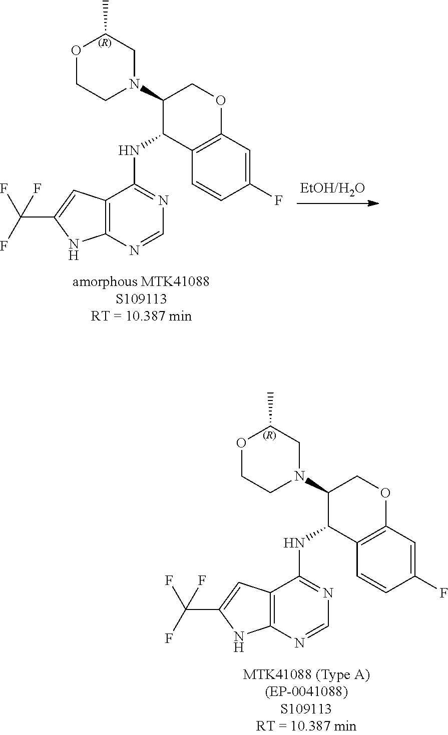

### Impurities

No impurity were observed in an amount of more than 0.15%.

### Observations/Process Changes/Deviations

Type A was obtained by slurry with IPA-H₂O system at a temperature of 50~60 °C to 20~30 °C. The process was quite robust.

### Batch Summary

| | Input, | | Amount | | | | | |
|---|---|---|---|---|---|---|---|---|
| Lot No. | SM | | Product | | Yield | Assay | Purity | Remarks |
| 21753-014 | 86.00 | g | 86.06 | g | 100.07% | N/A | 100.00% | Demo batch |
| S109113- | 3.49 | kg | 3.40 | kg | 97.42% | N/A | 100.00% | GMP batch |
| 2201 | | | | | | | | |

### Typical Operation Procedure

| | | | | Mol. | | Molar- | |
|---|---|---|---|---|---|---|---|
| No. | Raw-material | Amount | | wt. | Mol | ratio | |
| 1 | Crude MTK41088 | 3.49 | kg | N/A | N/A | 1.00 | equiv. |
| | (crude EP-0041088) | | | | | | |
| 2 | EtOH | 12.97 | kg | N/A | N/A | 3.56 | w/w |
| 3 | Purified water | 47.07 | kg | N/A | N/A | 13.7 | w/w |
| 4 | Seed MTK41088 | 124.00 | g | N/A | N/A | 0.035 | w/w |

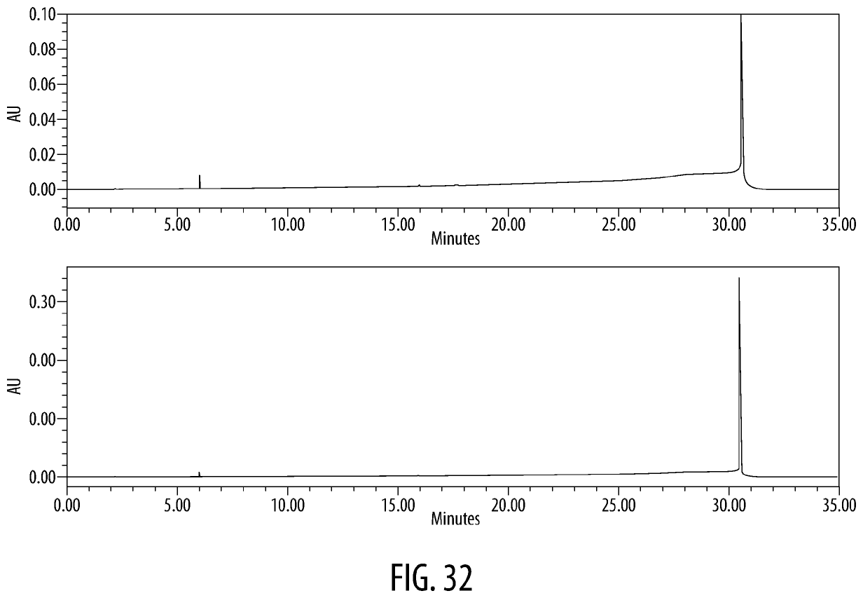

The following steps were performed:
1. Charge EtOH (11.57 kg, 3.16 w/w) into a glass jacket reactor 1 equipped with over-head agitator and thermometer at r.t. under N₂.
2. Charge crude amphorous end product (3.49 kg, 1.0 equiv.) into reactor 1 at a temperature of about 20 to about 30 °C under N₂.
3. Agitate at a temperature of about 20 to about 30 °C over 1 hr under N₂.
4. IPC1: a clear solution.
5. Filter the solution through in-line filter into the glass jacket reactor 2 equipped with over-head agitator and thermometer at a temperature of about 20 to about 30 °C.
6. Rinse reactor 1 with EtOH (1.40 kg, 0.40 w/w) and then through in-line filter into reactor 2.
7. Heat reactor 2 to a temperature of about 50 to about 60 °C under N₂.
   Note: a clear solution.
8. Charge purified water (9.11 kg, 2.50 w/w) dropwise into the reactor at a temperature of about 50 to about 60 °C under N₂.
   Note: the purified water was obtained through in-line filter.
9. IPC2: a clear solution.
10. Charge purified water (0.72 kg, 0.2 w/w) dropwise into the reactor at a temperature of about 50 to about 60 °C under N₂.
   Note: the purified water was obtained through in-line filter.
11. IPC3: a clear solution.
12. Charge purified water (0.74 kg, 0.2 w/w) dropwise into the reactor at a temperature of about 50 to about 60 °C under N₂.
   Note: the purified water was obtained through in-line filter.
13. IPC4: muddy.
14. Charge seed (124.00 g, 0.035 w/w) into the reactor at 50~60 °C under N₂.
   Note: much solid was precipitated.
15. Agitate for 2 hr at a temperature of about 50 to about 60 °C under N₂.
   Note: more solid was precipitated.
16. Charge purified water (29.50 kg, 8.8 w/w) dropwise into the reactor at a temperature of about 50 to about 60 °C under N₂.
   Note: the purified water was obtained through in-line filter.
17. Cool to a temperature of about 20 to about 30 °C over 4 to 5 hr under N₂.
18. Agitate at a temperature of about 20 to about 30 °C over 16 hr under N₂.
19. Filter the suspension and rinse the cake with purified water (7.00 kg, 2.00 w/w).
   Note: the purified water was obtained through in-line filter.
20. IPC5: KF: 1.76%, XPRD in progress.
21. Dry the solid under vacuum at a temperature of about 60 to about 65 °C to constant weight.
22. Measure weight: 3.40 kg, yield: 97.42%, purity: 99.92%, and HPLC. FIG. 32 shows a representative HPLC spectra at different scales for the desired end product of Scheme 9.

1. A compound having a structure represented by a formula: 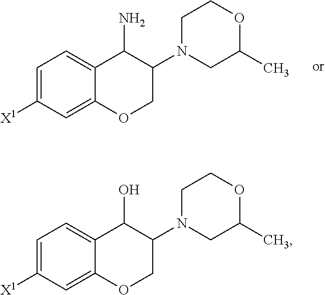
wherein X¹ is halogen,
or a salt thereof; 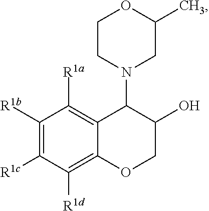
wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, provided that at least one of R^{1a}, R^{1b}, R^{1c}, and R^{1d} is not hydrogen, or a salt thereof; 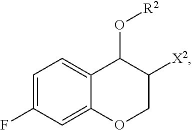
wherein X² is a halogen; and
wherein R² is —C(O)(C1-C4 alkyl),
or a salt thereof; or 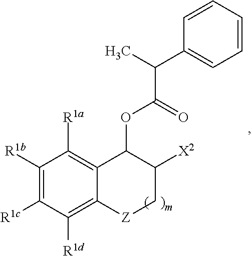
wherein m is 0 or 1;
wherein X² is a halogen;
wherein Z is selected from —CH₂—, —O—, and —NH—; and
wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a salt thereof.

2. The compound of claim 1, wherein X¹ is fluoro.

3. The compound of claim 1, wherein the compound has a structure represented by a formula: 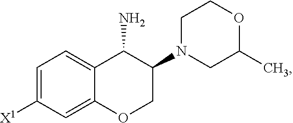
or a salt thereof.

4. The compound of claim 1, wherein the compound has a structure represented by a formula: 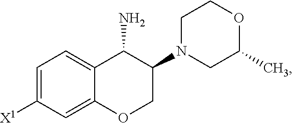
or a salt thereof.

5. The compound of claim 1, wherein the compound is: 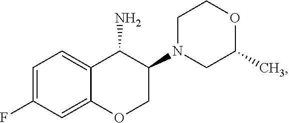 [SMILES: C[C@@H]1CN([C@H]2COc3cc(F)ccc3[C@@H]2N)CCO1]
or a salt thereof.

6. (canceled)

7. (canceled)

8. The compound of claim 1, wherein the compound is: 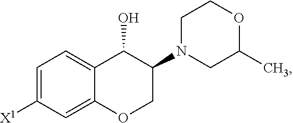
or a salt thereof.

9. The compound of claim 1, wherein the compound is: 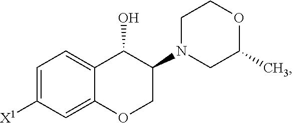
or a salt thereof.

10. The compound of claim 1, wherein the compound is: 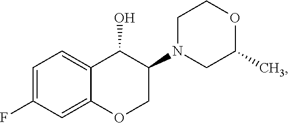 [SMILES: C[C@@H]1CN([C@H]2COc3cc(F)ccc3[C@@H]2O)CCO1]
or a salt thereof.

11-15. (canceled)

16. The compound of claim 1, wherein the compound has a structure represented by a formula: 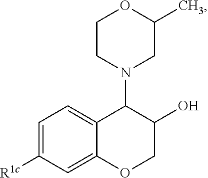
or a salt thereof.

17.-44. (canceled)

46. A method comprising:
(a) activating an alcohol having a structure represented by a formula: 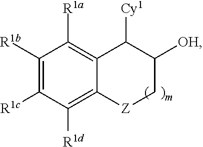
wherein m is 0 or 1;
wherein Z is selected from —CH₂—, —O—, and —NH—;
wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and
wherein Cy¹ is a 3- to 10-membered nitrogen-linked heterocycle substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino,
or a salt thereof, thereby forming an activated alcohol; and
(b) displacing the activated alcohol via addition of an amine, thereby providing a chromanamine derivative having a structure represented by a formula: 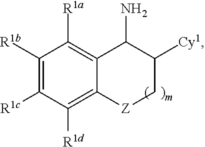
or a salt thereof.

47.-60. (canceled)

61. The method of claim 46, wherein the alcohol has a structure represented by a formula: 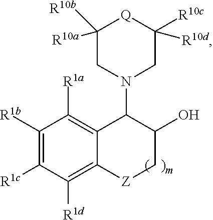
wherein Q is selected form —CH₂—, —O—, and —NR¹¹;
wherein R¹¹, when present, is selected from hydrogen and C1-C4 alkyl; and
wherein each of R^{10a}, R^{10b}, R^{10c}, and R^{10d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a salt thereof.

62.-89. (canceled)

90. The method of claim 46, further comprising epoxidizing a chromane derivative having a structure represented by a formula: 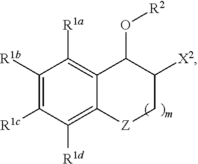
wherein X² is a halogen; and
wherein R² is —C(O)(C1-C4 alkyl) or a residue of a chiral auxiliary group; and
or a salt thereof, and reacting the epoxide with a cyclic amine having a structure represented by a formula:
Cy¹-H,
thereby providing the alcohol.

91. (canceled)

92. The method of claim 90, wherein epoxidizing is in the presence of a base.

93. The method of claim 92, wherein the base is sodium methoxide.

94. (canceled)

95. The method of claim 90, wherein the epoxidizing is in the presence diethyl ether.

96.-211. (canceled)

212. A method of synthesizing a compound having a structure according to formula: 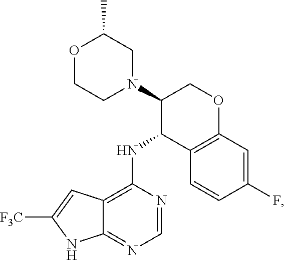 [SMILES: C[C@@H]1CN([C@H]2COc3cc(F)ccc3[C@@H]2Nc2ncnc3[nH]c(C(F)(F)F)cc23)CCO1]
or a pharmaceutically acceptable salt thereof, the method comprising
(a) activating an alcohol having a structure represented by a formula: 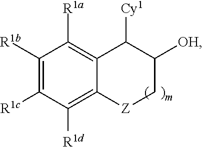
wherein m is 0 or 1;
wherein Z is selected from —CH₂—, —O—, and —NH—;
wherein each of R^{1a}, R^{1b}, R^{1c}, and R^{1d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and
wherein Cy¹ is a 3- to 10-membered nitrogen-linked heterocycle substituted with 0, 1, 2, 3, or 4 groups independently selected from halogen, CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino,
or a salt thereof, thereby forming an activated alcohol; and
(b) displacing the activated alcohol via addition of an amine, thereby providing a chromanamine derivative having a structure represented by a formula: 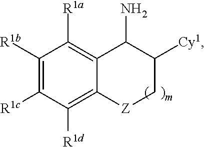
or a salt thereof,
wherein Cy¹ is a 3- to 10-membered nitrogen-linked heterocycle substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino

213. (canceled)

214. The method of claim 212, further comprising epoxidizing a chromane derivative having a structure represented by a formula: 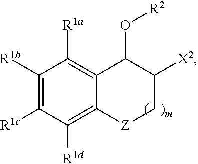
wherein X² is a halogen; and
wherein R² is —C(O)(C1-C4 alkyl) or a residue of a chiral auxiliary group; and
or a salt thereof, and reacting the epoxide with a cyclic amine having a structure represented by a formula:
Cy¹-H,
thereby providing the alcohol.

215. The method of claim 214, wherein epoxidizing is in the presence of a base.

216. The method of claim 215, wherein the base is sodium methoxide.

217. The method of claim 94, wherein the wherein epoxidizing is in the presence of diethyl ether.
